SEQUENCE LISTING (118 sequences)

SEQ ID NO: 1 - protein, 812 aa, synthetic construct
MDFSRLHMYSPPQCVPENTGYTYALSSSYSSDALDFETEHKLDPVFDSPRMSRRSLRLATTACTLGDGEAVGADSGTSSAVSLKNRAARTTKQRRSTNKSAFSINHVSRQVTSSGVSHGGTVSLQDAVTRRPPVLDESWIREQTTVDHFWGLDDDGDLKGGNKAAIQGNGDVGAAAATAHNGFSCSNCSMLSERKDVLTAHPAAPGPVSRVYSRDRNQKCDDCKGKRHLDAHPGRAGTLWHIWACAGYFLLQILRRIGAVGQAVSRTAWSALWLAVVAPGKAASGVFWWLGIGWYQFVTLISWLNVFLLTRCLRNICKFLVLLIPLFLLLAGLSLRGQGNFFSFLPVLNWASMHRTQRVDDPQDVFKPTTSRLKQPLQGDSEAFPWHWMSGVEQQVASLSGQCHHHGENLRELTTLLQKLQARVDQMEGGAAGPSASVRDAVGQPPRETDFMAFHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVK

SEQ ID NO: 2 - protein, 315 aa, synthetic construct
MDFSRLHMYSPPQCVPENTGYTYALSSSYSSDALDFETEHKLDPVFDSPRMSRRSLRLATTACTLGDGEAVGADSGTSSAVSLKNRAARTTKQRRSTNKSAFSINHVSRQVTSSGVSHGGTVSLQDAVTRRPPVLDESWIREQTTVDHFWGLDDDGDLKGGNKAAIQGNGDVGAAAATAHNGFSCSNCSMLSERKDVLTAHPAAPGPVSRVYSRDRNQKCDDCKGKRHLDAHPGRAGTLWHIWACAGYFLLQILRRIGAVGQAVSRTAWSALWLAVVAPGKAASGVFWWLGIGWYQFVTLISWLNVFLLTRCLRN

SEQ ID NO: 3 - protein, 20 aa, synthetic construct
ICKFLVLLIPLFLLLAGLSL

SEQ ID NO: 4 - protein, 477 aa, synthetic construct
RGQGNFFSFLPVLNWASMHRTQRVDDPQDVFKPTTSRLKQPLQGDSEAFPWHWMSGVEQQVASLSGQCHHHGENLRELTTLLQKLQARVDQMEGGAAGPSASVRDAVGQPPRETDFMAFHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVK

SEQ ID NO: 5 - protein, 138 aa, synthetic construct
MDFSRLHMYSPPQCVPENTGYTYALSSSYSSDALDFETEHKLDPVFDSPRMSRRSLRLATTACTLGDGEAVGADSGTSSAVSLKNRAARTTKQRRSTNKSAFSINHVSRQVTSSGVSHGGTVSLQDAVTRRPPVLDES

SEQ ID NO: 6 - protein, 94 aa, synthetic construct
SRVYSRDRNQKCDDCKGKRHLDAHPGRAGTLWHIWACAGYFLLQILRRIGAVGQAVSRTAWSALWLAVVAPGKAASGVFWWLGIGWYQFVTLIS

SEQ ID NO: 7 - protein, 80 aa, synthetic construct
CKGKRHLDAHPGRAGTLWHIWACAGYFLLQILRRIGAVGQAVSRTAWSALWLAVVAPGKAASGVFWWLGIGWYQFVTLIS

SEQ ID NO: 8 - protein, 163 aa, synthetic construct
GGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPV

SEQ ID NO: 9 - protein, 69 aa, synthetic construct
GEQLLPTVEHLQLELDQLKSELSSWRHVKTGCETVDAVQERVDVQVREMVKLLFSEDQQGGSLEQLLQR

SEQ ID NO: 10 - protein, 25 aa, synthetic construct
KGDLQTMLRDLQLQILRNVTHHVSV

SEQ ID NO: 11 - protein, 12 aa, synthetic construct
TSEAVVSAVSEA

SEQ ID NO: 12 - protein, 16 aa, synthetic construct
ITEAQARAIVNSALKL

SEQ ID NO: 13 - protein, 717 aa, synthetic construct
MSRRSQRLTRYSQGDDDGSSSSGGSSVAGSQSTLFKDSPLRTLKRKSSNMKRLSPAPQLGPSSDAHTSYYSESLVHESWFPPRSSLEELHGDANWGEDLRVRRRRGTGGSESSRASGLVGRKATEDFLGSSSGYSSEDDYVGYSDVDQQSSSSRLRSAVSRAGSLLWMVATSPGRLFRLLYWWAGTTWYRLTTAASLLDVFVLTRRFSSLKTFLWFLLPLLLLTCLTYGAWYFYPYGLQTFHPALVSWWAAKDSRRPDEGWEARDSSPHFQAEQRVMSRVHSLERRLEALAAEFSSNWQKEAMRLERLELRQGAPGQGGGGGLSHEDTLALLEGLVSRREAALKEDFRRETAARIQEELSALRAEHQQDSEDLFKKIVRASQESEARIQQLKSEWQSMTQESFQESSVKELRRLEDQLAGLQQELAALALKQSSVAEEVGLLPQQIQAVRDDVESQFPAWISQFLARGGGGRVGLLQREEMQAQLRELESKILTHVAEMQGKSAREAAASLSLTLQKEGVIGVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAH

SEQ ID NO: 14 - protein, 212 aa, synthetic construct
MSRRSQRLTRYSQGDDDGSSSSGGSSVAGSQSTLFKDSPLRTLKRKSSNMKRLSPAPQLGPSSDAHTSYYSESLVHESWFPPRSSLEELHGDANWGEDLRVRRRRGTGGSESSRASGLVGRKATEDFLGSSSGYSSEDDYVGYSDVDQQSSSSRLRSAVSRAGSLLWMVATSPGRLFRLLYWWAGTTWYRLTTAASLLDVFVLTRRFSSLKT

SEQ ID NO: 15 - protein, 21 aa, synthetic construct
FLWFLLPLLLLTCLTYGAWYF

SEQ ID NO: 16 - protein, 484 aa, synthetic construct
YPYGLQTFHPALVSWWAAKDSRRPDEGWEARDSSPHFQAEQRVMSRVHSLERRLEALAAEFSSNWQKEAMRLERLELRQGAPGQGGGGGLSHEDTLALLEGLVSRREAALKEDFRRETAARIQEELSALRAEHQQDSEDLFKKIVRASQESEARIQQLKSEWQSMTQESFQESSVKELRRLEDQLAGLQQELAALALKQSSVAEEVGLLPQQIQAVRDDVESQFPAWISQFLARGGGGRVGLLQREEMQAQLRELESKILTHVAEMQGKSAREAAASLSLTLQKEGVIGVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAH

SEQ ID NO: 17 - protein, 139 aa, synthetic construct
MSRRSQRLTRYSQGDDDGSSSSGGSSVAGSQSTLFKDSPLRTLKRKSSNMKRLSPAPQLGPSSDAHTSYYSESLVHESWFPPRSSLEELHGDANWGEDLRVRRRRGTGGSESSRASGLVGRKATEDFLGSSSGYSSEDD

SEQ ID NO: 18 - protein, 162 aa, synthetic construct
GASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPA

SEQ ID NO: 19 - protein, 67 aa, synthetic construct
QESFQESSVKELRRLEDQLAGLQQELAALALKQSSVAEEVGLLPQQIQAVRDDVESQFPAWISQFLA

SEQ ID NO: 20 - protein, 25 aa, synthetic construct
REEMQAQLRELESKILTHVAEMQGK

SEQ ID NO: 21 - protein, 12 aa, synthetic construct
AAASLSLTLQKE

SEQ ID NO: 22 - protein, 16 aa, synthetic construct
VTEEQVHHIVKQALQR

SEQ ID NO: 23 - protein, 357 aa, synthetic construct
MSGKTKARRAAMFFRRCSEDASGSASGNALLSEDENPDANGVTRSWKIILSTMLTLTFLLVGLLNHQWLKETDVPQKSRQLYAIIAEYGSRLYKYQARLRMPKEQLELLKKESQNLENNFRQILFLIEQIDVLKALLRDMKDGMDNNHNWNTHGDPVEDPDHTEEVSNLVNYVLKKLREDQVEMADYALKSAGASIIEAGTSESYKNNKAKLYWHGIGFLNHEMPPDIILQPDVYPGKCWAFPGSQGHTLIKLATKIIPTAVTMEHISEKVSPSGNISSAPKEFSVYGITKKCEGEEIFLGQFIYNKTGTTVQTFELQHAVSEYLLCVKLNIFSNWGHPKYTCLYRFRVHGTPGKHI

SEQ ID NO: 24 - protein, 354 aa, synthetic construct
MPRSSRSPGDPGALLEDVAHNPRPRRIAQRGRNTSRMAEDTSPNMTWFTCFACSLRTQAQQVLFNTCRCKLLCQKLMEKTGILLLCAFGFWMFSIHLPSKMKVWQDDSINGPLQSLRLYQEKVRHHSGEIQDLRGSMNQLIAKLQEMEAMSDEQKMAQKIMKMIHGDYIEKPDFALKSIGASIDFEHTSVTYNHEKAHSYWNWIQLWNYAQPPDVILEPNVTPGNCWAFEGDRGQVTIQLAQKVYLSNLTLQHIPKTISLSGSLDTAPKDFVIYGMEGSPKEEVFLGAFQFQPENIIQMFPLQNQPARAFSAVKVKISSNWGNPGFTCLYRVRVHGSVAPPREQPHQNPYPKRD

SEQ ID NO: 25 - protein, 437 aa, synthetic construct
MRRSSRPGSASSSRKHTPNFFSENSSMSITSEDSKGLRSAEPGPGEPEGRRARGPSCGEPALSAGVPGGTTWAGSSQQKPAPRSHNWQTACGAATVRGGASEPTGSPVVSEEPLDLLPTLDLRQEMPPPRVFKSFLSLLFQGLSVLLSLAGDVLVSMYREVCSIRFLFTAVSLLSLFLSAFWLGLLYLVSPLENEPKEMLTLSEYHERVRSQGQQLQQLQAELDKLHKEVSTVRAANSERVAKLVFQRLNEDFVRKPDYALSSVGASIDLQKTSHDYADRNTAYFWNRFSFWNYARPPTVILEPHVFPGNCWAFEGDQGQVVIQLPGRVQLSDITLQHPPPSVEHTGGANSAPRDFAVFGLQVYDETEVSLGKFTFDVEKSEIQTFHLQNDPPAAFPKVKIQILSNWGHPRFTCLYRVRAHGVRTSEGAEGSAQGPH

SEQ ID NO: 26 - protein, 1254 aa, synthetic construct
MKKHRRALALVSCLFLCSLVWLPSWRVCCKESSSASASSYYSQDDNCALENEDVQFQKKDEREGPINAESLGKSGSNLPISPKEHKLKDDSIVDVQNTESKKLSPPVVETLPTVDLHEESSNAVVDSETVENISSSSTSEITPISKLDEIEKSGTIPIAKPSETEQSETDCDVGEALDASAPIEQPSFVSPPDSLVGQHIENVSSSHGKGKITKSEFESKVSASEQGGGDPKSALNASDNLKNESSDYTKPGDIDPTSVASPKDPEDIPTFDEWKKKVMEVEKEKSQSMHASSNGGSHATKKVQKNRNNYASVECGAKILAANPEAKSTSAILIENMDLYMLNPCSTKIWFVIELCEPIQVKQLDIANYELFSSTPKDFLVSISDRYPTNKWIKLGTFHGRDERNVQSFPLDEQMYAKYVKMFIKYIKVELLSHFGSEHFCPLSLIRVFGTSMVEEYEEIADSQYHSERQELFDEDYDYPLDYNTGEDKSSKNLLGSATNAILNMVNIAANILGAKTEDLTEGNKSISENATATAAPKMPESTPVSTPVPSPEYVTTEVHTHDMEPSTPDTPKESPIVQLVQEEEEEASPSTVTLLGSGEQEDESSPWFESETQIFCSELTTICCISSFSEYIYKWCSVRVALYRQRSRTALSKGKDYLVLAQPPLLLPAESVDVSVLQPLSGELENTNIEREAETVVLGDLSSSMHQDDLVNHTVDAVELEPSHSQTLSQSLLLDITPEINPLPKIEVSESVEYEAGHIPSPVIPQESSVEIDNETEQKSESFSSIEKPSITYETNKVNELMDNIIKEDVNSMQIFTKLSETIVPPINTATVPDNEDGEAKMNIADTAKQTLISVVDSSSLPEVKEEEQSPEDALLRGLQRTATDFYAELQNSTDLGYANGNLVHGSNQKESVFMRLNNRIKALEVNMSLSGRYLEELSQRYRKQMEEMQKAFNKTIVKLQNTSRIAEEQDQRQTEAIQLLQAQLTNMTQLVSNLSATVAELKREVSDRQSYLVISLVLCVVLGLMLCMQRCRNTSQFDGDYISKLPKSNQYPSPKRCFSSYDDMNLKRRTSFPLMRSKSLQLTGKEVDPNDLYIVEPLKFSPEKKKKRCKYKIEKIETIKPEEPLHPIANGDIKGRKPFTNQRDFSNMGEVYHSSYKGPPSEGSSETSSQSEESYFCGISACTSLCNGQSQKTKTEKRALKRRRSKVQDQGKLIKTLIQTKSGSLPSLHDIIKGNKEITVGTFGVTAVSGHI

SEQ ID NO: 27 - protein, 162 aa, synthetic construct
GASIIEAGTSESYKNNKAKLYWHGIGFLNHEMPPDIILQPDVYPGKCWAFPGSQGHTLIKLATKIIPTAVTMEHISEKVSPSGNISSAPKEFSVYGITKKCEGEEIFLGQFIYNKTGTTVQTFELQHAVSEYLLCVKLNIFSNWGHPKYTCLYRFRVHGTPG

SEQ ID NO: 28 - protein, 160 aa, synthetic construct
GASIDFEHTSVTYNHEKAHSYWNWIQLWNYAQPPDVILEPNVTPGNCWAFEGDRGQVTIQLAQKVYLSNLTLQHIPKTISLSGSLDTAPKDFVIYGMEGSPKEEVFLGAFQFQPENIIQMFPLQNQPARAFSAVKVKISSNWGNPGFTCLYRVRVHGSVA

SEQ ID NO: 29 - protein, 161 aa, synthetic construct
GASIDLQKTSHDYADRNTAYFWNRFSFWNYARPPTVILEPHVFPGNCWAFEGDQGQVVIQLPGRVQLSDITLQHPPPSVEHTGGANSAPRDFAVFGLQVYDETEVSLGKFTFDVEKSEIQTFHLQNDPPAAFPKVKIQILSNWGHPRFTCLYRVRAHGVRT

SEQ ID NO: 30 - protein, 170 aa, synthetic construct
EKSQSMHASSNGGSHATKKVQKNRNNYASVECGAKILAANPEAKSTSAILIENMDLYMLNPCSTKIWFVIELCEPIQVKQLDIANYELFSSTPKDFLVSISDRYPTNKWIKLGTFHGRDERNVQSFPLDEQMYAKYVKMFIKYIKVELLSHFGSEHFCPLSLIRVFGTSM

SEQ ID NO: 31 - protein, 8797 aa, synthetic construct
MATSRGASRCPRDIANVMQRLQDEQEIVQKRTFTKWINSHLAKRKPPMVVDDLFEDMKDGVKLLALLEVLSGQKLPCEQGRRMKRIHAVANIGTALKFLEGRKIKLVNINSTDIADGRPSIVLGLMWTIILYFQIEELTSNLPQLQSLSSSASSVDSIVSSETPSPPSKRKVTTKIQGNAKKALLKWVQYTAGKQTGIEVKDFGKSWRSGVAFHSVIHAIRPELVDLETVKGRSNRENLEDAFTIAETELGIPRLLDPEDVDVDKPDEKSIMTYVAQFLKHYPDIHNASTDGQEDDEILPGFPSFANSVQNFKREDRVIFKEMKVWIEQFERDLTRAQMVESNLQDKYQSFKHFRVQYEMKRKQIEHLIQPLHRDGKLSLDQALVKQSWDRVTSRLFDWHIQLDKSLPAPLGTIGAWLYRAEVALREEITVQQVHEETANTIQRKLEQHKDLLQNTDAHKRAFHEIYRTRSVNGIPVPPDQLEDMAERFHFVSSTSELHLMKMEFLELKYRLLSLLVLAESKLKSWIIKYGRRESVEQLLQNYVSFIENSKFFEQYEVTYQILKQTAEMYVKADGSVEEAENVMKFMNETTAQWRNLSVEVRSVRSMLEEVISNWDRYGNTVASLQAWLEDAEKMLNQSENAKKDFFRNLPHWIQQHTAMNDAGNFLIETCDEMVSRDLKQQLLLLNGRWRELFMEVKQYAQADEMDRMKKEYTDCVVTLSAFATEAHKKLSEPLEVSFMNVKLLIQDLEDIEQRVPVMDAQYKIITKTAHLITKESPQEEGKEMFATMSKLKEQLTKVKECYSPLLYESQQLLIPLEELEKQMTSFYDSLGKINEIITVLEREAQSSALFKQKHQELLACQENCKKTLTLIEKGSQSVQKFVTLSNVLKHFDQTRLQRQIADIHVAFQSMVKKTGDWKKHVETNSRLMKKFEESRAELEKVLRIAQEGLEEKGDPEELLRRHTEFFSQLDQRVLNAFLKACDELTDILPEQEQQGLQEAVRKLHKQWKDLQGEAPYHLLHLKIDVEKNRFLASVEECRTELDRETKLMPQEGSEKIIKEHRVFFSDKGPHHLCEKRLQLIEELCVKLPVRDPVRDTPGTCHVTLKELRAAIDSTYRKLMEDPDKWKDYTSRFSEFSSWISTNETQLKGIKGEAIDTANHGEVKRAVEEIRNGVTKRGETLSWLKSRLKVLTEVSSENEAQKQGDELAKLSSSFKALVTLLSEVEKMLSNFGDCVQYKEIVKNSLEELISGSKEVQEQAEKILDTENLFEAQQLLLHHQQKTKRISAKKRDVQQQIAQAQQGEGGLPDRGHEELRKLESTLDGLERSRERQERRIQVTLRKWERFETNKETVVRYLFQTGSSHERFLSFSSLESLSSELEQTKEFSKRTESIAVQAENLVKEASEIPLGPQNKQLLQQQAKSIKEQVKKLEDTLEEDIKTMEMVKTKWDHFGSNFETLSVWITEKEKELNALETSSSAMDMQISQIKVTIQEIESKLSSIVGLEEEAQSFAQFVTTGESARIKAKLTQIRRYGEELREHAQCLEGTILGHLSQQQKFEENLRKIQQSVSEFEDKLAVPIKICSSATETYKVLQEHMDLCQALESLSSAITAFSASARKVVNRDSCVQEAAALQQQYEDILRRAKERQTALENLLAHWQRLEKELSSFLTWLERGEAKASSPEMDISADRVKVEGELQLIQALQNEVVSQASFYSKLLQLKESLFSVASKDDVKMMKLHLEQLDERWRDLPQIINKRINFLQSVVAEHQQFDELLLSFSVWIKLFLSELQTTSEISIMDHQVALTRHKDHAAEVESKKGELQSLQGHLAKLGSLGRAEDLHLLQGKAEDCFQLFEEASQVVERRQLALSHLAEFLQSHASLSGILRQLRQTVEATNSMNKNESDLIEKDLNDALQNAKALESAAVSLDGILSKAQYHLKIGSSEQRTSCRATADQLCGEVERIQNLLGTKQSEADALAVLKKAFQDQKEELLKSIEDIEERTDKERLKEPTRQALQQRLRVFNQLEDELNSHEHELCWLKDKAKQIAQKDVAFAPEVDREINRLEVTWDDTKRLIHENQGQCCGLIDLMREYQNLKSAVSKVLENASSVIVTRTTIKDQEDLKWAFSKHETAKNKMNYKQKDLDNFTSKGKHLLSELKKIHSSDFSLVKTDMESTVDKWLDVSEKLEENMDRLRVSLSIWDDVLSTRDEIEGWSNNCVPQMAENISNLDNHLRAEELLKEFESEVKNKALRLEELHSKVNDLKELTKNLETPPDLQFIEADLMQKLEHAKEITEVAKGTLKDFTAQSTQVEKFINDITTWFTKVEESLMNCAQNETCEALKKVKDIQKELQSQQSNISSTQENLNSLCRKYHSAELESLGRAMTGLIKKHEAVSQLCSKTQASLQESLEKHFSESMQEFQEWFLGAKAAAKESSDRTGDSKVLEAKLHDLQNILDSVSDGQSKLDAVTQEGQTLYAHLSKQIVSSIQEQITKANEEFQAFLKQCLKDKQALQDCASELGSFEDQHRKLNLWIHEMEERFNTENLGESKQHIPEKKNEVHKVEMFLGELLAARESLDKLSQRGQLLSEEGHGAGQEGRLCSQLLTSHQNLLRMTKEKLRSCQVALQEHEALEEALQSMWFWVKAIQDRLACAESTLGSKDTLEKRLSQIQDILLMKGEGEVKLNMAIGKGEQALRSSNKEGQRVIQTQLETLKEVWADIMSSSVHAQSTLESVISQWNDYVERKNQLEQWMESVDQKIEHPLQPQPGLKEKFVLLDHLQSILSEAEDHTRALHRLIAKSRELYEKTEDESFKDTAQEELKTQFNDIMTVAKEKMRKVEEIVKDHLMYLDAVHEFTDWLHSAKEELHRWSDMSGDSSATQKKLSKIKELIDSREIGASRLSRVESLAPEVKQNTTASGCELMHTEMQALRADWKQWEDSVFQTQSCLENLVSQMALSEQEFSGQVAQLEQALEQFSALLKTWAQQLTLLEGKNTDEEIVECWHKGQEILDALQKAEPRTEDLKSQLNELCRFSRDLSTYSGKVSGLIKEYNCLCLQASKGCQNKEQILQQRFRKAFRDFQQWLVNAKITTAKCFDIPQNISEVSTSLQKIQEFLSESENGQHKLNMMLSKGELLSTLLTKEKAKGIQAKVTAAKEDWKNFHSNLHQKESALENLKIQMKDFEVSAEPIQDWLSKTEKMVHESSNRLYDLPAKRREQQKLQSVLEEIHCYEPQLNRLKEKAQQLWEGQAASKSFRHRVSQLSSQYLALSNLTKEKVSRLDRIVAEHNQFSLGIKELQDWMTDAIHMLDSYCHPTSDKSVLDSRTLKLEALLSVKQEKEIQMKMIVTRGESVLQNTSPEGIPTIQQQLQSVKDMWASLLSAGIRCKSQLEGALSKWTSYQDGVRQFSGWMDSMEANLNESERQHAELRDKTTMLGKAKLLNEEVLSYSSLLETIEVKGAGMTEHYVTQLELQDLQERYRAIQERAKEAVTKSEKLVRLHQEYQRDLKAFEVWLGQEQEKLDQYSVLEGDAHTHETTLRDLQELQVHCAEGQALLNSVLHTREDVIPSGIPQAEDRALESLRQDWQAYQHRLSETRTQFNNVVNKLRLMEQKFQQVDEWLKTAEEKVSPRTRRQSNRATKEIQLHQMKKWHEEVTAYRDEVEEVGARAQEILDESHVNSRMGCQATQLTSRYQALLLQVLEQIKFLEEEIQSLEESESSLSSYSDWYGSTHKNFKNVATKIDKVDTVMMGKKLKTLEVLLKDMEKGHSLLKSAREKGERAVKYLEEGEAERLRKEIHDHMEQLKELTSTVRKEHMTLEKGLHLAKEFSDKCKALTQWIAEYQEILHVPEEPKMELYEKKAQLSKYKSLQQTVLSHEPSVKSVREKGEALLELVQDVTLKDKIDQLQSDYQDLCSIGKEHVFSLEAKVKDHEDYNSELQEVEKWLLQMSGRLVAPDLLETSSLETITQQLAHHKAMMEEIAGFEDRLNNLQMKGDTLIGQCADHLQAKLKQNVHAHLQGTKDSYSAICSTAQRMYQSLEHELQKHVSRQDTLQQCQAWLSAVQPDLEPSPQPPLSRAEAIKQVKHFRALQEQARTYLDLLCSMCDLSNASVKTTAKDIQQTEQTIEQKLVQAQNLTQGWEEIKHLKSELWIYLQDADQQLQNMKRRHSELELNIAQNMVSQVKDFVKKLQSKQASVNTIIEKVNKLTKKEESPEHKEINHLNDQWLDLCRQSNNLCLQREEDLQRTRDYHDCMNVVEVFLEKFTTEWDNLARSDAESTAVHLEALKKLALALQERKYAIEDLKDQKQKMIEHLNLDDKELVKEQTSHLEQRWFQLEDLIKRKIQVSVTNLEELNVVQSRFQELMEWAEEQQPNIAEALKQSPPPDMAQNLLMDHLAICSELEAKQMLLKSLIKDADRVMADLGLNERQVIQKALSDAQSHVNCLSDLVGQRRKYLNKALSEKTQFLMAVFQATSQIQQHERKIMFREHICLLPDDVSKQVKTCKSAQASLKTYQNEVTGLWAQGRELMKEVTEQEKSEVLGKLQELQSVYDSVLQKCSHRLQELEKNLVSRKHFKEDFDKACHWLKQADIVTFPEINLMNESSELHTQLAKYQNILEQSPEYENLLLTLQRTGQTILPSLNEVDHSYLSEKLNALPRQFNVIVALAKDKFYKVQEAILARKEYASLIELTTQSLSELEAQFLRMSKVPTDLAVEEALSLQDGCRAILDEVAGLGEAVDELNQKKEGFRSTGQPWQPDKMLHLVTLYHRLKRQTEQRVSLLEDTTSAYQEHEKMCQQLERQLKSVKEEQSKVNEETLPAEEKLKMYHSLAGSLQDSGIVLKRVTIHLEDLAPHLDPLAYEKARHQIQSWQGELKLLTSAIGETVTECESRMVQSIDFQTEMSRSLDWLRRVKAELSGPVYLDLNLQDIQEEIRKIQIHQEEVQSSLRIMNALSHKEKEKFTKAKELISADLEHSLAELSELDGDIQEALRTRQATLTEIYSQCQRYYQVFQAANDWLEDAQELLQLAGNGLDVESAEENLKSHMEFFSTEDQFHSNLEELHSLVATLDPLIKPTGKEDLEQKVASLELRSQRMSRDSGAQVDLLQRCTAQWHDYQKAREEVIELMNDTEKKLSEFSLLKTSSSHEAEEKLSEHKALVSVVNSFHEKIVALEEKASQLEKTGNDASKATLSRSMTTVWQRWTRLRAVAQDQEKILEDAVDEWTGFNNKVKKATEMIDQLQDKLPGSSAEKASKAELLTLLEYHDTFVLELEQQQSALGMLRQQTLSMLQDGAAPTPGEEPPLMQEITAMQDRCLNMQEKVKTNGKLVKQELKDREMVETQINSVKCWVQETKEYLGNPTIEIDAQLEELQILLTEATNHRQNIEKMAEEQKEKYLGLYTILPSELSLQLAEVALDLKIRDQIQDKIKEVEQSKATSQELSRQIQKLAKDLTTILTKLKAKTDNVVQAKTDQKVLGEELDGCNSKLMELDAAVQKFLEQNGQLGKPLAKKIGKLTELHQQTIRQAENRLSKLNQAASHLEEYNEMLELILKWIEKAKVLAHGTIAWNSASQLREQYILHQTLLEESKEIDSELEAMTEKLQYLTSVYCTEKMSQQVAELGRETEELRQMIKIRLQNLQDAAKDMKKFEAELKKLQAALEQAQATLTSPEVGRLSLKEQLSHRQHLLSEMESLKPKVQAVQLCQSALRIPEDVVASLPLCHAALRLQEEASRLQHTAIQQCNIMQEAVVQYEQYEQEMKHLQQLIEGAHREIEDKPVATSNIQELQAQISRHEELAQKIKGYQEQIASLNSKCKMLTMKAKHATMLLTVTEVEGLAEGTEDLDGELLPTPSAHPSVVMMTAGRCHTLLSPVTEESGEEGTNSEISSPPACRSPSPVANTDASVNQDIAYYQALSAERLQTDAAKIHPSTSASQEFYEPGLEPSATAKLGDLQRSWETLKNVISEKQRTLYEALERQQKYQDSLQSISTKMEAIELKLSESPEPGRSPESQMAEHQALMDEILMLQDEINELQSSLAEELVSESCEADPAEQLALQSTLTVLAERMSTIRMKASGKRQLLEEKLNDQLEEQRQEQALQRYRCEADELDSWLLSTKATLDTALSPPKEPMDMEAQLMDCQNMLVEIEQKVVALSELSVHNENLLLEGKAHTKDEAEQLAGKLRRLKGSLLELQRALHDKQLNMQGTAQEKEESDVDLTATQSPGVQEWLAQARTTWTQQRQSSLQQQKELEQELAEQKSLLRSVASRGEEILIQHSAAETSGDAGEKPDVLSQELGMEGEKSSAEDQMRMKWESLHQEFSTKQKLLQNVLEQEQEQVLYSRPNRLLSGVPLYKGDVPTQDKSAVTSLLDGLNQAFEEVSSQSGGAKRQSIHLEQKLYDGVSATSTWLDDVEERLFVATALLPEETETCLFNQEILAKDIKEMSEEMDKNKNLFSQAFPENGDNRDVIEDTLGCLLGRLSLLDSVVNQRCHQMKERLQQILNFQNDLKVLFTSLADNKYIILQKLANVFEQPVAEQIEAIQQAEDGLKEFDAGIIELKRRGDKLQVEQPSMQELSKLQDMYDELMMIIGSRRSGLNQNLTLKSQYERALQDLADLLETGQEKMAGDQKIIVSSKEEIQQLLDKHKEYFQGLESHMILTETLFRKIISFAVQKETQFHTELMAQASAVLKRAHKRGVELEYILETWSHLDEDQQELSRQLEVVESSIPSVGLVEENEDRLIDRITLYQHLKSSLNEYQPKLYQVLDDGKRLLISISCSDLESQLNQLGECWLSNTNKMSKELHRLETILKHWTRYQSESADLIHWLQSAKDRLEFWTQQSVTVPQELEMVRDHLNAFLEFSKEVDAQSSLKSSVLSTGNQLLRLKKVDTATLRSELSRIDSQWTDLLTNIPAVQEKLHQLQMDKLPSRHAISEVMSWISLMENVIQKDEDNIKNSIGYKAIHEYLQKYKGFKIDINCKQLTVDFVNQSVLQISSQDVESKRSDKTDFAEQLGAMNKSWQILQGLVTEKIQLLEGLLESWSEYENNVQCLKTWFETQEKRLKQQHRIGDQASVQNALKDCQDLEDLIKAKEKEVEKIEQNGLALIQNKKEDVSSIVMSTLRELGQTWANLDHMVGQLKILLKSVLDQWSSHKVAFDKINSYLMEARYSLSRFRLLTGSLEAVQVQVDNLQNLQDDLEKQERSLQKFGSITNQLLKECHPPVTETLTNTLKEVNMRWNNLLEEIAEQLQSSKALLQLWQRYKDYSKQCASTVQQQEDRTNELLKAATNKDIADDEVATWIQDCNDLLKGLGTVKDSLFFLHELGEQLKQQVDASAASAIQSDQLSLSQHLCALEQALCKQQTSLQAGVLDYETFAKSLEALEAWIVEAEEILQGQDPSHSSDLSTIQERMEELKGQMLKFSSMAPDLDRLNELGYRLPLNDKEIKRMQNLNRHWSLISSQTTERFSKLQSFLLQHQTFLEKCETWMEFLVQTEQKLAVEISGNYQHLLEQQRAHELFQAEMFSRQQILHSIIIDGQRLLEQGQVDDRDEFNLKLTLLSNQWQGVIRRAQQRRGIIDSQIRQWQRYREMAEKLRKWLVEVSYLPMSGLGSVPIPLQQARTLFDEVQFKEKVFLRQQGSYILTVEAGKQLLLSADSGAEAALQAELAEIQEKWKSASMRLEEQKKKLAFLLKDWEKCEKGIADSLEKLRTFKKKLSQSLPDHHEELHAEQMRCKELENAVGSWTDDLTQLSLLKDTLSAYISADDISILNERVELLQRQWEELCHQLSLRRQQIGERLNEWAVFSEKNKELCEWLTQMESKVSQNGDILIEEMIEKLKKDYQEEIAIAQENKIQLQQMGERLAKASHESKASEIEYKLGKVNDRWQHLLDLIAARVKKLKETLVAVQQLDKNMSSLRTWLAHIESELAKPIVYDSCNSEEIQRKLNEQQELQRDIEKHSTGVASVLNLCEVLLHDCDACATDAECDSIQQATRNLDRRWRNICAMSMERRLKIEETWRLWQKFLDDYSRFEDWLKSSERTAAFPSSSGVIYTVAKEELKKFEAFQRQVHECLTQLELINKQYRRLARENRTDSACSLKQMVHEGNQRWDNLQKRVTSILRRLKHFIGQREEFETARDSILVWLTEMDLQLTNIEHFSECDVQAKIKQLKAFQQEISLNHNKIEQIIAQGEQLIEKSEPLDAAIIEEELDELRRYCQEVFGRVERYHKKLIRLPLPDDEHDLSDRELELEDSAALSDLHWHDRSADSLLSPQPSSNLSLSLAQPLRSERSGRDTPASVDSIPLEWDHDYDLSRDLESAMSRALPSEDEEGQDDKDFYLRGAVGLSGDHSALESQIRQLGKALDDSRFQIQQTENIIRSKTPTGPELDTSYKGYMKLLGECSSSIDSVKRLEHKLKEEEESLPGFVNLHSTETQTAGVIDRWELLQAQALSKELRMKQNLQKWQQFNSDLNSIWAWLGDTEEELEQLQRLELSTDIQTIELQIKKLKELQKAVDHRKAIILSINLCSPEFTQADSKESRDLQDRLSQMNGRWDRVCSLLEEWRGLLQDALMQCQGFHEMSHGLLLMLENIDRRKNEIVPIDSNLDAEILQDHHKQLMQIKHELLESQLRVASLQDMSCQLLVNAEGTDCLEAKEKVHVIGNRLKLLLKEVSRHIKELEKLLDVSSSQQDLSSWSSADELDTSGSVSPTSGRSTPNRQKTPRGKCSLSQPGPSVSSPHSRSTKGGSDSSLSEPGPGRSGRGFLFRVLRAALPLQLLLLLLIGLACLVPMSEEDYSCALSNNFARSFHPMLRYTNGPPPL

SEQ ID NO: 32 - protein, 6885 aa, synthetic construct
MASSPELPTEDEQGSWGIDDLHISLQAEQEDTQKKAFTCWINSQLARHTSPSVISDLFTDIKKGHVLLDLLEVLSGQQLPRDKGSNTFQCRINIEHALTFLRNRSIKLINIHVTDIIDGNPSIILGLIWTIILHFHIEKLAQTLSCNYNQPSLDDVSVVDSSPASSPPAKKCSKVQARWQMSARKALLLWAQEQCATYESVNVTDFKSSWRNGMAFLAIIHALRPDLIDMKSVKHRSNKDNLREAFRIAEQELKIPRLLEPEDVDVVDPDEKSIMTYVAQFLQYSKDAPGTGEEAQGKVKDAMGWLTLQKEKLQKLLKDSENDTYFKKYNSLLSFMESFNEEKKSFLDVLSIKRDLDELDKDHLQLREAWDGLDHQINAWKIKLNYALPPPLHQTEAWLQEVEELMDEDLSASQDHSQAVTLIQEKMTLFKSLMDRFEHHSNILLTFENKDENHLPLVPPNKLEEMKRRINNILEKKFILLLEFHYYKCLVLGLVDEVKSKLDIWNIKYGSRESVELLLEDWHKFIEEKEFLARLDTSFQKCGEIYKNLAGECQNINKQYMMVKSDVCMYRKNIYNVKSTLQKVLACWATYVENLRLLRACFEETKKEEIKEVPFETLAQWNLEHATLNEAGNFLVEVSNDVVGSSISKELRRLNKRWRKLVSKTQLEMNLPLMIKKQDQPTFDNSGNILSKEEKATVEFSTDMSVELPENYNQNIKAGEKHEKENEEFTGQLKVAKDVEKLIGQVEIWEAEAKSVLDQDDVDTSMEESLKHLIAKGSMFDELMARSEDMLQMDIQNISSQESFQHVLTTGLQAKIQEAKEKVQINVVKLIAALKNLTDVSPDLDIRLKMEESQKELESYMMRAQQLLGQRESPGELISKHKEALIISNTKSLAKYLKAVEELKNNVTEDIKMSLEEKSRDVCAKWESLHHELSLYVQQLKIDIEKGKLSDNILKLEKQINKEKKLIRRGRTKGLIKEHEACFSEEGCLYQLNHHMEVLRELCEELPSQKSQQEVKRLLKDYEQKIERLLKCASEIHMTLQPTAGGTSKNEGTITTSENRGGDPHSEAPFAKSDNQPSTEKAMEPTMKFSLASVLRPLQEESIMEKDYSASINSLLERYDTYRDILEHHLQNNKFRITSDFSSEEDRSSSCLQAKLTDLQVIKNETDARWKEFEIISLKLENHVNDIKKPFVIKERDTLKERERELQMTLNTRMESLETALRLVLPVEKASLLLCGSDLPLHKMAIQGFHLIDADRIYQHLRNIQDSIAKQIEICNRLEEPGNFVLKELHPFDLHAMQNIILKYKTQFEGMNHRVQRSEDTLKALEDFLASLRTAKLSAEPVTDLSASDTQVAQENTLTVKNKEGEIHLMKDKAKHLDKCLKMLDMSFKDAERGDDTSCENLLDAFSIKLSETHGYGVQEEFTEENKLLEACIFKNNELLKNIQDVQSQISKIGLKDPTVPAVKHRKKSLIRLDKVLDEYEEEKRHLQEMANSLPHFKDGREKTVNQQCQNTVVLWENTKALVTECLEQCGRVLELLKQYQNFKSILTTLIQKEESVISLQASYMGKENLKKRIAEIEIVKEEFNEHLEVVDKINQVCKNLQFYLNKMKTFEEPPFEKEANIIVDRWLDINEKTEDYYENLGRALALWDKLFNLKNVIDEWTEKALQKMELHQLTEEDRERLKEELQVHEQKTSEFSRRVAEIQFLLQSSEIPLELQVMESSILNKMEHVQKCLTGESNCHALSGSTAELREDLDQAKTQIGMTESLLKALSPSDSLEIFTKLEEIQQQILQQKHSMILLENQIGCLTPELSELKKQYESVSDLFNTKKSVLQDHFSKLLNDQCKNFNDWFSNIKVNLKECFESSETKKSVEQKLQKLSDFLTLEGRNSKIKQVDSVLKHVKKHLPKAHVKELISWLVGQEFELEKMESICQARAKELEDSLQQLLRLQDDHRNLRKWLTNQEEKWKGMEEPGEKTELFCQALARKREQFESVAQLNNSLKEYGFTEEEEIIMEATCLMDRYQTLLRQLSEIEEEDKLLPTEDQSFNDLAHDVIHWIKEIKESLMVLNSSEGKMPLEERIQKIKEIILLKPEGDARIETIMKQAESSEAPLVQKTLTDISNQWDNTLHLASTYLSHQEKLLLEGEKYLQSKEDLRLMLIELKKKQEAGFALQHGLQEKKAQLKIYKKFLKKAQDLTSLLKELKSQGNYLLECTKNPSFSEEPWLEIKHLHESLLQQLQDSVQNLDGHVREHDSYQVCVTDLNTTLDNFSKEFVSFSDKPVDQIAVEEKLQKLQELENRLSLQDGTLKKILALAKSVKQNTSSVGQKIIKDDIKSLQCKQKDLENRLASAKQEMECCLNSILKSKRSTEKKGKFTLPGREKQATSDVQESTQESAAVEKLEEDWEINKDSAVEMAMSKQLSLNAQESMKNTEDERKVNELQNQPLELDTMLRNEQLEEIEKLYTQLEAKKAAIKPLEQTECLNKTETGALVLHNIGYSAQHLDNLLQALITLKKNKESQYCVLRDFQEYLAAVESSMKALLTDKESLKVGPLDSVTYLDKIKKFIASIEKEKDSLGNLKIKWENLSNHVTDMDKKLLESQIKQLEHGWEQVEQQIQKKYSQQVVEYDEFTTLMNKVQDTEISLQQQQQHLQLRLKSPEERAGNQSMIALTTDLQATKHGFSVLKGQAELQMKRIWGEKEKKNLEDGINNLKKQWETLEPLHLEAENQIKKCDIRNKMKETILWAKNLLGELNPSIPLLPDDILSQIRKCKVTHDGILARQQSVESLAEEVKDKVPSLTTYEGSDLNNTLEDLRNQYQMLVLKSTQRSQQLEFKLEERSNFFAIIRKFQLMVQESETLIIPRVETAATEAELKHHHVTLEASQKELQEIDSGISTHLQELTNIYEELNVFERLFLEDQLKNLKIRTNRIQRFIQNTCNEVEHKIKFCRQFHEKTSALQEEADSIQRNELLLNQEVNKGVKEEIYNLKDRLTAIKCCILQVLKLKKVFDYIGLNWDFSQLDQLQTQVFEKEKELEEKIKQLDTFEEEHGKYQALLSKMRAIDLQIKKMTEVVLKAPDSSPESRRLNAQILSQRIEKAKCLCDEIIKKLNENKTFDDSFKEKEILQIKLNAEENDKLYKVLQNMVLELSPKELDEKNCQDKLETSLHVLNQIKSQLQQPLLINLEIKHIQNEKDNCEAFQEQVWAEMCSIKAVTAIEKQREENSSEASDVETKLREFEDLQMQLNTSIDLRTNVLNDAYENLTRYKEAVTRAVESITSLEAIIIPYRVDVGNPEESLEMPLRKQEELESTVAHIQDLTEKLGMISSPEAKLQLQYTLQELVSKNSAMKEAFKAQETEAERYLENYKCYRKMEEDIYTNLSKMETVLGQSMSSLPLSYREALERLEQSKALVSNLISTKEELMKLRQILRLLRLRCTENDGICLLKIVSALWEKWLSLLEAAKEWEMWCEELKQEWKFVSEEIEREAIILDNLQEELPEISKTKEAATTEELSELLDCLCQYGENVEKQQLLLTLLLQRIRSIQNVPESSGAVETVPAFQEITSMKERCNKLLQKVQKNKELVQTEIQERHSFTKEIIALKNFFQQTTTSFQNMAFQDHPEKSEQFEELQSILKKGKLTFENIMEKLRIKYSEMYTIVPAEIESQVEECRKALEDIDEKISNEVLKSSPSYAMRRKIEEINNGLHNVEKMLQQKSKNIEKAQEIQKKMWDELDLWHSKLNELDSEVQDIVEQDPGQAQEWMDNLMIPFQQYQQVSQRAECRTSQLNKATVKMEEYSDLLKSTEAWIENTSHLLANPADYDSLRTLSHHASTVQMALEDSEQKHNLLHSIFMDLEDLSIIFETDELTQSIQELSNQVTALQQKIMESLPQIQRMADDVVAIESEVKSMEKRVSKIKTILLSKEIFDFSPEEHLKHGEVILENIRPMKKTIAEIVSYQVELRLPQTGMKPLPVFQRTNQLLQDIKLLENVTQEQNELLKVVIKQTNEWDEEIENLKQILNNYSAQFSLEHMSPDQADKLPQLQGEIERMEKQILSLNQRKEDLLVDLKATVLNLHQHLKQEQEGVERDRLPAVTSEEGGVAERDASERKLNRRGSMSYLAAVEEEVEESSVKSDNGDEKAEPSPQSWSSLWKHDKDMEEDRASSSSGTIVQEAYGKISTSDNSMAQILTPDSLNTEQGPECSLRPNQTEEGTTPPIEADTLDSSDAQGGLEPRVEKTRPEPTEVLHACKTQVAELELWLQQANVAVEPETLNADMQQVLEQQLVGCQAMLTEIEHKVAFLLETCKDQGLGDNGATQHEAEALSLKLKTVKCNLEKVQMMLQEKHSEDQHPTILKKSSEPEHQEALQPVNLSELESIVTERPQFSRQKDFQQQQVLELKPMEQKDFIKFIEFNAKKMWPQYCQHDNDTTQESSASNQASSPENDVPDSILSPQGQNGDKWQYLHHELSSKIKLPLPQLVEPQVSTNMGILPSVTMYNFRYPTTEELKTYTTQLEDLRQEASNLQTQENMTEEAYINLDKKLFELFLTLSQCLSSVEEMLEMPRLYREDGSGQQVHYETLALELKKLYLALSDKKGDLLKAMTWPGENTNLLLECFDNLQVCLEHTQAAAVCRSKSLKAGLDYNRSYQNEIKRLYHQLIKSKTSLQQSLNEISGQSVAEQLQKADAYTVELENAESRVAKLRDEGERLHLPYALLQEVYKLEDVLDSMWGMLRARYTELSSPFVTESQQDALLQGMVELVKIGKEKLAHGHLKQTKSKVALQAQIENHKVFFQKLVADMLLIQAYSAKILPSLLQNRETFWAEQVTEVKILEEKSRQCGMKLQSLLQKWEEFDENYASLEKDLEILISTLPSVSLVEETEERLVERISFYQQIKRNIGGKHARLYQTLNEGKQLVASVSCPELEGQIAKLEEQWLSLNKKIDHELHRLQALLKHLLSYNRDSDQLTKWLESSQHTLNYWKEQSLNVSQDLDTIRSNINNFFEFSKEVDEKSSLKTAVISIGNQLLHLKETDTATLRASLAQFEQKWTMLITQLPDIQEKLHQLQMEKLPSRKAITEMISWMNNVEHQTSDEDSVHSPSSASQVKHLLQKHKEFRMEMDYKQWIVDFVNQSLLQLSTCDVESKRYERTEFAEHLGEMNRQWHRVHGMLNRKIQHLEQLLESITESENKIQILNNWLEAQEERLKTLQKPESVISVQKLLLDCQDIENQLAIKSKALDELKQSYLTLESGAVPLLEDTASRIDELFQKRSSVLTQVNQLKTSMQSVLQEWKIYDQLYDEVNMMTIRFWYCMEHSKPVVLSLETLRCQVENLQSLQDEAESSEGSWEKLQEVIGKLKGLCPSVAEIIEEKCQNTHKRWTQVNQAIADQLQKAQSLLQLWKAYSNAHGEAAARLKQQEAKFQQLANISMSGNNLAEILPPALQDIKELQHDVQKTKEAFLQNSSVLDRLPQPAESSTHMLLPGPLHSLQRAAYLEKMLLVKANEFEFVLSQFKDFGVRLESLKGLIMHEEENLDRLHQQEKENPDSFLNHVLALTAQSPDIEHLNEVSLKLPLSDVAVKTLQNMNRQWIRATATALERCSELQGIGLNEKFLYCCEKWIQLLEKIEEALKVDVANSLPELLEQQKTYKMLEAEVSINQTIADSYVTQSLQLLDTTEIENRPEFITEFSKLTDRWQNAVQGVRQRKGDVDGLVRQWQDFTTSVENLFRFLTDTSHLLSAVKGQERFSLYQTRSLIHELKNKEIHFQRRRTTCALTLEAGEKLLLTTDLKTKESVGRRISQLQDSWKDMEPQLAEMIKQFQSTVETWDQCEKKIKELKSRLQVLKAQSEDPLPELHEDLHNEKELIKELEQSLASWTQNLKELQTMKADLTRHVLVEDVMVLKEQIEHLHRQWEDLCLRVAIRKQEIEDRLNTWVVFNEKNKELCAWLVQMENKVLQTADISIEEMIEKLQKDCMEEINLFSENKLQLKQMGDQLIKASNKSRAAEIDDKLNKINDRWQHLFDVIGSRVKKLKETFAFIQQLDKNMSNLRTWLARIESELSKPVVYDVCDDQEIQKRLAEQQDLQRDIEQHSAGVESVFNICDVLLHDSDACANETECDSIQQTTRSLDRRWRNICAMSMERRMKIEETWRLWQKFLDDYSRFEDWLKSAERTAACPNSSEVLYTSAKEELKRFEAFQRQIHERLTQLELINKQYRRLARENRTDTASRLKQMVHEGNQRWDNLQRRVTAVLRRLRHFTNQREEFEGTRESILVWLTEMDLQLTNVEHFSESDADDKMRQLNGFQQEITLNTNKIDQLIVFGEQLIQKSEPLDAVLIEDELEELHRYCQEVFGRVSRFHRRLTSCTPGLEDEKEASENETDMEDPREIQTDSWRKRGESEEPSSPQSLCHLVAPGHERSGCETPVSVDSIPLEWDHTGDVGGSSSHEEDEEGPYYSALSGKSISDGHSWHVPDSPSCPEHHYKQMEGDRNVPPVPPASSTPYKPPYGKLLLPPGTDGGKEGPRVLNGNPQQEDGGLAGITEQQSGAFDRWEMIQAQELHNKLKIKQNLQQLNSDISAITTWLKKTEAELEMLKMAKPPSDIQEIELRVKRLQEILKAFDTYKALVVSVNVSSKEFLQTESPESTELQSRLRQLSLLWEAAQGAVDSWRGGLRQSLMQCQDFHQLSQNLLLWLASAKNRRQKAHVTDPKADPRALLECRRELMQLEKELVERQPQVDMLQEISNSLLIKGHGEDCIEAEEKVHVIEKKLKQLREQVSQDLMALQGTQNPASPLPSFDEVDSGDQPPATSVPAPRAKQFRAVRTTEGEEETESRVPGSTRPQRSFLSRVVRAALPLQLLLLLLLLLACLLPSSEEDYSCTQANNFARSFYPMLRYTNGPPPT

SEQ ID NO: 33 - protein, 975 aa, synthetic construct
MTQQPQDDFDRSVEDAQAWMKAVQDQLQVNDNTQGPRAALEARLWETEKICQLEPEGRVRVDLVLRMAEALLACCPGDQKPGILARLKDIKAQWEETVTYMTHCHSRIEWVWLHWSEYLLARDEFYRWFQKMMVTLEPHIELQLGLKEKQWQLSHAQVLLHNVDNQAVLLDRLLEEAASLFNRIGDPSVDEDAQKRMKAEYDAVKAKAQKRVDLLEQVAREHEEYQAGVDEFQLWLKAVVEKVNGCLGRNCKLPITQRLSTLQDIAKDFPRGEESLETLEEQSAGVIRNTSPLGAEKITGELEEMRKVLEKLRALWEEEEERLRGLLRSRGAWEQQIKQLEAELSEFRMVLQRLAQEGLQPAAKAGTEDELVAHWRRYSATRAALASEEPRVDRLQAQLKELIVFPHNLKPLSDSVIATIQEYQSLKVKSARLRNAAAVELWQHFQRPLQDLQLWKALAQRLLEVTASLPDLPSLHTFLPQIEAALMESSRLKELLTMLQLKKDLLIGIFGQERATALLEQVAGSMRDRDLLHNSLLQRKSKLQSLLAQHKDFGAAFEPLQRKLLDLQVRVQAEKGLQRDLPGKQAQLSRLQGLQEEGLDLGAQMEAARPLVQENPNHQHKMDQLSSDFQALQRSLEDLVDRCRQSVQEHCTFSHQLLELRQWIVVTTQKLEAHRGEAGPGDAESQEAEFERLVAEFPEKEAQLSLVEAQGWLVMEKSSPEGAAVVQEELRELAESWRALRLLEESLLSLIRNWHLQRMEVDSGKKMVFTNNIPKSGFLINPMDPIPRHRRRANLLQEEEGSHEDFSQLLRNFGQWLQVENSKLVRIIAMRTSTAEDLRTRKSKLQELEARVPEGQHLFENLLRLGPARGTSDELEDLRYQWMLYKSKLKDSGHLLTQSSPGEPTGFQKTRRWRGLGSLFRRACCVALPLQLLLLLFLLLLFLLPIREEDRSCTLANNFARSFTLMLRYNGPPPT

SEQ ID NO: 34 - protein, 404 aa, synthetic construct
MALSLPLGPRLGSEPLNHPPGAPREADIVGCTVCPASGEESTSPEQAQTLGQDSLGPPEHFQGGPRGNEPAAHPPRWSTPSSYEDPAGGKHCEHPISGLEVLEAEQNSLHLCLLGLGRRLQDLEQGLGHWALAQSGMVQLQALQVDLRGAAERVEALLAFGEGLAQRSEPRAWAALEQILRALGAYRDSIFRRLWQLQAQLVSYSLVFEEANTLDQDLEVEGDSDWPGPGGVWGPWAPSSLPTSTELEWDPAGDIGGLGPLGQKTARTLGVPCELCGQRGPQGRGQGLEEADTSHSRQDMLESGLGHQKRLARHQRHSLLRKPQDKKRQASPHLQDVRLEGNPGAPDPASRQPLTFLLILFLLFLLLVGAMFLLPASGGPCCSHARIPRTPYLVLSYVNGLPPV

SEQ ID NO: 35 - protein, 562 aa, synthetic construct
MDLPEGPVGGPTAEMYLRERPEEARLGMPVSLEEQILNSTFEACDPQRTGTVAVAQVLAYLEAVTGQGPQDARLQTLANSLDPNGEGPKATVDLDTFLVVMRDWIAACQLHGGLELEEETAFQGALTSRQLPSGCPEAEEPANLESFGGEDPRPELQATADLLSSLEDLELSNRRLVGENAKLQRSMETAEEGSARLGEEILALRKQLHSTQQALQFAKAMDEELEDLKTLARSLEEQNRSLLAQARQAEKEQQHLVAEMETLQEENGKLLAERDGVKKRSQELAMEKDTLKRQLFECEHLICQRDTILSERTRDVESLAQTLEEYRVTTQELRLEISRLEEQLSQTYEGPDELPEGAQLRRVGWTELLPPSLGLEIEAIRQKQEVATADLSNPLCGVWQWEEVIHETSEETEFPSEAPAGGQRNFQGEPAHPEEGRKEPSMWLTRREEEEDAESQVTADLPVPLGAPRPGDIPENPPERPARRELQQALVPVMKKLVPVRRRAWGQLCLPPQRLRVTRHPLIPAPVLGLLLLLLLSVLLLGPSPPPTWPHLQLCYLQPPPV

SEQ ID NO: 36 - protein, 555 aa, synthetic construct
MESTPFSGVANQIHTLCERPTYGEVKDGALDVKRQHKCPGPTSGPSPGTNLSGCIRMNDDPSMEENGVERVCPESLLQSREYSSLPLPRHTSSTDGTITSSDPGLEILNMASCDLDRNSLCKKEEDTRSASPTIEAQGTSPAHDNIAFQDSTSKDKTILNLEAKEEPETIEEHKKEHASGDSVVSPLPVTTVKSVNLRQSENTSANEKEVEAEFLRLSLGFKCDWFTLEKRVKLEERSRDLAEENLKKEITNCLKLLESLTPLCEDDNQAQEIIKKLEKSIKFLSQCAARVASRAEMLGAINQESRVSKAVEVMIQHVENLKRMYAKEHAELEELKQVLLQNERSFNPLEDDDDCQIKKRSASLNSKPSSLRRVTIASLPRNIGNAGMVAGMENNDRFSRRSSSWRILGSKQSEHRPSLPRFISTYSWADAEEEKCELKTKDDSEPSGEETVERTRKPSLSEKKNNPSKWDVSSVYDTIASWATNLKSSIRKANKALWLSIAFIVLFAALMSFLTGQLFQKSVDAAPTQQEDSWTSLEHILWPFTRLRHNGPPPV

SEQ ID NO: 37 - protein, 60 aa, synthetic construct
RGFLFRVLRAALPLQLLLLLLIGLACLVPMSEEDYSCALSNNFARSFHPMLRYTNGPPPL

SEQ ID NO: 38 - protein, 60 aa, synthetic construct
RSFLSRVVRAALPLQLLLLLLLLLACLLPSSEEDYSCTQANNFARSFYPMLRYTNGPPPT

SEQ ID NO: 39 - protein, 59 aa, synthetic construct
GSLFRRACCVALPLQLLLLLFLLLLFLLPIREEDRSCTLANNFARSFTLMLRYNGPPPT

SEQ ID NO: 40 - protein, 58 aa, synthetic construct
DPASRQPLTFLLILFLLFLLLVGAMFLLPASGGPCCSHARIPRTPYLVLSYVNGLPPV

SEQ ID NO: 41 - protein, 41 aa, synthetic construct
LIPAPVLGLLLLLLLSVLLLGPSPPPTWPHLQLCYLQPPPV

SEQ ID NO: 42 - protein, 39 aa, synthetic construct
QLFQKSVDAAPTQQEDSWTSLEHILWPFTRLRHNGPPPV

SEQ ID NO: 43 - protein, 453 aa, synthetic construct
DDPQDVFKPTTSRLKQPLQGDSEAFPWHWMSGVEQQVASLSGQCHHHGENLRELTTLLQKLQARVDQMEGGAAGPSASVRDAVGQPPRETDFMAFHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVK

SEQ ID NO: 44 - protein, 197 aa, synthetic construct
GITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVK

SEQ ID NO: 45 - protein, 460 aa, synthetic construct
DEGWEARDSSPHFQAEQRVMSRVHSLERRLEALAAEFSSNWQKEAMRLERLELRQGAPGQGGGGGLSHEDTLALLEGLVSRREAALKEDFRRETAARIQEELSALRAEHQQDSEDLFKKIVRASQESEARIQQLKSEWQSMTQESFQESSVKELRRLEDQLAGLQQELAALALKQSSVAEEVGLLPQQIQAVRDDVESQFPAWISQFLARGGGGRVGLLQREEMQAQLRELESKILTHVAEMQGKSAREAAASLSLTLQKEGVIGVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAH

SEQ ID NO: 46 - protein, 196 aa, synthetic construct
GVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAH

SEQ ID NO: 47 - protein, 486 aa, synthetic construct
MKWVTFISLLFLFSSAYSRGVFRRDGPALDDPQDVFKPTTSRLKQPLQGDSEAFPWHWMSGVEQQVASLSGQCHHHGENLRELTTLLQKLQARVDQMEGGAAGPSASVRDAVGQPPRETDFMAFHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 48 - protein, 493 aa, synthetic construct
MKWVTFISLLFLFSSAYSRGVFRRDGPALDEGWEARDSSPHFQAEQRVMSRVHSLERRLEALAAEFSSNWQKEAMRLERLELRQGAPGQGGGGGLSHEDTLALLEGLVSRREAALKEDFRRETAARIQEELSALRAEHQQDSEDLFKKIVRASQESEARIQQLKSEWQSMTQESFQESSVKELRRLEDQLAGLQQELAALALKQSSVAEEVGLLPQQIQAVRDDVESQFPAWISQFLARGGGGRVGLLQREEMQAQLRELESKILTHVAEMQGKSAREAAASLSLTLQKEGVIGVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAHKDEL

SEQ ID NO: 49 - protein, 394 aa, synthetic construct
SHEDTLALLEGLVSRREAALKEDFRRETAARIQEELSALRAEHQQDSEDLFKKIVRASQESEARIQQLKSEWQSMTQESFQESSVKELRRLEDQLAGLQQELAALALKQSSVAEEVGLLPQQIQAVRDDVESQFPAWISQFLARGGGGRVGLLQREEMQAQLRELESKILTHVAEMQGKSAREAAASLSLTLQKEGVIGVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAH

SEQ ID NO: 50 - protein, 383 aa, synthetic construct
LVSRREAALKEDFRRETAARIQEELSALRAEHQQDSEDLFKKIVRASQESEARIQQLKSEWQSMTQESFQESSVKELRRLEDQLAGLQQELAALALKQSSVAEEVGLLPQQIQAVRDDVESQFPAWISQFLARGGGGRVGLLQREEMQAQLRELESKILTHVAEMQGKSAREAAASLSLTLQKEGVIGVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAH

SEQ ID NO: 51 - protein, 320 aa, synthetic construct [sequence omitted]

SEQ ID NO: 52 - protein, 285 aa, synthetic construct
SSVAEEVGLLPQQIQAVRDDVESQFPAWISQFLARGGGGRVGLLQREEMQAQLRELESKILTHVAEMQGKSAREAAASLSLTLQKEGVIGVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAH

SEQ ID NO: 53 - protein, 251 aa, synthetic construct
RGGGGRVGLLQREEMQAQLRELESKILTHVAEMQGKSAREAAASLSLTLQKEGVIGVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAH

SEQ ID NO: 54 - protein, 211 aa, synthetic construct
AAASLSLTLQKEGVIGVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAH

SEQ ID NO: 55 - protein, 204 aa, synthetic construct
TLQKEGVIGVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAH

SEQ ID NO: 56 - protein, 198 aa, synthetic construct
VIGVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAH

SEQ ID NO: 57 - protein, 197 aa, synthetic construct
IGVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAH

SEQ ID NO: 58 - protein, 456 aa, synthetic construct
DDSKGMHRPGPLPPSPPPKVDHKASQWPQESDMGQKVASLSAQCHNHDERLAELTVLLQKLQIRVDQVDDGREGLSLWVKNVVGQHLQEMGTIEPPDAKTDFMTFHHDHEVRLSNLEDVLRKLTEKSEAIQKELEETKLKAGSRDEEQPLLDRVQHLELELNLLKSQLSDWQHLKTSCEQAGARIQETVQLMFSEDQQGGSLEWLLEKLSSRFVSKDELQVLLHDLELKLLQNITHHITVTGQAPTSEAIVSAVNQAGISGITEAQAHIIVNNALKLYSQDKTGMVDFALESGGGSILSTRCSETYETKTALLSLFGVPLWYFSQSPRVVIQPDIYPGNCWAFKGSQGYLVVRLSMKIYPTTFTMEHIPKTLSPTGNISSAPKDFAVYGLETEYQEEGQPLGRFTYDQEGDSLQMFHTLERPDQAFQIVELRVLSNWGHPEYTCLYRFRVHGEPIQ

SEQ ID NO: 59 - protein, 246 aa, synthetic construct
SRFVSKDELQVLLHDLELKLLQNITHHITVTGQAPTSEAIVSAVNQAGISGITEAQAHIIVNNALKLYSQDKTGMVDFALESGGGSILSTRCSETYETKTALLSLFGVPLWYFSQSPRVVIQPDIYPGNCWAFKGSQGYLVVRLSMKIYPTTFTMEHIPKTLSPTGNISSAPKDFAVYGLETEYQEEGQPLGRFTYDQEGDSLQMFHTLERPDQAFQIVELRVLSNWGHPEYTCLYRFRVHGEPIQ

SEQ ID NO: 60 - protein, 491 aa, synthetic construct
LLLLLGAGVSLWGQGNFFSLLPVLNWTAMQPTQRVDDSKGMHRPGPLPPSPPPKVDHKASQWPQESDMGQKVASLSAQCHNHDERLAELTVLLQKLQIRVDQVDDGREGLSLWVKNVVGQHLQEMGTIEPPDAKTDFMTFHHDHEVRLSNLEDVLRKLTEKSEAIQKELEETKLKAGSRDEEQPLLDRVQHLELELNLLKSQLSDWQHLKTSCEQAGARIQETVQLMFSEDQQGGSLEWLLEKLSSRFVSKDELQVLLHDLELKLLQNITHHITVTGQAPTSEAIVSAVNQAGISGITEAQAHIIVNNALKLYSQDKTGMVDFALESGGGSILSTRCSETYETKTALLSLFGVPLWYFSQSPRVVIQPDIYPGNCWAFKGSQGYLVVRLSMKIYPTTFTMEHIPKTLSPTGNISSAPKDFAVYGLETEYQEEGQPLGRFTYDQEGDSLQMFHTLERPDQAFQIVELRVLSNWGHPEYTCLYRFRVHGEPIQ

SEQ ID NO: 61 - protein, 443 aa, synthetic construct
PSPPPKVDHKASQWPQESDMGQKVASLSAQCHNHDERLAELTVLLQKLQIRVDQVDDGREGLSLWVKNVVGQHLQEMGTIEPPDAKTDFMTFHHDHEVRLSNLEDVLRKLTEKSEAIQKELEETKLKAGSRDEEQPLLDRVQHLELELNLLKSQLSDWQHLKTSCEQAGARIQETVQLMFSEDQQGGSLEWLLEKLSSRFVSKDELQVLLHDLELKLLQNITHHITVTGQAPTSEAIVSAVNQAGISGITEAQAHIIVNNALKLYSQDKTGMVDFALESGGGSILSTRCSETYETKTALLSLFGVPLWYFSQSPRVVIQPDIYPGNCWAFKGSQGYLVVRLSMKIYPTTFTMEHIPKTLSPTGNISSAPKDFAVYGLETEYQEEGQPLGRFTYDQEGDSLQMFHTLERPDQAFQIVELRVLSNWGHPEYTCLYRFRVHGEPIQ

SEQ ID NO: 62 - protein, 419 aa, synthetic construct
ASLSAQCHNHDERLAELTVLLQKLQIRVDQVDDGREGLSLWVKNVVGQHLQEMGTIEPPDAKTDFMTFHHDHEVRLSNLEDVLRKLTEKSEAIQKELEETKLKAGSRDEEQPLLDRVQHLELELNLLKSQLSDWQHLKTSCEQAGARIQETVQLMFSEDQQGGSLEWLLEKLSSRFVSKDELQVLLHDLELKLLQNITHHITVTGQAPTSEAIVSAVNQAGISGITEAQAHIIVNNALKLYSQDKTGMVDFALESGGGSILSTRCSETYETKTALLSLFGVPLWYFSQSPRVVIQPDIYPGNCWAFKGSQGYLVVRLSMKIYPTTFTMEHIPKTLSPTGNISSAPKDFAVYGLETEYQEEGQPLGRFTYDQEGDSLQMFHTLERPDQAFQIVELRVLSNWGHPEYTCLYRFRVHGEPIQ

SEQ ID NO: 63 - protein, 409 aa, synthetic construct
HHHGENLRELTTLLQKLQARVDQMEGGAAGPSASVRDAVGQPPRETDFMAFHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVK

SEQ ID NO: 64 - protein, 358 aa, synthetic construct
HQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVK

SEQ ID NO: 65 - protein, 318 aa, synthetic construct
GEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVK

SEQ ID NO: 66 - protein, 251 aa, synthetic construct
QRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVK

SEQ ID NO: 67 - protein, 212 aa, synthetic construct
TSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVK

SEQ ID NO: 68 - protein, 177 aa, synthetic construct
DKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVK

SEQ ID NO: 69 - protein, 413 aa, synthetic construct
HHHGENLRELTTLLQKLQARVDQMEGGAAGPSASVRDAVGQPPRETDFMAFHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 70 - protein, 362 aa, synthetic construct
HQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 71 - protein, 322 aa, synthetic construct
GEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 72 - protein, 255 aa, synthetic construct
QRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 73 - protein, 216 aa, synthetic construct
TSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 74 - protein, 201 aa, synthetic construct
GITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 75 - protein, 200 aa, synthetic construct
GVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAHKDEL

SEQ ID NO: 76 - protein, 25 aa, synthetic construct
MKWVTFISLLFLFSSAYSRGVFRRD

SEQ ID NO: 77 - protein, 4 aa, synthetic construct
KDEL

SEQ ID NO: 78 - protein, 9 aa, synthetic construct
YPYDVPDYA

SEQ ID NO: 79 - protein, 13 aa, synthetic construct
SRLEEELRRRLTE

SEQ ID NO: 80 - protein, 510 aa, synthetic construct
MKWVTFISLLFLFSSAYSRGVFRRDGSYPYDVPDYAPSRLEEELRRRLTEPTGDDPQDVFKPTTSRLKQPLQGDSEAFPWHWMSGVEQQVASLSGQCHHHGENLRELTTLLQKLQARVDQMEGGAAGPSASVRDAVGQPPRETDFMAFHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 81 - protein, 466 aa, synthetic construct
MKWVTFISLLFLFSSAYSRGVFRRDGSYPYDVPDYAPSRLEEELRRRLTEPTGHHHGENLRELTTLLQKLQARVDQMEGGAAGPSASVRDAVGQPPRETDFMAFHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 82 - protein, 415 aa, synthetic construct
MKWVTFISLLFLFSSAYSRGVFRRDGSYPYDVPDYAPSRLEEELRRRLTEPTGHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 83 - protein, 375 aa, synthetic construct
MKWVTFISLLFLFSSAYSRGVFRRDGSYPYDVPDYAPSRLEEELRRRLTEPTGGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 84 - protein, 308 aa, synthetic construct
MKWVTFISLLFLFSSAYSRGVFRRDGSYPYDVPDYAPSRLEEELRRRLTEPTGQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 85 - protein, 269 aa, synthetic construct
MKWVTFISLLFLFSSAYSRGVFRRDGSYPYDVPDYAPSRLEEELRRRLTEPTGTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 86 - protein, 254 aa, synthetic construct
MKWVTFISLLFLFSSAYSRGVFRRDGSYPYDVPDYAPSRLEEELRRRLTEPTGGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 87 - protein, 234 aa, synthetic construct
MKWVTFISLLFLFSSAYSRGVFRRDGSYPYDVPDYAPSRLEEELRRRLTEPTGDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 88 - protein, 138 aa, synthetic construct
GEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKL

SEQ ID NO: 89 - protein, 62 aa, synthetic construct
KGDLQTMLRDLQLQILRNVTHHVSVTKQLPTSEAVVSAVSEAGASGITEAQARAIVNSALKL

SEQ ID NO: 90 - protein, 32 aa, synthetic construct
TSEAVVSAVSEAGASGITEAQARAIVNSALKL

SEQ ID NO: 91 - protein, 139 aa, synthetic construct
QESFQESSVKELRRLEDQLAGLQQELAALALKQSSVAEEVGLLPQQIQAVRDDVESQFPAWISQFLARGGGGRVGLLQREEMQAQLRELESKILTHVAEMQGKSAREAAASLSLTLQKEGVIGVTEEQVHHIVKQALQR

SEQ ID NO: 92 - protein, 61 aa, synthetic construct
REEMQAQLRELESKILTHVAEMQGKSAREAAASLSLTLQKEGVIGVTEEQVHHIVKQALQR

SEQ ID NO: 93 - protein, 32 aa, synthetic construct
AAASLSLTLQKEGVIGVTEEQVHHIVKQALQR

SEQ ID NO: 94 - protein, 330 aa, synthetic construct
LEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVK

SEQ ID NO: 95 - protein, 334 aa, synthetic construct
LEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 96 - protein, 387 aa, synthetic construct
MKWVTFISLLFLFSSAYSRGVFRRDGSYPYDVPDYAPSRLEEELRRRLTEPTGLEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 97 - protein, 150 aa, synthetic construct
LEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKL

SEQ ID NO: 98 - protein, 151 aa, synthetic construct
IQQLKSEWQSMTQESFQESSVKELRRLEDQLAGLQQELAALALKQSSVAEEVGLLPQQIQAVRDDVESQFPAWISQFLARGGGGRVGLLQREEMQAQLRELESKILTHVAEMQGKSAREAAASLSLTLQKEGVIGVTEEQVHHIVKQALQR

SEQ ID NO: 99 - protein, 457 aa, synthetic construct
DDPQDVFKPTTSRLKQPLQGDSEAFPWHWMSGVEQQVASLSGQCHHHGENLRELTTLLQKLQARVDQMEGGAAGPSASVRDAVGQPPRETDFMAFHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 100 - protein, 18 aa, synthetic construct
MKWVTFISLLFLFSSAYS

SEQ ID NO: 101 - protein, 475 aa, synthetic construct
MKWVTFISLLFLFSSAYSDDPQDVFKPTTSRLKQPLQGDSEAFPWHWMSGVEQQVASLSGQCHHHGENLRELTTLLQKLQARVDQMEGGAAGPSASVRDAVGQPPRETDFMAFHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 102 - protein, 475 aa, synthetic construct; identical to SEQ ID NO: 101

SEQ ID NO: 103 - protein, 380 aa, synthetic construct
MKWVTFISLLFLFSSAYSHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 104 - protein, 340 aa, synthetic construct
MKWVTFISLLFLFSSAYSGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 105 - protein, 352 aa, synthetic construct
MKWVTFISLLFLFSSAYSLEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 106 - protein, 273 aa, synthetic construct
MKWVTFISLLFLFSSAYSQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 107 - protein, 234 aa, synthetic construct
MKWVTFISLLFLFSSAYSTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 108 - protein, 219 aa, synthetic construct
MKWVTFISLLFLFSSAYSGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 109 - protein, 199 aa, synthetic construct
MKWVTFISLLFLFSSAYSDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVKKDEL

SEQ ID NO: 110 - protein, 466 aa, synthetic construct
KDSRRPDEGWEARDSSPHFQAEQRVMSRVHSLERRLEALAAEFSSNWQKEAMRLERLELRQGAPGQGGGGGLSHEDTLALLEGLVSRREAALKEDFRRETAARIQEELSALRAEHQQDSEDLFKKIVRASQESEARIQQLKSEWQSMTQESFQESSVKELRRLEDQLAGLQQELAALALKQSSVAEEVGLLPQQIQAVRDDVESQFPAWISQFLARGGGGRVGLLQREEMQAQLRELESKILTHVAEMQGKSAREAAASLSLTLQKEGVIGVTEEQVHHIVKQALQRYSEDRIGLADYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGKFTYDQDGEPIQTFHFQAPTMATYQVVELRILTNWGHPEYTCIYRFRVHGEPAH

SEQ ID NO: 111 - protein, 466 aa, synthetic construct
KESRKQPEVWESRDASQHFQAEQRVLSRVHSLERRLEALAADFSSNWQKEAIRLERLELRQGAAGHGGGSSLSHEDALSLLEGLVSRREATLKEDLRRDTVAHIQEELATLRAEHHQDSEDLFKKIVQASQESEARVQQLKTEWKSMTQEAFQESSVKELGRLEAQLASLRQELAALTLKQNSVADEVGLLPQKIQAARADVESQFPDWIRQFLLGDRGARSGLLQRDEMHAQLQELENKILTKMAEMQGKSAREAAASLGQILQKEGIVGVTEEQVHRIVKQALQRYSEDRIGMVDYALESGGASVISTRCSETYETKTALLSLFGIPLWYHSQSPRVILQPDVHPGNCWAFQGPQGFAVVRLSARIRPTAVTLEHVPKALSPNSTISSAPKDFAIFGFDEDLQQEGTLLGTFAYDQDGEPIQTFYFQASKMATYQVVELRILTNWGHPEYTCIYRFRVHGEPAH

SEQ ID NO: 112 - protein, 459 aa, synthetic construct
HRTQRVDDPQDVFKPTTSRLKQPLQGDSEAFPWHWMSGVEQQVASLSGQCHHHGENLRELTTLLQKLQARVDQMEGGAAGPSASVRDAVGQPPRETDFMAFHQEHEVRMSHLEDILGKLREKSEAIQKELEQTKQKTISAVGEQLLPTVEHLQLELDQLKSELSSWRHVKTGCETVDAVQERVDVQVREMVKLLFSEDQQGGSLEQLLQRFSSQFVSKGDLQTMLRDLQLQILRNVTHHVSVTKQLPTSEAVVSAVSEAGASGITEAQARAIVNSALKLYSQDKTGMVDFALESGGGSILSTRCSETYETKTALMSLFGIPLWYFSQSPRVVIQPDIYPGNCWAFKGSQGYLVVRLSMMIHPAAFTLEHIPKTLSPTGNISSAPKDFAVYGLENEYQEEGQLLGQFTYDQDGESLQMFQALKRPDDTAFQIVELRIFSNWGHPEYTCLYRFRVHGEPVK

SEQ ID NO: 113 - protein, 462 aa, synthetic construct
QPTQRVDDSKGMHRPGPLPPSPPPKVDHKASQWPQESDMGQKVASLSAQCHNHDERLAELTVLLQKLQIRVDQVDDGREGLSLWVKNVVGQHLQEMGTIEPPDAKTDFMTFHHDHEVRLSNLEDVLRKLTEKSEAIQKELEETKLKAGSRDEEQPLLDRVQHLELELNLLKSQLSDWQHLKTSCEQAGARIQETVQLMFSEDQQGGSLEWLLEKLSSRFVSKDELQVLLHDLELKLLQNITHHITVTGQAPTSEAIVSAVNQAGISGITEAQAHIIVNNALKLYSQDKTGMVDFALESGGGSILSTRCSETYETKTALLSLFGVPLWYFSQSPRVVLQPDIYPGNCWAFKGSQGYLVVRLSMKIYPTTFTMEHIPKTLSPTGNISSAPKDFAVYGLETEYQEEGQPLGRFTYDQEGDSLQMFHTLERPDQAFQIVELRVLSNWGHPEYTCLYRFRVHGEPIQ

SEQ ID NO: 114 - protein, 4 aa, synthetic construct
CDEL

SEQ ID NO: 115 - protein, 4 aa, synthetic construct
KCEL

SEQ ID NO: 116 - protein, 4 aa, synthetic construct
HVEL

SEQ ID NO: 117 - protein, 4 aa, synthetic construct
DENQ

SEQ ID NO: 118 - protein, 6 aa, synthetic construct
KRHQSA

(19) United States
(12) Patent Application Publication
(10) Pub. No.: US 2026/0001923 A1
(43) Pub. Date: Jan. 1, 2026

(54) LINC COMPLEX INHIBITING POLYPEPTIDES

(71) Applicants: AGENCY FOR SCIENCE, TECHNOLOGY AND RESEARCH, Singapore (SG); NUEVOCOR PTE. LTD., Singapore (SG)

(72) Inventors: Yin Loon LEE, Singapore (SG); Brian BURKE, Singapore (SG); Hendrikje WERNER, Singapore (SG); Yann Chong TAN, Singapore (SG); David W. RUSSELL, Singapore (SG)

(21) Appl. No.: 18/715,077

(22) PCT Filed: Nov. 30, 2022
(86) PCT No.: PCT/SG2022/050871

§ 371 Date: Apr. 21, 2025

(30) Foreign Application Priority Data
Dec. 1, 2021 (SG) 10202113391P

(51) Int. Cl. C07K 14/47; A61P 9/00; C12N 15/86

(52) U.S. Cl. CPC C07K 14/47; A61P 9/00; C12N 15/86; C12N 2750/14143; C12N 2830/008

(57) ABSTRACT

The invention relates to nucleic acids encoding dominant negative polypeptides comprising the a3 helix of CC2 region and the SUN domain of a SUN domain-containing protein that inhibit the LING complex. The specific embodiment relates to polypeptides of varying lengths derived from amino acids 404-812 of SUNI with a KDEL signal sequence. It also relates to methods of identifying a LING complex inhibitor and the use of said polypeptides for treating and preventing laminopathies, and diseases characterised by hyperlipidaemia.

This application claims priority from SG 10202113391P filed 1 Dec. 2021, the contents and elements of which are herein incorporated by reference for all purposes.

### TECHNICAL FIELD

The present disclosure relates to the field of molecular and cellular biology, and also relates to methods of medical treatment and prophylaxis.

### BACKGROUND

Lamin A/C is a component of the nuclear lamina that underlies the inner nuclear membrane, and is thought to provide mechanical support to the nucleus (Burke and Stewart, 2013). Mutations in LMNA, which encodes lamins A and C, result in a plethora of diseases known as laminopathies.

Disruption of Linker of Nucleoskeleton and Cytoskeleton (LINC) complexes has recently been proposed as a therapeutic strategy for the treatment of laminopathies. The LINC complex is comprised of SUN domain proteins of the inner nuclear membrane, and KASH domain proteins of the outer nuclear membrane (Lee and Burke, 2018). The paralogous Sun1 and Sun2 are the major SUN domain proteins, and are broadly expressed in mammalian tissues. Among the KASH domain proteins, Nesprin-1, Nesprin-2, and Nesprin-3 are the most widely expressed.

Crisp et al., 2006 describes LINC complex disruption via overexpression of a dominant-negative form of mouse Sun1 (DNSun1) protein. DNSun1 comprises the entire lumenal domain of Sun1, a N-terminal signal sequence, and a C-terminal KDEL Golgi-to-ER retrieval sequence. It is thought to function by disrupting SUN-KASH interactions within the LINC complex, presumably by competing with endogenous SUN domain-containing proteins (e.g. Sun1 and Sun2) for binding to KASH domain-containing proteins. Unlike wildtype Sun1, DNSun1 is not anchored to the nuclear lamina, hence forces from the cytoskeleton transmitted to KASH domain proteins are not further transmitted to the nuclear interior by DNSun1. A humanised version of DNSun1 is described in WO 2019/143300 A1.

The relatively large size of the coding sequence of DNSUN1 (˜1.5 kb) presents a challenge for its delivery e.g. as a gene therapy. By way of illustration, in self-complementary adeno-associated viral vector (scAAV) systems, the entire transgene, including the viral inverted terminal repeats (ITRs), promoters and other regulatory sequences, and the coding sequence itself, cannot exceed 2.3 kb.

The C-terminal SUN domain of SUN domain-containing proteins has been shown to be necessary but insufficient for interaction with KASH domain-containing proteins, and trimerization of the SUN domain mediated by upstream helical and coil regions is thought to be required for KASH interactions (Sosa et al., 2012; Wang et al., 2012; Zhou et al., 2012; Jahed et al., 2018b; Nie et al., 2016; Xu et al., 2018). Zhou et al., 2012 and Sosa et al., 2012 demonstrated that lumenal SUN constructs truncated before the start of the CC1 domain were able to associate with KASH domain proteins. Zhou et al., 2012, Wang et al., 2012, Jahed et al., 2018b and Nie et al., 2016 showed that constructs comprising CC1-CC2-SUN were able to either form trimers in gel filtration assays, or interact with KASH domains in pulldown assays. Zhou et al. 2012 and Jahed et al. 2018b further showed that a truncation beginning in the middle of the CC1 domain and encompassing CC2 and SUN did not interact with KASH in pulldown assays and formed a monomer in gel filtration assays. Wang et al. 2012 and Nie et al. 2016 showed that CC2-SUN constructs similarly did not interact with KASH in vitro, and formed only monomers in gel filtration assays.

While most of this work has focused on SUN2, there is a high degree of sequence conservation between SUN1 and SUN2, particularly in their lumenal domains, and where Sun1 itself has been investigated, there is clear conservation at the structural level between Sun1 and Sun2 (Xu et al., 2018; Gurusaran and Davies, 2021).

Importantly, there are contradictory reports as to whether SUN protein variants comprising certain truncations of the lumenal domain are able to form trimers and interact with KASH domain proteins (Jahed et al., 2018a).

In the studies of Zhou et al. 2012 and Wang et al. 2012, a Sun2 truncation variant consisting of positions G522 to H717 was able to interact with KASH domain-containing proteins (as determined by co-immunoprecipitation analysis), and a Sun2 truncation variant consisting of positions V520 to H717 was able to form trimers, and was used to solve the SUN trimeric structure by X-ray crystallography (formation of SUN trimers are thought to be required for binding to KASH domain-containing proteins). Nie et al. 2016 determined that removing α1 (α2-α3-SUN) or both α1 and α2 (α3-SUN) helices from CC2-SUN abolished the auto-inhibitory function of CC2, and that the resulting Sun2 constructs could interact with KASH domain proteins. Interestingly, while α2-α3-SUN formed a trimer in gel filtration assays, α3-SUN was a mixture of monomers and trimers.

By contrast, Sosa et al. 2012 reported that while a SUN2 truncation variant consisting of positions 507 to 717 (corresponding to α2-α3-SUN) was able bind to the KASH domain, further truncations led to weak (in the case of the SUN2 (514-717) truncation variant) or negligible (in the case of the SUN2 (521-717) truncation variant, which corresponds to α3-SUN) binding to KASH domain.

While the literature has examined how truncations of SUN protein lumenal domains impact KASH binding, it is unknown as to whether trimer formation and KASH domain binding in vitro is predictive of the ability to disrupt the LINC complex at the cellular level.

It is therefore as yet unknown what truncations of SUN lumenal regions can be employed as agents for LINC complex disruption.

### SUMMARY

In a first aspect, the present disclosure provides a nucleic acid encoding a LINC complex inhibiting polypeptide, wherein the LINC complex inhibiting polypeptide comprises: (i) an inhibitory region comprising an amino acid sequence corresponding to the α3 helix of the CC2 region and the SUN domain of a SUN domain-containing protein, and (ii) an endoplasmic reticulum retention motif; and wherein the LINC complex inhibiting polypeptide does not comprise the amino acid sequence of any one of SEQ ID NO:43, 45 or 58.

In some embodiments, the inhibitory region of the LINC complex inhibiting polypeptide does not consist essentially of the amino acid sequence of SEQ ID NO:44 or 46. In some embodiments, the inhibitory region of the LINC complex inhibiting polypeptide does not consist essentially of the amino acid sequence of any one of SEQ ID NOs: 49, 50, 51, 52, 53, 54, 55, 56, 57, 59, 60, 61 or 62.

In some embodiments, the inhibitory region of the LINC complex inhibiting polypeptide consists essentially of:
(i) an amino acid sequence having at least 80% amino acid sequence identity to the amino acid sequence of SEQ ID NO:66;
(ii) an amino acid sequence having at least 80% amino acid sequence identity to the amino acid sequence of SEQ ID NO:94; or
(iii) an amino acid sequence having at least 80% amino acid sequence identity to the amino acid sequence of SEQ ID NO:65; or
(iv) an amino acid sequence having at least 80% amino acid sequence identity to the amino acid sequence of SEQ ID NO:63; or
(v) an amino acid sequence having at least 80% amino acid sequence identity to the amino acid sequence of SEQ ID NO:64; or
(vi) an amino acid sequence having at least 80% amino acid sequence identity to the amino acid sequence of SEQ ID NO:67.

In some embodiments, the LINC complex inhibiting polypeptide comprises, or consists essentially of:
(i) an amino acid sequence having at least 80% amino acid sequence identity to the amino acid sequence of SEQ ID NO:72;
(ii) an amino acid sequence having at least 80% amino acid sequence identity to the amino acid sequence of SEQ ID NO:95; or
(iii) an amino acid sequence having at least 80% amino acid sequence identity to the amino acid sequence of SEQ ID NO:71; or
(iv) an amino acid sequence having at least 80% amino acid sequence identity to the amino acid sequence of SEQ ID NO:69; or
(v) an amino acid sequence having at least 80% amino acid sequence identity to the amino acid sequence of SEQ ID NO:70; or
(vi) an amino acid sequence having at least 80% amino acid sequence identity to the amino acid sequence of SEQ ID NO:73.

In some embodiments, the LINC complex inhibiting polypeptide comprises a signal peptide. In some embodiments, the nucleic acid is a vector suitable for delivering the nucleic acid encoding a LINC complex inhibiting polypeptide as a gene therapy. In some embodiments, the vector is an adeno-associated virus (AAV) vector.

The present disclosure also provides a LINC complex inhibiting polypeptide, comprising: (i) an inhibitory region comprising an amino acid sequence corresponding to the α3 helix of the CC2 region and the SUN domain of a SUN domain-containing protein, and (ii) an endoplasmic reticulum retention motif;

and wherein the LINC complex inhibiting polypeptide does not comprise the amino acid sequence of SEQ ID NO:43 or 45.

In some embodiments, the inhibitory region of the LINC complex inhibiting polypeptide does not consist essentially of the amino acid sequence of SEQ ID NO:44 or 46. In some embodiments, the LINC complex inhibiting polypeptide according to claim 9 or claim 10, wherein the inhibitory region of the LINC complex inhibiting polypeptide does not consist essentially of the amino acid sequence of any one of SEQ ID NOS: 49, 50, 51, 52, 53, 54, 55, 56, 57, 58, 59, 60, 61 or 62.

In some embodiments, the inhibitory region of the LINC complex inhibiting polypeptide consists essentially of:
(i) an amino acid sequence having at least 80% amino acid sequence identity to the amino acid sequence of SEQ ID NO:66;
(ii) an amino acid sequence having at least 80% amino acid sequence identity to the amino acid sequence of SEQ ID NO:94; or
(iii) an amino acid sequence having at least 80% amino acid sequence identity to the amino acid sequence of SEQ ID NO:65; or
(iv) an amino acid sequence having at least 80% amino acid sequence identity to the amino acid sequence of SEQ ID NO:63; or
(v) an amino acid sequence having at least 80% amino acid sequence identity to the amino acid sequence of SEQ ID NO:64; or
(vi) an amino acid sequence having at least 80% amino acid sequence identity to the amino acid sequence of SEQ ID NO:67.

In some embodiments, the LINC complex inhibiting polypeptide comprises, or consists essentially of:
(i) an amino acid sequence having at least 80% amino acid sequence identity to the amino acid sequence of SEQ ID NO:72;
(ii) an amino acid sequence having at least 80% amino acid sequence identity to the amino acid sequence of SEQ ID NO:95; or
(iii) an amino acid sequence having at least 80% amino acid sequence identity to the amino acid sequence of SEQ ID NO:71; or
(iv) an amino acid sequence having at least 80% amino acid sequence identity to the amino acid sequence of SEQ ID NO:69; or
(v) an amino acid sequence having at least 80% amino acid sequence identity to the amino acid sequence of SEQ ID NO:70; or
(vi) an amino acid sequence having at least 80% amino acid sequence identity to the amino acid sequence of SEQ ID NO:73.

In some embodiments, the LINC complex inhibiting polypeptide comprises a signal peptide.

The present disclosure also provides a method for identifying a LINC complex inhibiting polypeptide, comprising:
introducing nucleic acid encoding a candidate LINC complex inhibiting polypeptide, wherein the candidate LINC complex inhibiting polypeptide comprises an amino acid sequence corresponding to the SUN domain of a SUN domain-containing protein; and subsequently analysing the cells to determine the subcellular localisation of an interaction partner for a SUN domain-containing protein;
wherein a candidate LINC complex inhibiting polypeptide is determined to be a LINC complex inhibiting polypeptide where a change in the subcellular localisation of an interaction partner for a SUN domain-containing protein is detected.

The present disclosure also provides a method for identifying a LINC complex inhibiting polypeptide, comprising:
introducing nucleic acid encoding a candidate LINC complex inhibiting polypeptide, wherein the candidate LINC complex inhibiting polypeptide comprises an amino acid sequence corresponding to the KASH domain of a KASH domain-containing protein; and subsequently analysing the cells to determine the subcellular localisation of an interaction partner for a KASH domain-containing protein;
wherein a candidate LINC complex inhibiting polypeptide is determined to be a LINC complex inhibiting polypeptide where a change in the subcellular localisation of an interaction partner for a KASH domain-containing protein is detected.

In some embodiments, a candidate LINC complex inhibiting polypeptide is determined to be a LINC complex inhibiting polypeptide where an increase in the proportion of the interaction partner for a SUN/KASH domain-containing protein localised to the endoplasmic reticulum is detected.

The present disclosure also provides a LINC complex inhibiting polypeptide identified by the method according to the present disclosure.

The present disclosure also provides a nucleic acid encoding a LINC complex inhibiting polypeptide according to the present disclosure.

The present disclosure also provides a vector comprising a nucleic acid according to the present disclosure, wherein the vector is a vector suitable for delivering the nucleic acid encoding a LINC complex inhibiting polypeptide as a gene therapy.

In some embodiments, the vector is an adeno-associated virus (AAV) vector.

The present disclosure also provides a cell comprising a nucleic acid, LINC complex inhibiting polypeptide or vector according to the present disclosure.

The present disclosure also provides a pharmaceutical composition comprising a nucleic acid, LINC complex inhibiting polypeptide, vector or cell according to the present disclosure.

The present disclosure also provides a nucleic acid, LINC complex inhibiting polypeptide, vector, cell or pharmaceutical composition according to the present disclosure, for use in a method of medical treatment or prophylaxis.

The present disclosure also provides a nucleic acid, LINC complex inhibiting polypeptide, vector, cell or pharmaceutical composition according to the present disclosure, for use in a method of treating or preventing a laminopathy.

The present disclosure also provides the use of a nucleic acid, LINC complex inhibiting polypeptide, vector, cell or pharmaceutical composition according to the present disclosure in the manufacture of a medicament for treating or preventing a laminopathy.

The present disclosure also provides a method of treating or preventing a laminopathy, comprising administering a therapeutically-or prophylactically-effective amount of a nucleic acid, LINC complex inhibiting polypeptide, vector, cell or pharmaceutical composition according to the present disclosure to a subject.

In some embodiments in accordance with various aspects of the present disclosure, the laminopathy is characterised by one or more of myopathy, cardiomyopathy, dilated cardiomyopathy, muscular dystrophy, cardiac muscular dystrophy, skeletal muscular dystrophy, progeria, neuropathy, lipoatrophy, skeletal dysplasia, lipodystrophy, leukodystrophy or dermopathy. In some embodiments, the laminopathy is associated with mutation to LMNA. In some embodiments, the laminopathy is selected from: Hutchinson-Gilford Progeria Syndrome; Dilated Cardiomyopathy; Muscular Dystrophy, Congenital, Lmna-Related; Emery-Dreifuss Muscular Dystrophy 2, Autosomal Dominant; Muscular Dystrophy; Mandibuloacral Dysplasia with Type a Lipodystrophy; Cardiomyopathy, Dilated, 1a; Charcot-Marie-Tooth Disease; Limb-Girdle Muscular Dystrophy; Cardiomyopathy, Dilated, with Hypergonadotropic Hypogonadism; Emery-Dreifuss Muscular Dystrophy 3, Autosomal Recessive; Lipodystrophy, Familial Partial, Type 2; Emery-Dreifuss Muscular Dystrophy; Charcot-Marie-Tooth Disease, Axonal, Type 2b1; Heart-Hand Syndrome, Slovenian Type; Aging; Familial Partial Lipodystrophy; Restrictive Dermopathy, Lethal; Arrhythmogenic Right Ventricular Cardiomyopathy; Tooth Disease; Heart Disease; Werner Syndrome; Hypertrophic Cardiomyopathy; Left Ventricular Noncompaction; Atrioventricular Block; Calcinosis; Acroosteolysis; Autosomal Dominant Limb-Girdle Muscular Dystrophy; Diabetes Mellitus, Noninsulin-Dependent; Osteoporosis; Atrial Fibrillation; Atrial Standstill 1; Acanthosis Nigricans; Cardiac Conduction Defect; Catecholaminergic Polymorphic Ventricular Tachycardia; Mandibular Hypoplasia, Deafness, Progeroid Features, and Lipodystrophy Syndrome; Sick Sinus Syndrome; Pelger-Huet Anomaly; Charcot-Marie-Tooth Disease, Axonal, Type 2e; Congenital Generalized Lipodystrophy; Restrictive Cardiomyopathy; Congenital Fiber-Type Disproportion; Lipodystrophy, Congenital Generalized, Type 1; Myofibrillar Myopathy; Lipodystrophy, Familial Partial, Type 1; Axonal Neuropathy; Atypical Werner Syndrome; Ovarian Cystadenoma; Fanconi Anemia, Complementation Group a; Body Mass Index Quantitative Trait Locus 11; Skin Disease; Rigid Spine Muscular Dystrophy 1; Neuromuscular Disease; Hallermann-Streiff Syndrome; Bethlem Myopathy 1; Acquired Generalized Lipodystrophy; Cardiomyopathy, Dilated, 1e; Lipodystrophy, Congenital Generalized, Type 4; Undifferentiated Pleomorphic Sarcoma; Lipodystrophy, Familial Partial, Type 3; Muscular Dystrophy, Congenital Merosin-Deficient, 1a; Proximal Spinal Muscular Atrophy; Muscular Dystrophy-Dystroglycanopathy, Type B, 5; Muscular Dystrophy, Congenital, 1b; Reynolds Syndrome; Wiedemann-Rautenstrauch Syndrome; Emery-Dreifuss Muscular Dystrophy 1, X-Linked; Lipodystrophy, Congenital Generalized, Type 2; Monogenic Diabetes; Cardiomyopathy, Dilated, 1d; Myopathy, Proximal, and Ophthalmoplegia; Muscle Tissue Disease; Lipodystrophy, Familial Partial, Type 4; Cardiomyopathy, Dilated, 1h; Second-Degree Atrioventricular Block; Median Neuropathy; Intrinsic Cardiomyopathy; Prolapse of Female Genital Organ; Complete Generalized Lipodystrophy; Rigid Spine Muscular Dystrophy; Emerinopathy; Ulnar Nerve Lesion; Limb-Girdle Muscular Dystrophy Type 1b; Lmna-Related Dilated Cardiomyopathy; Pelvic Muscle Wasting; Generalized Lipodystrophy-Associated Progeroid Syndrome; Muscular Disease; Cardiomyopathy, Dilated, 1b; Autosomal Genetic Disease; Familial Isolated Arrhythmogenic Ventricular Dysplasia, Right Dominant Form; Familial Isolated Arrhythmogenic Ventricular Dysplasia, Biventricular Form; Familial Isolated Arrhythmogenic Ventricular Dysplasia, Left Dominant Form; Lmna-Related Cardiocutaneous Progeria Syndrome; and Autosomal Semi-Dominant Severe Lipodystrophic Laminopathy.

The present disclosure also provides a nucleic acid, LINC complex inhibiting polypeptide, vector, cell or pharmaceutical composition according to the present disclosure, for use in a method of treating or preventing a disease characterised by hyperlipidemia.

The present disclosure also provides the use of a nucleic acid, LINC complex inhibiting polypeptide, vector, cell or pharmaceutical composition according to the present disclosure in the manufacture of a medicament for treating or preventing a disease characterised by hyperlipidemia.

The present disclosure also provides a method of treating or preventing a disease characterised by hyperlipidemia, comprising administering a therapeutically-or prophylactically-effective amount of a nucleic acid, LINC complex inhibiting polypeptide, vector, cell or pharmaceutical composition according to the present disclosure to a subject.

In some embodiments in accordance with various aspects of the present disclosure, the disease characterised by hyperlipidemia is selected from atherosclerosis, cardiovascular disease, stroke and a familial hyperlipidemia.

### DESCRIPTION

The present disclosure is based on the inventors' unexpected finding that despite reports in the prior art that various truncated variants of the lumenal domain of SUN domain-containing proteins are unable to associate with KASH domain-containing proteins—and would therefore be expected not to function as LINC complex inhibitors-a wide range of such truncated variants do in fact function as LINC complex inhibitors.

Using a cell-based assay in which LINC complex disruption is determined through the observation of disruption of normal subcellular localisation of an endogenous KASH domain-containing protein (specifically, in which the KASH domain-containing protein localises to the endoplasmic reticulum rather than the nuclear envelope following LINC complex disruption), the inventors demonstrate that polypeptides corresponding to a variety of different truncations of the lumenal domain of a SUN domain-containing protein and comprising different structural motifs thereof (i.e. coiled-coil regions, a helices thereof) are able to effect LINC complex disruption. The inventors further confirm that the various different dominant-negative LINC complex inhibiting polypeptides delivered as a gene therapy via AAV reduce the pathology of dilated cardiomyopathy (DCM) in vivo in a mouse model of DCM established by cardiomyocyte-specific deletion of Lmna.

The present disclosure makes available a large number of LINC complex inhibiting polypeptides, which are suitable for application in connection with existing and future gene therapy technologies. In particular, the LINC complex inhibiting polypeptides which are smaller in size than the dominant-negative SUN domain-containing protein constructs described in WO 2019/143300 A1 are more suitable to be employed in vector systems having a modest packaging limit, such as scAAV vectors which have a ˜2.3 kb packing limit.

### LINC Complex Structure and Function

Linker of nucleoskeleton and cytoskeleton (LINC) complexes are polypeptide complexes comprising SUN domain-containing proteins and KASH domain-containing proteins. LINC complex structure is reviewed in e.g. in Sosa et al., Curr Opin Struct Biol. (2013) 23 (2): 285-91 and Hieda, Cells (2017) 6 (1): 3, both of which are hereby incorporated by reference in their entirety.

LINC complexes connect the inner nuclear membrane (INM) and the outer nuclear membrane (ONM) of the nuclear envelope. SUN domain-containing proteins span the INM, and are associated with nuclear lamins and chromatin-binding proteins on the nucleoplasmic side of the INM, and with KASH domain-containing proteins on the perinuclear side of the INM. KASH domain-containing proteins span the ONM, and are associated with cytoskeletal structural components such as actin filaments, microtubule motors and intermediate filaments on the cytoplasmic side of the ONM, and with SUN domain-containing proteins on the perinuclear side of the ONM. SUN domain proteins function as translumenal tethers for KASH domain proteins in the ONM.

Herein, a ‘SUN domain-containing protein’ refers to any polypeptide comprising a SUN domain. SUN (Sad1 and UNC-84) domain proteins are important INM components comprising conserved, carboxy terminal SUN domains which localise to the perinuclear space. SUN domains comprise ˜175 residues and are provided at the end of helical stalk regions. The nucleoplasmic domains of SUN proteins interact with structural components of the nucleoskeleton.

A SUN domain may comprise or consist of the amino acid sequence shown in SEQ ID NO:5, 14, 27, 28, 29 or 30, or an amino acid sequence having at least 60%, e.g. one of at least 65%, 70%, 75%, 80%, 85%, 90%, 91%, 92%, 93%, 94%, 95%, 96%, 97%, 98%, 99% or 100% amino acid sequence identity to the amino acid sequence shown in SEQ ID NO:5, 14, 27, 28, 29 or 30.

In some embodiments, a SUN domain-containing protein is selected from SUN1, SUN2, SUN3, SUN5, SPAG4 and SUCO. In some embodiments, a SUN domain-containing protein is SUN1 or SUN2.

In some embodiments, a SUN domain-containing protein is capable of forming a LINC complex. In some embodiments, a SUN domain-containing protein is capable of interacting with a KASH domain and/or a KASH domain-containing protein.

Human SUN1 is the polypeptide identified by UniProtKB O94901, the amino acid sequence of which is shown in SEQ ID NO:1. Human SUN2 is the polypeptide identified by UniProtKB Q9UH99, the amino acid sequence of which is shown in SEQ ID NO:13. Human SUN3 is the polypeptide identified by UniProtKB Q8TAQ9, the amino acid sequence of which is shown in SEQ ID NO:23. Human SUN5 is the polypeptide identified by UniProtKB A9Z1W8, the amino acid sequence of which is shown in SEQ ID NO: 24. Human SPAG4 is the polypeptide identified by UniProtKB Q9NPE6, the amino acid sequence of which is shown in SEQ ID NO:25. Human SUCO is the polypeptide identified by UniProtKB Q9UBS9, the amino acid sequence of which is shown in SEQ ID NO:26.

In this specification ‘SUN1’, ‘SUN2, ‘SUN3’, ‘SUN5″SPAG4’ and ‘SUCO’ respectively refer to SUN1, SUN2, SUN3, SUN5, SPAG4 and SUCO from any species and include isoforms, fragments, variants or homologues thereof.

As used herein, a ‘fragment’, ‘variant’ or ‘homologue’ of a protein may optionally be characterised as having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of the reference protein (e.g. a reference isoform of the reference protein). In some embodiments fragments/variants/isoforms/homologues may be characterised by ability to perform a function performed by the reference protein.

A ‘fragment’ generally refers to a fraction of the reference protein. A ‘variant’ generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An ‘isoform’ generally refers to a variant of the reference protein expressed by the same species as the species of the reference protein. A ‘homologue’ generally refers to a variant of the reference protein produced by a different species as compared to the species of the reference protein. Homologues include orthologues.

A ‘fragment’ may be of any length (by number of amino acids), although may optionally be at least 20% of the length of the reference protein (that is, the protein from which the fragment is derived) and may have a maximum length of one of 50%, 75%, 80%, 85%, 90%, 91%, 92%, 93%, 94%, 95%, 96%, 97%, 98%, or 99% of the length of the reference protein.

Isoforms, fragments, variants or homologues may optionally be functional isoforms, fragments, variants or homologues, e.g. having a functional property/activity of the reference protein, as determined by analysis by a suitable assay for the functional property/activity.

In this specification, reference to ‘SUN1’ refers to the protein having the amino acid sequence shown in SEQ ID NO:1, and fragments, variants or homologues thereof. In some embodiments SUN1 comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% amino acid sequence identity to SEQ ID NO:1.

In this specification, reference to ‘SUN2’ refers to the protein having the amino acid sequence shown in SEQ ID NO:13, and fragments, variants or homologues thereof. In some embodiments SUN2 comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% amino acid sequence identity to SEQ ID NO:13.

In this specification, reference to ‘SUN3’ refers to the protein having the amino acid sequence shown in SEQ ID NO:23, and fragments, variants or homologues thereof. In some embodiments SUN3 comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% amino acid sequence identity to SEQ ID NO:23.

In this specification, reference to ‘SUN5’ refers to the protein having the amino acid sequence shown in SEQ ID NO:24, and fragments, variants or homologues thereof. In some embodiments SUN5 comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% amino acid sequence identity to SEQ ID NO:24.

In this specification, reference to ‘SPAG4’ refers to the protein having the amino acid sequence shown in SEQ ID NO:25, and fragments, variants or homologues thereof. In some embodiments SPAG4 comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% amino acid sequence identity to SEQ ID NO:25.

In this specification, reference to ‘SUCO’ refers to the protein having the amino acid sequence shown in SEQ ID NO:26, and fragments, variants or homologues thereof. In some embodiments SUCO comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% amino acid sequence identity to SEQ ID NO:26.

Herein, a ‘KASH domain-containing protein’ refers to any polypeptide comprising a KASH domain. KASH (Klarsicht, ANC-1, Syne homology) domain proteins are carboxy terminal-anchored membrane proteins which are targeted to the nuclear envelope. The 50-60 amino acid KASH domain is found at the C-terminus. KASH domains are hydrophobic, and comprise a single-membrane spanning helix which spans the ONM, and a ˜30 amino acid region which extends into the perinuclear space.

A KASH domain may comprise or consist of the amino acid sequence shown in SEQ ID NO:37, 38, 39, 40, 41 or 42, or an amino acid sequence having at least 60%, e.g. one of at least 65%, 70%, 75%, 80%, 85%, 90%, 91%, 92%, 93%, 94%, 95%, 96%, 97%, 98%, 99% or 100% amino acid sequence identity to the amino acid sequence shown in SEQ ID NO:37, 38, 39, 40, 41 or 42.

In some embodiments, a KASH domain-containing protein is selected from Nesprin-1, Nesprin-2, Nesprin-3, Nesprin-4 (also known as SYNE1, SYNE2, SYNE3 and SYNE4, respectively), KASH5 and LRMP. In some embodiments, a KASH domain-containing protein is Nesprin-1, Nesprin-2 or Nesprin-3.

In some embodiments, a KASH domain-containing protein is capable of forming a LINC complex. In some embodiments, a KASH domain-containing protein is capable of interacting with a SUN domain and/or a SUN domain-containing protein.

Human Nesprin-1 is the polypeptide identified by UniProtKB Q8NF91, the amino acid sequence of which is shown in SEQ ID NO:31. Human Nesprin-2 is the polypeptide identified by UniProtKB Q8WXH0, the amino acid sequence of which is shown in SEQ ID NO:32. Human Nesprin-3 is the polypeptide identified by UniProtKB Q6ZMZ3, the amino acid sequence of which is shown in SEQ ID NO: 33. Human Nesprin-4 is the polypeptide identified by UniProtKB Q8N205, the amino acid sequence of which is shown in SEQ ID NO:34. Human KASH5 is the polypeptide identified by UniProtKB Q8N6L0, the amino acid sequence of which is shown in SEQ ID NO:35. Human LRMP is the polypeptide identified by UniProtKB Q12912, the amino acid sequence of which is shown in SEQ ID NO: 36.

In this specification ‘Nesprin-1’, ‘Nesprin-2’, ‘Nesprin-3’, ‘Nesprin-4’, ‘KASH5’ and ‘LRMP’ respectively refer to Nesprin-1, Nesprin-2, Nesprin-3, Nesprin-4, KASH5 and LRMP from any species and include isoforms, fragments, variants or homologues thereof.

In this specification, reference to ‘Nesprin-1’ refers to the protein having the amino acid sequence shown in SEQ ID NO:31, and fragments, variants or homologues thereof. In some embodiments Nesprin-1 comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% amino acid sequence identity to SEQ ID NO:31.

In this specification, reference to ‘Nesprin-2’ refers to the protein having the amino acid sequence shown in SEQ ID NO:32, and fragments, variants or homologues thereof. In some embodiments Nesprin-2 comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% amino acid sequence identity to SEQ ID NO:32.

In this specification, reference to ‘Nesprin-3’ refers to the protein having the amino acid sequence shown in SEQ ID NO:33, and fragments, variants or homologues thereof. In some embodiments Nesprin-3 comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% amino acid sequence identity to SEQ ID NO:33.

In this specification, reference to ‘Nesprin-4’ refers to the protein having the amino acid sequence shown in SEQ ID NO:34, and fragments, variants or homologues thereof. In some embodiments Nesprin-4 comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% amino acid sequence identity to SEQ ID NO:34.

In this specification, reference to ‘KASH5’ refers to the protein having the amino acid sequence shown in SEQ ID NO:35, and fragments, variants or homologues thereof. In some embodiments KASH5 comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% amino acid sequence identity to SEQ ID NO:35.

In this specification, reference to ‘LRMP’ refers to the protein having the amino acid sequence shown in SEQ ID NO:36, and fragments, variants or homologues thereof. In some embodiments LMRP comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% amino acid sequence identity to SEQ ID NO:36.

As used herein, a ‘LINC complex’ refers to a polypeptide complex comprising a SUN domain-containing protein and a KASH domain-containing protein.

LINC complexes are formed by protein-protein interactions between SUN domain-containing proteins and KASH domain-containing proteins. The LINC complex may comprise non-covalent and/or covalent interactions between SUN domains and KASH domains. Non-covalent interactions include e.g. hydrogen bonds, ionic interaction, Van der Waals forces and hydrophobic bonds. Covalent interactions include e.g. disulphide bonds.

SUN domain proteins are thought to oligomerise to form trimers via interaction between their stalk regions to form coiled-coil triple helix (Zhou et al., J. Biol. Chem. (2012) 287:5317-5326). Deletion of SUN domain protein stalk regions has been shown to disrupt LINC complex formation. SUN domains assume a B sandwich structure and SUN domains of the trimer interact extensively with one another in part through protruding B sheets known as KASH lids; the KASH lid of one SUN domain partially overlaps the β sandwich of the adjacent SUN domain (Sosa et al., Cell (2012) 149:1035-1047).

KASH domain proteins can also oligomerise, which may involve protein-protein interactions between the transmembrane helices. A single KASH domain interacts with two adjacent SUN domains along the groove formed between the KASH lid of one SUN domain and the upper region of the β sandwich of the adjacent SUN domain. In this way, SUN and KASH domains are thought to interact to form a 3:3 hexameric heterocomplex. The 2-3 proline residues immediately prior to the C-terminus of the KASH domain are thought to be accommodated in a deep pocket within the surface of a SUN domain. This region of KASH is important for SUN-KASH interactions; extension of the C-terminus by only a single amino acid disrupts LINC complex formation. Conserved cysteine residues of SUN and KASH domains form disulphide bonds, further stabilising the SUN-KASH complex. The disulphide bonds may be important for force transmission through the LINC complex (Jahed et al., Biophys. J. (2015) 109:501-509).

As explained hereinabove, LINC complexes are thought to form through the interaction of the SUN domains of three SUN domain-containing proteins and the KASH domains of three KASH domain-containing proteins. Interaction between the SUN and KASH domain proteins is thought to be promiscuous; SUN1 and SUN2 have been shown to interact with Nesprin-1, Nesprin-2 and Nesprin-3.

A LINC complex according to the present disclosure may comprise any SUN domain-containing protein and any KASH domain-containing protein. The SUN domain-containing proteins of the LINC complex may be identical or non-identical. The KASH domain-containing proteins of the LINC complex may be identical or non-identical.

LINC complex function is reviewed e.g. in Hieda, Cells (2017) 6 (1): 3 (incorporated by reference hereinabove), and Stroud, Biophys Rev. (2018) 10 (4): 1033-1051, hereby incorporated by reference its entirety.

The LINC complex performs diverse functions, including providing structural support to the nucleus, shaping and positioning the nucleus, maintaining connection between the centrosome and the nucleus and spacing of the nuclear membrane, DNA repair, cell migration and moving chromosomes within the nucleus during meiosis.

The LINC complex has a mechanosensory role to translate mechanical stimuli and changes in the extracellular matrix into signals allowing the cell to adapt to its environment by modulation of cytoskeleton organization, gene expression, nuclear organisation, and structure.

Integrins mediate the transduction of forces from the external microenvironment to the intracellular cytoskeleton, and nucleo-cytoskeletal molecular connections transmit the forces to chromosomal organisations in the nucleus. The nuclear lamina triggers the deformation of nuclear structures, and initiates changes in gene regulation.

The nuclear envelope is a key structure in such processes. On the nucleoplasmic side of the INM, the nuclear lamina (composed of A-type and B-type lamins) forms a lattice structure which contributes to the mechanical stress resistance of the nucleus, and which is essential to the structural integrity of the nuclear envelope. Nuclear lamins are involved in processes critical to cell function and viability, including maintenance of nuclear integrity, regulation of cell cycle, mechanotransduction, cell signalling and DNA repair.

Deviations from normal expression and/or function of nuclear envelope proteins, and deviations from normal expression and/or function of factors directly or indirectly associated with the nuclear envelope, are implicated in a variety of diseases including muscular dystrophies, cardiomyopathies, lipodystrophy, progeria, cancer, and neurological diseases.

LINC complex inhibiting polypeptides

The present disclosure relates to LINC complex inhibiting polypeptides. Herein, a ‘LINC complex inhibiting polypeptide’ refers to a polypeptide that inhibits the LINC complex formation and/or function.

A LINC complex inhibiting polypeptide according to the present disclosure may:
bind to a constituent protein of a LINC complex (e.g. a KASH domain-containing protein, e.g. one or more of Nesprin-1, Nesprin-2, Nesprin-3, Nesprin-4, KASH5 and LRMP);
inhibit interaction between constituent proteins of a LINC complex (e.g. interaction between a SUN domain-containing protein and a KASH domain-containing protein);
inhibit the formation of a LINC complex (i.e. it may inhibit assembly of a LINC complex; e.g. a LINC complex comprising a SUN domain-containing protein and a KASH domain-containing protein);
disrupt a LINC complex (e.g. cause or promote disassembly of a LINC complex, e.g. via displacement of one or more constituent proteins of a LINC complex; e.g. a LINC complex comprising a SUN domain-containing protein and a KASH domain-containing protein);
disrupt the normal subcellular localisation of a constituent protein of a LINC complex (e.g. a LINC complex comprising a SUN domain-containing protein and a KASH domain-containing protein);
increase localisation of a constituent protein of a LINC complex (e.g. a LINC complex comprising a SUN domain-containing protein and a KASH domain-containing protein) to the endoplasmic reticulum;
reduce the level of a LINC complex (e.g. a LINC complex comprising a SUN domain-containing protein and a KASH domain-containing protein);
inhibit a function/activity of a LINC complex (e.g. a LINC complex comprising a SUN domain-containing protein and a KASH domain-containing protein);
increase survival of a subject having a laminopathy;
increase the lifespan of a subject having a laminopathy;
increase, or inhibit the decline of, cardiac function in a subject having a laminopathy;
increase, or inhibit the reduction of, myocardial contractility in a subject having a laminopathy;
increase, or inhibit the reduction of, ejection fraction and/or fractional shortening in a subject having a laminopathy;
reduce, or inhibit the increase in, left ventricular inner diameter in a subject having a laminopathy; and/or
increase, or inhibit the reduction of, left ventricular posterior wall thickness in a subject having a laminopathy.

It will be appreciated that a LINC complex inhibiting polypeptide according to the present disclosure may display more than one of the properties recited in the preceding paragraph. A given polypeptide may be evaluated for the properties recited in the preceding paragraph using suitable assays. The assays may be e.g. in vitro assays, optionally cell-based assays or cell-free assays. The assays may be e.g. in vivo assays, i.e. performed in non-human animals.

Where assays are cell-based assays, they may comprise engineering the cells (e.g. via transfection/transduction) to express a putative LINC complex inhibiting polypeptide, in order to determine whether the polypeptide displays one or more of the recited properties. Assays may employ species labelled with detectable entities in order to facilitate their detection. It will be appreciated that the cells are preferably cells that express the constituent proteins of LINC complexes.

Constituent proteins of LINC complexes include SUN domain-containing proteins and KASH domain-containing proteins. For conciseness, in the present specification ‘a constituent protein of a LINC complex’ may be referred to simply as ‘a LINC complex protein’.

In preferred embodiments, a LINC complex comprises a SUN domain-containing protein and a KASH domain-containing protein. In some embodiments, a SUN domain-containing protein may be selected from SUN1, SUN2, SUN3, SUN5, SPAG4 and SUCO (more preferably selected from SUN1 and SUN2). In some embodiments, a KASH domain-containing protein may be selected from Nesprin-1, Nesprin-2, Nesprin-3, Nesprin-4, KASH5 and LRMP (more preferably selected from Nesprin-1, Nesprin-2 and Nesprin-3).

LINC complex inhibiting polypeptides according to the present disclosure preferably bind to a LINC complex protein. The binding may be characterised by non-covalent, protein: protein interaction between the LINC complex inhibiting polypeptide and the LINC complex protein. The interaction may comprise electrostatic interaction (e.g. ionic bonding, hydrogen bonding) and/or Van der Waals forces.

The present disclosure is particularly concerned with LINC complex inhibiting polypeptides which bind to LINC complex proteins that are interaction partners for SUN domain-containing proteins (e.g. SUN domain-containing proteins as described herein). In some embodiments, an interaction partner for a SUN domain-containing protein is a KASH domain-containing protein (e.g. a KASH domain-containing protein as described herein, e.g. selected from Nesprin-1, Nesprin-2, Nesprin-3, Nesprin-4, KASH5 and LRMP). In some embodiments, an interaction partner for a SUN domain-containing protein is a SUN domain-containing protein (e.g. a SUN domain-containing protein selected from SUN1, SUN2, SUN3, SUN5, SPAG4 and SUCO); SUN domain-containing proteins are known to associate to form multimers (e.g. trimers).

LINC complex inhibiting polypeptides according to the present disclosure may display specific binding to an interaction partner for a SUN domain-containing protein (e.g. a KASH domain-containing protein, e.g. selected from Nesprin-1, Nesprin-2, Nesprin-3, Nesprin-4, KASH5 and LRMP). As used herein, ‘specific binding’ refers to binding which is selective, and which can be discriminated from non-specific binding to non-target molecules. LINC complex inhibiting polypeptides that bind specifically to an interaction partner for a SUN domain-containing protein preferably bind to the relevant protein with greater affinity, and/or with greater duration, the affinity/duration of binding to other, non-target molecules.

The ability of a given polypeptide to bind specifically to a given target molecule can be determined by analysis according to methods known in the art, such as by ELISA, Surface Plasmon Resonance (SPR; see e.g. Hearty et al., Methods Mol Biol (2012) 907:411-442), Bio-Layer Interferometry (see e.g. Lad et al., (2015) J Biomol Screen 20 (4): 498-507), flow cytometry, or by a radiolabeled antigen-binding assay (RIA) enzyme-linked immunosorbent assay.

In some embodiments, a LINC complex inhibiting polypeptide according to the present disclosure binds to an interaction partner for a SUN domain-containing protein (e.g. a KASH domain-containing protein, e.g. selected from Nesprin-1, Nesprin-2, Nesprin-3, Nesprin-4, KASH5 and LRMP) with an affinity in the micromolar range, i.e. K_{D}=9.9×10⁻⁴ to 1×10⁻⁶ M. In some embodiments, a LINC complex inhibiting polypeptide binds to an interaction partner for a SUN domain-containing protein (e.g. a KASH domain-containing protein, e.g. selected from Nesprin-1, Nesprin-2, Nesprin-3, Nesprin-4, KASH5 and LRMP) with sub-micromolar affinity, i.e. K_{D}<1×10⁻⁶ M. In some embodiments, a LINC complex inhibiting polypeptide binds to an interaction partner for a SUN domain-containing protein (e.g. a KASH domain-containing protein, e.g. selected from Nesprin-1, Nesprin-2, Nesprin-3,

Nesprin-4, KASH5 and LRMP) with an affinity in the nanomolar range, i.e. K_{D}=9.9×10⁻⁷ to 1×10⁻⁹ M. In some embodiments, a LINC complex inhibiting polypeptide binds to an interaction partner for a SUN domain-containing protein (e.g. a KASH domain-containing protein, e.g. selected from Nesprin-1, Nesprin-2, Nesprin-3, Nesprin-4, KASH5 and LRMP) with sub-nanomolar affinity, i.e. K_{D}<1×10⁻⁹ M. In some embodiments, a LINC complex inhibiting polypeptide binds to an interaction partner for a

SUN domain-containing protein (e.g. a KASH domain-containing protein, e.g. selected from Nesprin-1, Nesprin-2, Nesprin-3, Nesprin-4, KASH5 and LRMP) with an affinity in the picomolar range, i.e. K_{D} =9.9×10⁻¹⁰ to 1×10⁻¹² M. In some embodiments, a LINC complex inhibiting polypeptide binds to an interaction partner for a SUN domain-containing protein (e.g. a KASH domain-containing protein, e.g. selected from Nesprin-1, Nesprin-2, Nesprin-3, Nesprin-4, KASH5 and LRMP) with sub-picomolar affinity, i.e. K_{D}<1×10⁻¹² M. LINC complex inhibiting polypeptides according to the present disclosure may bind to an interaction partner for a SUN domain-containing protein (e.g. a KASH domain-containing protein, e.g. selected from Nesprin-1, Nesprin-2, Nesprin-3, Nesprin-4, KASH5 and LRMP) in the manner of a SUN domain-containing protein (e.g. a SUN domain-containing protein selected from SUN1, SUN2, SUN3, SUN5, SPAG4 and SUCO). In some embodiments, a LINC complex inhibiting polypeptide may bind to the same region, or an overlapping region, of an interaction partner for a SUN domain-containing protein as the region which is bound by a SUN domain-containing protein.

The region of a given target molecule to which a polypeptide binds can be determined by the skilled person using various methods well known in the art, including X-ray co-crystallography analysis of antibody-antigen complexes, peptide scanning, mutagenesis mapping, hydrogen-deuterium exchange analysis by mass spectrometry, phage display, competition ELISA and proteolysis-based ‘protection’ methods. Such methods are described, for example, in Gershoni et al., BioDrugs, 2007, 21 (3): 145-156, which is hereby incorporated by reference in its entirety.

In some embodiments, a LINC complex inhibiting polypeptide according to the present disclosure inhibits interaction between a SUN domain-containing protein (e.g. a SUN domain-containing protein selected from SUN1, SUN2, SUN3, SUN5, SPAG4 and SUCO) and an interaction partner for a SUN domain-containing protein (e.g. a KASH domain-containing protein, e.g. selected from Nesprin-1, Nesprin-2, Nesprin-3, Nesprin-4, KASH5 and LRMP). Inhibition of interaction between a SUN domain-containing protein and an interaction partner for a SUN domain-containing protein herein encompasses inhibition of binding between a SUN domain-containing protein and an interaction partner for a SUN domain-containing protein (thereby inhibiting formation of a complex comprising such proteins), and disruption of complexes comprising a SUN domain-containing protein and an interaction partner for a SUN domain-containing protein (e.g. via displacement of a constituent protein of such complexes, and consequent disassembly of such complexes).

In some embodiments, a LINC complex inhibiting polypeptide inhibits binding of a SUN domain-containing protein (e.g. a SUN domain-containing protein selected from SUN1, SUN2, SUN3, SUN5, SPAG4 and SUCO) to an interaction partner for a SUN domain-containing protein (e.g. a KASH domain-containing protein, e.g. selected from Nesprin-1, Nesprin-2, Nesprin-3, Nesprin-4, KASH5 and LRMP). In some embodiments, a LINC complex inhibiting polypeptide is a competitive inhibitor of binding of a SUN domain-containing protein to an interaction partner for a SUN domain-containing protein. In some embodiments, a LINC complex inhibiting polypeptide blocks a SUN domain-containing protein from binding to an interaction partner for a SUN domain-containing protein. In some embodiments, a LINC complex inhibiting polypeptide occupies the region of an interaction partner for a SUN domain-containing protein to which a SUN domain-containing protein binds, thereby inhibiting interaction between the SUN domain-containing protein and the interaction partner therefor. In some embodiments, a LINC complex inhibiting polypeptide displaces a SUN domain-containing protein from a complex comprising a SUN domain-containing protein and an interaction partner for a SUN domain-containing protein.

The ability of a given polypeptide to inhibit interaction between two factors can be determined for example by analysis of a correlate of such interaction in the presence of, or following incubation of one or both of the interaction partners with, the polypeptide.

Analysis may comprise detecting a SUN domain-containing protein/an interaction partner for a SUN domain-containing protein/a complex comprising a SUN domain-containing protein and an interaction partner for a SUN domain-containing protein. Such techniques are well known to the skilled person, and include e.g. antibody/reporter-based methods (western blot, ELISA, immunohisto/cytochemistry, etc.).

A polypeptide that inhibits a given interaction (e.g. between a SUN domain-containing protein and an interaction partner therefor, e.g. a KASH domain-containing protein) is identified by the observation of a reduction/decrease in the level of a correlate of interaction between the interaction partners in the presence of—or following incubation of one or both of the interaction partners with—the polypeptide, as compared to the level observed in the absence of the polypeptide (or in the presence of an appropriate control polypeptide known not to inhibit interaction between the interaction partners).

Suitable analysis can be performed in vitro, e.g. using recombinant interaction partners or using cells expressing the interaction partners. Cells expressing interaction partners may do so endogenously, or may do so from nucleic acid introduced into the cell. For the purposes of such assays, one or both of the interaction partners and/or the polypeptide may be labelled or used in conjunction with a detectable entity for the purposes of detecting and/or measuring the level of interaction.

A correlate of interaction between two interaction partners may e.g. be the complex formed by association between the interaction partners, a functional property of the complex formed by association between the interaction partners, or a correlate of a downstream activity mediated by the complex formed by association between the interaction partners.

A polypeptide that inhibits interaction between a SUN domain-containing protein (e.g. a SUN domain-containing protein selected from SUN1, SUN2, SUN3, SUN5, SPAG4 and SUCO) and an interaction partner for a SUN domain-containing protein (e.g. a KASH domain-containing protein, e.g. selected from Nesprin-1, Nesprin-2, Nesprin-3, Nesprin-4, KASH5 and LRMP)—for example, a polypeptide that inhibits formation of complexes comprising a SUN domain-containing protein and an interaction partner for a SUN domain-containing protein, and/or a polypeptide that disrupts complexes comprising a SUN domain-containing protein and an interaction partner for a SUN domain-containing protein—may be identified by the observation of one or more of the following, in the presence of—or following incubation of one or both of the interaction partners with—the polypeptide, as compared to the level observed in the absence of the polypeptide (or in the presence of an appropriate control polypeptide known not to inhibit interaction between the interaction partners): (i) a decrease in the level of the complex comprising the SUN domain-containing protein and the interaction partner for a SUN domain-containing protein, (ii) a decrease in the level of a functional property of the complex comprising the SUN domain-containing protein and the interaction partner for a SUN domain-containing protein, (iii) a decrease in the level of a correlate of a downstream activity mediated by the complex comprising the SUN domain-containing protein and the interaction partner for a SUN domain-containing protein, (iv) an increase in the level of free (i.e. uncomplexed) SUN domain-containing protein, and/or (v) an increase in the level of free (i.e. uncomplexed) interaction partner for a SUN domain-containing protein.

LINC complex inhibiting polypeptides according to the present disclosure may disrupt the normal subcellular localisation of a LINC complex protein (e.g. a SUN domain-containing protein, or an interaction partner for a SUN domain-containing protein).

In some embodiments, a LINC complex inhibiting polypeptide reduces the level/proportion of a LINC complex protein localised to the nuclear envelope. In some embodiments, a LINC complex inhibiting polypeptide reduces the level/proportion of an interaction partner for the LINC complex inhibiting polypeptide localised to the nuclear envelope. In some embodiments, a LINC complex inhibiting polypeptide reduces the level/proportion of a KASH domain-containing protein associated with outer nuclear membrane. In some embodiments, a LINC complex inhibiting polypeptide reduces the level/proportion of a SUN domain-containing protein associated with inner nuclear membrane.

In some embodiments, a LINC complex inhibiting polypeptide increases the level/proportion of a LINC complex protein not localised to the nuclear envelope. In some embodiments, a LINC complex inhibiting polypeptide increases the level/proportion of a KASH domain-containing protein (e.g. selected from Nesprin-1, Nesprin-2, Nesprin-3, Nesprin-4, KASH5 and LRMP) not associated with outer nuclear membrane. In some embodiments, a LINC complex inhibiting polypeptide increases the level/proportion of a SUN domain-containing protein (e.g. selected from SUN1, SUN2, SUN3, SUN5, SPAG4 and SUCO) not associated with inner nuclear membrane.

In some embodiments, a LINC complex inhibiting polypeptide increases the level/proportion of a LINC complex protein localised to the endoplasmic reticulum. In some embodiments, a LINC complex inhibiting polypeptide increases the level/proportion of a KASH domain-containing protein (e.g. selected from Nesprin-1, Nesprin-2, Nesprin-3, Nesprin-4, KASH5 and LRMP) localised to the endoplasmic reticulum. In some embodiments, a LINC complex inhibiting polypeptide increases the level/proportion of a SUN domain-containing protein (e.g. selected from SUN1, SUN2, SUN3, SUN5, SPAG4 and SUCO) not associated with inner nuclear membrane.

The subcellular localisation of constituent proteins of LINC complexes within cells can be analysed using techniques known to the person skilled in the art. Such techniques include e.g. analysis by immunocytochemistry and reporter-based methods. For example, Boni et al., J. Cell Biology (2015) 209 (5): 705-720 and Smoyer et al., J. Cell Biology (2016) 215 (4): 575-590 describe a reporter system permitting imaging of proteins in the ER, INM and ONM. Such methods can be employed to analyse the levels/proportions of constituent proteins of LINC complexes in the nuclear envelope, inner nuclear membrane and an outer nuclear membrane.

LINC complex inhibiting polypeptides that disrupt the normal subcellular localisation of a LINC complex protein may be identified using assays comprising detecting the presence of, or determining the proportion of, the relevant protein(s) in a given subcellular location, e.g. using antibody/reporter-based methods (western blot, ELISA, immunohisto/cytochemistry, etc.). Subcellular localisation may be analysed e.g. by immunocytochemistry, or by western blot of extracts prepared from different cellular fractions, and may employ organelle markers and/or labelled proteins of known subcellular localisation.

Assays may comprise expressing a putative LINC complex inhibiting polypeptide in a cell (e.g. from nucleic acid encoding the polypeptide introduced (e.g. by transfection/transduction) into the cell), and subsequently comparing the subcellular localisation of the relevant LINC complex protein(s) in such cells to the subcellular localisation observed in cells of an appropriate control condition (e.g. non-transfected cells, cells transfected/transduced with empty vector, or cells transfected/transduced with nucleic acid encoding a polypeptide known not to affect subcellular localisation of the relevant LINC complex protein(s)).

In some embodiments, polypeptides may be evaluated for their ability to behave as LINC complex inhibiting polypeptides essentially as described in Example 1 herein.

Nucleic acid for expressing a putative LINC complex inhibiting polypeptide may be introduced (e.g. by transfection) into cells, and the cells may subsequently be evaluated in order to determine the subcellular localisation of one or more LINC complex proteins (e.g. a SUN domain-containing protein (e.g. selected from SUN1, SUN2, SUN3, SUN5, SPAG4 and SUCO) and/or an interaction partner for a SUN domain-containing protein (e.g. a KASH domain-containing protein, e.g. selected from Nesprin-1, Nesprin-2, Nesprin-3, Nesprin-4, KASH5 and LRMP)). A polypeptide may be identified as a LINC complex inhibiting polypeptide where its expression is determined in such an assay to reduce the level/proportion of a LINC complex protein localised to the nuclear envelope, or to increase the level/proportion of a LINC complex protein not localised to the nuclear envelope (e.g. where its expression is determined to reduce the level/proportion of a KASH domain-containing protein associated with outer nuclear membrane, or to increase the level/proportion of a KASH domain-containing protein not associated with outer nuclear membrane, or to increase the level/proportion of a KASH domain-containing protein localised to the endoplasmic reticulum) relative to the level/proportion observed in cells of an appropriate control condition (e.g. non-transfected cells, cells transfected/transduced with empty vector, or cells transfected/transduced with nucleic acid encoding a polypeptide known not to affect subcellular localisation of the relevant LINC complex protein(s)).

In some embodiments, cellular expression of a LINC complex inhibiting polypeptide according to the present disclosure: (i) reduces the level of interaction between constituent proteins of a LINC complex (e.g. between a SUN domain-containing protein and a KASH domain-containing protein), (ii) reduces the level of a LINC complex (e.g. a LINC complex comprising a SUN domain-containing protein and a KASH domain-containing protein), (iii) reduces the level of a correlate of a function/activity of a LINC complex (e.g. a LINC complex comprising a SUN domain-containing protein and a KASH domain-containing protein), (iv) reduces the level/proportion of a LINC complex protein (e.g. a SUN domain-containing protein or a KASH domain-containing protein) localised to the nuclear envelope, and/or (v) reduces the level/proportion of a KASH domain-containing protein (e.g. selected from Nesprin-1, Nesprin-2, Nesprin-3, Nesprin-4, KASH5 and LRMP) localised to the nuclear envelope/outer nuclear membrane to less than 1 times/less than 100%, e.g. one of ≤0.99 times/≤99%, ≤0.95 times/≤95%, ≤0.9 times/≤90%, ≤0.85 times/≤85%, ≤0.8 times/≤80%, ≤0.75 times/≤75%, ≤0.7 times/≤70%, ≤0.65 times/≤65%, ≤0.6 times/≤60%, ≤0.55 times/≤55%, ≤0.5 times/≤50%, ≤0.45 times/≤45%, ≤0.4 times/≤40%, ≤0.35 times/≤35%, ≤0.3 times/≤30%, ≤0.25 times/≤25%, ≤0.2 times/≤20%, ≤0.15 times/≤15%, ≤0.1 times/≤10% times, ≤0.05 times/≤5%, or ≤0.01 times/≤1% of the level observed in the absence of the LINC complex inhibiting polypeptide, or in an appropriate control condition, in a given assay.

Preferred levels of reduction in accordance with the preceding paragraph are reduction to less than 0.5 times/≤50%, e.g. one of ≤0.4 times/≤40%, ≤0.3 times/≤30%, ≤0.2 times/≤20%, ≤0.15 times/≤15%, or ≤0.1 times/≤10%.

In some embodiments, cellular expression of a LINC complex inhibiting polypeptide according to the present disclosure: (i) increases the level/proportion of a LINC complex protein (e.g. a SUN domain-containing protein or a KASH domain-containing protein) not localised to the nuclear envelope, and/or (ii) increases the level/proportion of a LINC complex protein (e.g. a SUN domain-containing protein or a KASH domain-containing protein) localised to the endoplasmic reticulum to greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, >4 times, >5 times, >6 times, >7 times, >8 times, >9 times, >10 times, >50 times or >100 times the level observed in the absence of the LINC complex inhibiting polypeptide, or in an appropriate control condition, in a given assay.

Polypeptides may be evaluated for certain functional properties in an appropriate in vivo model. For example, polypeptides may be evaluated for therapeutic/prophylactic effects in vivo in a non-human animal model of a disease/condition described herein. In such assays, the polypeptide may be delivered in the form of nucleic acid encoding the protein, e.g. using a viral vector having an appropriate tropism for cells/tissue in which LINC complex inhibition would confer therapeutic/prophylactic benefit. By way of illustration, in the experimental Examples of the present disclosure, putative LINC complex inhibiting polypeptides are evaluated in a mouse model of dilated cardiomyopathy established by inducible, cardiomyocyte-specific deletion of Lmna-see Example 5. The putative LINC complex inhibiting polypeptides are delivered as a gene therapy, in the form of AAV9 vector expressing the polypeptides under the control of a cardiomyocyte-specific promoter.

The lifespan and survival of subjects can be evaluated in such models by monitoring survival over time. Cardiac function and myocardial contractility can be evaluated by measuring correlates thereof, and ejection fraction, fractional shortening, left ventricular posterior wall thickness and/or left ventricular inner diameter can be measured by echocardiography/ultrasound.

It will be appreciated that subjects are preferably evaluated for one or more of the properties recited in the preceding paragraph at a specified time point, e.g. after a period of time sufficient for an effect of administration of the polypeptide in the relevant model to be observed. For example, subjects may be evaluated at 7 or more days after administration of viral vector encoding the putative LINC complex inhibiting polypeptide.

In some embodiments, administration of a LINC complex inhibiting polypeptide according to the present disclosure to a subject having a laminopathy (e.g. cardiomyopathy associated with mutation to LMNA; e.g. via a viral vector encoding the LINC complex inhibiting polypeptide) increases survival or the lifespan of the subject, and/or increases cardiac function, myocardial contractility, ejection fraction, fractional shortening and/or left ventricular posterior wall thickness in the subject, to greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, >4 times, >5 times, >6 times, >7 times, >8 times, ≥9 times, ≥10 times, ≥50 times or ≥100 times the level observed in the absence of such treatment, or following administration of an appropriate control polypeptide (e.g. a polypeptide known not to inhibit the LINC complex, e.g. delivered using the same viral vector).

In some embodiments, administration of a LINC complex inhibiting polypeptide according to the present disclosure to a subject having a laminopathy (e.g. cardiomyopathy associated with mutation to LMNA; e.g. via a viral vector encoding the LINC complex inhibiting polypeptide) reduces left ventricular inner diameter in the subject, to less than 1 times/less than 100%, e.g. one of ≤0.99 times/≤99%, ≤0.95 times/≤95%, ≤0.9 times/≤90%, ≤0.85 times/≤85%, ≤0.8 times/≤80%, ≤0.75 times/≤75%, ≤0.7 times/≤70%, ≤0.65 times/≤65%, ≤0.6 times/≤60%, ≤0.55 times/≤55%, ≤0.5 times/≤50%, ≤0.45 times/≤45%, ≤0.4 times/≤40%, ≤0.35 times/≤35%, ≤0.3 times/≤30%, ≤0.25 times/≤25%, ≤0.2 times/≤20%, ≤0.15 times/≤15%, ≤0.1 times/≤10% times, ≤0.05 times/≤5%, or ≤0.01 times/≤1% of the level observed in the absence of such treatment, or following administration of an appropriate control polypeptide (e.g. a polypeptide known not to inhibit the LINC complex, e.g. delivered using the same viral vector).

In some embodiments, a LINC complex inhibiting polypeptide according to the present disclosure may possess one or more novel or improved functional properties as compared to the LINC complex inhibiting polypeptide consisting of the amino acid sequence of SEQ ID NO:99.

In some embodiments, cellular expression of a LINC complex inhibiting polypeptide according to the present disclosure: (i) reduces the level of interaction between constituent proteins of a LINC complex (e.g. between a SUN domain-containing protein and a KASH domain-containing protein), (ii) reduces the level of a LINC complex (e.g. a LINC complex comprising a SUN domain-containing protein and a KASH domain-containing protein), (iii) reduces the level of a correlate of a function/activity of a LINC complex (e.g. a LINC complex comprising a SUN domain-containing protein and a KASH domain-containing protein), (iv) reduces the level/proportion of a LINC complex protein (e.g. a SUN domain-containing protein or a KASH domain-containing protein) localised to the nuclear envelope, and/or (v) reduces the level/proportion of a KASH domain-containing protein (e.g. selected from Nesprin-1, Nesprin-2, Nesprin-3, Nesprin-4, KASH5 and LRMP) localised to the nuclear envelope/outer nuclear membrane to less than 1 times/less than 100%, e.g. one of ≤0.99 times/≤99%, ≤0.95 times/≤95%, ≤0.9 times/≤90%, ≤0.85 times/≤85%, ≤0.8 times/≤80%, ≤0.75 times/≤75%, ≤0.7 times/≤70%, ≤0.65 times/≤65%, ≤0.6 times/≤60%, ≤0.55 times/≤55%, ≤0.5 times/≤50%, ≤0.45 times/≤45%, ≤0.4 times/≤40%, ≤0.35 times/≤35%, ≤0.3 times/≤30%, ≤0.25 times/≤25%, ≤0.2 times/≤20%, ≤0.15 times/≤15%, ≤0.1 times/≤10% times, ≤0.05 times/≤5%, or ≤0.01 times/≤1% of the level observed on cellular expression of a polypeptide consisting of the amino acid sequence of SEQ ID NO:99 (e.g. by a cell of the same type).

In some embodiments, cellular expression of a LINC complex inhibiting polypeptide according to the present disclosure: (i) increases the level/proportion of a LINC complex protein (e.g. a SUN domain-containing protein or a KASH domain-containing protein) not localised to the nuclear envelope, and/or (ii) increases the level/proportion of a LINC complex protein (e.g. a SUN domain-containing protein or a KASH domain-containing protein) localised to the endoplasmic reticulum to greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, >4 times, >5 times, >6 times, >7 times, >8 times, >9 times, >10 times, >50 times or >100 times the level observed on cellular expression of a polypeptide consisting of the amino acid sequence of SEQ ID NO: 99 (e.g. by a cell of the same type).

In some embodiments, administration of a LINC complex inhibiting polypeptide according to the present disclosure to a subject having a laminopathy (e.g. cardiomyopathy associated with mutation to LMNA; e.g. via a viral vector encoding the LINC complex inhibiting polypeptide) increases survival or the lifespan of the subject, and/or increases cardiac function, myocardial contractility, ejection fraction, fractional shortening and/or left ventricular posterior wall thickness in the subject, to greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, >3 times, >4 times, >5 times, >6 times, >7 times, >8 times, ≥9 times, ≥10 times, ≥50 times or ≥100 times the level observed following administration of a polypeptide consisting of the amino acid sequence of SEQ ID NO:99 (e.g. delivered using the same viral vector).

In some embodiments, administration of a LINC complex inhibiting polypeptide according to the present disclosure to a subject having a laminopathy (e.g. cardiomyopathy associated with mutation to LMNA; e.g. via a viral vector encoding the LINC complex inhibiting polypeptide) reduces left ventricular inner diameter in the subject, to less than 1 times/less than 100%, e.g. one of ≤0.99 times/≤99%, ≤0.95 times/≤95%, ≤0.9 times/≤90%, ≤0.85 times/≤85%, ≤0.8 times/≤80%, ≤0.75 times/≤75%, ≤0.7 times/≤70%, ≤0.65 times/≤65%, ≤0.6 times/≤60%, ≤0.55 times/≤55%, ≤0.5 times/≤50%, ≤0.45 times/≤45%, ≤0.4 times/≤40%, ≤0.35 times/≤35%, ≤0.3 times/≤30%, ≤0.25 times/≤25%, ≤0.2 times/≤20%, ≤0.15 times/≤15%, ≤0.1 times/≤10% times, ≤0.05 times/≤5%, or ≤0.01 times/≤1% of the level observed following administration of a polypeptide consisting of the amino acid sequence of SEQ ID NO:99 (e.g. delivered using the same viral vector).

LINC complex inhibiting polypeptides according to the present disclosure may be based on a SUN domain-containing protein, e.g. selected from SUN1, SUN2, SUN3, SUN5, SPAG4 and SUCO. In some embodiments, a LINC complex inhibiting polypeptide according to the present disclosure comprises an inhibitory region which is based on a SUN domain-containing protein, e.g. selected from SUN1, SUN2, SUN3, SUN5, SPAG4 and SUCO.

As used herein, a peptide/polypeptide/amino acid sequence which is ‘based on’ a reference protein comprises or consists of an amino acid sequence having high sequence identity (e.g. at least 80%, 85% 86%, 87%, 88%, 89%, 90%, 91%, 92%, 93%, 94%, 95%, 96%, 97%, 98%, 99%, or 100% sequence identity) to all or part of the amino acid of the reference protein.

An ‘inhibitory region’ of a LINC complex inhibiting polypeptide refers to the region of the polypeptide through which LINC complex inhibition is achieved. An inhibitory region according to the present disclosure typically has a high degree of sequence identity to part of the amino acid sequence of a SUN domain-containing protein. The inhibitory region of a LINC complex inhibiting polypeptide according to the present disclosure may be the region through which the LINC complex inhibiting polypeptide binds to an interaction partner for a SUN domain-containing protein (e.g. a KASH domain-containing protein, e.g. one or more of Nesprin-1, Nesprin-2, Nesprin-3, Nesprin-4, KASH5 and LRMP). In aspects and embodiments of the present disclosure, an inhibitory region of a LINC complex inhibiting polypeptide comprises an amino acid sequence corresponding to the α3 helix of the CC2 region and the SUN domain of a SUN domain-containing protein.

LINC complex inhibiting polypeptides according to the present disclosure may comprise or consist essentially of an amino acid sequence having a high degree of sequence identity (e.g. at least 80%, 85% 86%, 87%, 88%, 89%, 90%, 91%, 92%, 93%, 94%, 95%, 96%, 97%, 98%, 99%, or 100% sequence identity) to the amino acid sequence of all or part of a SUN domain-containing protein, e.g. selected from SUN1, SUN2, SUN3, SUN5, SPAG4 and SUCO. LINC complex inhibiting polypeptides according to the present disclosure may comprise an inhibitory region comprising or consisting of an amino acid sequence having a high degree of sequence identity (e.g. at least 80%, 85% 86%, 87%, 88%, 89%, 90%, 91%, 92%, 93%, 94%, 95%, 96%, 97%, 98%, 99%, or 100% sequence identity) to the amino acid sequence of all or part of a SUN domain-containing protein, e.g. selected from SUN1, SUN2, SUN3, SUN5, SPAG4 and SUCO.

Such LINC complex inhibiting polypeptides preferably (i) retain the ability of the SUN domain-containing protein on which they are based to bind to a KASH domain-containing protein (e.g. one or more of Nesprin-1, Nesprin-2, Nesprin-3, Nesprin-4, KASH5 and LRMP), but (ii) lack, or display a reduced level of, one or more other properties of the SUN domain-containing protein on which they are based. In preferred embodiments, a LINC complex inhibiting polypeptide lacks or displays a reduced ability to bind to nuclear lamins and/or chromatin-binding proteins, and/or lacks or displays a reduced ability to associate with (e.g. localise to) the inner nuclear membrane, relative to the SUN domain-containing protein on which it is based.

For example, a LINC complex inhibiting polypeptide/inhibitory region thereof according to the present disclosure may comprise the amino acid sequence(s) required for binding to a KASH domain-containing protein (e.g. one or more of Nesprin-1, Nesprin-2, Nesprin-3, Nesprin-4, KASH5 and LRMP), and may lack the amino acid sequence(s) required for one or more other properties of the SUN domain-containing protein upon which it is based (e.g. the amino acid sequence(s) required for binding to nuclear lamins and/or chromatin-binding proteins, and/or the amino acid sequence(s) required for association with the inner nuclear membrane).

It will be appreciated that in some embodiments, a LINC complex inhibiting polypeptide according to the present disclosure consists essentially of a KASH domain-containing protein-binding fragment of a SUN domain-containing protein. It will similarly be appreciated that in some embodiments, the inhibitory region of a LINC complex inhibiting polypeptide according to the present disclosure consists of a KASH domain-containing protein-binding fragment of a SUN domain-containing protein.

Such LINC complex inhibiting polypeptides may be referred to as ‘decoy’, ‘dominant-negative’ or ‘mimetic’ versions of the SUN domain-containing proteins on which they are based. That is, in aspects and embodiments of the present disclosure, a LINC complex inhibiting polypeptide may be a dominant-negative SUN domain-containing polypeptide.

LINC complex inhibiting polypeptides according to the present disclosure preferably display competitive inhibition of interaction between a SUN domain-containing protein (e.g. selected from SUN1, SUN2, SUN3, SUN5, SPAG4 and SUCO) and a KASH domain-containing protein (e.g. selected from Nesprin-1, Nesprin-2, Nesprin-3, Nesprin-4, KASH5 and LRMP). Decoy/dominant negative/mimetic LINC complex inhibiting polypeptides preferably bind to a KASH domain-containing protein (e.g. selected from Nesprin-1, Nesprin-2, Nesprin-3, Nesprin-4, KASH5 and LRMP), occupying the site required for interaction with, and thereby in making these species unavailable for binding to, endogenous SUN domain-containing proteins.

Such LINC complex inhibiting polypeptides may inhibit the formation of LINC complexes and/or disrupt existing LINC complexes via inhibition of assembly of the endogenous interaction partners and/or via displacement of the endogenous interaction partners. The decoy/dominant negative/mimetic LINC complex inhibiting polypeptides form non-functional LINC complexes, or LINC complexes having a reduced level of function compared to LINC complexes formed by wildtype, endogenous SUN domain-containing proteins and KASH domain-containing proteins.

As noted hereinabove, dominant-negative versions of SUN-domain containing proteins have been described previously. For example, Crisp et al. J Cell Biol. (2006) 172 (1): 41-53 describes a dominant-negative version of mouse Sun1 protein, and WO 2019/143300 A1 describes a humanised version thereof.

In aspects and embodiments according to the present disclosure, a LINC complex inhibiting polypeptide comprises an amino acid sequence corresponding to the α3 helix of the CC2 region and the SUN domain of a SUN domain-containing protein. It will be appreciated that amino acid sequence(s) of LINC complex inhibiting polypeptides described herein that correspond to one or more regions of a SUN domain-containing protein may be comprised in the inhibitory region of the LINC complex inhibiting polypeptide.

As used herein, an amino acid sequence which ‘corresponds’ to a specified region of a reference polypeptide or amino acid sequence has at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of the polypeptide/amino acid sequence. An amino acid sequence which ‘corresponds’ to a specified region of a reference polypeptide or amino acid sequence can be identified by sequence alignment of the subject sequence to the reference sequence, e.g. using sequence alignment software such as ClustalOmega (Soding, J. 2005, Bioinformatics 21, 951-960). By way of illustration, the amino acid sequence from positions 522 to 717 of human SUN2 corresponds to the amino acid sequence from positions 616 to 812 of human SUN1.

In some embodiments, a LINC complex inhibiting polypeptides according to the present disclosure consists essentially of a human amino acid sequence. In some embodiments, the inhibitory region of a LINC complex inhibiting polypeptide according to the present disclosure consists essentially of a human amino acid sequence. As used herein, ‘a human amino acid sequence’ refers to an amino acid sequence that is encoded by nucleic acid of the genome of a human. That is, in some embodiments a LINC complex inhibiting polypeptide or the inhibitory region of a LINC complex inhibiting polypeptide consists essentially of an amino acid sequence having 100% amino acid sequence identity to an amino acid sequence encoded by the genome of a human subject. It will be appreciated that in some embodiments, the amino acid sequence encoded by the genome of a human subject is an amino acid sequence of a human SUN domain-containing protein (e.g. selected from SUN1, SUN2, SUN3, SUN5, SPAG4 and SUCO; e.g. SUN1 or SUN2). Such embodiments of LINC complex inhibiting polypeptides are contemplated in particular where administration to a human subject is intended, e.g. in the context of therapeutic/prophylactic intervention according to the present disclosure.

In some embodiments, a LINC complex inhibiting polypeptide comprises an amino acid sequence corresponding to the α3 helix of the CC2 region of a SUN domain-containing protein. In some embodiments, a LINC complex inhibiting polypeptide comprises an amino acid sequence corresponding to the α3 helix of the CC2 region of human SUN1. In some embodiments, a LINC complex inhibiting polypeptide comprises an amino acid sequence corresponding to the α3 helix of the CC2 region of human SUN2. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:12. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:22.

In some embodiments, a LINC complex inhibiting polypeptide comprises an amino acid sequence corresponding to the SUN domain of a SUN domain-containing protein. In some embodiments, a LINC complex inhibiting polypeptide comprises an amino acid sequence corresponding to the SUN domain of human SUN1. In some embodiments, a LINC complex inhibiting polypeptide comprises an amino acid sequence corresponding to the SUN domain of human SUN2. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:8. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:18.

In some embodiments, a LINC complex inhibiting polypeptide comprises an amino acid sequence corresponding to the α3 helix of the CC2 region and the SUN domain of a SUN domain-containing protein. In some embodiments, a LINC complex inhibiting polypeptide comprises an amino acid sequence corresponding to the α3 helix of the CC2 region and the SUN domain of human SUN1. In some embodiments, a LINC complex inhibiting polypeptide comprises an inhibitory region comprising an amino acid sequence corresponding to the α3 helix of the CC2 region and the SUN domain of human SUN2. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:44. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of

SEQ ID NO:46.

In some embodiments, a LINC complex inhibiting polypeptide comprises an amino acid sequence corresponding to the α2 helix of the CC2 region of a SUN domain-containing protein. In some embodiments, a LINC complex inhibiting polypeptide comprises an amino acid sequence corresponding to the α2 helix of the CC2 region of human SUN1. In some embodiments, a LINC complex inhibiting polypeptide comprises an amino acid sequence corresponding to the α2 helix of the CC2 region of human SUN2. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:11. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:21.

In some embodiments, a LINC complex inhibiting polypeptide comprises an amino acid sequence corresponding to the α1 helix of the CC2 region of a SUN domain-containing protein. In some embodiments, a LINC complex inhibiting polypeptide comprises an amino acid sequence corresponding to the α1 helix of the CC2 region of human SUN1. In some embodiments, a LINC complex inhibiting polypeptide comprises an amino acid sequence corresponding to the α1 helix of the CC2 region of human SUN2. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:10. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:20.

In some embodiments, a LINC complex inhibiting polypeptide comprises an amino acid sequence corresponding to the CC1 region of a SUN domain-containing protein. In some embodiments, a LINC complex inhibiting polypeptide comprises an amino acid sequence corresponding to the CC1 region of human SUN1. In some embodiments, a LINC complex inhibiting polypeptide comprises an amino acid sequence corresponding to the CC1 region of human SUN2. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:9. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:19.

In some embodiments, a LINC complex inhibiting polypeptide comprises an amino acid sequence corresponding to the CC1 region and CC2 region of a SUN domain-containing protein. In some embodiments, a LINC complex inhibiting polypeptide comprises an amino acid sequence corresponding to the CC1 region and CC2 region of human SUN1. In some embodiments, a LINC complex inhibiting polypeptide comprises an amino acid sequence corresponding to the CC1 region and CC2 region of human SUN2. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:88. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:91.

In some embodiments, a LINC complex inhibiting polypeptide comprises an amino acid sequence corresponding to the CC2 region of a SUN domain-containing protein. In some embodiments, a LINC complex inhibiting polypeptide comprises an amino acid sequence corresponding to the CC2 region of human SUN1. In some embodiments, a LINC complex inhibiting polypeptide comprises an amino acid sequence corresponding to the CC2 region of human SUN2. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:89. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:92.

In some embodiments, a LINC complex inhibiting polypeptide comprises an amino acid sequence corresponding to the α2 and α3 helices of the CC2 region of a SUN domain-containing protein. In some embodiments, a LINC complex inhibiting polypeptide comprises an amino acid sequence corresponding to the α2 and α3 helices of the CC2 region of human SUN1. In some embodiments, a LINC complex inhibiting polypeptide comprises an amino acid sequence corresponding to the α2 and α3 helices of the CC2 region of human SUN2. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:90. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:93.

In some embodiments, a LINC complex inhibiting polypeptide comprises an amino acid sequence corresponding to part of the SMART coil-coiled 2 region, the CC1 region and CC2 region of a SUN domain-containing protein. In some embodiments, a LINC complex inhibiting polypeptide comprises an amino acid sequence corresponding to positions 483 to 632 of human SUN1 (numbered according to SEQ ID NO:1). In some embodiments, a LINC complex inhibiting polypeptide comprises an amino acid sequence corresponding to positions 388 to 538 of human SUN2 (numbered according to SEQ ID NO: 13). In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:97. In some embodiments, a LINC complex inhibiting polypeptide comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:98.

In some embodiments, a LINC complex inhibiting polypeptide does not consist essentially of the amino acid sequence of a dominant-negative version of a SUN domain-containing protein disclosed in the prior art. In some embodiments, a LINC complex inhibiting polypeptide does not consist essentially of the amino acid sequence of a truncated variant of a SUN domain-containing protein disclosed in the prior art. In some embodiments, a LINC complex inhibiting polypeptide does not comprise an inhibitory region consisting of the amino acid sequence of a dominant-negative version of a SUN domain-containing protein disclosed in the prior art. In some embodiments, a LINC complex inhibiting polypeptide does not comprise an inhibitory region consisting of the amino acid sequence of a truncated variant of a SUN domain-containing protein disclosed in the prior art.

As used herein, a polypeptide or amino acid sequence that ‘consists essentially of a reference amino acid sequence either (i) consists of the reference amino acid sequence, or (ii) comprises the reference amino acid sequence, wherein the reference amino acid sequence constitutes at least 80% (e.g. one of ≥85%≥86%, ≥87%, ≥88%, ≥89%, ≥90%, ≥91%, ≥92%, ≥93%, ≥94%, ≥95%, ≥96%, ≥97%, ≥98%, ≥99%) of the polypeptide/amino acid sequence. It will be appreciated that polypeptide or an amino acid sequence that ‘consists essentially of’ a reference amino acid sequence may comprise the reference amino acid sequence and additional amino acid(s) at one or both of the N-terminal and C-terminal ends of the reference amino acid sequence, provided that the additional amino acid(s) constitute≤20% of the polypeptide/amino acid sequence.

By way of illustration, a polypeptide consisting of the amino acid sequence of SEQ ID NO:74 consists essentially of the amino acid sequence of SEQ ID NO:44. SEQ ID NO:74 comprises the amino acid sequence of SEQ ID NO:44 (i.e. from positions 1 to 197 of SEQ ID NO:74), and the amino acid sequence of SEQ ID NO:44 constitutes ˜98% of the polypeptide (i.e. 197/201 residues). SEQ ID NO: 74 comprises the KDEL retention motif (SEQ ID NO:77)C-terminal to the amino acid sequence of SEQ ID NO:44.

In some embodiments, an amino acid sequence which consists essentially of a reference amino acid sequence consists of the reference amino acid sequence. That is, in some embodiments, an amino acid sequence which consists essentially of a reference amino acid sequence is identical to the reference amino acid sequence. In some embodiments, an amino acid sequence which consists essentially of a reference amino acid sequence comprises an amino acid sequence which is identical to the reference amino acid sequence, and further comprises one or more additional amino acid(s) (e.g. 1-5, 1-10 or 1-20 amino acids) at one or both ends of the reference sequence.

In some embodiments, a LINC complex inhibiting polypeptide does not consist essentially of the amino acid sequence of SEQ ID NO:43 or 45. In some embodiments, a LINC complex inhibiting polypeptide does not comprise an inhibitory region consisting of the amino acid sequence of any one of SEQ ID NOs: 43 or 45.

In some embodiments, a LINC complex inhibiting polypeptide does not consist essentially of the amino acid sequence of SEQ ID NO:44 or 46. In some embodiments, a LINC complex inhibiting polypeptide does not comprise an inhibitory region consisting of the amino acid sequence of any one of SEQ ID NOs: 44 or 46.

In some embodiments, a LINC complex inhibiting polypeptide does not consist essentially of the amino acid sequence of SEQ ID NO:43, 44, 45 or 46. In some embodiments, a LINC complex inhibiting polypeptide does not comprise an inhibitory region consisting of the amino acid sequence of any one of SEQ ID NOs: 43, 44, 45 or 46.

In some embodiments, a LINC complex inhibiting polypeptide does not consist essentially of the amino acid sequence of any one of SEQ ID NOs: 43, 44, 45, 46, 47 or 48.

In some embodiments, a LINC complex inhibiting polypeptide does not consist essentially of the amino acid sequence of any one of SEQ ID NOs: 43, 44, 45, 46, 47, 48, 74 or 75.

In some embodiments, a LINC complex inhibiting polypeptide does not consist essentially of the amino acid sequence of any one of SEQ ID NOs: 43, 44, 45, 46, 47, 48, 58, 49, 50, 51, 52, 53, 54, 55, 56, 57, 74 or 75. In some embodiments, a LINC complex inhibiting polypeptide does not comprise an inhibitory region consisting of the amino acid sequence of any one of SEQ ID NOs: 43, 44, 45, 46, 47, 48, 58, 49, 50, 51, 52, 53, 54, 55, 56, 57, 74 or 75.

In some embodiments, a LINC complex inhibiting polypeptide does not consist essentially of the amino acid sequence of any one of SEQ ID NOs: 43, 44, 45, 46, 47, 48, 49, 50, 51, 52, 53, 54, 55, 56, 57, 58, 59, 60, 61, 62, 74 or 75. In some embodiments, a LINC complex inhibiting polypeptide does not comprise an inhibitory region consisting of the amino acid sequence of any one of SEQ ID NOs: 43, 44, 45, 46, 47, 48, 49, 50, 51, 52, 53, 54, 55, 56, 57, 58, 59, 60, 61, 62, 74 or 75.

The present disclosure specifically contemplates disclaiming LINC complex inhibiting polypeptides consisting essentially of the amino acid sequence of any one of SEQ ID NOs: 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 94 or 95 individually, or along with one or more other of the specified sequences. The present disclosure also specifically contemplates disclaiming LINC complex inhibiting polypeptides comprising an inhibitory region consisting of the amino acid sequence of any one of SEQ ID NOs: 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 94 or 95, either individually, or along with one or more other of the specified sequences.

However, in connection with certain aspects and embodiments of the present disclosure, a LINC complex inhibiting polypeptide may comprise or consist essentially of the amino acid sequence of any one of SEQ ID NOs: 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 94 or 95. Similarly, in connection with certain aspects and embodiments of the present disclosure, a LINC complex inhibiting polypeptide may comprise an inhibitory region consisting of the amino acid sequence of any one of SEQ ID NOs: 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 94 or 95.

In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:63. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:64. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:65. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:66. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:67. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:68. In some embodiments, a

LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID SEQ ID NO:NO: 69. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:70. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:71. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:72. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:73. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:74. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of

SEQ ID NO:94. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:95.

In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:101. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:102. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:103.

In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:104. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:105. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO: 106. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:107. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of

SEQ ID NO:108. In some embodiments, a LINC complex inhibiting polypeptide, or an inhibitory region thereof, comprises or consists essentially of an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:109.

In some embodiments, a LINC complex inhibiting polypeptide according to the present disclosure, or an inhibitory region thereof, comprises or consists essentially of the amino acid sequence corresponding to one of: positions 360-812 of SEQ ID NO:1; positions 361-812 of SEQ ID NO:1; positions 362-812 of SEQ ID NO:1; positions 363-812 of SEQ ID NO:1; positions 364-812 of SEQ ID NO: 1; positions 365-812 of SEQ ID NO:1; positions 366-812 of SEQ ID NO:1; positions 367-812 of

SEQ ID NO:1; positions 368-812 of SEQ ID NO:1; positions 369-812 of SEQ ID NO:1; positions 370-812 of SEQ ID NO:1; positions 371-812 of SEQ ID NO:1; positions 372-812 of SEQ ID NO:1; positions 373-812 of SEQ ID NO:1; positions 374-812 of SEQ ID NO:1; positions 375-812 of SEQ ID NO: 1; positions 376-812 of SEQ ID NO:1; positions 377-812 of SEQ ID NO:1; positions 378-812 of SEQ ID NO:1; positions 379-812 of SEQ ID NO:1; positions 380-812 of SEQ ID NO:1; positions 381-812 of SEQ ID NO:1; positions 382-812 of SEQ ID NO:1; positions 383-812 of SEQ ID NO:1; positions 384-812 of SEQ ID NO:1; positions 385-812 of SEQ ID NO:1; positions 386-812 of SEQ ID NO: 1; positions 387-812 of SEQ ID NO:1; positions 388-812 of SEQ ID NO:1; positions 389-812 of SEQ ID NO:1; positions 390-812 of SEQ ID NO:1; positions 391-812 of SEQ ID NO:1; positions 392-812 of SEQ ID NO: 1; positions 393-812 of SEQ ID NO:1; positions 394-812 of SEQ ID NO:1;

positions 395-812 of SEQ ID NO:1; positions 396-812 of SEQ ID NO:1; positions 397-812 of SEQ ID NO: 1; positions 398-812 of SEQ ID NO:1; positions 399-812 of SEQ ID NO: 1; positions 400-812 of SEQ ID NO:1; positions 401-812 of SEQ ID NO:1; positions 402-812 of SEQ ID NO:1; positions 403-812 of SEQ ID NO:1; positions 404-812 of SEQ ID NO:1; positions 405-812 of SEQ ID NO:1;

positions 406-812 of SEQ ID NO:1; positions 407-812 of SEQ ID NO:1; positions 408-812 of SEQ ID NO: 1; positions 409-812 of SEQ ID NO:1; positions 410-812 of SEQ ID NO:1; positions 411-812 of SEQ ID NO:1; positions 412-812 of SEQ ID NO:1; positions 413-812 of SEQ ID NO:1; positions 414-812 of SEQ ID NO:1; positions 415-812 of SEQ ID NO:1; positions 416-812 of SEQ ID NO:1; positions 417-812 of SEQ ID NO:1; positions 418-812 of SEQ ID NO:1; positions 419-812 of SEQ ID NO: 1; positions 420-812 of SEQ ID NO:1; positions 421-812 of SEQ ID NO:1; positions 422-812 of

SEQ ID NO:1; positions 423-812 of SEQ ID NO:1; positions 424-812 of SEQ ID NO:1; positions 425-812 of SEQ ID NO:1; positions 426-812 of SEQ ID NO:1; positions 427-812 of SEQ ID NO:1; positions 428-812 of SEQ ID NO:1; positions 429-812 of SEQ ID NO: 1; positions 430-812 of SEQ ID NO: 1; positions 431-812 of SEQ ID NO:1; positions 432-812 of SEQ ID NO:1; positions 433-812 of SEQ ID NO:1; positions 434-812 of SEQ ID NO:1; positions 435-812 of SEQ ID NO:1; positions 436-812 of SEQ ID NO:1; positions 437-812 of SEQ ID NO:1; positions 438-812 of SEQ ID NO:1; positions 439-812 of SEQ ID NO:1; positions 440-812 of SEQ ID NO:1; positions 441-812 of SEQ ID NO: 1; positions 442-812 of SEQ ID NO:1; positions 443-812 of SEQ ID NO:1; positions 444-812 of SEQ ID NO:1; positions 445-812 of SEQ ID NO:1; positions 446-812 of SEQ ID NO:1; positions 447-812 of SEQ ID NO: 1; positions 448-812 of SEQ ID NO:1; positions 449-812 of SEQ ID NO:1; positions 450-812 of SEQ ID NO:1; positions 451-812 of SEQ ID NO:1; positions 452-812 of SEQ ID NO: 1; positions 453-812 of SEQ ID NO:1; positions 454-812 of SEQ ID NO:1; positions 455-812 of SEQ ID NO:1; positions 456-812 of SEQ ID NO:1; positions 457-812 of SEQ ID NO:1; positions 458-812 of SEQ ID NO:1; positions 459-812 of SEQ ID NO:1; positions 460-812 of SEQ ID NO:1; positions 461-812 of SEQ ID NO:1; positions 462-812 of SEQ ID NO:1; positions 463-812 of SEQ ID NO: 1; positions 464-812 of SEQ ID NO:1; positions 465-812 of SEQ ID NO:1; positions 466-812 of SEQ ID NO:1; positions 467-812 of SEQ ID NO:1; positions 468-812 of SEQ ID NO: 1; positions 469-812 of SEQ ID NO:1; positions 470-812 of SEQ ID NO:1; positions 471-812 of SEQ ID NO:1; positions 472-812 of SEQ ID NO:1; positions 473-812 of SEQ ID NO:1; positions 474-812 of SEQ ID NO: 1; positions 475-812 of SEQ ID NO:1; positions 476-812 of SEQ ID NO:1; positions 477-812 of SEQ ID NO:1; positions 478-812 of SEQ ID NO:1; positions 479-812 of SEQ ID NO:1; positions 480-812 of SEQ ID NO:1; positions 481-812 of SEQ ID NO:1; positions 482-812 of SEQ ID NO:1; positions 483-812 of SEQ ID NO:1; positions 484-812 of SEQ ID NO: 1; positions 485-812 of SEQ ID NO: 1; positions 486-812 of SEQ ID NO:1; positions 487-812 of SEQ ID NO:1; positions 488-812 of SEQ ID NO:1; positions 489-812 of SEQ ID NO:1; positions 490-812 of SEQ ID NO:1; positions 491-812 of SEQ ID NO:1; positions 492-812 of SEQ ID NO:1; positions 493-812 of SEQ ID NO:1; positions 494-812 of SEQ ID NO:1; positions 495-812 of SEQ ID NO:1; positions 496-812 of SEQ ID NO: 1; positions 497-812 of SEQ ID NO:1; positions 498-812 of SEQ ID NO:1; positions 499-812 of SEQ ID NO:1; positions 500-812 of SEQ ID NO:1; positions 501-812 of SEQ ID NO:1; positions 502-812 of SEQ ID NO:1; positions 503-812 of SEQ ID NO:1; positions 504-812 of SEQ ID NO:1; positions 505-812 of SEQ ID NO:1; positions 506-812 of SEQ ID NO:1; positions 507-812 of SEQ ID NO: 1; positions 508-812 of SEQ ID NO:1; positions 509-812 of SEQ ID NO:1; positions 510-812 of SEQ ID NO:1; positions 511-812 of SEQ ID NO:1; positions 512-812 of SEQ ID NO:1; positions 513-812 of SEQ ID NO:1; positions 514-812 of SEQ ID NO:1; positions 515-812 of SEQ ID NO:1; positions 516-812 of SEQ ID NO:1; positions 517-812 of SEQ ID NO:1; positions 518-812 of SEQ ID NO: 1; positions 519-812 of SEQ ID NO:1; positions 520-812 of SEQ ID NO:1; positions 521-812 of SEQ ID NO:1; positions 522-812 of SEQ ID NO:1; positions 523-812 of SEQ ID NO: 1; positions 524-812 of SEQ ID NO:1; positions 525-812 of SEQ ID NO:1; positions 526-812 of SEQ ID NO:1; positions 527-812 of SEQ ID NO:1; positions 528-812 of SEQ ID NO:1; positions 529-812 of SEQ ID NO: 1; positions 530-812 of SEQ ID NO:1; positions 531-812 of SEQ ID NO:1; positions 532-812 of SEQ ID NO:1; positions 533-812 of SEQ ID NO:1; positions 534-812 of SEQ ID NO:1; positions 535-812 of SEQ ID NO:1; positions 536-812 of SEQ ID NO:1; positions 537-812 of SEQ ID NO:1; positions 538-812 of SEQ ID NO:1; positions 539-812 of SEQ ID NO:1; positions 540-812 of SEQ ID NO: 1; positions 541-812 of SEQ ID NO:1; positions 542-812 of SEQ ID NO:1; positions 543-812 of SEQ ID NO:1; positions 544-812 of SEQ ID NO:1; positions 545-812 of SEQ ID NO:1; positions 546-812 of SEQ ID NO:1; positions 547-812 of SEQ ID NO:1; positions 548-812 of SEQ ID NO:1; positions 549-812 of SEQ ID NO:1; positions 550-812 of SEQ ID NO:1; positions 551-812 of SEQ ID NO: 1; positions 552-812 of SEQ ID NO:1; positions 553-812 of SEQ ID NO:1; positions 554-812 of SEQ ID NO:1; positions 555-812 of SEQ ID NO:1; positions 556-812 of SEQ ID NO:1; positions 557-812 of SEQ ID NO:1; positions 558-812 of SEQ ID NO:1; positions 559-812 of SEQ ID NO:1; positions 560-812 of SEQ ID NO:1; positions 561-812 of SEQ ID NO:1; positions 562-812 of SEQ ID NO: 1; positions 563-812 of SEQ ID NO:1; positions 564-812 of SEQ ID NO:1; positions 565-812 of SEQ ID NO:1; positions 566-812 of SEQ ID NO:1; positions 567-812 of SEQ ID NO:1; positions 568-812 of SEQ ID NO:1; positions 569-812 of SEQ ID NO:1; positions 570-812 of SEQ ID NO:1; positions 571-812 of SEQ ID NO:1; positions 572-812 of SEQ ID NO:1; positions 573-812 of SEQ ID NO: 1; positions 574-812 of SEQ ID NO:1; positions 575-812 of SEQ ID NO:1; positions 576-812 of SEQ ID NO:1; positions 577-812 of SEQ ID NO:1; positions 578-812 of SEQ ID NO:1; positions 579-812 of SEQ ID NO:1; positions 580-812 of SEQ ID NO:1; positions 581-812 of SEQ ID NO:1; positions 582-812 of SEQ ID NO:1; positions 583-812 of SEQ ID NO:1; positions 584-812 of SEQ ID NO: 1; positions 585-812 of SEQ ID NO:1; positions 586-812 of SEQ ID NO:1; positions 587-812 of SEQ ID NO:1; positions 588-812 of SEQ ID NO:1; positions 589-812 of SEQ ID NO:1; positions 590-812 of SEQ ID NO:1; positions 591-812 of SEQ ID NO:1; positions 592-812 of SEQ ID NO:1; positions 593-812 of SEQ ID NO:1; positions 594-812 of SEQ ID NO:1; positions 595-812 of SEQ ID NO: 1; positions 596-812 of SEQ ID NO:1; positions 597-812 of SEQ ID NO:1; positions 598-812 of SEQ ID NO:1; positions 599-812 of SEQ ID NO:1; positions 600-812 of SEQ ID NO:1; positions 601-812 of SEQ ID NO:1; positions 602-812 of SEQ ID NO:1; positions 603-812 of SEQ ID NO:1; positions 604-812 of SEQ ID NO:1; positions 605-812 of SEQ ID NO:1; positions 606-812 of SEQ ID NO: 1; positions 607-812 of SEQ ID NO:1; positions 608-812 of SEQ ID NO:1; positions 609-812 of SEQ ID NO:1; positions 610-812 of SEQ ID NO:1; positions 611-812 of SEQ ID NO:1; positions 612-812 of SEQ ID NO:1; positions 613-812 of SEQ ID NO:1; positions 614-812 of SEQ ID NO:1; positions 615-812 of SEQ ID NO:1; and positions 616-812 of SEQ ID NO:1.

In some embodiments, a LINC complex inhibiting polypeptide according to the present disclosure, or an inhibitory region thereof, comprises or consists essentially of the amino acid sequence corresponding to one of: positions 258-717 of SEQ ID NO:13; positions 259-717 of SEQ ID NO:13; positions 260-717 of SEQ ID NO:13; positions 261-717 of SEQ ID NO:13; positions 262-717 of SEQ ID NO: 13; positions 263-717 of SEQ ID NO:13; positions 264-717 of SEQ ID NO:13; positions 265-717 of SEQ ID NO:13; positions 266-717 of SEQ ID NO: 13; positions 267-717 of SEQ ID NO:13; positions 268-717 of SEQ ID NO:13; positions 269-717 of SEQ ID NO:13; positions 270-717 of SEQ ID NO: 13; positions 271-717 of SEQ ID NO:13; positions 272-717 of SEQ ID NO:13; positions 273-717 of SEQ ID NO: 13; positions 274-717 of SEQ ID NO:13; positions 275-717 of SEQ ID NO:13; positions 276-717 of SEQ ID NO:13; positions 277-717 of SEQ ID NO:13; positions 278-717 of SEQ ID NO: 13; positions 279-717 of SEQ ID NO:13; positions 280-717 of SEQ ID NO: 13; positions 281-717 of SEQ ID NO:13; positions 282-717 of SEQ ID NO: 13; positions 283-717 of SEQ ID NO:13; positions 284-717 of SEQ ID NO:13; positions 285-717 of SEQ ID NO:13; positions 286-717 of SEQ ID NO: 13; positions 287-717 of SEQ ID NO:13; positions 288-717 of SEQ ID NO:13; positions 289-717 of SEQ ID NO: 13; positions 290-717 of SEQ ID NO:13; positions 291-717 of SEQ ID NO: 13; positions 292-717 of SEQ ID NO:13; positions 293-717 of SEQ ID NO:13; positions 294-717 of SEQ ID NO: 13; positions 295-717 of SEQ ID NO:13; positions 296-717 of SEQ ID NO: 13; positions 297-717 of SEQ ID NO:13; positions 298-717 of SEQ ID NO: 13; positions 299-717 of SEQ ID NO: 13; positions 300-717 of SEQ ID NO:13; positions 301-717 of SEQ ID NO:13; positions 302-717 of SEQ ID NO: 13; positions 303-717 of SEQ ID NO:13; positions 304-717 of SEQ ID NO:13; positions 305-717 of SEQ ID NO:13; positions 306-717 of SEQ ID NO: 13; positions 307-717 of SEQ ID NO:13; positions 308-717 of SEQ ID NO:13; positions 309-717 of SEQ ID NO:13; positions 310-717 of SEQ ID NO: 13; positions 311-717 of SEQ ID NO:13; positions 312-717 of SEQ ID NO:13; positions 313-717 of SEQ ID NO: 13; positions 314-717 of SEQ ID NO: 13; positions 315-717 of SEQ ID NO:13; positions 316-717 of SEQ ID NO:13; positions 317-717 of SEQ ID NO:13; positions 318-717 of SEQ ID NO: 13; positions 319-717 of SEQ ID NO:13; positions 320-717 of SEQ ID NO: 13; positions 321-717 of SEQ ID NO: 13; positions 322-717 of SEQ ID NO: 13; positions 323-717 of SEQ ID NO:13; positions 324-717 of SEQ ID NO:13; positions 325-717 of SEQ ID NO: 13; positions 326-717 of SEQ ID NO: 13; positions 327-717 of SEQ ID NO:13; positions 328-717 of SEQ ID NO: 13; positions 329-717 of SEQ ID NO:13; positions 330-717 of SEQ ID NO:13; positions 331-717 of SEQ ID NO:13; positions 332-717 of SEQ ID NO:13; positions 333-717 of SEQ ID NO:13; positions 334-717 of SEQ ID NO: 13; positions 335-717 of SEQ ID NO:13; positions 336-717 of SEQ ID NO: 13; positions 337-717 of SEQ ID NO:13; positions 338-717 of SEQ ID NO:13; positions 339-717 of SEQ ID NO:13; positions 340-717 of SEQ ID NO:13; positions 341-717 of SEQ ID NO: 13; positions 342-717 of SEQ ID NO: 13; positions 343-717 of SEQ ID NO:13; positions 344-717 of SEQ ID NO:13; positions 345-717 of SEQ ID NO:13; positions 346-717 of SEQ ID NO: 13; positions 347-717 of SEQ ID NO: 13; positions 348-717 of SEQ ID NO: 13; positions 349-717 of SEQ ID NO: 13; positions 350-717 of SEQ ID NO: 13; positions 351-717 of SEQ ID NO:13; positions 352-717 of SEQ ID NO:13; positions 353-717 of SEQ ID NO:13; positions 354-717 of SEQ ID NO: 13; positions 355-717 of SEQ ID NO:13; positions 356-717 of SEQ ID NO:13; positions 357-717 of SEQ ID NO:13; positions 358-717 of SEQ ID NO: 13; positions 359-717 of SEQ ID NO:13; positions 360-717 of SEQ ID NO: 13; positions 361-717 of SEQ ID NO: 13; positions 362-717 of SEQ ID NO:13; positions 363-717 of SEQ ID NO:13; positions 364-717 of SEQ ID NO:13; positions 365-717 of SEQ ID NO:13; positions 366-717 of SEQ ID NO: 13; positions 367-717 of SEQ ID NO:13; positions 368-717 of SEQ ID NO: 13; positions 369-717 of SEQ ID NO:13; positions 370-717 of SEQ ID NO: 13; positions 371-717 of SEQ ID NO:13; positions 372-717 of SEQ ID NO:13; positions 373-717 of SEQ ID NO:13; positions 374-717 of SEQ ID NO: 13; positions 375-717 of SEQ ID NO:13; positions 376-717 of SEQ ID NO: 13; positions 377-717 of SEQ ID NO:13; positions 378-717 of SEQ ID NO: 13; positions 379-717 of SEQ ID NO:13; positions 380-717 of SEQ ID NO:13; positions 381-717 of SEQ ID NO:13; positions 382-717 of SEQ ID NO: 13; positions 383-717 of SEQ ID NO:13; positions 384-717 of SEQ ID NO:13; positions 385-717 of SEQ ID NO:13; positions 386-717 of SEQ ID NO:13; positions 387-717 of SEQ ID NO:13; positions 388-717 of SEQ ID NO:13; positions 389-717 of SEQ ID NO:13; positions 390-717 of SEQ ID NO: 13; positions 391-717 of SEQ ID NO:13; positions 392-717 of SEQ ID NO:13; positions 393-717 of SEQ ID NO: 13; positions 394-717 of SEQ ID NO:13; positions 395-717 of SEQ ID NO:13; positions 396-717 of SEQ ID NO:13; positions 397-717 of SEQ ID NO:13; positions 398-717 of SEQ ID NO: 13; positions 399-717 of SEQ ID NO:13; positions 400-717 of SEQ ID NO:13; positions 401-717 of SEQ ID NO:13; positions 402-717 of SEQ ID NO:13; positions 403-717 of SEQ ID NO:13; positions 404-717 of SEQ ID NO:13; positions 405-717 of SEQ ID NO:13; positions 406-717 of SEQ ID NO: 13; positions 407-717 of SEQ ID NO:13; positions 408-717 of SEQ ID NO:13; positions 409-717 of SEQ ID NO:13; positions 410-717 of SEQ ID NO: 13; positions 411-717 of SEQ ID NO:13; positions 412-717 of SEQ ID NO:13; positions 413-717 of SEQ ID NO:13; positions 414-717 of SEQ ID NO: 13; positions 415-717 of SEQ ID NO:13; positions 416-717 of SEQ ID NO:13; positions 417-717 of SEQ ID NO:13; positions 418-717 of SEQ ID NO: 13; positions 419-717 of SEQ ID NO:13; positions 420-717 of SEQ ID NO:13; positions 421-717 of SEQ ID NO: 13; positions 422-717 of SEQ ID NO: 13; positions 423-717 of SEQ ID NO: 13; positions 424-717 of SEQ ID NO: 13; positions 425-717 of SEQ ID NO: 13; positions 426-717 of SEQ ID NO:13; positions 427-717 of SEQ ID NO:13; positions 428-717 of SEQ ID NO:13; positions 429-717 of SEQ ID NO:13; positions 430-717 of SEQ ID NO: 13; positions 431-717 of SEQ ID NO:13; positions 432-717 of SEQ ID NO: 13; positions 433-717 of SEQ ID NO: 13; positions 434-717 of SEQ ID NO: 13; positions 435-717 of SEQ ID NO:13; positions 436-717 of SEQ ID NO:13; positions 437-717 of SEQ ID NO:13; positions 438-717 of SEQ ID NO: 13; positions 439-717 of SEQ ID NO:13; positions 440-717 of SEQ ID NO:13; positions 441-717 of SEQ ID NO: 13; positions 442-717 of SEQ ID NO: 13; positions 443-717 of SEQ ID NO:13; positions 444-717 of SEQ ID NO:13; positions 445-717 of SEQ ID NO:13; positions 446-717 of SEQ ID NO: 13; positions 447-717 of SEQ ID NO:13; positions 448-717 of SEQ ID NO: 13; positions 449-717 of SEQ ID NO: 13; positions 450-717 of SEQ ID NO:13; positions 451-717 of SEQ ID NO:13; positions 452-717 of SEQ ID NO:13; positions 453-717 of SEQ ID NO:13; positions 454-717 of SEQ ID NO: 13; positions 455-717 of SEQ ID NO:13; positions 456-717 of SEQ ID NO: 13; positions 457-717 of SEQ ID NO: 13; positions 458-717 of SEQ ID NO: 13; positions 459-717 of SEQ ID NO:13; positions 460-717 of SEQ ID NO:13; positions 461-717 of SEQ ID NO:13; positions 462-717 of SEQ ID NO: 13; positions 463-717 of SEQ ID NO:13; positions 464-717 of SEQ ID NO: 13; positions 465-717 of SEQ ID NO:13; positions 466-717 of SEQ ID NO:13; positions 467-717 of SEQ ID NO: 13; positions 468-717 of SEQ ID NO:13; positions 469-717 of SEQ ID NO:13; positions 470-717 of SEQ ID NO: 13; positions 471-717 of SEQ ID NO:13; positions 472-717 of SEQ ID NO: 13; positions 473-717 of SEQ ID NO: 13; positions 474-717 of SEQ ID NO: 13; positions 475-717 of SEQ ID NO:13; positions 476-717 of SEQ ID NO:13; positions 477-717 of SEQ ID NO:13; positions 478-717 of SEQ ID NO: 13; positions 479-717 of SEQ ID NO:13; positions 480-717 of SEQ ID NO: 13; positions 481-717 of SEQ ID NO:13; positions 482-717 of SEQ ID NO: 13; positions 483-717 of SEQ ID NO:13; positions 484-717 of SEQ ID NO:13; positions 485-717 of SEQ ID NO:13; positions 486-717 of SEQ ID NO: 13; positions 487-717 of SEQ ID NO: 13; positions 488-717 of SEQ ID NO: 13; positions 489-717 of SEQ ID NO: 13; positions 490-717 of SEQ ID NO: 13; positions 491-717 of SEQ ID NO: 13; positions 492-717 of SEQ ID NO:13; positions 493-717 of SEQ ID NO:13; positions 494-717 of SEQ ID NO: 13; positions 495-717 of SEQ ID NO:13; positions 496-717 of SEQ ID NO:13; positions 497-717 of SEQ ID NO:13; positions 498-717 of SEQ ID NO:13; positions 499-717 of SEQ ID NO:13; positions 500-717 of SEQ ID NO:13; positions 501-717 of SEQ ID NO:13; positions 502-717 of SEQ ID NO: 13; positions 503-717 of SEQ ID NO:13; positions 504-717 of SEQ ID NO:13; positions 505-717 of SEQ ID NO: 13; positions 506-717 of SEQ ID NO: 13; positions 507-717 of SEQ ID NO:13; positions 508-717 of SEQ ID NO:13; positions 509-717 of SEQ ID NO:13; positions 510-717 of SEQ ID NO: 13; positions 511-717 of SEQ ID NO:13; positions 512-717 of SEQ ID NO:13; positions 513-717 of SEQ ID NO: 13; positions 514-717 of SEQ ID NO:13; positions 515-717 of SEQ ID NO:13; positions 516-717 of SEQ ID NO:13; positions 517-717 of SEQ ID NO:13; positions 518-717 of SEQ ID NO: 13; positions 519-717 of SEQ ID NO:13; positions 520-717 of SEQ ID NO:13; positions 521-717 of SEQ ID NO:13; and positions 522-717 of SEQ ID NO:13.

In some embodiments, a LINC complex inhibiting polypeptide according to the present disclosure, or an inhibitory region thereof, does not comprise, or does not consist essentially of, the amino acid sequence corresponding to one of: positions 360-812 of SEQ ID NO:1; positions 361-812 of SEQ ID NO: 1; positions 362-812 of SEQ ID NO:1; positions 363-812 of SEQ ID NO:1; positions 364-812 of SEQ ID NO:1; positions 365-812 of SEQ ID NO:1; positions 366-812 of SEQ ID NO:1; positions 367-812 of SEQ ID NO:1; positions 368-812 of SEQ ID NO:1; positions 369-812 of SEQ ID NO:1; positions 370-812 of SEQ ID NO:1; positions 371-812 of SEQ ID NO:1; positions 372-812 of SEQ ID NO: 1; positions 373-812 of SEQ ID NO:1; positions 374-812 of SEQ ID NO:1; positions 375-812 of SEQ ID NO:1; positions 376-812 of SEQ ID NO:1; positions 377-812 of SEQ ID NO:1; positions 378-812 of SEQ ID NO:1; positions 379-812 of SEQ ID NO:1; positions 380-812 of SEQ ID NO:1; positions 381-812 of SEQ ID NO:1; positions 382-812 of SEQ ID NO:1; positions 383-812 of SEQ ID NO: 1; positions 384-812 of SEQ ID NO:1; positions 385-812 of SEQ ID NO:1; positions 386-812 of SEQ ID NO:1; positions 387-812 of SEQ ID NO:1; positions 388-812 of SEQ ID NO:1; positions 389-812 of SEQ ID NO:1; positions 390-812 of SEQ ID NO:1; positions 391-812 of SEQ ID NO:1; positions 392-812 of SEQ ID NO:1; positions 393-812 of SEQ ID NO:1; positions 394-812 of SEQ ID NO: 1; positions 395-812 of SEQ ID NO:1; positions 396-812 of SEQ ID NO:1; positions 397-812 of SEQ ID NO:1; positions 398-812 of SEQ ID NO:1; positions 399-812 of SEQ ID NO:1; positions 400-812 of SEQ ID NO:1; positions 401-812 of SEQ ID NO:1; positions 402-812 of SEQ ID NO:1; positions 403-812 of SEQ ID NO:1; positions 404-812 of SEQ ID NO:1; positions 405-812 of SEQ ID NO: 1; positions 406-812 of SEQ ID NO:1; positions 407-812 of SEQ ID NO:1; positions 408-812 of SEQ ID NO:1; positions 409-812 of SEQ ID NO:1; positions 410-812 of SEQ ID NO:1; positions 411-812 of SEQ ID NO:1; positions 412-812 of SEQ ID NO:1; positions 413-812 of SEQ ID NO:1; positions 414-812 of SEQ ID NO:1; positions 415-812 of SEQ ID NO:1; positions 416-812 of SEQ ID NO: 1; positions 417-812 of SEQ ID NO:1; positions 418-812 of SEQ ID NO:1; positions 419-812 of SEQ ID NO:1; positions 420-812 of SEQ ID NO:1; positions 421-812 of SEQ ID NO:1; positions 422-812 of SEQ ID NO:1; positions 423-812 of SEQ ID NO:1; positions 424-812 of SEQ ID NO:1; positions 425-812 of SEQ ID NO:1; positions 426-812 of SEQ ID NO:1; positions 427-812 of SEQ ID NO: 1; positions 428-812 of SEQ ID NO:1; positions 429-812 of SEQ ID NO:1; positions 430-812 of SEQ ID NO:1; positions 431-812 of SEQ ID NO:1; positions 432-812 of SEQ ID NO:1; positions 433-812 of SEQ ID NO:1; positions 434-812 of SEQ ID NO:1; positions 435-812 of SEQ ID NO:1; positions 436-812 of SEQ ID NO:1; positions 437-812 of SEQ ID NO:1; positions 438-812 of SEQ ID NO: 1; positions 439-812 of SEQ ID NO:1; positions 440-812 of SEQ ID NO:1; positions 441-812 of SEQ ID NO:1; positions 442-812 of SEQ ID NO:1; positions 443-812 of SEQ ID NO:1; positions 444-812 of SEQ ID NO:1; positions 445-812 of SEQ ID NO:1; positions 446-812 of SEQ ID NO:1; positions 447-812 of SEQ ID NO:1; positions 448-812 of SEQ ID NO: 1; positions 449-812 of SEQ ID NO: 1; positions 450-812 of SEQ ID NO:1; positions 451-812 of SEQ ID NO:1; positions 452-812 of SEQ ID NO:1; positions 453-812 of SEQ ID NO:1; positions 454-812 of SEQ ID NO:1; positions 455-812 of SEQ ID NO:1; positions 456-812 of SEQ ID NO:1; positions 457-812 of SEQ ID NO:1; positions 458-812 of SEQ ID NO:1; positions 459-812 of SEQ ID NO:1; positions 460-812 of SEQ ID NO: 1; positions 461-812 of SEQ ID NO:1; positions 462-812 of SEQ ID NO: 1; positions 463-812 of SEQ ID NO:1; positions 464-812 of SEQ ID NO:1; positions 465-812 of SEQ ID NO: 1; positions 466-812 of SEQ ID NO:1; positions 467-812 of SEQ ID NO:1; positions 468-812 of SEQ ID NO:1; positions 469-812 of SEQ ID NO:1; positions 470-812 of SEQ ID NO:1; positions 471-812 of SEQ ID NO: 1; positions 472-812 of SEQ ID NO:1; positions 473-812 of SEQ ID NO:1; positions 474-812 of SEQ ID NO:1; positions 475-812 of SEQ ID NO:1; positions 476-812 of SEQ ID NO:1; positions 477-812 of SEQ ID NO:1; positions 478-812 of SEQ ID NO:1; positions 479-812 of SEQ ID NO:1; positions 480-812 of SEQ ID NO:1; positions 481-812 of SEQ ID NO:1; positions 482-812 of SEQ ID NO: 1; positions 483-812 of SEQ ID NO:1; positions 484-812 of SEQ ID NO:1; positions 485-812 of SEQ ID NO:1; positions 486-812 of SEQ ID NO:1; positions 487-812 of SEQ ID NO:1; positions 488-812 of SEQ ID NO:1; positions 489-812 of SEQ ID NO:1; positions 490-812 of SEQ ID NO:1; positions 491-812 of SEQ ID NO:1; positions 492-812 of SEQ ID NO:1; positions 493-812 of SEQ ID NO: 1; positions 494-812 of SEQ ID NO:1; positions 495-812 of SEQ ID NO:1; positions 496-812 of SEQ ID NO:1; positions 497-812 of SEQ ID NO:1; positions 498-812 of SEQ ID NO:1; positions 499-812 of SEQ ID NO:1; positions 500-812 of SEQ ID NO:1; positions 501-812 of SEQ ID NO:1; positions 502-812 of SEQ ID NO:1; positions 503-812 of SEQ ID NO:1; positions 504-812 of SEQ ID NO: 1; positions 505-812 of SEQ ID NO:1; positions 506-812 of SEQ ID NO:1; positions 507-812 of SEQ ID NO:1; positions 508-812 of SEQ ID NO:1; positions 509-812 of SEQ ID NO:1; positions 510-812 of SEQ ID NO:1; positions 511-812 of SEQ ID NO:1; positions 512-812 of SEQ ID NO:1; positions 513-812 of SEQ ID NO:1; positions 514-812 of SEQ ID NO:1; positions 515-812 of SEQ ID NO: 1; positions 516-812 of SEQ ID NO:1; positions 517-812 of SEQ ID NO:1; positions 518-812 of SEQ ID NO:1; positions 519-812 of SEQ ID NO:1; positions 520-812 of SEQ ID NO:1; positions 521-812 of SEQ ID NO:1; positions 522-812 of SEQ ID NO:1; positions 523-812 of SEQ ID NO:1; positions 524-812 of SEQ ID NO:1; positions 525-812 of SEQ ID NO:1; positions 526-812 of SEQ ID NO: 1; positions 527-812 of SEQ ID NO:1; positions 528-812 of SEQ ID NO:1; positions 529-812 of SEQ ID NO:1; positions 530-812 of SEQ ID NO:1; positions 531-812 of SEQ ID NO:1; positions 532-812 of SEQ ID NO:1; positions 533-812 of SEQ ID NO:1; positions 534-812 of SEQ ID NO:1; positions 535-812 of SEQ ID NO:1; positions 536-812 of SEQ ID NO:1; positions 537-812 of SEQ ID NO: 1; positions 538-812 of SEQ ID NO:1; positions 539-812 of SEQ ID NO:1; positions 540-812 of SEQ ID NO:1; positions 541-812 of SEQ ID NO:1; positions 542-812 of SEQ ID NO:1; positions 543-812 of SEQ ID NO:1; positions 544-812 of SEQ ID NO:1; positions 545-812 of SEQ ID NO:1; positions 546-812 of SEQ ID NO:1; positions 547-812 of SEQ ID NO:1; positions 548-812 of SEQ ID NO: 1; positions 549-812 of SEQ ID NO:1; positions 550-812 of SEQ ID NO:1; positions 551-812 of SEQ ID NO:1; positions 552-812 of SEQ ID NO:1; positions 553-812 of SEQ ID NO:1; positions 554-812 of SEQ ID NO:1; positions 555-812 of SEQ ID NO:1; positions 556-812 of SEQ ID NO:1; positions 557-812 of SEQ ID NO:1; positions 558-812 of SEQ ID NO:1; positions 559-812 of SEQ ID NO: 1; positions 560-812 of SEQ ID NO:1; positions 561-812 of SEQ ID NO:1; positions 562-812 of SEQ ID NO:1; positions 563-812 of SEQ ID NO:1; positions 564-812 of SEQ ID NO:1; positions 565-812 of SEQ ID NO:1; positions 566-812 of SEQ ID NO:1; positions 567-812 of SEQ ID NO:1; positions 568-812 of SEQ ID NO:1; positions 569-812 of SEQ ID NO:1; positions 570-812 of SEQ ID NO: 1; positions 571-812 of SEQ ID NO:1; positions 572-812 of SEQ ID NO:1; positions 573-812 of SEQ ID NO:1; positions 574-812 of SEQ ID NO:1; positions 575-812 of SEQ ID NO:1; positions 576-812 of SEQ ID NO:1; positions 577-812 of SEQ ID NO:1; positions 578-812 of SEQ ID NO:1; positions 579-812 of SEQ ID NO:1; positions 580-812 of SEQ ID NO: 1; positions 581-812 of SEQ ID NO: 1; positions 582-812 of SEQ ID NO:1; positions 583-812 of SEQ ID NO:1; positions 584-812 of SEQ ID NO:1; positions 585-812 of SEQ ID NO:1; positions 586-812 of SEQ ID NO:1; positions 587-812 of SEQ ID NO:1; positions 588-812 of SEQ ID NO:1; positions 589-812 of SEQ ID NO:1; positions 590-812 of SEQ ID NO:1; positions 591-812 of SEQ ID NO: 1; positions 592-812 of SEQ ID NO: 1; positions 593-812 of SEQ ID NO:1; positions 594-812 of SEQ ID NO:1; positions 595-812 of SEQ ID NO:1; positions 596-812 of SEQ ID NO:1; positions 597-812 of SEQ ID NO:1; positions 598-812 of SEQ ID NO:1; positions 599-812 of SEQ ID NO:1; positions 600-812 of SEQ ID NO:1; positions 601-812 of SEQ ID NO:1; positions 602-812 of SEQ ID NO:1; positions 603-812 of SEQ ID NO: 1; positions 604-812 of SEQ ID NO:1; positions 605-812 of SEQ ID NO:1; positions 606-812 of SEQ ID NO:1; positions 607-812 of SEQ ID NO:1; positions 608-812 of SEQ ID NO:1; positions 609-812 of SEQ ID NO:1; positions 610-812 of SEQ ID NO:1; positions 611-812 of SEQ ID NO:1; positions 612-812 of SEQ ID NO:1; positions 613-812 of SEQ ID NO:1; positions 614-812 of SEQ ID NO: 1; positions 615-812 of SEQ ID NO:1; and positions 616-812 of SEQ ID NO:1.

In some embodiments, a LINC complex inhibiting polypeptide according to the present disclosure, or an inhibitory region thereof, does not comprise, or does not consist essentially of, the amino acid sequence corresponding to one of: positions 258-717 of SEQ ID NO:13; positions 259-717 of SEQ ID NO: 13; positions 260-717 of SEQ ID NO: 13; positions 261-717 of SEQ ID NO: 13; positions 262-717 of SEQ ID NO:13; positions 263-717 of SEQ ID NO:13; positions 264-717 of SEQ ID NO: 13; positions 265-717 of SEQ ID NO: 13; positions 266-717 of SEQ ID NO: 13; positions 267-717 of SEQ ID NO:13; positions 268-717 of SEQ ID NO:13; positions 269-717 of SEQ ID NO:13; positions 270-717 of SEQ ID NO: 13; positions 271-717 of SEQ ID NO:13; positions 272-717 of SEQ ID NO:13; positions 273-717 of SEQ ID NO:13; positions 274-717 of SEQ ID NO: 13; positions 275-717 of SEQ ID NO:13; positions 276-717 of SEQ ID NO:13; positions 277-717 of SEQ ID NO:13; positions 278-717 of SEQ ID NO: 13; positions 279-717 of SEQ ID NO:13; positions 280-717 of SEQ ID NO:13; positions 281-717 of SEQ ID NO:13; positions 282-717 of SEQ ID NO: 13; positions 283-717 of SEQ ID NO:13; positions 284-717 of SEQ ID NO:13; positions 285-717 of SEQ ID NO:13; positions 286-717 of SEQ ID NO: 13; positions 287-717 of SEQ ID NO:13; positions 288-717 of SEQ ID NO:13; positions 289-717 of SEQ ID NO: 13; positions 290-717 of SEQ ID NO:13; positions 291-717 of SEQ ID NO: 13; positions 292-717 of SEQ ID NO:13; positions 293-717 of SEQ ID NO:13; positions 294-717 of SEQ ID NO: 13; positions 295-717 of SEQ ID NO:13; positions 296-717 of SEQ ID NO: 13; positions 297-717 of SEQ ID NO:13; positions 298-717 of SEQ ID NO: 13; positions 299-717 of SEQ ID NO: 13; positions 300-717 of SEQ ID NO:13; positions 301-717 of SEQ ID NO:13; positions 302-717 of SEQ ID NO: 13; positions 303-717 of SEQ ID NO:13; positions 304-717 of SEQ ID NO:13; positions 305-717 of SEQ ID NO:13; positions 306-717 of SEQ ID NO: 13; positions 307-717 of SEQ ID NO:13; positions 308-717 of SEQ ID NO:13; positions 309-717 of SEQ ID NO:13; positions 310-717 of SEQ ID NO: 13; positions 311-717 of SEQ ID NO:13; positions 312-717 of SEQ ID NO:13; positions 313-717 of SEQ ID NO: 13; positions 314-717 of SEQ ID NO: 13; positions 315-717 of SEQ ID NO:13; positions 316-717 of SEQ ID NO:13; positions 317-717 of SEQ ID NO:13; positions 318-717 of SEQ ID NO: 13; positions 319-717 of SEQ ID NO:13; positions 320-717 of SEQ ID NO: 13; positions 321-717 of SEQ ID NO: 13; positions 322-717 of SEQ ID NO: 13; positions 323-717 of SEQ ID NO:13; positions 324-717 of SEQ ID NO:13; positions 325-717 of SEQ ID NO:13; positions 326-717 of SEQ ID NO: 13; positions 327-717 of SEQ ID NO:13; positions 328-717 of SEQ ID NO: 13; positions 329-717 of SEQ ID NO:13; positions 330-717 of SEQ ID NO:13; positions 331-717 of SEQ ID NO:13; positions 332-717 of SEQ ID NO:13; positions 333-717 of SEQ ID NO:13; positions 334-717 of SEQ ID NO: 13; positions 335-717 of SEQ ID NO:13; positions 336-717 of SEQ ID NO: 13; positions 337-717 of SEQ ID NO:13; positions 338-717 of SEQ ID NO:13; positions 339-717 of SEQ ID NO:13; positions 340-717 of SEQ ID NO:13; positions 341-717 of SEQ ID NO: 13; positions 342-717 of SEQ ID NO: 13; positions 343-717 of SEQ ID NO:13; positions 344-717 of SEQ ID NO:13; positions 345-717 of SEQ ID NO:13; positions 346-717 of SEQ ID NO: 13; positions 347-717 of SEQ ID NO: 13; positions 348-717 of SEQ ID NO: 13; positions 349-717 of SEQ ID NO: 13; positions 350-717 of SEQ ID NO: 13; positions 351-717 of SEQ ID NO:13; positions 352-717 of SEQ ID NO:13; positions 353-717 of SEQ ID NO:13; positions 354-717 of SEQ ID NO: 13; positions 355-717 of SEQ ID NO:13; positions 356-717 of SEQ ID NO:13; positions 357-717 of SEQ ID NO:13; positions 358-717 of SEQ ID NO: 13; positions 359-717 of SEQ ID NO:13; positions 360-717 of SEQ ID NO: 13; positions 361-717 of SEQ ID NO: 13; positions 362-717 of SEQ ID NO:13; positions 363-717 of SEQ ID NO:13; positions 364-717 of SEQ ID NO:13; positions 365-717 of SEQ ID NO:13; positions 366-717 of SEQ ID NO: 13; positions 367-717 of SEQ ID NO:13; positions 368-717 of SEQ ID NO: 13; positions 369-717 of SEQ ID NO:13; positions 370-717 of SEQ ID NO: 13; positions 371-717 of SEQ ID NO:13; positions 372-717 of SEQ ID NO:13; positions 373-717 of SEQ ID NO:13; positions 374-717 of SEQ ID NO: 13; positions 375-717 of SEQ ID NO:13; positions 376-717 of SEQ ID NO: 13; positions 377-717 of SEQ ID NO:13; positions 378-717 of SEQ ID NO: 13; positions 379-717 of SEQ ID NO:13; positions 380-717 of SEQ ID NO:13; positions 381-717 of SEQ ID NO:13; positions 382-717 of SEQ ID NO: 13; positions 383-717 of SEQ ID NO:13; positions 384-717 of SEQ ID NO:13; positions 385-717 of SEQ ID NO:13; positions 386-717 of SEQ ID NO:13; positions 387-717 of SEQ ID NO:13; positions 388-717 of SEQ ID NO:13; positions 389-717 of SEQ ID NO:13; positions 390-717 of SEQ ID NO: 13; positions 391-717 of SEQ ID NO:13; positions 392-717 of SEQ ID NO:13; positions 393-717 of SEQ ID NO: 13; positions 394-717 of SEQ ID NO:13; positions 395-717 of SEQ ID NO:13; positions 396-717 of SEQ ID NO:13; positions 397-717 of SEQ ID NO:13; positions 398-717 of SEQ ID NO: 13; positions 399-717 of SEQ ID NO:13; positions 400-717 of SEQ ID NO:13; positions 401-717 of SEQ ID NO:13; positions 402-717 of SEQ ID NO:13; positions 403-717 of SEQ ID NO:13; positions 404-717 of SEQ ID NO:13; positions 405-717 of SEQ ID NO:13; positions 406-717 of SEQ ID NO: 13; positions 407-717 of SEQ ID NO:13; positions 408-717 of SEQ ID NO:13; positions 409-717 of SEQ ID NO:13; positions 410-717 of SEQ ID NO: 13; positions 411-717 of SEQ ID NO:13; positions 412-717 of SEQ ID NO:13; positions 413-717 of SEQ ID NO:13; positions 414-717 of SEQ ID NO: 13; positions 415-717 of SEQ ID NO:13; positions 416-717 of SEQ ID NO:13; positions 417-717 of SEQ ID NO:13; positions 418-717 of SEQ ID NO: 13; positions 419-717 of SEQ ID NO:13; positions 420-717 of SEQ ID NO:13; positions 421-717 of SEQ ID NO: 13; positions 422-717 of SEQ ID NO: 13; positions 423-717 of SEQ ID NO: 13; positions 424-717 of SEQ ID NO: 13; positions 425-717 of SEQ ID NO: 13; positions 426-717 of SEQ ID NO:13; positions 427-717 of SEQ ID NO:13; positions 428-717 of SEQ ID NO:13; positions 429-717 of SEQ ID NO:13; positions 430-717 of SEQ ID NO: 13; positions 431-717 of SEQ ID NO:13; positions 432-717 of SEQ ID NO: 13; positions 433-717 of SEQ ID NO:13; positions 434-717 of SEQ ID NO: 13; positions 435-717 of SEQ ID NO:13; positions 436-717 of SEQ ID NO:13; positions 437-717 of SEQ ID NO:13; positions 438-717 of SEQ ID NO: 13; positions 439-717 of SEQ ID NO:13; positions 440-717 of SEQ ID NO:13; positions 441-717 of SEQ ID NO: 13; positions 442-717 of SEQ ID NO: 13; positions 443-717 of SEQ ID NO:13; positions 444-717 of SEQ ID NO:13; positions 445-717 of SEQ ID NO:13; positions 446-717 of SEQ ID NO: 13; positions 447-717 of SEQ ID NO:13; positions 448-717 of SEQ ID NO: 13; positions 449-717 of SEQ ID NO: 13; positions 450-717 of SEQ ID NO:13; positions 451-717 of SEQ ID NO:13; positions 452-717 of SEQ ID NO:13; positions 453-717 of SEQ ID NO:13; positions 454-717 of SEQ ID NO: 13; positions 455-717 of SEQ ID NO:13; positions 456-717 of SEQ ID NO: 13; positions 457-717 of SEQ ID NO: 13; positions 458-717 of SEQ ID NO: 13; positions 459-717 of SEQ ID NO:13; positions 460-717 of SEQ ID NO:13; positions 461-717 of SEQ ID NO:13; positions 462-717 of SEQ ID NO: 13; positions 463-717 of SEQ ID NO:13; positions 464-717 of SEQ ID NO: 13; positions 465-717 of SEQ ID NO:13; positions 466-717 of SEQ ID NO:13; positions 467-717 of SEQ ID NO: 13; positions 468-717 of SEQ ID NO:13; positions 469-717 of SEQ ID NO:13; positions 470-717 of SEQ ID NO: 13; positions 471-717 of SEQ ID NO:13; positions 472-717 of SEQ ID NO: 13; positions 473-717 of SEQ ID NO: 13; positions 474-717 of SEQ ID NO: 13; positions 475-717 of SEQ ID NO:13; positions 476-717 of SEQ ID NO:13; positions 477-717 of SEQ ID NO:13; positions 478-717 of SEQ ID NO: 13; positions 479-717 of SEQ ID NO:13; positions 480-717 of SEQ ID NO: 13; positions 481-717 of SEQ ID NO:13; positions 482-717 of SEQ ID NO: 13; positions 483-717 of SEQ ID NO:13; positions 484-717 of SEQ ID NO:13; positions 485-717 of SEQ ID NO:13; positions 486-717 of SEQ ID NO: 13; positions 487-717 of SEQ ID NO: 13; positions 488-717 of SEQ ID NO: 13; positions 489-717 of SEQ ID NO: 13; positions 490-717 of SEQ ID NO: 13; positions 491-717 of SEQ ID NO: 13; positions 492-717 of SEQ ID NO:13; positions 493-717 of SEQ ID NO:13; positions 494-717 of SEQ ID NO: 13; positions 495-717 of SEQ ID NO:13; positions 496-717 of SEQ ID NO:13; positions 497-717 of SEQ ID NO:13; positions 498-717 of SEQ ID NO:13; positions 499-717 of SEQ ID NO:13; positions 500-717 of SEQ ID NO:13; positions 501-717 of SEQ ID NO:13; positions 502-717 of SEQ ID NO: 13; positions 503-717 of SEQ ID NO:13; positions 504-717 of SEQ ID NO:13; positions 505-717 of SEQ ID NO: 13; positions 506-717 of SEQ ID NO: 13; positions 507-717 of SEQ ID NO:13; positions 508-717 of SEQ ID NO:13; positions 509-717 of SEQ ID NO:13; positions 510-717 of SEQ ID NO: 13; positions 511-717 of SEQ ID NO:13; positions 512-717 of SEQ ID NO:13; positions 513-717 of SEQ ID NO: 13; positions 514-717 of SEQ ID NO:13; positions 515-717 of SEQ ID NO:13; positions 516-717 of SEQ ID NO:13; positions 517-717 of SEQ ID NO:13; positions 518-717 of SEQ ID NO: 13; positions 519-717 of SEQ ID NO:13; positions 520-717 of SEQ ID NO:13; positions 521-717 of SEQ ID NO:13; and positions 522-717 of SEQ ID NO:13.

LINC complex inhibiting polypeptides according to the present disclosure may comprise one or more additional amino acids or sequences of amino acids. That is, the LINC complex inhibiting polypeptides may comprise one or more amino acids or sequences of amino acids in addition to the inhibitory region of the polypeptide (i.e. the region comprising an amino acid sequence corresponding to the α3 helix of the CC2 region and the SUN domain of a SUN domain-containing protein).

For example, the LINC complex inhibiting polypeptides may comprise amino acid sequence(s) to facilitate expression, folding, trafficking, processing, purification or detection.

In accordance with various aspects and embodiments according to the present disclosure, LINC complex inhibiting polypeptides may comprise a sequence preventing secretion of the polypeptide from a cell expressing the polypeptide. In some embodiments the polypeptide comprises an endoplasmic reticulum (ER) retention motif. Such sequences may be provided C-terminal to the inhibitory region of a LINC complex inhibiting polypeptide according to the present disclosure. In some embodiments, a sequence for preventing secretion of the polypeptide from a cell expressing the polypeptide (e.g. an ER retention motif) is provided at the C-terminus of the amino acid sequence of the polypeptide. In some embodiments, a sequence for preventing secretion of the polypeptide from a cell expressing the polypeptide (e.g. an ER retention motif) is not followed at the C-terminus of the polypeptide by any other amino acids.

Endoplasmic reticulum retention sequences are known in the art. In some embodiments the ER retention motif is a KDEL sequence. In some embodiments, an ER retention motif is a KDEL motif or a variant thereof effective to retain a protein comprising the motif at its C-terminus in the endoplasmic reticulum. KDEL variants may comprise or consist of an amino acid sequence conforming to the prosite motif: [K/R/H/Q/S/A]-[D/E/N/Q]-E-L (described e.g. in Hulo et al., 2006, Nucleic acids research 34: D227-D230), or a variant described by in Raykhel et al., 2007 (J. Cell Biol. 179 (6): 1193-1204) who proposed an expanded prosite motif definition. Raykhel et al. demonstrated endoplasmic retention with variants in which the 4 position (i.e. the K position) included F, WY as an alternative to KRHQSA, a range of 3 position (i.e. the D position) residues that extended far beyond DENQ, F or M in the 1 position (the L position), and D in the 2 position (i.e. the E position).

For example, a variant of the KDEL motif may e.g. be one of CDEL, KCEL or HVEL, as proposed by Raykhel et al. Thus, in some aspects disclosed herein the endoplasmic reticulum (ER) retention motif is KDEL or a variant thereof which exhibits ER retention activity.

In some embodiments, the ER retention motif comprises, or consists of, the amino acid sequence of SEQ ID NO:77, or a variant comprising one or more (e.g. 1 or 2) substitutions to the amino acid sequence of SEQ ID NO:77.

In some embodiments, LINC complex inhibiting polypeptides may comprise a signal peptide. A signal peptide may be provided N-terminal to the inhibitory region of a LINC complex inhibiting polypeptide according to the present disclosure. In some embodiments, a signal peptide is provided at the N-terminus of the amino acid sequence of the polypeptide. In some embodiments, the signal peptide is not preceded at the N-terminus of the polypeptide by any other amino acids. Signal peptides normally consist of a sequence of 5-30 hydrophobic amino acids, which form a single alpha helix. Secreted proteins and proteins expressed at the cell surface often comprise signal peptides. The signal peptide may be present at the N-terminus of the peptide/polypeptide, and may be present in the newly synthesised peptide/polypeptide. Signal peptides are often removed by cleavage, and thus are not comprised in the mature peptide/polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8:785-786) or Signal-BLAST (Frank and Sippl, 2008 Bioinformatics 24:2172-2176). In some embodiments, an N-terminal signal sequence is derived from a secretory protein or a type I transmembrane protein. In some embodiments, the secretory protein or type I transmembrane protein is selected from: human serum albumin, proinsulin, transferrin receptor, EGF receptor, pre-pro-opiomelanocortin, a carboxypeptidase, a complement protein, fibrinogen, a cytokine, a chemokine, fibrinogen, a pancreatic digestive enzyme (e.g. a protease, amylase or lipase) or an endoplasmic reticulum lumenal protein (e.g. a protein disulphide isomerase or GRP94). In some embodiments the N-terminal signal peptide is derived from human serum albumin.

In some embodiments, the signal peptide comprises a signal peptidase cleavage site. The signal peptidase cleavage site provides for removal of the signal peptide from the mature polypeptide. In some embodiments, a LINC complex inhibiting polypeptide according to the present disclosure comprises a signal peptidase cleavage site derived from a secretory protein or a type I transmembrane protein, e.g. a secretory protein or a type I transmembrane protein described hereinabove.

In some embodiments, the signal peptide comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:76. In some embodiments, the signal peptide comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of SEQ ID NO:100.

In some embodiments, LINC complex inhibiting polypeptides may comprise a detectable moiety, e.g. a fluorescent label, phosphorescent label, luminescent label, immuno-detectable label (e.g. an epitope tag), radiolabel, chemical, nucleic acid or enzymatic label. The LINC complex inhibiting polypeptide may be covalently or non-covalently labelled with the detectable moiety.

In some embodiments, the detectable moiety is provided at the N-terminus of the polypeptide (either before or after processing of the polypeptide by signal peptidase to remove any signal peptide). In some embodiments, the detectable moiety is provided at the C-terminus of the polypeptide, e.g.

downstream of (after) a sequence for preventing secretion of the polypeptide from a cell expressing the polypeptide (e.g. downstream of an ER retention motif).

In some embodiments, the detectable moiety is or comprises an epitope tag. In some embodiments, an epitope tag is selected from: a haemagglutinin A (HA), ALFA, histidine (His; e.g. 6XHis), c-Myc, glutathione S-transferase (GST), green fluorescent protein (GFP), maltose-binding protein (MBP), FLAG, E, Biotin, Protein A, Protein G, streptavidin, T7, thioredoxin, V5, or vesicular stomatitis virus glycoprotein (VSV-G) tag. In some embodiments, the detectable moiety is or comprises a moiety having detectable activity, e.g. an enzymatic activity on a given substrate. Examples of such moieties include e.g. horseradish peroxidase (HRP) and luciferase moieties.

In some embodiments, the detectable moiety is or comprises a HA tag. In some embodiments, the detectable moiety is or comprises an ALFA tag.

In some embodiments, the detectable moiety comprises, or consists of, the amino acid sequence of SEQ ID NO:78, or a variant comprising one or more (e.g. 1, 2, 3 or 4) substitutions to the amino acid sequence of SEQ ID NO:78. In some embodiments, the detectable moiety comprises, or consists of, the amino acid sequence of SEQ ID NO:79, or a variant comprising one or more (e.g. 1, 2, 3 or 4) substitutions to the amino acid sequence of SEQ ID NO:79.

In some embodiments, a LINC complex inhibiting polypeptide according to the present disclosure is provided with one of the following structures:

N-term-[signal peptide]-[detectable entity]-[inhibitory region comprising an amino acid sequence corresponding to the α3 helix of the CC2 region and the SUN domain of a SUN domain-containing protein]-[sequence preventing secretion of the polypeptide from a cell expressing the polypeptide]-C-term

N-term-[detectable entity]-[signal peptide]-[inhibitory region comprising an amino acid sequence corresponding to the α3 helix of the CC2 region and the SUN domain of a SUN domain-containing protein]-[sequence preventing secretion of the polypeptide from a cell expressing the polypeptide]-C-term

N-term-[detectable entity]-[inhibitory region comprising an amino acid sequence corresponding to the α3 helix of the CC2 region and the SUN domain of a SUN domain-containing protein]-[sequence preventing secretion of the polypeptide from a cell expressing the polypeptide]-C-term

N-term-[inhibitory region comprising an amino acid sequence corresponding to the α3 helix of the CC2 region and the SUN domain of a SUN domain-containing protein]-[sequence preventing secretion of the polypeptide from a cell expressing the polypeptide]-C-term

In some embodiments, LINC complex inhibiting polypeptides according to the present disclosure comprise one or more linker sequences between amino acid sequences. In some embodiments, a linker sequence has a length of 1-2, 1-3, 1-4, 1-5 or 1-10 amino acids. In some embodiments, a linker sequence may be provided at one or both ends of one or more of: an inhibitory region comprising an amino acid sequence corresponding to the α3 helix of the CC2 region and the SUN domain of a SUN domain-containing protein; signal peptide; sequence preventing secretion of the polypeptide from a cell expressing the polypeptide; and/or detectable entity of the LINC complex inhibiting polypeptide.

Linker sequences are known to the skilled person, and are described e.g. in Chen et al., Adv Drug Deliv Rev (2013) 65 (10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65 (10): 1357-1369. Flexible linker sequences often comprise high proportions of glycine and/or serine residues. In some embodiments, the linker sequence comprises at least one glycine residue and/or at least one serine residue. In some embodiments, the linker sequence consists of glycine and serine residues.

In preferred embodiments, the LINC complex inhibiting polypeptide is of a size (i.e. in terms of the number of amino acids making up the LINC complex inhibiting polypeptide) permitting delivery of the LINC complex inhibiting polypeptide as a gene therapy, i.e. in the form of nucleic acid encoding the polypeptide.

In some embodiments, the LINC complex inhibiting polypeptide has a size such that a polynucleotide encoding the polypeptide has a size (i.e. in terms of the number of nucleotides making up the polynucleotide) within the packaging limit of a vector for delivering the polynucleotide. In some embodiments, the LINC complex inhibiting polypeptide has a size such that a polynucleotide encoding the polypeptide has a size within the packaging limit of a vector described herein. In some embodiments, the LINC complex inhibiting polypeptide has a size such that a polynucleotide encoding the polypeptide has a size within the packaging limit of an adeno-associated virus (AAV) vector, e.g.

an AAV vector described herein. In some embodiments, the LINC complex inhibiting polypeptide has a size such that a polynucleotide encoding the polypeptide has a size within the packaging limit of a scAAV vector.

In some embodiments, the LINC complex inhibiting polypeptide consists of an amino acid sequence comprising fewer than 510 amino acids. In some embodiments, the LINC complex inhibiting polypeptide consists of an amino acid sequence comprising fewer than 457 amino acids.

In some embodiments, the LINC complex inhibiting polypeptide consists of an amino acid sequence comprising fewer than 600 amino acids, e.g. one of ≤550, ≤500, ≤450, ≤400, ≤350, ≤340, ≤330, ≤320, ≤310, ≤300, ≤290, ≤280, ≤270, ≤260, ≤250, ≤240, ≤230, ≤220 or ≤210 amino acids.

LINC complex inhibiting polypeptides according to the present disclosure may be prepared according to methods for the production of polypeptides known to the skilled person.

Polypeptides may be prepared by chemical synthesis, e.g. liquid or solid phase synthesis. For example, peptides/polypeptides can be synthesised using the methods described in, for example, Chandrudu et al., Molecules (2013), 18:4373-4388, which is hereby incorporated by reference in its entirety. Alternatively, antigen-binding molecules and polypeptides may be produced by recombinant expression. Molecular biology techniques suitable for recombinant production of polypeptides are well known in the art, such as those set out in Green and Sambrook, Molecular Cloning: A Laboratory Manual (4th Edition), Cold Spring Harbor Press, 2012, and in Nat Methods. (2008); 5 (2): 135-146 both of which are hereby incorporated by reference in their entirety. Methods for the recombinant production of antigen-binding molecules are also described in Frenzel et al., Front Immunol. (2013); 4:217 and Kunert and Reinhart, Appl Microbiol Biotechnol. (2016) 100:3451-3461, both of which are hereby incorporated by reference in their entirety.

Nucleic acids and vectors encoding the LINC complex inhibiting polypeptides

The present disclosure provides nucleic acids encoding LINC complex inhibiting polypeptides according to the present disclosure. In some embodiments, the nucleic acids comprise or consist of DNA and/or RNA. In some embodiments, the nucleic acid is or comprises a polynucleotide. The nucleic acid may comprise or consist of a polynucleotide having a nucleotide sequence encoding a polypeptide according to the present disclosure.

A LINC complex inhibiting polypeptide according to the present disclosure may be produced within a cell by translation of RNA encoding the polypeptide. A LINC complex inhibiting polypeptide according to the present disclosure may be produced within a cell by transcription from nucleic acid encoding the polypeptide, and subsequent translation of the transcribed RNA.

In preferred embodiments, the nucleic acid has a size permitting its delivery as a gene therapy, i.e. in a suitable vector.

In some embodiments, the nucleic acid of the present disclosure consists of a nucleotide sequence having a size within the packaging limit of a vector for delivering the polynucleotide. In some embodiments, the nucleic acid consists of a nucleotide sequence having a size within the packaging limit of a vector described herein. In some embodiments, the nucleic acid consists of a nucleotide sequence having a size within the packaging limit of an adeno-associated virus (AAV) vector, e.g. an

AAV vector described herein. In some embodiments, the nucleic acid consists of a nucleotide sequence having a size within the packaging limit of a scAAV vector.

The nucleic acid according to the present disclosure may comprise a polypeptide-encoding nucleotide sequence, and may additionally comprise one or more non-polypeptide-encoding nucleotide sequence(s). Non-polypeptide-encoding nucleotide sequence(s) may be e.g. be 5′ cap, 5′ UTR, 3′ UTR and/or PolyA tail sequences.

In some embodiments, the nucleotide sequence of the nucleic acid consists of fewer than 6,000 nucleotides, e.g. one of ≤5,000, ≤4,500, ≤4,000, ≤3,500, ≤3,000, ≤2,500, ≤2,400 or ≤2,300 nucleotides.

In some embodiments, the protein-encoding nucleotide sequence of the nucleic acid of the present disclosure consists of fewer than 1,530 nucleotides. In some embodiments, the protein-encoding nucleotide sequence of the nucleic acid of the present disclosure consists of fewer than 1,371 nucleotides.

In some embodiments, the protein-encoding nucleotide sequence of the nucleic acid consists of fewer than 1,800 nucleotides, e.g. one of ≤1,650, ≤1,500, ≤1,350, ≤1,200, ≤1,050, ≤1,020, ≤990, ≤960, ≤930, ≤900, ≤870, ≤840, ≤810, ≤780, ≤750, ≤720, ≤690, ≤660 or ≤630 nucleotides.

In some embodiments, the nucleic acid is, or is comprised in, a vector. Accordingly, the present disclosure also provides vectors comprising nucleic acid encoding a LINC complex inhibiting polypeptide according to the present disclosure.

Nucleic acids and vectors according to the present disclosure may be provided in purified or isolated form, i.e. from other nucleic acid, or naturally-occurring biological material.

A “vector” as used herein refers to a nucleic acid used as a vehicle to transfer exogenous nucleic acid into a cell. The vector may be a vector for expression of the nucleic acid in the cell (i.e. the vector may be an expression vector). Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector according to the present disclosure.

The term “operably linked” may include the situation where a selected nucleic acid sequence and regulatory nucleic acid sequence (e.g. promoter and/or enhancer) are covalently linked in such a way that the expression of nucleic acid sequence under the influence or control of the regulatory sequence (thereby forming an expression cassette). Thus, a regulatory sequence is operably linked to the selected nucleic acid sequence if the regulatory sequence is capable of effecting transcription of the nucleic acid sequence. The resulting transcript(s) may then be translated into a polypeptide, e.g. a LINC complex inhibiting polypeptide.

Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 or Morgan and Boyerinas,

Biomedicines 2016 4, 9, which are both hereby incorporated by reference in their entirety. In preferred embodiments, the vector is an adeno-associated virus vector or a lentiviral vector.

In some embodiments, a vector is selected based on tropism for a cell type/tissue/organ to which it is desired to deliver the nucleic acid. In some embodiments, a vector is selected based on tropism for a cell type/tissue/organ in which it is desired to express the LINC complex inhibiting polypeptide. For example, it may be desired to deliver the nucleic acid/express the LINC complex inhibiting polypeptide in a cell type/tissue/organ affected by a disease to be treated/prevented in accordance with the present disclosure (e.g. a cell type/tissue/organ in which the symptoms of the disease manifest).

in some embodiments it is desired to deliver nucleic acid encoding a LINC complex inhibiting polypeptide to muscle cells/tissue (e.g. cardiac and/or skeletal muscle cells/tissue), and vectors having a tropism for such cells/tissue may be employed in such embodiments. In some embodiments, a vector may be cardiotropic. In some embodiments, a vector may be myotropic.

In preferred embodiments, the vector is an adeno-associated virus vector. Adeno-associated virus vectors and their use to vector gene therapy is reviewed e.g. in Wang et al., Nat. Rev. Drug Discov. (2019) 18:358-378 and Li and Samulski, Nat. Rev. Genet. (2020) 12:255-272, both of which are hereby incorporated by reference in their entirety. In some embodiments, a vector may be an adeno-associated virus vector described in Wang et al., Nat. Rev. Drug Discov. (2019) 18:358-378. In some embodiments, a vector may be an adeno-associated virus vector described in Li and Samulski, Nat. Rev. Genet. (2020) 12:255-272.

In some embodiments, the vector is a self-complementary adeno-associated virus (scAAV) vector. Self-complementary adeno-associated virus vectors are described e.g. in McCarty, Mol Ther. (2008) 16 (10): 1648-56, which is hereby incorporated by reference in its entirety. Conventional AAV have a single-stranded DNA genome, and depend on the DNA replication machinery of a transduced cell to synthesise the complementary strand, delaying transgene expression. By contrast, scAAV contain complementary sequences that spontaneously anneal upon infection, eliminating the requirement for DNA synthesis in the transduced host cell. Compared to classical, single-stranded AAV vectors, scAAV vectors have been shown to provide for accelerated onset of transgene expression, and an increased level of transgene expression.

In some embodiments, a vector may be an adeno-associated viral vector of one of the following serotypes: AAV1, AAV2, AAV2i8, AAV5, AAV6, AAV8, AAV9, AAV9.45, AAV10 or AAVrh74. In some embodiments, the vector is an AAV9 vector.

In some embodiments, a vector may be a cardiotropic adeno-associated viral vector. In some embodiments, a vector may be an adeno-associated viral vector of one of the following serotypes: AAV1, AAV8, AAV9, AAV9.45.

In some embodiments, a vector may be a skeletal muscle tropic adeno-associated viral vector. In some embodiments, a vector may be an adeno-associated viral vector of one of the following serotypes: AAV1, AAV6, AAV7, AAV8, AAV9, AAV9.45.

In some embodiments a vector comprises modification to increase binding to and/or transduction of a cell-type of interest (i.e. as compared to the level of binding/transduction by the unmodified vector). In some embodiments modification is to a capsid protein.

In some embodiments a vector comprises a capsid protein comprising a cell-targeting peptide. In some embodiments the cell-targeting peptide is a cell-targeting peptide described in Büning and Srivastava, Molecular Therapy: Methods & Clinical Development (2019) 12:248-265, which is hereby incorporated by reference in its entirety, e.g. a cell-targeting peptide shown in Table 1, 2, 3 or 4 thereof.

In some embodiments a vector comprises a capsid protein comprising substitution to one or more tyrosine residues, e.g. one or more surface-exposed tyrosine residues. In some embodiments, one or more tyrosine residues of the capsid protein are substituted with phenylalanine. In some embodiments a vector comprises a capsid protein in which one or more tyrosine residues are substituted with another amino acid as described in lida et al., Biomed Res Int. (2013) 2013:974819, which is hereby incorporated by reference in its entirety.

In some embodiments, a vector may be an adeno-associated virus vector described in Buning and Srivastava, supra. In some embodiments, a vector may be an adeno-associated virus vector described in lida et al., supra.

In some embodiments the nucleic acid/vector comprises one or more sequences for controlling expression of the nucleic acid. Accordingly, in some embodiments the nucleic acid/vector comprises a control element for inducible expression of the nucleic acid.

A sequence for controlling expression of the nucleic acid may provide for expression of the nucleic acid by cells of a particular type or tissue. For example, expression may be under the control of a cell type- or tissue-specific promoter.

Promoters for cell type- or tissue-specific expression of a nucleic acid in accordance with the present invention can be selected in accordance with the disease to be treated/prevented. For example, the promoter may drive expression in a cell type/tissue/an organ affected by the disease (e.g. a cell type/tissue/an organ in which the symptoms of the disease manifest).

In some embodiments, a promoter may provide for expression in muscle cells/tissue (e.g. cardiac and/or skeletal muscle cells/tissue). In some embodiments, a promoter may be a cardiac or cardiomyocte-specific promoter (e.g. a cTNT, a-MHC or MLC2v promoter). In some embodiments, a promoter may be a skeletal muscle/striated muscle cell-specific promoter (e.g. a MCK, MHCK7 or desmin promoter). In some embodiments, the promoter is cTNT.

In some embodiments, a promoter may be a vascular endothelial cell-specific promoter (e.g. a Tie2 promoter). In some embodiments, a promoter may be a vascular smooth muscle cell-specific promoter (e.g. a SM22a promoter). In some embodiments, a promoter may be a monocyte/macrophage-specific promoter (e.g. a LysM promoter).

A sequence for controlling expression of the nucleic acid may provide for expression of the nucleic acid in response to e.g. a given agent/signal. For example, expression may be under the control of inducible promoter. The agent may provide for inducible expression of the nucleic acid in vivo by administration of the agent to a subject having been administered with a modified cell according to the disclosure, or ex vivo/in vitro by administration of the agent to cells in culture ex vivo or in vitro.

In some embodiments a nucleic acid or vector according to the present disclosure may employ a conditional expression system for controlling expression of the nucleic acid encoding a LINC complex inhibiting polypeptide by cells comprising the nucleic acid/vector. “Conditional expression” may also be referred to herein as “inducible expression”, and refers to expression contingent on certain conditions, e.g. the presence of a particular agent. Conditional expression systems are well known in the art and are reviewed e.g. in Ryding et al. Journal of Endocrinology (2001) 171, 1-14, which is hereby incorporated by reference in its entirety.

### Cells Encoding/Comprising/Expressing the LINC Complex Inhibiting Polypeptides

The present disclosure also provides a cell comprising or expressing a LINC complex inhibiting polypeptide according to the present disclosure. Also provided is a cell comprising or expressing a nucleic acid or vector according to the present disclosure.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate). In preferred embodiments, the cell may be a human cell.

The present disclosure also provides a method for producing a cell comprising a nucleic acid or vector according to the present disclosure, or a cell comprising/expressing a LINC complex inhibiting polypeptide according to the present disclosure, the method comprising introducing a nucleic acid or vector according to the present disclosure into a cell. In some embodiments, introducing a nucleic acid/vector according to the present disclosure into a cell comprises transformation, transfection, electroporation or transduction (e.g. adeno-associated viral transduction). In some embodiments, the nucleic acid/vector is introduced to the cell in vivo, e.g. following administration of a vector according to the present disclosure (e.g. a viral vector, e.g. an adeno-associated viral vector) to a subject. In some embodiments, the nucleic acid/vector is introduced into cells in culture ex vivo or in vitro.

In some embodiments, the methods additionally comprise culturing the cell under conditions suitable for expression of the nucleic acid or vector by the cell.

The present disclosure also provides cells obtained or obtainable by the methods according to the present disclosure.

### Compositions

The present disclosure also provides compositions comprising the LINC complex inhibiting polypeptides, nucleic acids, vectors and cells described herein. In particular, the present disclosure provides pharmaceutical compositions and medicaments comprising the LINC complex inhibiting polypeptides, nucleic acids, vectors and cells of the present disclosure.

Such compositions may comprise the relevant article (i.e. the LINC complex inhibiting polypeptide/nucleic acid/vector/cell) in a formulation suitable for clinical use. The present disclosure is concerned in particular with pharmaceutical compositions/medicaments comprising nucleic acids and vectors according to the present disclosure.

The compositions of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilisers, solubilisers, surfactants (e.g., wetting agents), masking agents or colouring agents (e.g. titanium oxide).

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabiliser, solubiliser, surfactant, masking agent, colouring agent, flavouring agent or sweetening agent of a composition according to the present disclosure must also be ‘acceptable’ in the sense of being compatible with the other ingredients of the formulation. Suitable carriers, diluents, excipients, adjuvants, fillers, buffers, preservatives, anti-oxidants, lubricants, binders, stabilisers, solubilisers, surfactants, masking agents, colouring agents, flavouring agents or sweetening agents can be found in standard pharmaceutical texts, for example, Remington's ‘The Science and Practice of Pharmacy’ (Ed. A. Adejare), 23rd Edition (2020), Academic Press.

The pharmaceutical compositions/medicaments according to the present disclosure may be formulated for administration to a subject, e.g. administration via a route of administration as appropriate for the nature of the therapeutic agent and the disease to be treated/prevented. In some embodiments, a pharmaceutical composition/medicament may be formulated for parenteral, systemic, topical, intracavitary, intravascular, intravenous, intra-arterial, intramuscular, intrathecal, intraocular, intraconjunctival, intratumoral, subcutaneous, intradermal, oral or transdermal administration. In some embodiments, a pharmaceutical composition/medicament may be formulated for administration by injection or infusion, or administration by ingestion.

Medicaments and pharmaceutical compositions may be formulated for administration to a blood vessel, or to a tissue/organ of interest (e.g. a tissue/organ affected by the disease/condition affected by the condition (e.g. a tissue/organ in which symptoms of the disease/condition manifest).

The pharmaceutical compositions/medicaments may comprise the LINC complex inhibiting polypeptide/nucleic acid/vector/cell in a sterile or isotonic medium. The pharmaceutical compositions/medicaments may be provided in fluid, including gel, form. Fluid formulations may be formulated for administration by injection or infusion (e.g. via cannula) to a blood vessel, or a selected region of the human or animal body. The pharmaceutical compositions/medicaments may be provided in solid form, e.g. in lyophilised form.

The present disclosure also provides methods for producing pharmaceutical compositions/medicaments according to the present disclosure. Such methods may comprise mixing a LINC complex inhibiting polypeptide/nucleic acid/vector/cell described herein with a pharmaceutically-acceptable carrier, diluent, excipient, adjuvant, filler, buffer, preservative, anti-oxidant, lubricant, binder, stabiliser, solubiliser, surfactant, masking agent, colouring agent, flavouring agent or sweetening agent. Such methods generally include the step of bringing into association the LINC complex inhibiting polypeptide/nucleic acid/vector/cell with a carrier which constitutes one or more accessory ingredients. In general, the compositions are prepared by uniformly and intimately bringing into association the active compound with carriers (e.g., liquid carriers, finely divided solid carrier, etc.), and then shaping the product, if necessary.

LINC complex inhibiting polypeptides, nucleic acids, vectors, cells and compositions according to the present disclosure may be modified and/or formulated to facilitate delivery to, and/or uptake by, a cell/tissue of interest (e.g. cardiac and/or skeletal muscle cells/tissue).

Strategies for targeted delivery of such species are reviewed e.g. in Li et al., Int. J. Mol. Sci. (2015) 16:19518-19536 and Fu et al., Bioconjug Chem. (2014) 25 (9): 1602-1608, which are hereby incorporated by reference in their entirety.

In some embodiments, articles of the present disclosure may be encapsulated in a nanoparticle or a liposome. In some embodiments, articles of the present disclosure may be (covalently or non-covalently) associated with a cell-penetrating peptide (e.g. a protein transduction domain, trojan peptide, arginine-rich peptide, vectocell peptide), a cationic polymer, a cationic lipid or a viral carrier.

Nanoparticles may be organic, e.g. micelles, liposomes, proteins, solid-lipid particles, solid polymer particles, dendrimers, and polymer therapeutics. Nanoparticles may be inorganic, e.g. such as nanotubes or metal particles, optionally with organic molecules added. In some embodiments, a nanoparticle is a nanoparticle described in Chen et al., Mol Ther Methods Clin Dev. (2016) 3:16023, which is hereby incorporated by reference in its entirety. In some embodiments, a nanoparticle is a PLGA, polypeptide, poly (β-amino ester), DOPE, β-cyclodextrin-containing polycation, linear PEI, PAMAM dendrimer, branched PEI, chitosan or polyphosophoester nanoparticle.

In some embodiments, LINC complex inhibiting polypeptides, nucleic acids and vectors according to the present disclosure comprise modification to incorporate one or more moieties facilitating delivery to, and/or uptake by, a cell type or tissue of interest (e.g. cardiac and/or skeletal muscle cells/tissue). In some embodiments, LINC complex inhibiting polypeptides, nucleic acids and vectors according to the present disclosure are linked (e.g. chemically conjugated to) one or more moieties facilitating delivery to, and/or uptake by, a cell type or tissue of interest.

Moieties facilitating delivery to, and/or uptake by, cell types or tissues of interest are described e.g. in Benizri et al., Bioconjug Chem. (2019) 30 (2): 366-383, which is hereby incorporated by reference in its entirety. Such moieties include e.g. N-acetylgalactosamine (GalNAc), a-tocopherol, cell-penetrating peptides, nucleic acid aptamers, antibodies and antigen-binding fragments/derivatives thereof, cholesterol, squalene, polyethylene glycol (PEG), fatty acids (e.g. palmitic acid) and nucleolipid moieties.

Articles of the present disclosure may be formulated in a sustained release delivery system, in order to release the LINC complex inhibiting polypeptide, nucleic acid, vector, cell or composition at a predetermined rate. Sustained release delivery systems may maintain a constant drug/therapeutic/prophylactic concentration for a specified period of time. In some embodiments, articles of the present disclosure are formulated in a liposome, gel, implant, device, or drug-polymer conjugate e.g. hydrogel.

### Methods for Identifying LINC Complex Inhibiting Polypeptides

The present disclosure also provides methods for identifying LINC complex inhibiting polypeptides. The methods generally comprise introducing nucleic acid encoding a candidate LINC complex inhibiting polypeptide comprising an amino acid sequence corresponding to the SUN domain of a SUN domain-containing protein, or corresponding to the KASH domain of a KASH domain-containing protein, and analysing the subcellular localisation of an interaction partner for a SUN domain-containing protein (in the case where the candidate LINC complex inhibiting polypeptide comprises an amino acid sequence corresponding to the SUN domain of a SUN domain-containing protein), or analysing the subcellular localisation of an interaction partner for a KASH domain-containing protein (in the case where the candidate LINC complex inhibiting polypeptide comprises an amino acid sequence corresponding to the KASH domain of a KASH domain-containing protein), in order to determine whether the candidate LINC complex inhibiting polypeptide alters/disrupts the normal subcellular localisation of the relevant interaction partner.

More particularly, the present disclosure provides a method for identifying a LINC complex inhibiting polypeptide, comprising:
introducing nucleic acid encoding a candidate LINC complex inhibiting polypeptide, wherein the candidate LINC complex inhibiting polypeptide comprises an amino acid sequence corresponding to the SUN domain of a SUN domain-containing protein (e.g. SUN1, SUN2, SUN3, SUN5, SPAG4 or SUCO); and
subsequently analysing the cells to determine the subcellular localisation of an interaction partner for a SUN domain-containing protein (e.g. a KASH domain-containing protein, e.g. Nesprin-1, Nesprin-2, Nesprin-3, Nesprin-4, KASH5 or LRMP);
wherein a candidate LINC complex inhibiting polypeptide is determined to be a LINC complex inhibiting polypeptide where a change in the subcellular localisation of an interaction partner for a SUN domain-containing protein is detected.

Also provided is a method for identifying a LINC complex inhibiting polypeptide, comprising:

introducing nucleic acid encoding a candidate LINC complex inhibiting polypeptide, wherein the candidate LINC complex inhibiting polypeptide comprises an amino acid sequence corresponding to the KASH domain of a KASH domain-containing protein (e.g. Nesprin-1, Nesprin-2, Nesprin-3, Nesprin-4, KASH5 or LRMP); and
subsequently analysing the cells to determine the subcellular localisation of an interaction partner for a KASH domain-containing protein (e.g. a SUN domain-containing protein, e.g. SUN1, SUN2, SUN3, SUN5, SPAG4 or SUCO);
wherein a candidate LINC complex inhibiting polypeptide is determined to be a LINC complex inhibiting polypeptide where a change in the subcellular localisation of an interaction partner for a KASH domain-containing protein is detected.

It will be appreciated that a change in the subcellular localisation of the relevant interaction partner as referred to in the preceding two paragraphs refers to a change relative to the subcellular localisation of the interaction partner in control cells into which nucleic acid encoding a candidate LINC complex inhibiting polypeptide has not been introduced, or control cells into which nucleic acid encoding a control polypeptide known not to affect subcellular localisation of the relevant interaction partner has been introduced.

Such methods are preferably performed using cells in culture in vitro.

In some embodiments, introducing a nucleic acid into a cell may comprise transformation, transfection, electroporation or transduction (e.g. adeno-associated viral transduction).

In some embodiments, the nucleic acid encoding a candidate LINC complex inhibiting polypeptide may be provided in a vector, e.g. a vector according to an embodiment described herein.

The nucleic acid encoding a candidate LINC complex inhibiting polypeptide preferably comprises regulatory elements providing for expression of the candidate LINC complex inhibiting polypeptide in the cell into which the nucleic acid has been introduced. Following introduction of the nucleic acid into a cell, the cell is preferably maintained in conditions suitable for expression of the candidate LINC complex inhibiting polypeptide from the nucleic acid in the cell.

It will also be appreciated that following introduction of the nucleic acid into a cell, a period of time sufficient for a change in the subcellular localisation of the relevant interaction partner is preferably allowed to elapse before the cell is analysed in order to evaluate the subcellular localisation of the relevant interaction partner.

Subcellular localisation of a given polypeptide (e.g. a given interaction partner for a SUN domain-containing protein or a KASH domain-containing protein) may be analysed by methods that are well known to the skilled person. Such methods include antibody/reporter-based methods (western blot,

ELISA, immunohisto/cytochemistry, etc.). For example, subcellular localisation may be analysed e.g. by immunocytochemistry, or by western blot of extracts prepared from different cellular fractions. Such analysis may employ organelle markers and/or labelled proteins of known subcellular localisation.

Such methods may employ an interaction partner for a SUN domain-containing protein or a KASH domain-containing protein conjugated to a fluorescent-label, and may comprise analysis of the subcellular localisation of such species by fluorescence microscopy. Such methods may employ antibody-based detection of an interaction partner for a SUN domain-containing protein or a KASH domain-containing protein, and may comprise analysis of the subcellular localisation of such species by immunofluorescence microscopy.

In some embodiments, a candidate LINC complex inhibiting polypeptide is determined to be a LINC complex inhibiting polypeptide where a decrease in the proportion of the relevant interaction partner localised to the nuclear envelope is detected (relative to the proportion localised to the nuclear envelope in control cells into which nucleic acid encoding a candidate LINC complex inhibiting polypeptide has not been introduced, or control cells into which nucleic acid encoding a control polypeptide known not to affect subcellular localisation of the relevant interaction partner has been introduced).

In some embodiments, a candidate LINC complex inhibiting polypeptide is determined to be a LINC complex inhibiting polypeptide where an increase in the proportion of the relevant interaction partner not localised to the nuclear envelope, or an increase in the proportion of the relevant interaction partner localised to the endoplasmic reticulum is detected (relative to the proportion localised to the nuclear envelope in control cells into which nucleic acid encoding a candidate LINC complex inhibiting polypeptide has not been introduced, or control cells into which nucleic acid encoding a control polypeptide known not to affect subcellular localisation of the relevant interaction partner has been introduced).

In some embodiments, a method for identifying a LINC complex inhibiting polypeptide may comprise analysis of a candidate LINC complex inhibiting polypeptide essentially as described in Example 1 herein.

In some embodiments, a method for identifying a LINC complex inhibiting polypeptide comprises:

introducing nucleic acid encoding a candidate LINC complex inhibiting polypeptide comprising an amino acid sequence corresponding to the SUN domain of a SUN domain-containing protein into a cell; and
subsequently analysing the cells to determine the subcellular localisation of Nesprin-2; wherein a candidate LINC complex inhibiting polypeptide is determined to be a LINC complex inhibiting polypeptide where an increase in the proportion of Nesprin-2 localised to the endoplasmic reticulum is detected.

The present disclosure also provides LINC complex inhibiting polypeptides identified according to the methods of the present disclosure.

### Therapeutic and Prophylactic Applications

The LINC complex inhibiting polypeptides, nucleic acids, vectors, cells and compositions described herein find use in therapeutic and prophylactic methods.

The present disclosure provides LINC complex inhibiting polypeptides, nucleic acids, vectors, cells and compositions described herein for use in methods of medical treatment or prophylaxis. Also provided is the use of LINC complex inhibiting polypeptides, nucleic acids, vectors, cells and compositions described herein in the manufacture of medicaments for treating or preventing diseases/conditions. Also provided are methods of treating or preventing diseases/conditions, comprising administering to a subject a therapeutically-or prophylactically-effective amount of LINC complex inhibiting polypeptides, nucleic acids, vectors, cells and compositions described herein.

The terms ‘disorder’, ‘disease’ and ‘condition’ may be used interchangeably and refer to a pathological issue of a body part, organ or system which may be characterised by an identifiable group of signs or symptoms.

Therapeutic or prophylactic intervention in accordance with the present disclosure may be effective to reduce the development or progression of a disease/condition, alleviate the symptoms of a disease/condition or reduce the pathology of a disease/condition. The intervention may be effective to prevent progression of the disease/condition, e.g. to prevent worsening of, or to slow the rate of development of, the disease/condition. In some embodiments the intervention may lead to an improvement in the disease/condition, e.g. a reduction in the symptoms of the disease/condition or reduction in some other correlate of the severity/activity of the disease/condition. In some embodiments the intervention may prevent development of the disease/condition to a later stage (e.g. a more severe stage, or a chronic stage).

The terms ‘develop’, ‘developing’, and ‘development’, e.g. of a disorder, as used herein refer both to the onset of a disease as well as the progression, exacerbation or worsening of a disease state/correlate thereof.

Aspects of the present disclosure are concerned with the treatment/prevention of diseases in which LINC complex dysfunction is pathologically-implicated. Such diseases include e.g. nuclear envelopathies (e.g. laminopathies). The therapeutic and prophylactic utility of the agents and methods of the present disclosure extend to the treatment and/or prevention of any disease that would derive therapeutic/prophylactic benefit from LINC complex inhibition.

Aspects of the disclosure concern the treatment of diseases associated with mutation to a given gene or plurality of genes. Herein, diseases which are “associated with” mutations to a given gene/genes are diseases which are caused or exacerbated by such mutations, or for which such mutations are a risk factor for the development or progression of the disease. In some embodiments, the mutation gives rise to one or more of the following in cells comprising one or more copies of the mutant allele of the gene as compared to cells comprising two copies of (i.e. homozygous for) the non-mutated (wildtype) reference allele of the gene: a reduced level of a gene product (e.g. RNA and/or protein (or particular isoform thereof)) of the wildtype allele; an increased level of a gene product of a non-wildtype allele; an increased level of a gene product of the wildtype allele.

Aspects of the present disclosure concern the treatment/prevention of nuclear envelopathies. Nuclear envelopathies are diseases/pathological conditions associated with mutations to genes encoding nuclear envelope proteins (i.e. proteins contained in, or directly/indirectly associated with, the ONM, perinuclear space or INM). Nuclear envelopathies are reviewed e.g. by Chi et al., Journal of Biomedical Science (2009) 16:96, which is hereby incorporated by reference in its entirety. Nuclear envelopathies include diseases/pathological conditions associated with mutations in LMNA, LMNB1, LMNB2, EMD, LAP2, LBR, ZMPSTE24, SYNE-1 and NUP62.

Accordingly, in some embodiments, the disease/condition to be treated/prevented in accordance with the present disclosure is characterised by mutation to one or more of LMNA, LMNB1, LMNB2, EMD, LAP2, LBR, ZMPSTE24, SYNE-1 and NUP62.

In particular, aspects of the present disclosure are concerned with the treatment/prevention of laminopathies.

Laminopathies are reviewed e.g. by Burke and Stewart, Nat Rev Mol Cell Biol. (2013) 14 (1): 13-24, and Hah and Kim, Cells (2019) 8 (3): 231, which are both hereby incorporated by reference in their entirety. Laminopathies are commonly associated with tissue-specific defects in load bearing at the nuclear level, which can reduce the tolerance of cells to physical forces. In the experimental examples herein, the inventors demonstrate that LINC complex inhibition ameliorates the symptoms of a range of laminopathies.

As used herein, a “laminopathy” is a disease/pathological condition associated with mutation to a gene encoding a lamin.

Genes encoding lamins include LMNA (which encodes lamins A and C), and LMNB1, LMNB2, which encode lamins B1 and B2. Accordingly, aspects of the present disclosure concern the treatment/prevention of diseases associated with mutation to LMNA, LMNB1 and/or LMNB2.

In some embodiments the mutation is known or predicted to reduce the level of a lamin isoform encoded by the wildtype allele of a gene encoding a lamin (e.g. LMNA, LMNB1 or LMNB2). In some embodiments the mutation is a missense mutation. In some embodiments the mutation is known or predicted to result in the production of a truncated version of a lamin encoded by the wildtype allele of a gene encoding a lamin. In some embodiments the mutation is known or predicted to result in the production of a lamin which is misfolded and/or degraded.

In some embodiments the mutation is known or predicted to increase the level of a lamin isoform encoded by the wildtype allele of a gene encoding a lamin (e.g. LMNA, LMNB1 or LMNB2).

In some embodiments the mutation is known or predicted to increase the level of a disease-associated lamin variant (e.g. progerin). In some embodiments the mutation is known or predicted to increase the level of a lamin encoded by a disease-associated allele of a gene encoding a lamin.

In some embodiments the laminopathy is a skeletal muscle laminopathy. In some embodiments the laminopathy is a myopathy. In some embodiments the laminopathy is a LMNA mutation-associated myopathy.

In some embodiments, the disease to be treated/prevented in accordance with the present disclosure is characterised by one or more of myopathy, cardiomyopathy, dilated cardiomyopathy, muscular dystrophy, cardiac muscular dystrophy, skeletal muscular dystrophy, progeria, neuropathy, lipoatrophy, skeletal dysplasia, lipodystrophy, leukodystrophy or dermopathy.

In some embodiments, the disease to be treated/prevented in accordance with the present disclosure is characterised by one or more of muscular dystrophy, cardiac muscular dystrophy or skeletal muscular dystrophy.

In some embodiments the laminopathy is associated with mutation to LMNA, LMNB1 and/or LMNB2. In some embodiments the laminopathy is selected from Hutchinson-Gilford Progeria Syndrome; Emery-Dreifuss Muscular Dystrophy; Emery-Dreifuss Muscular Dystrophy 2, Autosomal Dominant; Lipodystrophy, Partial, Acquired; Epilepsy, Progressive Myoclonic, 9; Charcot-Marie-Tooth Disease, Axonal, Type 2e; Muscular Dystrophy; Lipodystrophy, Familial Partial, Type 2; Cardiomyopathy, Dilated, 1h; Pelger-Huet Anomaly; Reynolds Syndrome; Muscular Disease; Leukodystrophy; Dilated Cardiomyopathy; Muscular Dystrophy, Congenital, Lmna-Related; Mandibuloacral Dysplasia with Type a Lipodystrophy; Cardiomyopathy, Dilated, 1a; Restrictive Dermopathy, Lethal; Familial Partial Lipodystrophy; Epilepsy; Lipoatrophy with Diabetes, Leukomelanodermic Papules, Liver Steatosis, and Hypertrophic Cardiomyopathy; Leukodystrophy, Demyelinating, Adult-Onset, Autosomal Dominant; Acquired Generalized Lipodystrophy; Emery-Dreifuss Muscular Dystrophy 3, Autosomal Recessive; Charcot-Marie-Tooth Disease; Charcot-Marie-Tooth Disease, Axonal, Type 2b1; Cardiomyopathy, Dilated, 1b; Atrial Standstill 1; Limb-Girdle Muscular Dystrophy; Cardiomyopathy, Dilated, with Hypergonadotropic Hypogonadism; Heart-Hand Syndrome, Slovenian Type; Monogenic Diabetes; Arrhythmogenic Right Ventricular Cardiomyopathy; Cardiomyopathy, Dilated, 1e; Aging; Mandibular Hypoplasia, Deafness, Progeroid Features, and Lipodystrophy Syndrome; Adrenomyodystrophy; Atypical Werner Syndrome; Endometriosis; Spinocerebellar Ataxia 31; Progressive Muscular Atrophy; Neurogenic Bowel; Autosomal Dominant Leukodystrophy with Autonomic Disease; Werner Syndrome; Myopathy; Lmna-Related Dilated Cardiomyopathy; Muscular Dystrophy, Congenital, 1b; Hypertrophic Cardiomyopathy; Left Ventricular Noncompaction; Diabetes Mellitus, Noninsulin-Dependent; Arrhythmogenic Right Ventricular Dysplasia, Familial, 9; Heart Disease; Atrial Fibrillation; Cardiac Conduction Defect; Myoclonus; Progressive Myoclonus Epilepsy; Myoclonus Epilepsy; Peripheral Nervous System Disease; Tooth Disease; Atrioventricular Block; Myofibrillar Myopathy; Autosomal Dominant Limb-Girdle Muscular Dystrophy; Lmna-Related Cardiocutaneous Progeria Syndrome; Amyotrophic Lateral Sclerosis 1; Neural Tube Defects; Cervical Cancer; Neural Tube Defects, Folate-Sensitive; Cerebral Degeneration; Acanthosis Nigricans; 3-Hydroxyacyl-Coa Dehydrogenase Deficiency; Congenital Fiber-Type Disproportion; Acroosteolysis; Wolff-Parkinson-White Syndrome; Sick Sinus Syndrome; Calcinosis; Undifferentiated Pleomorphic Sarcoma; Ventricular Tachycardia, Catecholaminergic Polymorphic, 1, with or without Atrial Dysfunction and/or Dilated Cardiomyopathy; Lipodystrophy, Familial Partial, Type 1; Axonal Neuropathy; Paroxysmal Ventricular Fibrillation; Brugada Syndrome 5; Rigid Spine Muscular Dystrophy; Limb-Girdle Muscular Dystrophy Type 1b; Insulin-Resistant Acanthosis Nigricans, Type a; Generalized Lipodystrophy-Associated Progeroid Syndrome; Osteoporosis; Rigid Spine Muscular Dystrophy 1; Neuropathy; Catecholaminergic Polymorphic Ventricular Tachycardia; Cataract; Bethlem Myopathy 1; Congenital Generalized Lipodystrophy; Restrictive Cardiomyopathy; Muscular Dystrophy, Congenital Merosin-Deficient, 1a; Proximal Spinal Muscular Atrophy; Muscular Dystrophy-Dystroglycanopathy, Type B, 5; Lipodystrophy, Congenital Generalized, Type 1; Emery-Dreifuss Muscular Dystrophy 1, X-Linked; Cardiomyopathy, Dilated, 1d; Myopathy, Proximal, and Ophthalmoplegia; Muscle Tissue Disease; Ovarian Cystadenoma; Emerinopathy; Fanconi Anemia, Complementation Group a; Body Mass Index Quantitative Trait Locus 11; Myelodysplastic Syndrome; Skin Disease; Anorexia Nervosa; Spinal Muscular Atrophy; Inclusion Body Myositis; Aniridia 1; Myositis; Trichohepatoenteric Syndrome 1; Neuromuscular Disease; Nutritional Deficiency Disease; Thoracic Outlet Syndrome; Muscle Disorders; Muscular Atrophy; Hallermann-Streiff Syndrome; Rere-Related Disorders; Miller-Dieker Lissencephaly Syndrome; Lipodystrophy, Congenital Generalized, Type 4; Lipodystrophy, Familial Partial, Type 3; Wiedemann-Rautenstrauch Syndrome; Lipodystrophy, Congenital Generalized, Type 2; Ataxia Neuropathy Spectrum; Alopecia, Neurologic Defects, and Endocrinopathy Syndrome; Lipodystrophy, Familial Partial, Type 4; Second-Degree Atrioventricular Block; Acute Necrotizing Encephalopathy; Median Neuropathy; Intrinsic Cardiomyopathy; Familial Isolated Arrhythmogenic Ventricular Dysplasia, Right Dominant Form; Prolapse of Female Genital Organ; Familial Isolated Arrhythmogenic Ventricular Dysplasia, Biventricular Form; Familial Isolated Arrhythmogenic Ventricular Dysplasia, Left Dominant Form; Complete Generalized Lipodystrophy; Blood Group—Ahonen; Autosomal Semi-Dominant Severe Lipodystrophic Laminopathy; Ulnar Nerve Lesion; Pelvic Muscle Wasting; Alzheimer Disease; Stroke, Ischemic; Ataxia-Telangiectasia; Spondyloarthropathy 1; Human Immunodeficiency Virus Type 1; Neuroblastoma; Vascular Disease; Nervous System Disease; Respiratory Failure; Turner Syndrome; Carpal Tunnel Syndrome; Barrett Esophagus; Sleep Apnea; Cerebrovascular Disease; Proteasome-Associated Autoinflammatory Syndrome 1; Joubert Syndrome 1; Viral Infectious Disease; Dementia; Personality Disorder; Neuropathy, Hereditary Sensory and Autonomic, Type lii; Lowe Oculocerebrorenal Syndrome; Diabetes Mellitus; Fatty Liver Disease; Leigh Syndrome; Muscular Dystrophy, Duchenne Type; Hydrocephalus; Dermatomyositis; Hirschsprung Disease 1; Long Qt Syndrome; Angelman Syndrome; Central Nervous System Disease; Congenital Disorder of Glycosylation, Type in; Alacrima, Achalasia, and Mental Retardation Syndrome; Polycystic Ovary Syndrome; Hypoglycemia; Muscle Hypertrophy; Kearns-Sayre Syndrome; Cone-Rod Dystrophy 2; Aicardi-Goutieres Syndrome; Andersen Cardiodysrhythmic Periodic Paralysis; Muscular Dystrophy, Becker Type; Legg-Calve-Perthes Disease; Androgen Insensitivity Syndrome; Ehlers-Danlos Syndrome; Axenfeld-Rieger Syndrome; Muscular Dystrophy-Dystroglycanopathy, Type C, 5; Glomerulonephritis; Seizure Disorder; Chikungunya; West Syndrome; Ullrich Congenital Muscular Dystrophy 1; Focal Segmental Glomerulosclerosis; Walker-Warburg Syndrome; Renal Hypodysplasia/aplasia 1; Popliteal Pterygium Syndrome; Microcephaly; Childhood Type Dermatomyositis; Distal Arthrogryposis; Myocarditis; Arterial Tortuosity Syndrome; Scoliosis; Membranous Nephropathy; Microvascular Complications of Diabetes 3; Epidermolysis Bullosa; Short Syndrome; Hyperekplexia; Nonalcoholic Fatty Liver Disease; Muscular Dystrophy-Dystroglycanopathy, Type a, 4; Congenital Hydrocephalus; Ataxia, Combined Cerebellar and Peripheral, with Hearing Loss and Diabetes Mellitus; Cardiac Arrhythmia; Muscular Dystrophy-Dystroglycanopathy, Type a, 1; Ptosis; Laryngitis; Ablepharon-Macrostomia Syndrome; Supravalvular Aortic Stenosis; Myopathy, Congenital; Metabolic Encephalomyopathic Crises, Recurrent, with Rhabdomyolysis, Cardiac Arrhythmias, and Neurodegeneration; Lissencephaly 1; Polycystic Liver Disease 1 with or without Kidney Cysts; Idiopathic Inflammatory Myopathy; Epidermolysis Bullosa Simplex; Focal Segmental Glomerulosclerosis 1; Gonadal Dysgenesis; Gyrate Atrophy of Choroid and Retina; Syringomyelia; Ichthyosis Vulgaris; Arthrogryposis, Distal, Type 1a; Acute Insulin Response; Brachydactyly; Cerebellar Hypoplasia; Craniometaphyseal Dysplasia, Autosomal Dominant; Alport Syndrome 1, X-Linked; Lissencephaly; Muscular Dystrophy-Dystroglycanopathy, Type B, 6; Diarrhea 5, with Tufting Enteropathy, Congenital; Junctional Epidermolysis Bullosa; Aicardi-Goutieres Syndrome 1; Miyoshi Muscular Dystrophy; Retinitis; Marden-Walker Syndrome; Neuroretinitis; Polyglucosan Body Myopathy 1 with or without Immunodeficiency; Epidermolysis Bullosa, Junctional, Herlitz Type; Macroglossia; Parkinson Disease 15, Autosomal Recessive Early-Onset; Myopathy, Myofibrillar, 3; Microvascular Complications of Diabetes 7; Muscle Eye Brain Disease; Melkersson-Rosenthal Syndrome; Myopathy, X-Linked, with Excessive Autophagy; Choroiditis; Muscular Dystrophy, Limb-Girdle, Autosomal Recessive 8; Crouzon Syndrome with Acanthosis Nigricans; Muscular Dystrophy, Limb-Girdle, Autosomal Recessive 6; Polymicrogyria; Dystrophinopathies; Microvascular Complications of Diabetes 6; Microvascular Complications of Diabetes 4; Hypotonia; Pontocerebellar Hypoplasia; Congenital Fibrosarcoma; Intrauterine Growth Retardation, Metaphyseal Dysplasia, Adrenal Hypoplasia Congenita, and Genital Anomalies; Muscular Dystrophy, Limb-Girdle, Autosomal Recessive 7; Myopathy, Congenital, with Fiber-Type Disproportion; Amelogenesis Imperfecta, Type Ig; Refractory Anemia; Fibrosis of Extraocular Muscles, Congenital, 1; Ataxia and Polyneuropathy, Adult-Onset; Al-Ragad Syndrome; Senile Cataract; Muscular Dystrophy-Dystroglycanopathy, Type C, 1; Neuronal Migration Disorders; Ayme-Gripp Syndrome; Primary Agammaglobulinemia; Autosomal Recessive Limb-Girdle Muscular Dystrophy Type 2a; Cerebritis; Muscular Dystrophy, Congenital, Megaconial Type; Autosomal Recessive Limb-Girdle Muscular Dystrophy; Alkuraya-Kucinskas Syndrome; Muscular Dystrophy-Dystroglycanopathy, Type C, 4; Congenital Muscular Dystrophy Type 1a; Behr Syndrome; Dandy-Walker Complex; Muscular Dystrophy-Dystroglycanopathy, Type C, 2; Emery-Dreifuss Muscular Dystrophy, X-Linked; Muscular Dystrophy, Limb-Girdle, Autosomal Recessive 3; Autosomal Recessive Limb-Girdle Muscular Dystrophy Type 2d; Brain Small Vessel Disease 1 with or without Ocular Anomalies; Familial Isolated Dilated Cardiomyopathy; Epithelial Recurrent Erosion Dystrophy; Muscular Dystrophy-Dystroglycanopathy; Mycobacterium Avium Complex Infections; Autosomal Recessive Limb-Girdle Muscular Dystrophy Type 21; Visual Epilepsy; Aneurysm of Sinus of Valsalva; Autosomal Recessive Limb-Girdle Muscular Dystrophy Type 2b; Creatine Phosphokinase, Elevated Serum; Spastic Paraplegia, Ataxia, and Mental Retardation; Multinucleated Neurons, Anhydramnios, Renal Dysplasia, Cerebellar Hypoplasia, and Hydranencephaly; Patulous Eustachian Tube; Autosomal Genetic Disease; Ck Syndrome; Neuronitis; Hyperekplexia 1; Reducing Body Myopathy; Polymicrogyria, Bilateral Temporooccipital; Isolated Hyperckemia; Charcot-Marie-Tooth Disease, Axonal, Type 2b2; Cardioneuromyopathy with Hyaline Masses and Nemaline Rods; Congenital Muscular Dystrophy without Intellectual Disability; Blood Group, I System; Salih Myopathy; Adducted Thumbs Syndrome; Dural Sinus Malformation; Blood Group, Dombrock System; Blood Group, Colton System; Arthrochalasia Ehlers-Danlos Syndrome; Lama2-Related Muscular Dystrophy; Muscular Dystrophy, Congenital, with Infantile Cataract and Hypogonadism; Intrauterine Infections; Muscular Dystrophy, Congenital, Merosin-Positive; Congenital Muscular Dystrophy with Cerebellar Involvement; Fukuyama Type Muscular Dystrophy; Chronic Lymphoproliferative Disorder of Natural Killer Cells; Congenital Muscular Dystrophy with Intellectual Disability; Amelogenesis Imperfecta Hypoplastic Type, Ig; Androgen Insensitivity Syndrome, Mild; Muscular Dystrophy, Congenital, Producing Arthrogryposis; Congenital Muscular Alpha-Dystroglycanopathy with Brain and Eye Anomalies; Collagen Vi-Related Myopathy; Emery-Dreifuss Muscular Dystrophy, Dominant Type; Congenital Muscular Dystrophy Due to Dystroglycanopathy; Proximal Myopathy with Focal Depletion of Mitochondria; Infantile Scoliosis.

In some embodiments the laminopathy is a laminopathy associated with mutation to LMNA. In some embodiments the laminopathy is selected from Hutchinson-Gilford Progeria Syndrome; Dilated Cardiomyopathy; Muscular Dystrophy, Congenital, Lmna-Related; Emery-Dreifuss Muscular Dystrophy 2, Autosomal Dominant; Muscular Dystrophy; Mandibuloacral Dysplasia with Type a Lipodystrophy; Cardiomyopathy, Dilated, 1a; Charcot-Marie-Tooth Disease; Limb-Girdle Muscular Dystrophy; Cardiomyopathy, Dilated, with Hypergonadotropic Hypogonadism; Emery-Dreifuss Muscular Dystrophy 3, Autosomal Recessive; Lipodystrophy, Familial Partial, Type 2; Emery-Dreifuss Muscular Dystrophy; Charcot-Marie-Tooth Disease, Axonal, Type 2b1; Heart-Hand Syndrome, Slovenian Type; Aging; Familial Partial Lipodystrophy; Restrictive Dermopathy, Lethal; Arrhythmogenic Right Ventricular Cardiomyopathy; Tooth Disease; Heart Disease; Werner Syndrome; Hypertrophic Cardiomyopathy; Left Ventricular Noncompaction; Atrioventricular Block; Calcinosis; Acroosteolysis; Autosomal Dominant Limb-Girdle Muscular Dystrophy; Diabetes Mellitus, Noninsulin-Dependent; Osteoporosis; Atrial Fibrillation; Atrial Standstill 1; Acanthosis Nigricans; Cardiac Conduction Defect; Catecholaminergic Polymorphic Ventricular Tachycardia; Mandibular Hypoplasia, Deafness, Progeroid Features, and Lipodystrophy Syndrome; Sick Sinus Syndrome; Pelger-Huet Anomaly; Charcot-Marie-Tooth Disease, Axonal, Type 2e; Congenital Generalized Lipodystrophy; Restrictive Cardiomyopathy; Congenital Fiber-Type Disproportion; Lipodystrophy, Congenital Generalized, Type 1; Myofibrillar Myopathy; Lipodystrophy, Familial Partial, Type 1; Axonal Neuropathy; Atypical Werner Syndrome; Ovarian Cystadenoma; Fanconi Anemia, Complementation Group a; Body Mass Index Quantitative Trait Locus 11; Skin Disease; Rigid Spine Muscular Dystrophy 1; Neuromuscular Disease; Hallermann-Streiff Syndrome; Bethlem Myopathy 1; Acquired Generalized Lipodystrophy; Cardiomyopathy, Dilated, 1e; Lipodystrophy, Congenital Generalized, Type 4; Undifferentiated Pleomorphic Sarcoma; Lipodystrophy, Familial Partial, Type 3; Muscular Dystrophy, Congenital Merosin-Deficient, 1a; Proximal Spinal Muscular Atrophy; Muscular Dystrophy-Dystroglycanopathy, Type B, 5; Muscular Dystrophy, Congenital, 1b; Reynolds Syndrome; Wiedemann-Rautenstrauch Syndrome; Emery-Dreifuss Muscular Dystrophy 1, X-Linked; Lipodystrophy, Congenital Generalized, Type 2; Monogenic Diabetes; Cardiomyopathy, Dilated, 1d; Myopathy, Proximal, and Ophthalmoplegia; Muscle Tissue Disease; Lipodystrophy, Familial Partial, Type 4; Cardiomyopathy, Dilated, 1h; Second-Degree Atrioventricular Block; Median Neuropathy; Intrinsic Cardiomyopathy; Prolapse of Female Genital Organ; Complete Generalized Lipodystrophy; Rigid Spine Muscular Dystrophy; Emerinopathy; Ulnar Nerve Lesion; Limb-Girdle Muscular Dystrophy Type 1b; Lmna-Related Dilated Cardiomyopathy; Pelvic Muscle Wasting; Generalized Lipodystrophy-Associated Progeroid Syndrome; Muscular Disease; Cardiomyopathy, Dilated, 1b; Autosomal Genetic Disease; Familial Isolated Arrhythmogenic Ventricular Dysplasia, Right Dominant Form; Familial Isolated Arrhythmogenic Ventricular Dysplasia, Biventricular Form; Familial Isolated Arrhythmogenic Ventricular Dysplasia, Left Dominant Form; Lmna-Related Cardiocutaneous Progeria Syndrome; Autosomal Semi-Dominant Severe Lipodystrophic Laminopathy.

In some embodiments, the disease to the treated/prevented in accordance with the present disclosure is selected from a disease associated with a cDNA or protein variant indicated in Table 1 (which begins on the next page).

In some embodiments, the disease to the treated/prevented in accordance with the present disclosure is selected from a disease indicated in Table 1.

In some embodiments, the disease to the treated/prevented in accordance with the present disclosure is selected from a disease indicated in normal font in Table 1.

In some embodiments, the disease to the treated/prevented in accordance with the present disclosure is selected from a disease indicated in bold font in Table 1.

Further aspects of the present disclosure concern the treatment/prevention of diseases characterised by dyslipidemia, e.g. diseases characterised by hyperlipidemia. LINC complex inhibition has recently been shown to be an appropriate therapeutic/prophylactic intervention for dyslipidemias, particularly hyperlipidemias such as hypercholesterolemias-see e.g. WO 2021/010898 A1, e.g. at Example 18.

Further aspects of the present disclosure concern the treatment/prevention of diseases associated with LDL receptor deficiency (i.e. a reduced level of LDL receptor protein and/or function).

In some embodiments, the disease/condition to be treated/prevented according to the present disclosure is dyslipidemia and/or hypercholesterolemia. In some embodiments, the disease/condition is a disease/condition characterised by dyslipidemia and/or hypercholesterolemia. In some embodiments, the disease/condition is a disease/condition associated with dyslipidemia and/or hypercholesterolemia (e.g. a disease/condition for which dyslipidemia and/or hypercholesterolemia is a risk factor for the onset, development or progression of the disease/condition).

Dyslipidemia is defined as having blood lipid level that is too high or too low. The present disclosure is particularly concerned with the treatment of hyperlipidemias, where the level of lipid or lipoprotein in the blood is elevated. Hyperlipidemias includes hypertriglyceridemia, hypercholesterolemia and combined hyperlipidemia (combination of hypertriglyceridemia and hypercholesterolemia). Hyperlipidemia is associated e.g. with atherosclerosis, hypertension and cardiovascular disease.

Hyperlipidemia refers to an elevated level of lipid or lipoprotein in the blood. Hyperlipidemia includes hypertriglyceridemia, hypercholesterolemia and combined hyperlipidemia (combination of hypertriglyceridemia and hypercholesterolemia). Hyperlipidemia is associated e.g. with atherosclerosis, hypertension and cardiovascular disease.

Hypercholesterolemia refers to a high level of cholesterol in the blood. In many cases, hypercholesterolemia arises as a consequence of a high-fat diet and inactive lifestyle in combination with genetic risk factors. Hypercholesterolemia may also occur as a consequence of genetic mutation (e.g. in the case of familial hypercholesterolemia), type 2 diabetes, hypothyroidism, renal disease, or as a side effect of treatment with certain drugs, e.g. corticosteroids.

Hypercholesterolemia is described e.g. in Bhatnagar et al., BMJ (2008) 337: α993. The UK NHS defines hypercholesterolemia as blood total cholesterol level of >5 mmol/L or blood low-density lipoprotein (LDL) level of ≥3 mmol/L. The US NIH defines hypercholesterolemia as blood total cholesterol level of ≥240 mg/dL. Hypertriglyceridemia is described e.g. in Berglund et al., J. Clin. Endocrinol. Metab. (2012) 97 (9): 2969-89, and is defined by blood triglyceride level ≥150 mg/dl (≥1.7 mmol/L).

Hypercholesterolemia is a well-recognised risk factor for the development of cardiovascular disease, in particular cardiovascular disease arising as a consequence of atherosclerosis-see e.g. Nelson Prim

Care. (2013) Mar; 40 (1): 195-211. Hypercholesterolemia has also been reported to lead to steatosis and non-alcoholic liver disease-see e.g. Arguello et al., Biochim Biophys Acta (2015) 1852 (9): 1765-78.

In some embodiments, the disease characterised by hyperlipidemia may be a familial hyperlipidemia or an acquired (secondary) hyperlipidemia.

In some embodiments a familial hyperlipidemia is selected from Buerger-Gruetz syndrome, familial apoprotein CII deficiency, type Ic hyperlipoproteinemia, familial hypercholesterolemia, familial combined hyperlipidemia, familial dysbetalipoproteinemia, familial hypertriglyceridemia and type V hyperlipoproteinemia. In some embodiments a familial hyperlipidemia is familial hypercholesterolemia.

LDL receptor deficiency may arise e.g. as a result of mutation to LDLR. Accordingly, aspects of the present disclosure concern the treatment/prevention of diseases associated with mutation to LDLR. In some embodiments the mutation is known or predicted to reduce the level of one or more LDL receptor isoforms encoded by the wildtype LDLR allele and/or increase the level of one or more disease-associated LDL receptor variants. In some embodiments the disease associated with mutation to LDLR is familial hypercholesterolemia.

In some embodiments, the disease to be treated/prevented in accordance with the present disclosure is characterised by one or more of hyperlipidemia, hypercholesterolemia, atherosclerosis, stenosis or hypertension. In some embodiments, the disease to be treated/prevented in accordance with the present disclosure is characterised by atherosclerosis.

In some embodiments, the disease to be treated/prevented is selected from atherosclerosis, cardiovascular disease, stroke and a familial hyperlipidemia.

In accordance with various aspects of the present disclosure, a method of treating and/or preventing a disease/condition according to the present disclosure may comprise one or more of the following: Increasing survival of a subject having the disease;
Increasing the lifespan of a subject having the disease;
Increasing, or inhibiting the decline of, cardiac function;
Delaying the onset of decline of cardiac function;
Increasing, or inhibiting the reduction of, myocardial contractility;
Increasing, or inhibiting the reduction of, ejection fraction and/or fractional shortening;
Reducing, or inhibiting the increase in, left ventricular inner diameter;
Increasing, or inhibiting the reduction of, left ventricular posterior wall thickness; and/or
Reducing, or inhibiting the development of, atherosclerosis.

In accordance with various aspects of the present disclosure, methods are provided which are for, or which comprise (e.g. in the context of treatment/prevention of a disease/condition described herein), one or more of the following: inhibiting interaction between a SUN domain-containing protein and a KASH domain-containing protein; inhibiting formation of a LINC complex comprising a SUN domain-containing protein and a KASH domain-containing protein; disrupting a LINC complex comprising a SUN domain-containing protein and a KASH domain-containing protein; disrupting the normal subcellular localisation of a SUN domain-containing protein or a KASH domain-containing protein; increasing localisation of a constituent protein of a LINC complex (e.g. a KASH domain-containing protein) to the endoplasmic reticulum; reducing the level of a LINC complex comprising a SUN domain-containing protein and a KASH domain-containing protein; and/or inhibiting a function/activity of a LINC complex comprising a SUN domain-containing protein and a KASH domain-containing protein. Also provided are agents according to the present disclosure for use in such methods, and the use of agents according to the present disclosure in manufacture of pharmaceutical compositions or medicaments for use in such methods. It will be appreciated that the methods typically comprise administering a LINC complex inhibiting polypeptide, nucleic acid, vector, cell or composition described herein to a subject.

Similarly, one or more of the following may be observed in a subject following therapeutic or prophylactic intervention in accordance with the present disclosure (e.g. compared to the level prior to intervention): inhibition of interaction between a SUN domain-containing protein and a KASH domain-containing protein; inhibition of formation of a LINC complex comprising a SUN domain-containing protein and a KASH domain-containing protein; disruption of a LINC complex comprising a SUN domain-containing protein and a KASH domain-containing protein; disruption of the normal subcellular localisation of a SUN domain-containing protein or a KASH domain-containing protein; increased localisation of a constituent protein of a LINC complex (e.g. a KASH domain-containing protein) to the endoplasmic reticulum; a reduction in the level of a LINC complex comprising a SUN domain-containing protein and a KASH domain-containing protein; and/or inhibition of a function/activity of a LINC complex comprising a SUN domain-containing protein and a KASH domain-containing protein.

In some embodiments, therapeutic/prophylactic intervention in accordance with the present disclosure may be described as being ‘associated with’ one or more of the effects described in the preceding paragraph. The skilled person is readily able to evaluate such properties using techniques that are routinely practiced in the art.

Administration of the LINC complex inhibiting polypeptides, nucleic acids, vectors, cells and compositions of the present disclosure is preferably in a ‘therapeutically-effective’ or ‘prophylactically-effective’ amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (Ed. A. Adejare), 23rd Edition (2020), Academic Press.

In preferred embodiments, administration is of a nucleic acid/vector, or of a compositions comprising a nucleic acid/vector according to the present disclosure. In preferred embodiments, administration results in modification of a cell or cells to comprise/express a nucleic acid/vector, and/or to comprise/express a LINC complex inhibiting polypeptide according to the present disclosure.

Administration of the articles of the present disclosure may be e.g. parenteral, systemic, topical, intracavitary, intravascular, intravenous, intra-arterial, intramuscular, intrathecal, intraocular, intraconjunctival, intratumoral, subcutaneous, intradermal, oral or transdermal. Administration may be by injection, infusion or ingestion.

In some aspects and embodiments, articles of the present disclosure may be administered to a tissue/organ of interest (e.g. a tissue/organ affected by the disease/condition affected by the condition (e.g. a tissue/organ in which symptoms of the disease/condition manifest). In some aspects and embodiments, articles of the present disclosure may be administered to the blood (i.e. intravenous/intra-arterial administration) by injection or infusion (e.g. via cannula), or may be administered subcutaneously or orally.

In some aspects and embodiments in accordance with the present disclosure there may be targeted delivery of articles of the present disclosure, i.e, wherein the concentration of the relevant agent in the subject is increased in a given tissue(s)/organ(s) relative to other parts of the body. In some embodiments, the methods comprise intravascular (e.g. intravenous or intra-arterial), intramuscular or subcutaneous administration and wherein the relevant article is formulated in a targeted agent delivery system (e.g. as described hereinabove)

The particular mode and/or site of administration may be selected in accordance with the location where LINC complex inhibition is required, e.g. cardiac and/or skeletal muscle cells/tissue.

In some embodiments, therapeutic or prophylactic intervention according to the present disclosure may further comprise administering another agent for the treatment/prevention of the relevant disease/condition.

Administration of LINC complex inhibiting polypeptides, nucleic acids, vectors, cells and compositions described herein may be alone or in combination with other treatments, either simultaneously or sequentially dependent upon the condition to be treated. Simultaneous administration refers to administration with another therapeutic agent together, for example as a pharmaceutical composition containing both agents (combined preparation), or immediately after each other and optionally via the same route of administration (e.g. to the same tissue, artery, vein or other blood vessel). Sequential administration refers to administration of one agent followed after a given time interval by separate administration of another agent. It is not required that the two agents are administered by the same route, although this is the case in some embodiments. The time interval may be any time interval.

Multiple doses of the LINC complex inhibiting polypeptides, nucleic acids, vectors, cells and compositions of the present disclosure may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

In some embodiments, methods according to the present disclosure may comprise determining whether a subject has a disease described herein. In some embodiments the methods comprise diagnosing a disease described herein. Determining whether a subject has a disease described herein may comprise analysing a subject for one or more symptoms/correlates of the disease.

In some embodiments, the subject may be suspected of having or suffering from a disease, e.g. based on the presence of other symptoms indicative of the disease in the subject or in a cell/tissue/organ of the subject. In some embodiments the subject may be considered at risk of developing the disease, e.g. because of genetic predisposition or other risk factors for the disease.

In some embodiments, the methods comprise determining whether a subject has a mutation to a gene described herein. In some embodiments the methods comprise detecting a mutation to a gene described herein

Determination of mutation to a gene described herein may confirm a diagnosis or suspected diagnosis, or may confirm that the subject is at risk of developing the disease. The determination may diagnose a disease, or predisposition to a disease, for treatment/prevention with a LINC complex inhibiting polypeptide, nucleic acid, vector, cell or composition described herein.

Genetic factors may be assayed by methods known to those of ordinary skill in the art, including PCR based and sequencing assays. By determining the presence of genetic factors, e.g. in a sample obtained from a subject, a diagnosis may be confirmed, and/or a subject may be classified as being at risk of developing a disease described herein, and/or a subject may be identified as being suitable for treatment with a LINC complex inhibiting polypeptide, nucleic acid, vector, cell or composition described herein.

Assays may be performed in vitro on a sample obtained from a subject, or following processing of a sample obtained from a subject. The sample obtained from a subject may be of any kind. A biological sample may be taken from any tissue or bodily fluid, e.g. a blood sample, blood-derived sample, serum sample, lymph sample, semen sample, saliva sample, synovial fluid sample. A blood-derived sample may be a selected fraction of a patient's blood, e.g. a selected cell-containing fraction or a plasma or serum fraction. A sample may comprise a tissue sample or biopsy; or cells isolated from a subject.

In some embodiments, the methods comprise determining whether a subject comprises mutation to an allele of one or more of LMNA, LMNB1, LMNB2, EMD, LAP2, LBR, ZMPSTE24, SYNE-1 and NUP62. In some embodiments, the methods comprise determining whether a subject comprises mutation to an allele of one or more of LMNA, LMNB1 and LMNB2. In some embodiments, the methods comprise determining whether a subject comprises mutation to an allele of LMNA. In some embodiments, the methods comprise determining whether a subject comprises mutation to an allele of LDLR.

In such embodiments, detection of mutation may identify a subject as a subject to be administered a LINC complex inhibiting polypeptide, nucleic acid, vector, cell or composition described herein. Accordingly, in some embodiments the methods comprise selecting a subject determined to comprise mutation to one or more of LMNA, LMNB1, LMNB2, EMD, LAP2, LBR, ZMPSTE24, SYNE-1 and NUP62 for administration with a LINC complex inhibiting polypeptide, nucleic acid, vector, cell or composition described herein. In some embodiments the methods comprise selecting a subject determined to comprise mutation to one or more of LMNA, LMNB1 and LMNB2 for administration with a LINC complex inhibiting polypeptide, nucleic acid, vector, cell or composition described herein. In some embodiments the methods comprise selecting a subject determined to comprise mutation to LMNA for administration with a LINC complex inhibiting polypeptide, nucleic acid, vector, cell or composition described herein. In some embodiments the methods comprise selecting a subject determined to comprise mutation to LDLR for administration with a LINC complex inhibiting polypeptide, nucleic acid, vector, cell or composition described herein.

In some embodiments, the methods comprise testing a sample obtained from a subject suspected of having a disease for the presence or absence of at least one LMNA mutation; wherein the presence of at least one LMNA mutation indicates that the subject is to be administered a LINC complex inhibiting polypeptide, nucleic acid, vector, cell or composition described herein.

### Subjects

A subject in accordance with the various aspects of the present disclosure may be any animal or human. Therapeutic and prophylactic applications may be in human or animals (veterinary use). The subject to be treated with a LINC complex inhibiting polypeptide, nucleic acid, vector, cell or composition described herein may be a subject in need thereof. The subject is preferably mammalian, more preferably human. The subject may be a non-human mammal, but is more preferably human. The subject may be male or female. The subject may be a patient.

A subject may have been diagnosed with a disease or condition described herein, may be suspected of having such a disease/condition, or may be at risk of developing/contracting such a disease/condition. In embodiments according to the present disclosure, a subject may be selected for treatment according to the methods based on characterisation for certain markers of such disease/condition.

In some embodiments, a subject comprises a mutation according to any embodiment described hereinabove. In some embodiments, the subject comprises a mutation giving rise to a disease/condition described herein (e.g. a laminopathy).

### Kits

In some aspects of the present disclosure a kit of parts is provided. In some embodiments the kit may have at least one container having a predetermined quantity of a LINC complex inhibiting polypeptide, nucleic acid, vector, cell or composition described herein.

In some embodiments, the kit may comprise materials for producing a LINC complex inhibiting polypeptide, nucleic acid, vector, cell or composition described herein.

The kit may provide the LINC complex inhibiting polypeptide, nucleic acid, vector, cell or composition together with instructions for administration to a patient in order to treat a specified disease/condition.

In some embodiments the kit may further comprise at least one container having a predetermined quantity of another therapeutic agent. In such embodiments, the kit may also comprise a second medicament or pharmaceutical composition such that the two medicaments or pharmaceutical compositions may be administered simultaneously or separately such that they provide a combined treatment for the specific disease or condition.

Kits according to the present disclosure may include instructions for use, e.g. in the form of an instruction booklet or leaflet. The instructions may include a protocol for performing any one or more of the methods described herein.

### Sequence Identity

Pairwise and multiple sequence alignment for the purposes of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6 (298)) and MAFFT (Katoh and Standley 2013, Molecular Biology and Evolution, 30 (4) 772-780) software. When using such software, the default parameters, e.g. for gap penalty and extension penalty, are preferably used.

Sequences

| SEQ ID | | |
|---|---|---|
| NO: | DESCRIPTION | SEQUENCE |
| 1 | Human SUN1 | MDFSRLHMYSPPQCVPENTGYTYALSSSYSSDALDFETEHKLDPVFDSPRMSR |
| | | RSLRLATTACTLGDGEAVGADSGTSSAVSLKNRAARTTKQRRSTNKSAFSINHV |
| | | SRQVTSSGVSHGGTVSLQDAVTRRPPVLDESWIREQTTVDHFWGLDDDGDLKG |
| | | GNKAAIQGNGDVGAAAATAHNGFSCSNCSMLSERKDVLTAHPAAPGPVSRVYS |
| | | RDRNQKCDDCKGKRHLDAHPGRAGTLWHIWACAGYFLLQILRRIGAVGQAVSR |
| | | TAWSALWLAVVAPGKAASGVFWWLGIGWYQFVTLISWLNVFLLTRCLRNICKFL |
| | | VLLIPLFLLLAGLSLRGQGNFFSFLPVLNWASMHRTQRVDDPQDVFKPTTSRLKQ |
| | | PLQGDSEAFPWHWMSGVEQQVASLSGQCHHHGENLRELTTLLQKLQARVDQM |
| | | EGGAAGPSASVRDAVGQPPRETDFMAFHQEHEVRMSHLEDILGKLREKSEAIQK |
| | | ELEQTKQKTISAVGEQLLPTVEHLQLELDQLKSELSSWRHVKTGCETVDAVQER |
| | | VDVQVREMVKLLFSEDQQGGSLEQLLQRFSSQFVSKGDLQTMLRDLQLQILRNV |
| | | THHVSVTKQLPTSEAVVSAVSEAGASGITEAQARAIVNSALKLYSQDKTGMVDFA |
| | | LESGGGSILSTRCSETYETKTALMSLFGIPLWYFSQSPRVVIQPDIYPGNCWAFK |
| | | GSQGYLVVRLSMMIHPAAFTLEHIPKTLSPTGNISSAPKDFAVYGLENEYQEEGQ |
| | | LLGQFTYDQDGESLQMFQALKRPDDTAFQIVELRIFSNWGHPEYTCLYRFRVHG |
| | | EPVK |
| 2 | Human SUN1 | MDFSRLHMYSPPQCVPENTGYTYALSSSYSSDALDFETEHKLDPVFDSPRMSR |
| | nuclear region | RSLRLATTACTLGDGEAVGADSGTSSAVSLKNRAARTTKQRRSTNKSAFSINHV |
| | | SRQVTSSGVSHGGTVSLQDAVTRRPPVLDESWIREQTTVDHFWGLDDDGDLKG |
| | | GNKAAIQGNGDVGAAAATAHNGFSCSNCSMLSERKDVLTAHPAAPGPVSRVYS |
| | | RDRNQKCDDCKGKRHLDAHPGRAGTLWHIWACAGYFLLQILRRIGAVGQAVSR |
| | | TAWSALWLAVVAPGKAASGVFWWLGIGWYQFVTLISWLNVFLLTRCLRN |
| 3 | Human SUN1 | ICKFLVLLIPLFLLLAGLSL |
| | helical region | |
| 4 | Human SUN1 | RGQGNFFSFLPVLNWASMHRTQRVDDPQDVFKPTTSRLKQPLQGDSEAFPWH |
| | perinuclear space | WMSGVEQQVASLSGQCHHHGENLRELTTLLQKLQARVDQMEGGAAGPSASVR |
| | region | DAVGQPPRETDFMAFHQEHEVRMSHLEDILGKLREKSEAIQKELEQTKQKTISAV |
| | | GEQLLPTVEHLQLELDQLKSELSSWRHVKTGCETVDAVQERVDVQVREMVKLLF |
| | | SEDQQGGSLEQLLQRFSSQFVSKGDLQTMLRDLQLQILRNVTHHVSVTKQLPTS |
| | | EAVVSAVSEAGASGITEAQARAIVNSALKLYSQDKTGMVDFALESGGGSILSTRC |
| | | SETYETKTALMSLFGIPLWYFSQSPRVVIQPDIYPGNCWAFKGSQGYLVVRLSM |
| | | MIHPAAFTLEHIPKTLSPTGNISSAPKDFAVYGLENEYQEEGQLLGQFTYDQDGE |
| | | SLQMFQALKRPDDTAFQIVELRIFSNWGHPEYTCLYRFRVHGEPVK |
| 5 | Human SUN1 | MDFSRLHMYSPPQCVPENTGYTYALSSSYSSDALDFETEHKLDPVFDSPRMSR |
| | LMNA-binding | RSLRLATTACTLGDGEAVGADSGTSSAVSLKNRAARTTKQRRSTNKSAFSINHV |
| | region | SRQVTSSGVSHGGTVSLQDAVTRRPPVLDES |
| 6 | Human SUN1 | SRVYSRDRNQKCDDCKGKRHLDAHPGRAGTLWHIWACAGYFLLQILRRIGAVG |
| | SYNE2-binding | QAVSRTAWSALWLAVVAPGKAASGVFWWLGIGWYQFVTLIS |
| | region | |
| 7 | Human SUN1 EMD- | CKGKRHLDAHPGRAGTLWHIWACAGYFLLQILRRIGAVGQAVSRTAWSALWLAV |
| | binding region | VAPGKAASGVFWWLGIGWYQFVTLIS |
| 8 | Human SUN1 SUN | GGSILSTRCSETYETKTALMSLFGIPLWYFSQSPRVVIQPDIYPGNCWAFKGSQG |
| | domain | YLVVRLSMMIHPAAFTLEHIPKTLSPTGNISSAPKDFAVYGLENEYQEEGQLLGQF |
| | | TYDQDGESLQMFQALKRPDDTAFQIVELRIFSNWGHPEYTCLYRFRVHGEPV |
| 9 | Human SUN1 CC1 | GEQLLPTVEHLQLELDQLKSELSSWRHVKTGCETVDAVQERVDVQVREMVKLLF |
| | | SEDQQGGSLEQLLQR |
| 10 | Human SUN1 CC2 | KGDLQTMLRDLQLQILRNVTHHVSV |
| | α1 | |
| 11 | Human SUN1 CC2 | TSEAVVSAVSEA |
| | α2 | |
| 12 | Human SUN1 CC2 | ITEAQARAIVNSALKL |
| | α3 | |
| 13 | Human SUN2 | MSRRSQRLTRYSQGDDDGSSSSGGSSVAGSQSTLFKDSPLRTLKRKSSNMKRL |
| | | SPAPQLGPSSDAHTSYYSESLVHESWFPPRSSLEELHGDANWGEDLRVRRRRG |
| | | TGGSESSRASGLVGRKATEDFLGSSSGYSSEDDYVGYSDVDQQSSSSRLRSAV |
| | | SRAGSLLWMVATSPGRLFRLLYWWAGTTWYRLTTAASLLDVFVLTRRFSSLKTF |
| | | LWFLLPLLLLTCLTYGAWYFYPYGLQTFHPALVSWWAAKDSRRPDEGWEARDS |
| | | SPHFQAEQRVMSRVHSLERRLEALAAEFSSNWQKEAMRLERLELRQGAPGQG |
| | | GGGGLSHEDTLALLEGLVSRREAALKEDFRRETAARIQEELSALRAEHQQDSED |
| | | LFKKIVRASQESEARIQQLKSEWQSMTQESFQESSVKELRRLEDQLAGLQQELA |
| | | ALALKQSSVAEEVGLLPQQIQAVRDDVESQFPAWISQFLARGGGGRVGLLQREE |
| | | MQAQLRELESKILTHVAEMQGKSAREAAASLSLTLQKEGVIGVTEEQVHHIVKQA |
| | | LQRYSEDRIGLADYALESGGASVISTRCSETYETKTALLSLFGIPLWYHSQSPRVI |
| | | LQPDVHPGNCWAFQGPQGFAVVRLSARIRPTAVTLEHVPKALSPNSTISSAPKD |
| | | FAIFGFDEDLQQEGTLLGKFTYDQDGEPIQTFHFQAPTMATYQVVELRILTNWGH |
| | | PEYTCIYRFRVHGEPAH |
| 14 | Human SUN2 | MSRRSQRLTRYSQGDDDGSSSSGGSSVAGSQSTLFKDSPLRTLKRKSSNMKRL |
| | nuclear region | SPAPQLGPSSDAHTSYYSESLVHESWFPPRSSLEELHGDANWGEDLRVRRRRG |
| | | TGGSESSRASGLVGRKATEDFLGSSSGYSSEDDYVGYSDVDQQSSSSRLRSAV |
| | | SRAGSLLWMVATSPGRLFRLLYWWAGTTWYRLTTAASLLDVFVLTRRFSSLKT |
| 15 | Human SUN2 | FLWFLLPLLLLTCLTYGAWYF |
| | helical region | |
| 16 | Human SUN2 | YPYGLQTFHPALVSWWAAKDSRRPDEGWEARDSSPHFQAEQRVMSRVHSLER |
| | perinuclear space | RLEALAAEFSSNWQKEAMRLERLELRQGAPGQGGGGGLSHEDTLALLEGLVSR |
| | region | REAALKEDFRRETAARIQEELSALRAEHQQDSEDLFKKIVRASQESEARIQQLKS |
| | | EWQSMTQESFQESSVKELRRLEDQLAGLQQELAALALKQSSVAEEVGLLPQQIQ |
| | | AVRDDVESQFPAWISQFLARGGGGRVGLLQREEMQAQLRELESKILTHVAEMQ |
| | | GKSAREAAASLSLTLQKEGVIGVTEEQVHHIVKQALQRYSEDRIGLADYALESGG |
| | | ASVISTRCSETYETKTALLSLFGIPLWYHSQSPRVILQPDVHPGNCWAFQGPQGF |
| | | AVVRLSARIRPTAVTLEHVPKALSPNSTISSAPKDFAIFGFDEDLQQEGTLLGKFT |
| | | YDQDGEPIQTFHFQAPTMATYQVVELRILTNWGHPEYTCIYRFRVHGEPAH |
| 17 | Human SUN2 | MSRRSQRLTRYSQGDDDGSSSSGGSSVAGSQSTLFKDSPLRTLKRKSSNMKRL |
| | LMNA-binding | SPAPQLGPSSDAHTSYYSESLVHESWFPPRSSLEELHGDANWGEDLRVRRRRG |
| | region | TGGSESSRASGLVGRKATEDFLGSSSGYSSEDD |
| 18 | Human SUN2 SUN | GASVISTRCSETYETKTALLSLFGIPLWYHSQSPRVILQPDVHPGNCWAFQGPQ |
| | domain | GFAVVRLSARIRPTAVTLEHVPKALSPNSTISSAPKDFAIFGFDEDLQQEGTLLGK |
| | | FTYDQDGEPIQTFHFQAPTMATYQVVELRILTNWGHPEYTCIYRFRVHGEPA |
| 19 | Human SUN2 CC1 | QESFQESSVKELRRLED |
| | | QLAGLQQELAALALKQSSVAEEVGLLPQQIQAVRDDVESQFPAWISQFLA |
| 20 | Human SUN2 CC2 | REEMQAQLRELESKILTHVAEMQGK |
| | α1 | |
| 21 | Human SUN2 CC2 | AAASLSLTLQKE |
| | α2 | |
| 22 | Human SUN2 CC2 | VTEEQVHHIVKQALQR |
| | α3 | |
| 23 | Human SUN3 | MSGKTKARRAAMFFRRCSEDASGSASGNALLSEDENPDANGVTRSWKIILSTML |
| | | TLTFLLVGLLNHQWLKETDVPQKSRQLYAIIAEYGSRLYKYQARLRMPKEQLELL |
| | | KKESQNLENNFRQILFLIEQIDVLKALLRDMKDGMDNNHNWNTHGDPVEDPDHT |
| | | EEVSNLVNYVLKKLREDQVEMADYALKSAGASIIEAGTSESYKNNKAKLYWHGIG |
| | | FLNHEMPPDIILQPDVYPGKCWAFPGSQGHTLIKLATKIIPTAVTMEHISEKVSPSG |
| | | NISSAPKEFSVYGITKKCEGEEIFLGQFIYNKTGTTVQTFELQHAVSEYLLCVKLNI |
| | | FSNWGHPKYTCLYRFRVHGTPGKHI |
| 24 | Human SUN5 | MPRSSRSPGDPGALLEDVAHNPRPRRIAQRGRNTSRMAEDTSPNMTWFTCFAC |
| | | SLRTQAQQVLFNTCRCKLLCQKLMEKTGILLLCAFGFWMFSIHLPSKMKVWQDD |
| | | SINGPLQSLRLYQEKVRHHSGEIQDLRGSMNQLIAKLQEMEAMSDEQKMAQKIM |
| | | KMIHGDYIEKPDFALKSIGASIDFEHTSVTYNHEKAHSYWNWIQLWNYAQPPDVIL |
| | | EPNVTPGNCWAFEGDRGQVTIQLAQKVYLSNLTLQHIPKTISLSGSLDTAPKDFVI |
| | | YGMEGSPKEEVFLGAFQFQPENIIQMFPLQNQPARAFSAVKVKISSNWGNPGFT |
| | | CLYRVRVHGSVAPPREQPHQNPYPKRD |
| 25 | Human SPAG4 | MRRSSRPGSASSSRKHTPNFFSENSSMSITSEDSKGLRSAEPGPGEPEGRRAR |
| | | GPSCGEPALSAGVPGGTTWAGSSQQKPAPRSHNWQTACGAATVRGGASEPT |
| | | GSPVVSEEPLDLLPTLDLRQEMPPPRVFKSFLSLLFQGLSVLLSLAGDVLVSMYR |
| | | EVCSIRFLFTAVSLLSLFLSAFWLGLLYLVSPLENEPKEMLTLSEYHERVRSQGQ |
| | | QLQQLQAELDKLHKEVSTVRAANSERVAKLVFQRLNEDFVRKPDYALSSVGASI |
| | | DLQKTSHDYADRNTAYFWNRFSFWNYARPPTVILEPHVFPGNCWAFEGDQGQV |
| | | VIQLPGRVQLSDITLQHPPPSVEHTGGANSAPRDFAVFGLQVYDETEVSLGKFTF |
| | | DVEKSEIQTFHLQNDPPAAFPKVKIQILSNWGHPRFTCLYRVRAHGVRTSEGAEG |
| | | SAQGPH |
| 26 | Human SUCO | MKKHRRALALVSCLFLCSLVWLPSWRVCCKESSSASASSYYSQDDNCALENED |
| | | VQFQKKDEREGPINAESLGKSGSNLPISPKEHKLKDDSIVDVQNTESKKLSPPVV |
| | | ETLPTVDLHEESSNAVVDSETVENISSSSTSEITPISKLDEIEKSGTIPIAKPSETEQ |
| | | SETDCDVGEALDASAPIEQPSFVSPPDSLVGQHIENVSSSHGKGKITKSEFESKV |
| | | SASEQGGGDPKSALNASDNLKNESSDYTKPGDIDPTSVASPKDPEDIPTFDEWK |
| | | KKVMEVEKEKSQSMHASSNGGSHATKKVQKNRNNYASVECGAKILAANPEAKS |
| | | TSAILIENMDLYMLNPCSTKIWFVIELCEPIQVKQLDIANYELFSSTPKDFLVSISDR |
| | | YPTNKWIKLGTFHGRDERNVQSFPLDEQMYAKYVKMFIKYIKVELLSHFGSEHFC |
| | | PLSLIRVFGTSMVEEYEEIADSQYHSERQELFDEDYDYPLDYNTGEDKSSKNLLG |
| | | SATNAILNMVNIAANILGAKTEDLTEGNKSISENATATAAPKMPESTPVSTPVPSP |
| | | EYVTTEVHTHDMEPSTPDTPKESPIVQLVQEEEEEASPSTVTLLGSGEQEDESSP |
| | | WFESETQIFCSELTTICCISSFSEYIYKWCSVRVALYRQRSRTALSKGKDYLVLAQ |
| | | PPLLLPAESVDVSVLQPLSGELENTNIEREAETVVLGDLSSSMHQDDLVNHTVDA |
| | | VELEPSHSQTLSQSLLLDITPEINPLPKIEVSESVEYEAGHIPSPVIPQESSVEIDNE |
| | | TEQKSESFSSIEKPSITYETNKVNELMDNIIKEDVNSMQIFTKLSETIVPPINTATVP |
| | | DNEDGEAKMNIADTAKQTLISVVDSSSLPEVKEEEQSPEDALLRGLQRTATDFYA |
| | | ELQNSTDLGYANGNLVHGSNQKESVFMRLNNRIKALEVNMSLSGRYLEELSQRY |
| | | RKQMEEMQKAFNKTIVKLQNTSRIAEEQDQRQTEAIQLLQAQLTNMTQLVSNLSA |
| | | TVAELKREVSDRQSYLVISLVLCVVLGLMLCMQRCRNTSQFDGDYISKLPKSNQY |
| | | PSPKRCFSSYDDMNLKRRTSFPLMRSKSLQLTGKEVDPNDLYIVEPLKFSPEKKK |
| | | KRCKYKIEKIETIKPEEPLHPIANGDIKGRKPFTNQRDFSNMGEVYHSSYKGPPSE |
| | | GSSETSSQSEESYFCGISACTSLQNGQSQKTKTEKRALKRRRSKVQDQGKLIKT |
| | | LIQTKSGSLPSLHDIIKGNKEITVGTFGVTAVSGHI |
| 27 | Human SUN3 SUN | GASIIEAGTSESYKNNKAKLYWHGIGFLNHEMPPDIILQPDVYPGKCWAFPGSQG |
| | domain | HTLIKLATKIIPTAVTMEHISEKVSPSGNISSAPKEFSVYGITKKCEGEEIFLGQFIYN |
| | | KTGTTVQTFELQHAVSEYLLCVKLNIFSNWGHPKYTCLYRFRVHGTPG |
| 28 | Human SUN5 SUN | GASIDFEHTSVTYNHEKAHSYWNWIQLWNYAQPPDVILEPNVTPGNCWAFEGD |
| | domain | RGQVTIQLAQKVYLSNLTLQHIPKTISLSGSLDTAPKDFVIYGMEGSPKEEVFLGA |
| | | FQFQPENIIQMFPLQNQPARAFSAVKVKISSNWGNPGFTCLYRVRVHGSVA |
| 29 | Human SPAG4 | GASIDLQKTSHDYADRNTAYFWNRFSFWNYARPPTVILEPHVFPGNCWAFEGD |
| | SUN domain | QGQVVIQLPGRVQLSDITLQHPPPSVEHTGGANSAPRDFAVFGLQVYDETEVSL |
| | | GKFTFDVEKSEIQTFHLQNDPPAAFPKVKIQILSNWGHPRFTCLYRVRAHGVRT |
| 30 | Human SUCO SUN | EKSQSMHASSNGGSHATKKVQKNRNNYASVECGAKILAANPEAKSTSAILIENM |
| | domain | DLYMLNPCSTKIWFVIELCEPIQVKQLDIANYELFSSTPKDFLVSISDRYPTNKWIK |
| | | LGTFHGRDERNVQSFPLDEQMYAKYVKMFIKYIKVELLSHFGSEHFCPLSLIRVF |
| | | GTSM |
| 31 | Human Nesprin-1 | MATSRGASRCPRDIANVMQRLQDEQEIVQKRTFTKWINSHLAKRKPPMVVDDLF |
| | | EDMKDGVKLLALLEVLSGQKLPCEQGRRMKRIHAVANIGTALKFLEGRKIKLVNIN |
| | | STDIADGRPSIVLGLMWTIILYFQIEELTSNLPQLQSLSSSASSVDSIVSSETPSPPS |
| | | KRKVTTKIQGNAKKALLKWVQYTAGKQTGIEVKDFGKSWRSGVAFHSVIHAIRPE |
| | | LVDLETVKGRSNRENLEDAFTIAETELGIPRLLDPEDVDVDKPDEKSIMTYVAQFL |
| | | KHYPDIHNASTDGQEDDEILPGFPSFANSVQNFKREDRVIFKEMKVWIEQFERDL |
| | | TRAQMVESNLQDKYQSFKHFRVQYEMKRKQIEHLIQPLHRDGKLSLDQALVKQS |
| | | WDRVTSRLFDWHIQLDKSLPAPLGTIGAWLYRAEVALREEITVQQVHEETANTIQ |
| | | RKLEQHKDLLQNTDAHKRAFHEIYRTRSVNGIPVPPDQLEDMAERFHFVSSTSEL |
| | | HLMKMEFLELKYRLLSLLVLAESKLKSWIIKYGRRESVEQLLQNYVSFIENSKFFE |
| | | QYEVTYQILKQTAEMYVKADGSVEEAENVMKFMNETTAQWRNLSVEVRSVRSM |
| | | LEEVISNWDRYGNTVASLQAWLEDAEKMLNQSENAKKDFFRNLPHWIQQHTAM |
| | | NDAGNFLIETCDEMVSRDLKQQLLLLNGRWRELFMEVKQYAQADEMDRMKKEY |
| | | TDCVVTLSAFATEAHKKLSEPLEVSFMNVKLLIQDLEDIEQRVPVMDAQYKIITKTA |
| | | HLITKESPQEEGKEMFATMSKLKEQLTKVKECYSPLLYESQQLLIPLEELEKQMTS |
| | | FYDSLGKINEIITVLEREAQSSALFKQKHQELLACQENCKKTLTLIEKGSQSVQKF |
| | | VTLSNVLKHFDQTRLQRQIADIHVAFQSMVKKTGDWKKHVETNSRLMKKFEESR |
| | | AELEKVLRIAQEGLEEKGDPEELLRRHTEFFSQLDQRVLNAFLKACDELTDILPEQ |
| | | EQQGLQEAVRKLHKQWKDLQGEAPYHLLHLKIDVEKNRFLASVEECRTELDRET |
| | | KLMPQEGSEKIIKEHRVFFSDKGPHHLCEKRLQLIEELCVKLPVRDPVRDTPGTC |
| | | HVILKELRAAIDSTYRKLMEDPDKWKDYTSRFSEFSSWISTNETQLKGIKGEAIDT |
| | | ANHGEVKRAVEEIRNGVTKRGETLSWLKSRLKVLTEVSSENEAQKQGDELAKLS |
| | | SSFKALVTLLSEVEKMLSNFGDCVQYKEIVKNSLEELISGSKEVQEQAEKILDTEN |
| | | LFEAQQLLLHHQQKTKRISAKKRDVQQQIAQAQQGEGGLPDRGHEELRKLESTL |
| | | DGLERSRERQERRIQVTLRKWERFETNKETVVRYLFQTGSSHERFLSFSSLESL |
| | | SSELEQTKEFSKRTESIAVQAENLVKEASEIPLGPQNKQLLQQQAKSIKEQVKKLE |
| | | DTLEEDIKTMEMVKTKWDHFGSNFETLSVWITEKEKELNALETSSSAMDMQISQI |
| | | KVTIQEIESKLSSIVGLEEEAQSFAQFVTTGESARIKAKLTQIRRYGEELREHAQCL |
| | | EGTILGHLSQQQKFEENLRKIQQSVSEFEDKLAVPIKICSSATETYKVLQEHMDLC |
| | | QALESLSSAITAFSASARKVVNRDSCVQEAAALQQQYEDILRRAKERQTALENLL |
| | | AHWQRLEKELSSFLTWLERGEAKASSPEMDISADRVKVEGELQLIQALQNEVVS |
| | | QASFYSKLLQLKESLFSVASKDDVKMMKLHLEQLDERWRDLPQIINKRINFLQSV |
| | | VAEHQQFDELLLSFSVWIKLFLSELQTTSEISIMDHQVALTRHKDHAAEVESKKGE |
| | | LQSLQGHLAKLGSLGRAEDLHLLQGKAEDCFQLFEEASQVVERRQLALSHLAEF |
| | | LQSHASLSGILRQLRQTVEATNSMNKNESDLIEKDLNDALQNAKALESAAVSLDGI |
| | | LSKAQYHLKIGSSEQRTSCRATADQLCGEVERIQNLLGTKQSEADALAVLKKAFQ |
| | | DQKEELLKSIEDIEERTDKERLKEPTRQALQQRLRVFNQLEDELNSHEHELCWLK |
| | | DKAKQIAQKDVAFAPEVDREINRLEVTWDDTKRLIHENQGQCCGLIDLMREYQNL |
| | | KSAVSKVLENASSVIVTRTTIKDQEDLKWAFSKHETAKNKMNYKQKDLDNFTSKG |
| | | KHLLSELKKIHSSDFSLVKTDMESTVDKWLDVSEKLEENMDRLRVSLSIWDDVLS |
| | | TRDEIEGWSNNCVPQMAENISNLDNHLRAEELLKEFESEVKNKALRLEELHSKVN |
| | | DLKELTKNLETPPDLQFIEADLMQKLEHAKEITEVAKGTLKDFTAQSTQVEKFINDI |
| | | TTWFTKVEESLMNCAQNETCEALKKVKDIQKELQSQQSNISSTQENLNSLCRKY |
| | | HSAELESLGRAMTGLIKKHEAVSQLCSKTQASLQESLEKHFSESMQEFQEWFLG |
| | | AKAAAKESSDRTGDSKVLEAKLHDLQNILDSVSDGQSKLDAVTQEGQTLYAHLS |
| | | KQIVSSIQEQITKANEEFQAFLKQCLKDKQALQDCASELGSFEDQHRKLNLWIHE |
| | | MEERFNTENLGESKQHIPEKKNEVHKVEMFLGELLAARESLDKLSQRGQLLSEE |
| | | GHGAGQEGRLCSQLLTSHQNLLRMTKEKLRSCQVALQEHEALEEALQSMWFW |
| | | VKAIQDRLACAESTLGSKDTLEKRLSQIQDILLMKGEGEVKLNMAIGKGEQALRSS |
| | | NKEGQRVIQTQLETLKEVWADIMSSSVHAQSTLESVISQWNDYVERKNQLEQW |
| | | MESVDQKIEHPLQPQPGLKEKFVLLDHLQSILSEAEDHTRALHRLIAKSRELYEKT |
| | | EDESFKDTAQEELKTQFNDIMTVAKEKMRKVEEIVKDHLMYLDAVHEFTDWLHS |
| | | AKEELHRWSDMSGDSSATQKKLSKIKELIDSREIGASRLSRVESLAPEVKQNTTA |
| | | SGCELMHTEMQALRADWKQWEDSVFQTQSCLENLVSQMALSEQEFSGQVAQL |
| | | EQALEQFSALLKTWAQQLTLLEGKNTDEEIVECWHKGQEILDALQKAEPRTEDLK |
| | | SQLNELCRFSRDLSTYSGKVSGLIKEYNCLCLQASKGCQNKEQILQQRFRKAFR |
| | | DFQQWLVNAKITTAKCFDIPQNISEVSTSLQKIQEFLSESENGQHKLNMMLSKGE |
| | | LLSTLLTKEKAKGIQAKVTAAKEDWKNFHSNLHQKESALENLKIQMKDFEVSAEPI |
| | | QDWLSKTEKMVHESSNRLYDLPAKRREQQKLQSVLEEIHCYEPQLNRLKEKAQ |
| | | QLWEGQAASKSFRHRVSQLSSQYLALSNLTKEKVSRLDRIVAEHNQFSLGIKELQ |
| | | DWMTDAIHMLDSYCHPTSDKSVLDSRTLKLEALLSVKQEKEIQMKMIVTRGESVL |
| | | QNTSPEGIPTIQQQLQSVKDMWASLLSAGIRCKSQLEGALSKWTSYQDGVRQFS |
| | | GWMDSMEANLNESERQHAELRDKTTMLGKAKLLNEEVLSYSSLLETIEVKGAGM |
| | | TEHYVTQLELQDLQERYRAIQERAKEAVTKSEKLVRLHQEYQRDLKAFEVWLGQ |
| | | EQEKLDQYSVLEGDAHTHETTLRDLQELQVHCAEGQALLNSVLHTREDVIPSGIP |
| | | QAEDRALESLRQDWQAYQHRLSETRTQFNNVVNKLRLMEQKFQQVDEWLKTA |
| | | EEKVSPRTRRQSNRATKEIQLHQMKKWHEEVTAYRDEVEEVGARAQEILDESHV |
| | | NSRMGCQATQLTSRYQALLLQVLEQIKFLEEEIQSLEESESSLSSYSDWYGSTHK |
| | | NFKNVATKIDKVDTVMMGKKLKTLEVLLKDMEKGHSLLKSAREKGERAVKYLEE |
| | | GEAERLRKEIHDHMEQLKELTSTVRKEHMTLEKGLHLAKEFSDKCKALTQWIAEY |
| | | QEILHVPEEPKMELYEKKAQLSKYKSLQQTVLSHEPSVKSVREKGEALLELVQDV |
| | | TLKDKIDQLQSDYQDLCSIGKEHVFSLEAKVKDHEDYNSELQEVEKWLLQMSGR |
| | | LVAPDLLETSSLETITQQLAHHKAMMEEIAGFEDRLNNLQMKGDTLIGQCADHLQ |
| | | AKLKQNVHAHLQGTKDSYSAICSTAQRMYQSLEHELQKHVSRQDTLQQCQAWL |
| | | SAVQPDLEPSPQPPLSRAEAIKQVKHFRALQEQARTYLDLLCSMCDLSNASVKTT |
| | | AKDIQQTEQTIEQKLVQAQNLTQGWEEIKHLKSELWIYLQDADQQLQNMKRRHS |
| | | ELELNIAQNMVSQVKDFVKKLQSKQASVNTIIEKVNKLTKKEESPEHKEINHLNDQ |
| | | WLDLCRQSNNLCLQREEDLQRTRDYHDCMNVVEVFLEKFTTEWDNLARSDAES |
| | | TAVHLEALKKLALALQERKYAIEDLKDQKQKMIEHLNLDDKELVKEQTSHLEQRW |
| | | FQLEDLIKRKIQVSVTNLEELNVVQSRFQELMEWAEEQQPNIAEALKQSPPPDMA |
| | | QNLLMDHLAICSELEAKQMLLKSLIKDADRVMADLGLNERQVIQKALSDAQSHVN |
| | | CLSDLVGQRRKYLNKALSEKTQFLMAVFQATSQIQQHERKIMFREHICLLPDDVS |
| | | KQVKTCKSAQASLKTYQNEVTGLWAQGRELMKEVTEQEKSEVLGKLQELQSVY |
| | | DSVLQKCSHRLQELEKNLVSRKHFKEDFDKACHWLKQADIVTFPEINLMNESSEL |
| | | HTQLAKYQNILEQSPEYENLLLTLQRTGQTILPSLNEVDHSYLSEKLNALPRQFNV |
| | | IVALAKDKFYKVQEAILARKEYASLIELTTQSLSELEAQFLRMSKVPTDLAVEEALS |
| | | LQDGCRAILDEVAGLGEAVDELNQKKEGFRSTGQPWQPDKMLHLVTLYHRLKR |
| | | QTEQRVSLLEDTTSAYQEHEKMCQQLERQLKSVKEEQSKVNEETLPAEEKLKMY |
| | | HSLAGSLQDSGIVLKRVTIHLEDLAPHLDPLAYEKARHQIQSWQGELKLLTSAIGE |
| | | TVTECESRMVQSIDFQTEMSRSLDWLRRVKAELSGPVYLDLNLQDIQEEIRKIQIH |
| | | QEEVQSSLRIMNALSHKEKEKFTKAKELISADLEHSLAELSELDGDIQEALRTRQA |
| | | TLTEIYSQCQRYYQVFQAANDWLEDAQELLQLAGNGLDVESAEENLKSHMEFFS |
| | | TEDQFHSNLEELHSLVATLDPLIKPTGKEDLEQKVASLELRSQRMSRDSGAQVDL |
| | | LQRCTAQWHDYQKAREEVIELMNDTEKKLSEFSLLKTSSSHEAEEKLSEHKALVS |
| | | VVNSFHEKIVALEEKASQLEKTGNDASKATLSRSMTTVWQRWTRLRAVAQDQE |
| | | KILEDAVDEWTGFNNKVKKATEMIDQLQDKLPGSSAEKASKAELLTLLEYHDTFV |
| | | LELEQQQSALGMLRQQTLSMLQDGAAPTPGEEPPLMQEITAMQDRCLNMQEKV |
| | | KTNGKLVKQELKDREMVETQINSVKCWVQETKEYLGNPTIEIDAQLEELQILLTEA |
| | | TNHRQNIEKMAEEQKEKYLGLYTILPSELSLQLAEVALDLKIRDQIQDKIKEVEQSK |
| | | ATSQELSRQIQKLAKDLTTILTKLKAKTDNVVQAKTDQKVLGEELDGCNSKLMEL |
| | | DAAVQKFLEQNGQLGKPLAKKIGKLTELHQQTIRQAENRLSKLNQAASHLEEYNE |
| | | MLELILKWIEKAKVLAHGTIAWNSASQLREQYILHQTLLEESKEIDSELEAMTEKLQ |
| | | YLTSVYCTEKMSQQVAELGRETEELRQMIKIRLQNLQDAAKDMKKFEAELKKLQA |
| | | ALEQAQATLTSPEVGRLSLKEQLSHRQHLLSEMESLKPKVQAVQLCQSALRIPED |
| | | VVASLPLCHAALRLQEEASRLQHTAIQQCNIMQEAVVQYEQYEQEMKHLQQLIE |
| | | GAHREIEDKPVATSNIQELQAQISRHEELAQKIKGYQEQIASLNSKCKMLTMKAKH |
| | | ATMLLTVTEVEGLAEGTEDLDGELLPTPSAHPSVVMMTAGRCHTLLSPVTEESG |
| | | EEGTNSEISSPPACRSPSPVANTDASVNQDIAYYQALSAERLQTDAAKIHPSTSA |
| | | SQEFYEPGLEPSATAKLGDLQRSWETLKNVISEKQRTLYEALERQQKYQDSLQSI |
| | | STKMEAIELKLSESPEPGRSPESQMAEHQALMDEILMLQDEINELQSSLAEELVS |
| | | ESCEADPAEQLALQSTLTVLAERMSTIRMKASGKRQLLEEKLNDQLEEQRQEQA |
| | | LQRYRCEADELDSWLLSTKATLDTALSPPKEPMDMEAQLMDCQNMLVEIEQKVV |
| | | ALSELSVHNENLLLEGKAHTKDEAEQLAGKLRRLKGSLLELQRALHDKQLNMQG |
| | | TAQEKEESDVDLTATQSPGVQEWLAQARTTWTQQRQSSLQQQKELEQELAEQ |
| | | KSLLRSVASRGEEILIQHSAAETSGDAGEKPDVLSQELGMEGEKSSAEDQMRMK |
| | | WESLHQEFSTKQKLLQNVLEQEQEQVLYSRPNRLLSGVPLYKGDVPTQDKSAVT |
| | | SLLDGLNQAFEEVSSQSGGAKRQSIHLEQKLYDGVSATSTWLDDVEERLFVATA |
| | | LLPEETETCLFNQEILAKDIKEMSEEMDKNKNLFSQAFPENGDNRDVIEDTLGCLL |
| | | GRLSLLDSVVNQRCHQMKERLQQILNFQNDLKVLFTSLADNKYIILQKLANVFEQ |
| | | PVAEQIEAIQQAEDGLKEFDAGIIELKRRGDKLQVEQPSMQELSKLQDMYDELMM |
| | | IIGSRRSGLNQNLTLKSQYERALQDLADLLETGQEKMAGDQKIIVSSKEEIQQLLD |
| | | KHKEYFQGLESHMILTETLFRKIISFAVQKETQFHTELMAQASAVLKRAHKRGVEL |
| | | EYILETWSHLDEDQQELSRQLEVVESSIPSVGLVEENEDRLIDRITLYQHLKSSLN |
| | | EYQPKLYQVLDDGKRLLISISCSDLESQLNQLGECWLSNTNKMSKELHRLETILK |
| | | HWTRYQSESADLIHWLQSAKDRLEFWTQQSVTVPQELEMVRDHLNAFLEFSKE |
| | | VDAQSSLKSSVLSTGNQLLRLKKVDTATLRSELSRIDSQWTDLLTNIPAVQEKLH |
| | | QLQMDKLPSRHAISEVMSWISLMENVIQKDEDNIKNSIGYKAIHEYLQKYKGFKIDI |
| | | NCKQLTVDFVNQSVLQISSQDVESKRSDKTDFAEQLGAMNKSWQILQGLVTEKI |
| | | QLLEGLLESWSEYENNVQCLKTWFETQEKRLKQQHRIGDQASVQNALKDCQDL |
| | | EDLIKAKEKEVEKIEQNGLALIQNKKEDVSSIVMSTLRELGQTWANLDHMVGQLKI |
| | | LLKSVLDQWSSHKVAFDKINSYLMEARYSLSRFRLLTGSLEAVQVQVDNLQNLQ |
| | | DDLEKQERSLQKFGSITNQLLKECHPPVTETLTNTLKEVNMRWNNLLEEIAEQLQ |
| | | SSKALLQLWQRYKDYSKQCASTVQQQEDRTNELLKAATNKDIADDEVATWIQDC |
| | | NDLLKGLGTVKDSLFFLHELGEQLKQQVDASAASAIQSDQLSLSQHLCALEQALC |
| | | KQQTSLQAGVLDYETFAKSLEALEAWIVEAEEILQGQDPSHSSDLSTIQERMEEL |
| | | KGQMLKFSSMAPDLDRLNELGYRLPLNDKEIKRMQNLNRHWSLISSQTTERFSK |
| | | LQSFLLQHQTFLEKCETWMEFLVQTEQKLAVEISGNYQHLLEQQRAHELFQAEM |
| | | FSRQQILHSIIIDGQRLLEQGQVDDRDEFNLKLTLLSNQWQGVIRRAQQRRGIIDS |
| | | QIRQWQRYREMAEKLRKWLVEVSYLPMSGLGSVPIPLQQARTLFDEVQFKEKVF |
| | | LRQQGSYILTVEAGKQLLLSADSGAEAALQAELAEIQEKWKSASMRLEEQKKKLA |
| | | FLLKDWEKCEKGIADSLEKLRTFKKKLSQSLPDHHEELHAEQMRCKELENAVGS |
| | | WTDDLTQLSLLKDTLSAYISADDISILNERVELLQRQWEELCHQLSLRRQQIGERL |
| | | NEWAVFSEKNKELCEWLTQMESKVSQNGDILIEEMIEKLKKDYQEEIAIAQENKIQ |
| | | LQQMGERLAKASHESKASEIEYKLGKVNDRWQHLLDLIAARVKKLKETLVAVQQL |
| | | DKNMSSLRTWLAHIESELAKPIVYDSCNSEEIQRKLNEQQELQRDIEKHSTGVAS |
| | | VLNLCEVLLHDCDACATDAECDSIQQATRNLDRRWRNICAMSMERRLKIEETWR |
| | | LWQKFLDDYSRFEDWLKSSERTAAFPSSSGVIYTVAKEELKKFEAFQRQVHECL |
| | | TQLELINKQYRRLARENRTDSACSLKQMVHEGNQRWDNLQKRVTSILRRLKHFI |
| | | GQREEFETARDSILVWLTEMDLQLTNIEHFSECDVQAKIKQLKAFQQEISLNHNKI |
| | | EQIIAQGEQLIEKSEPLDAAIIEEELDELRRYCQEVFGRVERYHKKLIRLPLPDDEH |
| | | DLSDRELELEDSAALSDLHWHDRSADSLLSPQPSSNLSLSLAQPLRSERSGRDT |
| | | PASVDSIPLEWDHDYDLSRDLESAMSRALPSEDEEGQDDKDFYLRGAVGLSGD |
| | | HSALESQIRQLGKALDDSRFQIQQTENIIRSKTPTGPELDTSYKGYMKLLGECSSS |
| | | IDSVKRLEHKLKEEEESLPGFVNLHSTETQTAGVIDRWELLQAQALSKELRMKQN |
| | | LQKWQQFNSDLNSIWAWLGDTEEELEQLQRLELSTDIQTIELQIKKLKELQKAVD |
| | | HRKAIILSINLCSPEFTQADSKESRDLQDRLSQMNGRWDRVCSLLEEWRGLLQD |
| | | ALMQCQGFHEMSHGLLLMLENIDRRKNEIVPIDSNLDAEILQDHHKQLMQIKHELL |
| | | ESQLRVASLQDMSCQLLVNAEGTDCLEAKEKVHVIGNRLKLLLKEVSRHIKELEK |
| | | LLDVSSSQQDLSSWSSADELDTSGSVSPTSGRSTPNRQKTPRGKCSLSQPGPS |
| | | VSSPHSRSTKGGSDSSLSEPGPGRSGRGFLFRVLRAALPLQLLLLLLIGLACLVP |
| | | MSEEDYSCALSNNFARSFHPMLRYTNGPPPL |
| 32 | Human Nesprin-2 | MASSPELPTEDEQGSWGIDDLHISLQAEQEDTQKKAFTCWINSQLARHTSPSVIS |
| | | DLFTDIKKGHVLLDLLEVLSGQQLPRDKGSNTFQCRINIEHALTFLRNRSIKLINIHV |
| | | TDIIDGNPSIILGLIWTIILHFHIEKLAQTLSCNYNQPSLDDVSVVDSSPASSPPAKK |
| | | CSKVQARWQMSARKALLLWAQEQCATYESVNVTDFKSSWRNGMAFLAIIHALR |
| | | PDLIDMKSVKHRSNKDNLREAFRIAEQELKIPRLLEPEDVDVVDPDEKSIMTYVAQ |
| | | FLQYSKDAPGTGEEAQGKVKDAMGWLTLQKEKLQKLLKDSENDTYFKKYNSLLS |
| | | FMESFNEEKKSFLDVLSIKRDLDELDKDHLQLREAWDGLDHQINAWKIKLNYALP |
| | | PPLHQTEAWLQEVEELMDEDLSASQDHSQAVTLIQEKMTLFKSLMDRFEHHSNI |
| | | LLTFENKDENHLPLVPPNKLEEMKRRINNILEKKFILLLEFHYYKCLVLGLVDEVKS |
| | | KLDIWNIKYGSRESVELLLEDWHKFIEEKEFLARLDTSFQKCGEIYKNLAGECQNI |
| | | NKQYMMVKSDVCMYRKNIYNVKSTLQKVLACWATYVENLRLLRACFEETKKEEI |
| | | KEVPFETLAQWNLEHATLNEAGNFLVEVSNDVVGSSISKELRRLNKRWRKLVSK |
| | | TQLEMNLPLMIKKQDQPTFDNSGNILSKEEKATVEFSTDMSVELPENYNQNIKAG |
| | | EKHEKENEEFTGQLKVAKDVEKLIGQVEIWEAEAKSVLDQDDVDTSMEESLKHLI |
| | | AKGSMFDELMARSEDMLQMDIQNISSQESFQHVLTTGLQAKIQEAKEKVQINVVK |
| | | LIAALKNLTDVSPDLDIRLKMEESQKELESYMMRAQQLLGQRESPGELISKHKEA |
| | | LIISNTKSLAKYLKAVEELKNNVTEDIKMSLEEKSRDVCAKWESLHHELSLYVQQL |
| | | KIDIEKGKLSDNILKLEKQINKEKKLIRRGRTKGLIKEHEACFSEEGCLYQLNHHME |
| | | VLRELCEELPSQKSQQEVKRLLKDYEQKIERLLKCASEIHMTLQPTAGGTSKNEG |
| | | TITTSENRGGDPHSEAPFAKSDNQPSTEKAMEPTMKFSLASVLRPLQEESIMEKD |
| | | YSASINSLLERYDTYRDILEHHLQNNKFRITSDFSSEEDRSSSCLQAKLTDLQVIK |
| | | NETDARWKEFEIISLKLENHVNDIKKPFVIKERDTLKERERELQMTLNTRMESLET |
| | | ALRLVLPVEKASLLLCGSDLPLHKMAIQGFHLIDADRIYQHLRNIQDSIAKQIEICNR |
| | | LEEPGNFVLKELHPFDLHAMQNIILKYKTQFEGMNHRVQRSEDTLKALEDFLASL |
| | | RTAKLSAEPVTDLSASDTQVAQENTLTVKNKEGEIHLMKDKAKHLDKCLKMLDM |
| | | SFKDAERGDDTSCENLLDAFSIKLSETHGYGVQEEFTEENKLLEACIFKNNELLKN |
| | | IQDVQSQISKIGLKDPTVPAVKHRKKSLIRLDKVLDEYEEEKRHLQEMANSLPHFK |
| | | DGREKTVNQQCQNTVVLWENTKALVTECLEQCGRVLELLKQYQNFKSILTTLIQK |
| | | EESVISLQASYMGKENLKKRIAEIEIVKEEFNEHLEVVDKINQVCKNLQFYLNKMK |
| | | TFEEPPFEKEANIIVDRWLDINEKTEDYYENLGRALALWDKLFNLKNVIDEWTEKA |
| | | LQKMELHQLTEEDRERLKEELQVHEQKTSEFSRRVAEIQFLLQSSEIPLELQVME |
| | | SSILNKMEHVQKCLTGESNCHALSGSTAELREDLDQAKTQIGMTESLLKALSPSD |
| | | SLEIFTKLEEIQQQILQQKHSMILLENQIGCLTPELSELKKQYESVSDLFNTKKSVL |
| | | QDHFSKLLNDQCKNFNDWFSNIKVNLKECFESSETKKSVEQKLQKLSDFLTLEG |
| | | RNSKIKQVDSVLKHVKKHLPKAHVKELISWLVGQEFELEKMESICQARAKELEDS |
| | | LQQLLRLQDDHRNLRKWLTNQEEKWKGMEEPGEKTELFCQALARKREQFESVA |
| | | QLNNSLKEYGFTEEEEIIMEATCLMDRYQTLLRQLSEIEEEDKLLPTEDQSFNDLA |
| | | HDVIHWIKEIKESLMVLNSSEGKMPLEERIQKIKEIILLKPEGDARIETIMKQAESSE |
| | | APLVQKTLTDISNQWDNTLHLASTYLSHQEKLLLEGEKYLQSKEDLRLMLIELKKK |
| | | QEAGFALQHGLQEKKAQLKIYKKFLKKAQDLTSLLKELKSQGNYLLECTKNPSFS |
| | | EEPWLEIKHLHESLLQQLQDSVQNLDGHVREHDSYQVCVTDLNTTLDNFSKEFV |
| | | SFSDKPVDQIAVEEKLQKLQELENRLSLQDGTLKKILALAKSVKQNTSSVGQKIIK |
| | | DDIKSLQCKQKDLENRLASAKQEMECCLNSILKSKRSTEKKGKFTLPGREKQATS |
| | | DVQESTQESAAVEKLEEDWEINKDSAVEMAMSKQLSLNAQESMKNTEDERKVN |
| | | ELQNQPLELDTMLRNEQLEEIEKLYTQLEAKKAAIKPLEQTECLNKTETGALVLHN |
| | | IGYSAQHLDNLLQALITLKKNKESQYCVLRDFQEYLAAVESSMKALLTDKESLKVG |
| | | PLDSVTYLDKIKKFIASIEKEKDSLGNLKIKWENLSNHVTDMDKKLLESQIKQLEHG |
| | | WEQVEQQIQKKYSQQVVEYDEFTTLMNKVQDTEISLQQQQQHLQLRLKSPEER |
| | | AGNQSMIALTTDLQATKHGFSVLKGQAELQMKRIWGEKEKKNLEDGINNLKKQW |
| | | ETLEPLHLEAENQIKKCDIRNKMKETILWAKNLLGELNPSIPLLPDDILSQIRKCKVT |
| | | HDGILARQQSVESLAEEVKDKVPSLTTYEGSDLNNTLEDLRNQYQMLVLKSTQR |
| | | SQQLEFKLEERSNFFAIIRKFQLMVQESETLIIPRVETAATEAELKHHHVTLEASQK |
| | | ELQEIDSGISTHLQELTNIYEELNVFERLFLEDQLKNLKIRTNRIQRFIQNTCNEVEH |
| | | KIKFCRQFHEKTSALQEEADSIQRNELLLNQEVNKGVKEEIYNLKDRLTAIKCCILQ |
| | | VLKLKKVFDYIGLNWDFSQLDQLQTQVFEKEKELEEKIKQLDTFEEEHGKYQALL |
| | | SKMRAIDLQIKKMTEVVLKAPDSSPESRRLNAQILSQRIEKAKCLCDEIIKKLNENK |
| | | TFDDSFKEKEILQIKLNAEENDKLYKVLQNMVLELSPKELDEKNCQDKLETSLHVL |
| | | NQIKSQLQQPLLINLEIKHIQNEKDNCEAFQEQVWAEMCSIKAVTAIEKQREENSS |
| | | EASDVETKLREFEDLQMQLNTSIDLRTNVLNDAYENLTRYKEAVTRAVESITSLEA |
| | | IIIPYRVDVGNPEESLEMPLRKQEELESTVAHIQDLTEKLGMISSPEAKLQLQYTLQ |
| | | ELVSKNSAMKEAFKAQETEAERYLENYKCYRKMEEDIYTNLSKMETVLGQSMSS |
| | | LPLSYREALERLEQSKALVSNLISTKEELMKLRQILRLLRLRCTENDGICLLKIVSAL |
| | | WEKWLSLLEAAKEWEMWCEELKQEWKFVSEEIEREAIILDNLQEELPEISKTKEA |
| | | ATTEELSELLDCLCQYGENVEKQQLLLTLLLQRIRSIQNVPESSGAVETVPAFQEI |
| | | TSMKERCNKLLQKVQKNKELVQTEIQERHSFTKEIIALKNFFQQTTTSFQNMAFQ |
| | | DHPEKSEQFEELQSILKKGKLTFENIMEKLRIKYSEMYTIVPAEIESQVEECRKALE |
| | | DIDEKISNEVLKSSPSYAMRRKIEEINNGLHNVEKMLQQKSKNIEKAQEIQKKMW |
| | | DELDLWHSKLNELDSEVQDIVEQDPGQAQEWMDNLMIPFQQYQQVSQRAECRT |
| | | SQLNKATVKMEEYSDLLKSTEAWIENTSHLLANPADYDSLRTLSHHASTVQMALE |
| | | DSEQKHNLLHSIFMDLEDLSIIFETDELTQSIQELSNQVTALQQKIMESLPQIQRMA |
| | | DDVVAIESEVKSMEKRVSKIKTILLSKEIFDFSPEEHLKHGEVILENIRPMKKTIAEIV |
| | | SYQVELRLPQTGMKPLPVFQRTNQLLQDIKLLENVTQEQNELLKVVIKQTNEWDE |
| | | EIENLKQILNNYSAQFSLEHMSPDQADKLPQLQGEIERMEKQILSLNQRKEDLLVD |
| | | LKATVLNLHQHLKQEQEGVERDRLPAVTSEEGGVAERDASERKLNRRGSMSYL |
| | | AAVEEEVEESSVKSDNGDEKAEPSPQSWSSLWKHDKDMEEDRASSSSGTIVQE |
| | | AYGKISTSDNSMAQILTPDSLNTEQGPECSLRPNQTEEGTTPPIEADTLDSSDAQ |
| | | GGLEPRVEKTRPEPTEVLHACKTQVAELELWLQQANVAVEPETLNADMQQVLE |
| | | QQLVGCQAMLTEIEHKVAFLLETCKDQGLGDNGATQHEAEALSLKLKTVKCNLE |
| | | KVQMMLQEKHSEDQHPTILKKSSEPEHQEALQPVNLSELESIVTERPQFSRQKD |
| | | FQQQQVLELKPMEQKDFIKFIEFNAKKMWPQYCQHDNDTTQESSASNQASSPE |
| | | NDVPDSILSPQGQNGDKWQYLHHELSSKIKLPLPQLVEPQVSTNMGILPSVTMY |
| | | NFRYPTTEELKTYTTQLEDLRQEASNLQTQENMTEEAYINLDKKLFELFLTLSQCL |
| | | SSVEEMLEMPRLYREDGSGQQVHYETLALELKKLYLALSDKKGDLLKAMTWPGE |
| | | NTNLLLECFDNLQVCLEHTQAAAVCRSKSLKAGLDYNRSYQNEIKRLYHQLIKSK |
| | | TSLQQSLNEISGQSVAEQLQKADAYTVELENAESRVAKLRDEGERLHLPYALLQE |
| | | VYKLEDVLDSMWGMLRARYTELSSPFVTESQQDALLQGMVELVKIGKEKLAHGH |
| | | LKQTKSKVALQAQIENHKVFFQKLVADMLLIQAYSAKILPSLLQNRETFWAEQVTE |
| | | VKILEEKSRQCGMKLQSLLQKWEEFDENYASLEKDLEILISTLPSVSLVEETEERL |
| | | VERISFYQQIKRNIGGKHARLYQTLNEGKQLVASVSCPELEGQIAKLEEQWLSLN |
| | | KKIDHELHRLQALLKHLLSYNRDSDQLTKWLESSQHTLNYWKEQSLNVSQDLDTI |
| | | RSNINNFFEFSKEVDEKSSLKTAVISIGNQLLHLKETDTATLRASLAQFEQKWTMLI |
| | | TQLPDIQEKLHQLQMEKLPSRKAITEMISWMNNVEHQTSDEDSVHSPSSASQVK |
| | | HLLQKHKEFRMEMDYKQWIVDFVNQSLLQLSTCDVESKRYERTEFAEHLGEMN |
| | | RQWHRVHGMLNRKIQHLEQLLESITESENKIQILNNWLEAQEERLKTLQKPESVIS |
| | | VQKLLLDCQDIENQLAIKSKALDELKQSYLTLESGAVPLLEDTASRIDELFQKRSS |
| | | VLTQVNQLKTSMQSVLQEWKIYDQLYDEVNMMTIRFWYCMEHSKPVVLSLETLR |
| | | CQVENLQSLQDEAESSEGSWEKLQEVIGKLKGLCPSVAEIIEEKCQNTHKRWTQ |
| | | VNQAIADQLQKAQSLLQLWKAYSNAHGEAAARLKQQEAKFQQLANISMSGNNLA |
| | | EILPPALQDIKELQHDVQKTKEAFLQNSSVLDRLPQPAESSTHMLLPGPLHSLQR |
| | | AAYLEKMLLVKANEFEFVLSQFKDFGVRLESLKGLIMHEEENLDRLHQQEKENPD |
| | | SFLNHVLALTAQSPDIEHLNEVSLKLPLSDVAVKTLQNMNRQWIRATATALERCS |
| | | ELQGIGLNEKFLYCCEKWIQLLEKIEEALKVDVANSLPELLEQQKTYKMLEAEVSI |
| | | NQTIADSYVTQSLQLLDTTEIENRPEFITEFSKLTDRWQNAVQGVRQRKGDVDGL |
| | | VRQWQDFTTSVENLFRFLTDTSHLLSAVKGQERFSLYQTRSLIHELKNKEIHFQR |
| | | RRTTCALTLEAGEKLLLTTDLKTKESVGRRISQLQDSWKDMEPQLAEMIKQFQST |
| | | VETWDQCEKKIKELKSRLQVLKAQSEDPLPELHEDLHNEKELIKELEQSLASWTQ |
| | | NLKELQTMKADLTRHVLVEDVMVLKEQIEHLHRQWEDLCLRVAIRKQEIEDRLNT |
| | | WVVFNEKNKELCAWLVQMENKVLQTADISIEEMIEKLQKDCMEEINLFSENKLQL |
| | | KQMGDQLIKASNKSRAAEIDDKLNKINDRWQHLFDVIGSRVKKLKETFAFIQQLDK |
| | | NMSNLRTWLARIESELSKPVVYDVCDDQEIQKRLAEQQDLQRDIEQHSAGVESV |
| | | FNICDVLLHDSDACANETECDSIQQTTRSLDRRWRNICAMSMERRMKIEETWRL |
| | | WQKFLDDYSRFEDWLKSAERTAACPNSSEVLYTSAKEELKRFEAFQRQIHERLT |
| | | QLELINKQYRRLARENRTDTASRLKQMVHEGNQRWDNLQRRVTAVLRRLRHFT |
| | | NQREEFEGTRESILVWLTEMDLQLTNVEHFSESDADDKMRQLNGFQQEITLNTN |
| | | KIDQLIVFGEQLIQKSEPLDAVLIEDELEELHRYCQEVFGRVSRFHRRLTSCTPGL |
| | | EDEKEASENETDMEDPREIQTDSWRKRGESEEPSSPQSLCHLVAPGHERSGCE |
| | | TPVSVDSIPLEWDHTGDVGGSSSHEEDEEGPYYSALSGKSISDGHSWHVPDSP |
| | | SCPEHHYKQMEGDRNVPPVPPASSTPYKPPYGKLLLPPGTDGGKEGPRVLNGN |
| | | PQQEDGGLAGITEQQSGAFDRWEMIQAQELHNKLKIKQNLQQLNSDISAITTWLK |
| | | KTEAELEMLKMAKPPSDIQEIELRVKRLQEILKAFDTYKALVVSVNVSSKEFLQTE |
| | | SPESTELQSRLRQLSLLWEAAQGAVDSWRGGLRQSLMQCQDFHQLSQNLLLW |
| | | LASAKNRRQKAHVTDPKADPRALLECRRELMQLEKELVERQPQVDMLQEISNSL |
| | | LIKGHGEDCIEAEEKVHVIEKKLKQLREQVSQDLMALQGTQNPASPLPSFDEVDS |
| | | GDQPPATSVPAPRAKQFRAVRTTEGEEETESRVPGSTRPQRSFLSRVVRAALPL |
| | | QLLLLLLLLLACLLPSSEEDYSCTQANNFARSFYPMLRYTNGPPPT |
| 33 | Human Nesprin-3 | MTQQPQDDFDRSVEDAQAWMKAVQDQLQVNDNTQGPRAALEARLWETEKICQ |
| | | LEPEGRVRVDLVLRMAEALLACCPGDQKPGILARLKDIKAQWEETVTYMTHCHS |
| | | RIEWVWLHWSEYLLARDEFYRWFQKMMVTLEPHIELQLGLKEKQWQLSHAQVL |
| | | LHNVDNQAVLLDRLLEEAASLFNRIGDPSVDEDAQKRMKAEYDAVKAKAQKRVD |
| | | LLEQVAREHEEYQAGVDEFQLWLKAVVEKVNGCLGRNCKLPITQRLSTLQDIAK |
| | | DFPRGEESLETLEEQSAGVIRNTSPLGAEKITGELEEMRKVLEKLRALWEEEEER |
| | | LRGLLRSRGAWEQQIKQLEAELSEFRMVLQRLAQEGLQPAAKAGTEDELVAHW |
| | | RRYSATRAALASEEPRVDRLQAQLKELIVFPHNLKPLSDSVIATIQEYQSLKVKSA |
| | | RLRNAAAVELWQHFQRPLQDLQLWKALAQRLLEVTASLPDLPSLHTFLPQIEAAL |
| | | MESSRLKELLTMLQLKKDLLIGIFGQERATALLEQVAGSMRDRDLLHNSLLQRKS |
| | | KLQSLLAQHKDFGAAFEPLQRKLLDLQVRVQAEKGLQRDLPGKQAQLSRLQGLQ |
| | | EEGLDLGAQMEAARPLVQENPNHQHKMDQLSSDFQALQRSLEDLVDRCRQSV |
| | | QEHCTFSHQLLELRQWIVVTTQKLEAHRGEAGPGDAESQEAEFERLVAEFPEKE |
| | | AQLSLVEAQGWLVMEKSSPEGAAVVQEELRELAESWRALRLLEESLLSLIRNWH |
| | | LQRMEVDSGKKMVFTNNIPKSGFLINPMDPIPRHRRRANLLQEEEGSHEDFSQLL |
| | | RNFGQWLQVENSKLVRIIAMRTSTAEDLRTRKSKLQELEARVPEGQHLFENLLRL |
| | | GPARGTSDELEDLRYQWMLYKSKLKDSGHLLTQSSPGEPTGFQKTRRWRGLG |
| | | SLFRRACCVALPLQLLLLLFLLLLFLLPIREEDRSCTLANNFARSFTLMLRYNGPPP |
| | | T |
| 34 | Human Nesprin-4 | MALSLPLGPRLGSEPLNHPPGAPREADIVGCTVCPASGEESTSPEQAQTLGQDS |
| | | LGPPEHFQGGPRGNEPAAHPPRWSTPSSYEDPAGGKHCEHPISGLEVLEAEQN |
| | | SLHLCLLGLGRRLQDLEQGLGHWALAQSGMVQLQALQVDLRGAAERVEALLAF |
| | | GEGLAQRSEPRAWAALEQILRALGAYRDSIFRRLWQLQAQLVSYSLVFEEANTL |
| | | DQDLEVEGDSDWPGPGGVWGPWAPSSLPTSTELEWDPAGDIGGLGPLGQKTA |
| | | RTLGVPCELCGQRGPQGRGQGLEEADTSHSRQDMLESGLGHQKRLARHQRHS |
| | | LLRKPQDKKRQASPHLQDVRLEGNPGAPDPASRQPLTFLLILFLLFLLLVGAMFLL |
| | | PASGGPCCSHARIPRTPYLVLSYVNGLPPV |
| 35 | Human KASH5 | MDLPEGPVGGPTAEMYLRERPEEARLGMPVSLEEQILNSTFEACDPQRTGTVAV |
| | | AQVLAYLEAVTGQGPQDARLQTLANSLDPNGEGPKATVDLDTFLVVMRDWIAAC |
| | | QLHGGLELEEETAFQGALTSRQLPSGCPEAEEPANLESFGGEDPRPELQATADL |
| | | LSSLEDLELSNRRLVGENAKLQRSMETAEEGSARLGEEILALRKQLHSTQQALQF |
| | | AKAMDEELEDLKTLARSLEEQNRSLLAQARQAEKEQQHLVAEMETLQEENGKLL |
| | | AERDGVKKRSQELAMEKDTLKRQLFECEHLICQRDTILSERTRDVESLAQTLEEY |
| | | RVTTQELRLEISRLEEQLSQTYEGPDELPEGAQLRRVGWTELLPPSLGLEIEAIRQ |
| | | KQEVATADLSNPLCGVWQWEEVIHETSEETEFPSEAPAGGQRNFQGEPAHPEE |
| | | GRKEPSMWLTRREEEEDAESQVTADLPVPLGAPRPGDIPENPPERPARRELQQ |
| | | ALVPVMKKLVPVRRRAWGQLCLPPQRLRVTRHPLIPAPVLGLLLLLLLSVLLLGPS |
| | | PPPTWPHLQLCYLQPPPV |
| 36 | Human LRMP | MESTPFSGVANQIHTLCERPTYGEVKDGALDVKRQHKCPGPTSGPSPGTNLSG |
| | | CIRMNDDPSMEENGVERVCPESLLQSREYSSLPLPRHTSSTDGTITSSDPGLEIL |
| | | NMASCDLDRNSLCKKEEDTRSASPTIEAQGTSPAHDNIAFQDSTSKDKTILNLEA |
| | | KEEPETIEEHKKEHASGDSVVSPLPVTTVKSVNLRQSENTSANEKEVEAEFLRLS |
| | | LGFKCDWFTLEKRVKLEERSRDLAEENLKKEITNCLKLLESLTPLCEDDNQAQEII |
| | | KKLEKSIKFLSQCAARVASRAEMLGAINQESRVSKAVEVMIQHVENLKRMYAKEH |
| | | AELEELKQVLLQNERSFNPLEDDDDCQIKKRSASLNSKPSSLRRVTIASLPRNIGN |
| | | AGMVAGMENNDRFSRRSSSWRILGSKQSEHRPSLPRFISTYSWADAEEEKCEL |
| | | KTKDDSEPSGEETVERTRKPSLSEKKNNPSKWDVSSVYDTIASWATNLKSSIRK |
| | | ANKALWLSIAFIVLFAALMSFLTGQLFQKSVDAAPTQQEDSWTSLEHILWPFTRLR |
| | | HNGPPPV |
| 37 | Human Nesprin-1 | RGFLFRVLRAALPLQLLLLLLIGLACLVPMSEEDYSCALSNNFARSFHPMLRYTNG |
| | KASH domain | PPPL |
| 38 | Human Nesprin-2 | RSFLSRVVRAALPLQLLLLLLLLLACLLPSSEEDYSCTQANNFARSFYPMLRYTNG |
| | KASH domain | PPPT |
| 39 | Human Nesprin-3 | GSLFRRACCVALPLQLLLLLFLLLLFLLPIREEDRSCTLANNFARSFTLMLRYNGPP |
| | KASH domain | PT |
| 40 | Human Nesprin-4 | DPASRQPLTFLLILFLLFLLLVGAMFLLPASGGPCCSHARIPRTPYLVLSYVNGLPP |
| | KASH domain | V |
| 41 | Human KASH5 | LIPAPVLGLLLLLLLSVLLLGPSPPPTWPHLQLCYLQPPPV |
| | KASH domain | |
| 42 | Human LRMP | QLFQKSVDAAPTQQEDSWTSLEHILWPFTRLRHNGPPPV |
| | KASH domain | |
| 43 | SUN1(360-812); | DDPQDVFKPTTSRLKQPLQGDSEAFPWHWMSGVEQQVASLSGQCHHHGENLR |
| | DNSUN1 | ELTTLLQKLQARVDQMEGGAAGPSASVRDAVGQPPRETDFMAFHQEHEVRMS |
| | | HLEDILGKLREKSEAIQKELEQTKQKTISAVGEQLLPTVEHLQLELDQLKSELSSW |
| | | RHVKTGCETVDAVQERVDVQVREMVKLLFSEDQQGGSLEQLLQRFSSQFVSKG |
| | | DLQTMLRDLQLQILRNVTHHVSVTKQLPTSEAVVSAVSEAGASGITEAQARAIVN |
| | | SALKLYSQDKTGMVDFALESGGGSILSTRCSETYETKTALMSLFGIPLWYFSQSP |
| | | RVVIQPDIYPGNCWAFKGSQGYLVVRLSMMIHPAAFTLEHIPKTLSPTGNISSAPK |
| | | DFAVYGLENEYQEEGQLLGQFTYDQDGESLQMFQALKRPDDTAFQIVELRIFSN |
| | | WGHPEYTCLYRFRVHGEPVK |
| 44 | SUN1(616-812) | GITEAQARAIVNSALKLYSQDKTGMVDFALESGGGSILSTRCSETYETKTALMSLF |
| | | GIPLWYFSQSPRVVIQPDIYPGNCWAFKGSQGYLVVRLSMMIHPAAFTLEHIPKT |
| | | LSPTGNISSAPKDFAVYGLENEYQEEGQLLGQFTYDQDGESLQMFQALKRPDDT |
| | | AFQIVELRIFSNWGHPEYTCLYRFRVHGEPVK |
| 45 | SUN2(258-717); | DEGWEARDSSPHFQAEQRVMSRVHSLERRLEALAAEFSSNWQKEAMRLERLEL |
| | DNSUN2 | RQGAPGQGGGGGLSHEDTLALLEGLVSRREAALKEDFRRETAARIQEELSALRA |
| | | EHQQDSEDLFKKIVRASQESEARIQQLKSEWQSMTQESFQESSVKELRRLEDQL |
| | | AGLQQELAALALKQSSVAEEVGLLPQQIQAVRDDVESQFPAWISQFLARGGGGR |
| | | VGLLQREEMQAQLRELESKILTHVAEMQGKSAREAAASLSLTLQKEGVIGVTEEQ |
| | | VHHIVKQALQRYSEDRIGLADYALESGGASVISTRCSETYETKTALLSLFGIPLWY |
| | | HSQSPRVILQPDVHPGNCWAFQGPQGFAVVRLSARIRPTAVTLEHVPKALSPNS |
| | | TISSAPKDFAIFGFDEDLQQEGTLLGKFTYDQDGEPIQTFHFQAPTMATYQVVEL |
| | | RILTNWGHPEYTCIYRFRVHGEPAH |
| 46 | SUN2(522-717) | GVTEEQVHHIVKQALQRYSEDRIGLADYALESGGASVISTRCSETYETKTALLSLF |
| | | GIPLWYHSQSPRVILQPDVHPGNCWAFQGPQGFAVVRLSARIRPTAVTLEHVPK |
| | | ALSPNSTISSAPKDFAIFGFDEDLQQEGTLLGKFTYDQDGEPIQTFHFQAPTMAT |
| | | YQVVELRILTNWGHPEYTCIYRFRVHGEPAH |
| 47 | DNSUN1 w/SP and | MKWVTFISLLFLFSSAYSRGVFRRDGPALDDPQDVFKPTTSRLKQPLQGDSEAF |
| | KDEL | PWHWMSGVEQQVASLSGQCHHHGENLRELTTLLQKLQARVDQMEGGAAGPS |
| | | ASVRDAVGQPPRETDFMAFHQEHEVRMSHLEDILGKLREKSEAIQKELEQTKQK |
| | | TISAVGEQLLPTVEHLQLELDQLKSELSSWRHVKTGCETVDAVQERVDVQVREM |
| | | VKLLFSEDQQGGSLEQLLQRFSSQFVSKGDLQTMLRDLQLQILRNVTHHVSVTK |
| | | QLPTSEAVVSAVSEAGASGITEAQARAIVNSALKLYSQDKTGMVDFALESGGGSI |
| | | LSTRCSETYETKTALMSLFGIPLWYFSQSPRVVIQPDIYPGNCWAFKGSQGYLVV |
| | | RLSMMIHPAAFTLEHIPKTLSPTGNISSAPKDFAVYGLENEYQEEGQLLGQFTYD |
| | | QDGESLQMFQALKRPDDTAFQIVELRIFSNWGHPEYTCLYRFRVHGEPVKKDEL |
| 48 | DNSUN2 w/SP and | MKWVTFISLLFLFSSAYSRGVFRRDGPALDEGWEARDSSPHFQAEQRVMSRVH |
| | KDEL | SLERRLEALAAEFSSNWQKEAMRLERLELRQGAPGQGGGGGLSHEDTLALLEG |
| | | LVSRREAALKEDFRRETAARIQEELSALRAEHQQDSEDLFKKIVRASQESEARIQ |
| | | QLKSEWQSMTQESFQESSVKELRRLEDQLAGLQQELAALALKQSSVAEEVGLLP |
| | | QQIQAVRDDVESQFPAWISQFLARGGGGRVGLLQREEMQAQLRELESKILTHVA |
| | | EMQGKSAREAAASLSLTLQKEGVIGVTEEQVHHIVKQALQRYSEDRIGLADYALE |
| | | SGGASVISTRCSETYETKTALLSLFGIPLWYHSQSPRVILQPDVHPGNCWAFQGP |
| | | QGFAVVRLSARIRPTAVTLEHVPKALSPNSTISSAPKDFAIFGFDEDLQQEGTLLG |
| | | KFTYDQDGEPIQTFHFQAPTMATYQVVELRILTNWGHPEYTCIYRFRVHGEPAHK |
| | | DEL |
| 49 | SUN2(324-717) | SHEDTLALLEGLVSRREAALKEDFRRETAARIQEELSALRAEHQQDSEDLFKKIV |
| | (Zhou et al., 2012) | RASQESEARIQQLKSEWQSMTQESFQESSVKELRRLEDQLAGLQQELAALALKQ |
| | | SSVAEEVGLLPQQIQAVRDDVESQFPAWISQFLARGGGGRVGLLQREEMQAQL |
| | | RELESKILTHVAEMQGKSAREAAASLSLTLQKEGVIGVTEEQVHHIVKQALQRYS |
| | | EDRIGLADYALESGGASVISTRCSETYETKTALLSLFGIPLWYHSQSPRVILQPDV |
| | | HPGNCWAFQGPQGFAVVRLSARIRPTAVTLEHVPKALSPNSTISSAPKDFAIFGF |
| | | DEDLQQEGTLLGKFTYDQDGEPIQTFHFQAPTMATYQVVELRILTNWGHPEYTCI |
| | | YRFRVHGEPAH |
| 50 | SUN2(335-717) | LVSRREAALKEDFRRETAARIQEELSALRAEHQQDSEDLFKKIVRASQESEARIQ |
| | (Sosa et al., 2012) | QLKSEWQSMTQESFQESSVKELRRLEDQLAGLQQELAALALKQSSVAEEVGLLP |
| | | QQIQAVRDDVESQFPAWISQFLARGGGGRVGLLQREEMQAQLRELESKILTHVA |
| | | EMQGKSAREAAASLSLTLQKEGVIGVTEEQVHHIVKQALQRYSEDRIGLADYALE |
| | | SGGASVISTRCSETYETKTALLSLFGIPLWYHSQSPRVILQPDVHPGNCWAFQGP |
| | | QGFAVVRLSARIRPTAVTLEHVPKALSPNSTISSAPKDFAIFGFDEDLQQEGTLLG |
| | | KFTYDQDGEPIQTFHFQAPTMATYQVVELRILTNWGHPEYTCIYRFRVHGEPAH |
| 51 | SUN2(398-717) | MTQESFQESSVKELRRLEDQLAGLQQELAALALKQSSVAEEVGLLPQQIQAVRD |
| | (Zhou et al., 2012) | DVESQFPAWISQFLARGGGGRVGLLQREEMQAQLRELESKILTHVAEMQGKSA |
| | | REAAASLSLTLQKEGVIGVTEEQVHHIVKQALQRYSEDRIGLADYALESGGASVIS |
| | | TRCSETYETKTALLSLFGIPLWYHSQSPRVILQPDVHPGNCWAFQGPQGFAVVR |
| | | LSARIRPTAVTLEHVPKALSPNSTISSAPKDFAIFGFDEDLQQEGTLLGKFTYDQD |
| | | GEPIQTFHFQAPTMATYQVVELRILTNWGHPEYTCIYRFRVHGEPAH |
| 52 | SUN2(433-717) | SSVAEEVGLLPQQIQAVRDDVESQFPAWISQFLARGGGGRVGLLQREEMQAQL |
| | (Zhou et al., 2012) | RELESKILTHVAEMQGKSAREAAASLSLTLQKEGVIGVTEEQVHHIVKQALQRYS |
| | | EDRIGLADYALESGGASVISTRCSETYETKTALLSLFGIPLWYHSQSPRVILQPDV |
| | | HPGNCWAFQGPQGFAVVRLSARIRPTAVTLEHVPKALSPNSTISSAPKDFAIFGF |
| | | DEDLQQEGTLLGKFTYDQDGEPIQTFHFQAPTMATYQVVELRILTNWGHPEYTCI |
| | | YRFRVHGEPAH |
| 53 | SUN2(467-717) | RGGGGRVGLLQREEMQAQLRELESKILTHVAEMQGKSAREAAASLSLTLQKEG |
| | (Wang et al., 2012) | VIGVTEEQVHHIVKQALQRYSEDRIGLADYALESGGASVISTRCSETYETKTALLS |
| | | LFGIPLWYHSQSPRVILQPDVHPGNCWAFQGPQGFAVVRLSARIRPTAVTLEHV |
| | | PKALSPNSTISSAPKDFAIFGFDEDLQQEGTLLGKFTYDQDGEPIQTFHFQAPTM |
| | | ATYQVVELRILTNWGHPEYTCIYRFRVHGEPAH |
| 54 | SUN2(507-717) | AAASLSLTLQKEGVIGVTEEQVHHIVKQALQRYSEDRIGLADYALESGGASVISTR |
| | (Sosa et al., 2012) | CSETYETKTALLSLFGIPLWYHSQSPRVILQPDVHPGNCWAFQGPQGFAVVRLS |
| | | ARIRPTAVTLEHVPKALSPNSTISSAPKDFAIFGFDEDLQQEGTLLGKFTYDQDGE |
| | | PIQTFHFQAPTMATYQVVELRILTNWGHPEYTCIYRFRVHGEPAH |
| 55 | SUN2(514-717) | TLQKEGVIGVTEEQVHHIVKQALQRYSEDRIGLADYALESGGASVISTRCSETYET |
| | (Sosa et al., 2012) | KTALLSLFGIPLWYHSQSPRVILQPDVHPGNCWAFQGPQGFAVVRLSARIRPTAV |
| | | TLEHVPKALSPNSTISSAPKDFAIFGFDEDLQQEGTLLGKFTYDQDGEPIQTFHFQ |
| | | APTMATYQVVELRILTNWGHPEYTCIYRFRVHGEPAH |
| 56 | SUN2(520-717) | VIGVTEEQVHHIVKQALQRYSEDRIGLADYALESGGASVISTRCSETYETKTALLS |
| | (Zhou et al., 2012) | LFGIPLWYHSQSPRVILQPDVHPGNCWAFQGPQGFAVVRLSARIRPTAVTLEHV |
| | | PKALSPNSTISSAPKDFAIFGFDEDLQQEGTLLGKFTYDQDGEPIQTFHFQAPTM |
| | | ATYQWVELRILTNWGHPEYTCIYRFRVHGEPAH |
| 57 | SUN2(521-717) | IGVTEEQVHHIVKQALQRYSEDRIGLADYALESGGASVISTRCSETYETKTALLSL |
| | (Sosa et al., 2012) | FGIPLWYHSQSPRVILQPDVHPGNCWAFQGPQGFAVVRLSARIRPTAVTLEHVP |
| | | KALSPNSTISSAPKDFAIFGFDEDLQQEGTLLGKFTYDQDGEPIQTFHFQAPTMA |
| | | TYQWVELRILTNWGHPEYTCIYRFRVHGEPAH |
| 58 | Sun1(458-913); | DDSKGMHRPGPLPPSPPPKVDHKASQWPQESDMGQKVASLSAQCHNHDERLA |
| | DnSun1 (Crisp et | ELTVLLQKLQIRVDQVDDGREGLSLWVKNVVGQHLQEMGTIEPPDAKTDFMTFH |
| | al., 2006) | HDHEVRLSNLEDVLRKLTEKSEAIQKELEETKLKAGSRDEEQPLLDRVQHLELEL |
| | | NLLKSQLSDWQHLKTSCEQAGARIQETVQLMFSEDQQGGSLEWLLEKLSSRFV |
| | | SKDELQVLLHDLELKLLQNITHHITVTGQAPTSEAIVSAVNQAGISGITEAQAHIIVN |
| | | NALKLYSQDKTGMVDFALESGGGSILSTRCSETYETKTALLSLFGVPLWYFSQSP |
| | | RWVIQPDIYPGNCWAFKGSQGYLVVRLSMKIYPTTFTMEHIPKTLSPTGNISSAPK |
| | | DFAVYGLETEYQEEGQPLGRFTYDQEGDSLQMFHTLERPDQAFQIVELRVLSN |
| | | WGHPEYTCLYRFRVHGEPIQ |
| 59 | Sun1(668-913) (Xu | SRFVSKDELQVLLHDLELKLLQNITHHITVTGQAPTSEAIVSAVNQAGISGITEAQA |
| | et al., 2018) | HIIVNNALKLYSQDKTGMVDFALESGGGSILSTRCSETYETKTALLSLFGVPLWYF |
| | | SQSPRVVIQPDIYPGNCWAFKGSQGYLVVRLSMKIYPTTFTMEHIPKTLSPTGNIS |
| | | SAPKDFAVYGLETEYQEEGQPLGRFTYDQEGDSLQMFHTLERPDQAFQIVELRV |
| | | LSNWGHPEYTCLYRFRVHGEPIQ |
| 60 | Sun2(410-731) (Nie | LLLLLGAGVSLWGQGNFFSLLPVLNWTAMQPTQRVDDSKGMHRPGPLPPSPPP |
| | et al., 2016) | KVDHKASQWPQESDMGQKVASLSAQCHNHDERLAELTVLLQKLQIRVDQVDDG |
| | | REGLSLWVKNVVGQHLQEMGTIEPPDAKTDFMTFHHDHEVRLSNLEDVLRKLTE |
| | | KSEAIQKELEETKLKAGSRDEEQPLLDRVQHLELELNLLKSQLSDWQHLKTSCEQ |
| | | AGARIQETVQLMFSEDQQGGSLEWLLEKLSSRFVSKDELQVLLHDLELKLLQNIT |
| | | HHITVTGQAPTSEAIVSAVNQAGISGITEAQAHIIVNNALKLYSQDKTGMVDFALES |
| | | GGGSILSTRCSETYETKTALLSLFGVPLWYFSQSPRVVIQPDIYPGNCWAFKGSQ |
| | | GYLVVRLSMKIYPTTFTMEHIPKTLSPTGNISSAPKDFAVYGLETEYQEEGQPLGR |
| | | FTYDQEGDSLQMFHTLERPDQAFQIVELRVLSNWGHPEYTCLYRFRVHGEPIQ |
| 61 | Sun2(458-731) | PSPPPKVDHKASQWPQESDMGQKVASLSAQCHNHDERLAELTVLLQKLQIRVD |
| | (Jahed et al., | QVDDGREGLSLWVKNVVGQHLQEMGTIEPPDAKTDFMTFHHDHEVRLSNLEDV |
| | 2016) | LRKLTEKSEAIQKELEETKLKAGSRDEEQPLLDRVQHLELELNLLKSQLSDWQHL |
| | | KTSCEQAGARIQETVQLMFSEDQQGGSLEWLLEKLSSRFVSKDELQVLLHDLEL |
| | | KLLQNITHHITVTGQAPTSEAIVSAVNQAGISGITEAQAHIIVNNALKLYSQDKTGM |
| | | VDFALESGGGSILSTRCSETYETKTALLSLFGVPLWYFSQSPRVVIQPDIYPGNC |
| | | WAFKGSQGYLVVRLSMKIYPTTFTMEHIPKTLSPTGNISSAPKDFAVYGLETEYQ |
| | | EEGQPLGRFTYDQEGDSLQMFHTLERPDQAFQIVELRVLSNWGHPEYTCLYRF |
| | | RVHGEPIQ |
| 62 | Sun2(482-731) (Nie | ASLSAQCHNHDERLAELTVLLQKLQIRVDQVDDGREGLSLWVKNVVGQHLQEM |
| | et al., 2016) | GTIEPPDAKTDFMTFHHDHEVRLSNLEDVLRKLTEKSEAIQKELEETKLKAGSRD |
| | | EEQPLLDRVQHLELELNLLKSQLSDWQHLKTSCEQAGARIQETVQLMFSEDQQG |
| | | GSLEWLLEKLSSRFVSKDELQVLLHDLELKLLQNITHHITVTGQAPTSEAIVSAVN |
| | | QAGISGITEAQAHIIVNNALKLYSQDKTGMVDFALESGGGSILSTRCSETYETKTA |
| | | LLSLFGVPLWYFSQSPRVVIQPDIYPGNCWAFKGSQGYLVVRLSMKIYPTTFTME |
| | | HIPKTLSPTGNISSAPKDFAVYGLETEYQEEGQPLGRFTYDQEGDSLQMFHTLER |
| | | PDQAFQIVELRVLSNWGHPEYTCLYRFRVHGEPIQ |
| 63 | SUN1(404-812) | HHHGENLRELTTLLQKLQARVDQMEGGAAGPSASVRDAVGQPPRETDFMAFH |
| | | QEHEVRMSHLEDILGKLREKSEAIQKELEQTKQKTISAVGEQLLPTVEHLQLELDQ |
| | | LKSELSSWRHVKTGCETVDAVQERVDVQVREMVKLLFSEDQQGGSLEQLLQRF |
| | | SSQFVSKGDLQTMLRDLQLQILRNVTHHVSVTKQLPTSEAVVSAVSEAGASGITE |
| | | AQARAIVNSALKLYSQDKTGMVDFALESGGGSILSTRCSETYETKTALMSLFGIPL |
| | | WYFSQSPRVVIQPDIYPGNCWAFKGSQGYLVVRLSMMIHPAAFTLEHIPKTLSPT |
| | | GNISSAPKDFAVYGLENEYQEEGQLLGQFTYDQDGESLQMFQALKRPDDTAFQI |
| | | VELRIFSNWGHPEYTCLYRFRVHGEPVK |
| 64 | SUN1(455-812) | HQEHEVRMSHLEDILGKLREKSEAIQKELEQTKQKTISAVGEQLLPTVEHLQLELD |
| | | QLKSELSSWRHVKTGCETVDAVQERVDVQVREMVKLLFSEDQQGGSLEQLLQR |
| | | FSSQFVSKGDLQTMLRDLQLQILRNVTHHVSVTKQLPTSEAVVSAVSEAGASGIT |
| | | EAQARAIVNSALKLYSQDKTGMVDFALESGGGSILSTRCSETYETKTALMSLFGIP |
| | | LWYFSQSPRVVIQPDIYPGNCWAFKGSQGYLVVRLSMMIHPAAFTLEHIPKTLSP |
| | | TGNISSAPKDFAVYGLENEYQEEGQLLGQFTYDQDGESLQMFQALKRPDDTAF |
| | | QIVELRIFSNWGHPEYTCLYRFRVHGEPVK |
| 65 | SUN1(495-812) | GEQLLPTVEHLQLELDQLKSELSSWRHVKTGCETVDAVQERVDVQVREMVKLLF |
| | | SEDQQGGSLEQLLQRFSSQFVSKGDLQTMLRDLQLQILRNVTHHVSVTKQLPTS |
| | | EAVVSAVSEAGASGITEAQARAIVNSALKLYSQDKTGMVDFALESGGGSILSTRC |
| | | SETYETKTALMSLFGIPLWYFSQSPRVVIQPDIYPGNCWAFKGSQGYLVVRLSM |
| | | MIHPAAFTLEHIPKTLSPTGNISSAPKDFAVYGLENEYQEEGQLLGQFTYDQDGE |
| | | SLQMFQALKRPDDTAFQIVELRIFSNWGHPEYTCLYRFRVHGEPVK |
| 66 | SUN1(562-812) | QRFSSQFVSKGDLQTMLRDLQLQILRNVTHHVSVTKQLPTSEAVVSAVSEAGAS |
| | | GITEAQARAIVNSALKLYSQDKTGMVDFALESGGGSILSTRCSETYETKTALMSLF |
| | | GIPLWYFSQSPRVVIQPDIYPGNCWAFKGSQGYLVVRLSMMIHPAAFTLEHIPKT |
| | | LSPTGNISSAPKDFAVYGLENEYQEEGQLLGQFTYDQDGESLQMFQALKRPDDT |
| | | AFQIVELRIFSNWGHPEYTCLYRFRVHGEPVK |
| 67 | SUN1(601-812) | TSEAVVSAVSEAGASGITEAQARAIVNSALKLYSQDKTGMVDFALESGGGSILST |
| | | RCSETYETKTALMSLFGIPLWYFSQSPRVVIQPDIYPGNCWAFKGSQGYLVVRLS |
| | | MMIHPAAFTLEHIPKTLSPTGNISSAPKDFAVYGLENEYQEEGQLLGQFTYDQDG |
| | | ESLQMFQALKRPDDTAFQIVELRIFSNWGHPEYTCLYRFRVHGEPVK |
| 68 | SUN1(636-812) | DKTGMVDFALESGGGSILSTRCSETYETKTALMSLFGIPLWYFSQSPRVVIQPDIY |
| | | PGNCWAFKGSQGYLVVRLSMMIHPAAFTLEHIPKTLSPTGNISSAPKDFAVYGLE |
| | | NEYQEEGQLLGQFTYDQDGESLQMFQALKRPDDTAFQIVELRIFSNWGHPEYTC |
| | | LYRFRVHGEPVK |
| 69 | SUN1(404-812) | HHHGENLRELTTLLQKLQARVDQMEGGAAGPSASVRDAVGQPPRETDFMAFH |
| | w/KDEL | QEHEVRMSHLEDILGKLREKSEAIQKELEQTKQKTISAVGEQLLPTVEHLQLELDQ |
| | | LKSELSSWRHVKTGCETVDAVQERVDVQVREMVKLLFSEDQQGGSLEQLLQRF |
| | | SSQFVSKGDLQTMLRDLQLQILRNVTHHVSVTKQLPTSEAVVSAVSEAGASGITE |
| | | AQARAIVNSALKLYSQDKTGMVDFALESGGGSILSTRCSETYETKTALMSLFGIPL |
| | | WYFSQSPRVVIQPDIYPGNCWAFKGSQGYLVVRLSMMIHPAAFTLEHIPKTLSPT |
| | | GNISSAPKDFAVYGLENEYQEEGQLLGQFTYDQDGESLQMFQALKRPDDTAFQI |
| | | VELRIFSNWGHPEYTCLYRFRVHGEPVKKDEL |
| 70 | SUN1(455-812) | HQEHEVRMSHLEDILGKLREKSEAIQKELEQTKQKTISAVGEQLLPTVEHLQLELD |
| | w/KDEL | QLKSELSSWRHVKTGCETVDAVQERVDVQVREMVKLLFSEDQQGGSLEQLLQR |
| | | FSSQFVSKGDLQTMLRDLQLQILRNVTHHVSVTKQLPTSEAVVSAVSEAGASGIT |
| | | EAQARAIVNSALKLYSQDKTGMVDFALESGGGSILSTRCSETYETKTALMSLFGIP |
| | | LWYFSQSPRVVIQPDIYPGNCWAFKGSQGYLVVRLSMMIHPAAFTLEHIPKTLSP |
| | | TGNISSAPKDFAVYGLENEYQEEGQLLGQFTYDQDGESLQMFQALKRPDDTAF |
| | | QIVELRIFSNWGHPEYTCLYRFRVHGEPVKKDEL |
| 71 | SUN1(495-812) | GEQLLPTVEHLQLELDQLKSELSSWRHVKTGCETVDAVQERVDVQVREMVKLLF |
| | w/KDEL | SEDQQGGSLEQLLQRFSSQFVSKGDLQTMLRDLQLQILRNVTHHVSVTKQLPTS |
| | | EAVVSAVSEAGASGITEAQARAIVNSALKLYSQDKTGMVDFALESGGGSILSTRC |
| | | SETYETKTALMSLFGIPLWYFSQSPRVVIQPDIYPGNCWAFKGSQGYLVVRLSM |
| | | MIHPAAFTLEHIPKTLSPTGNISSAPKDFAVYGLENEYQEEGQLLGQFTYDQDGE |
| | | SLQMFQALKRPDDTAFQIVELRIFSNWGHPEYTCLYRFRVHGEPVKKDEL |
| 72 | SUN1(562-812) | QRFSSQFVSKGDLQTMLRDLQLQILRNVTHHVSVTKQLPTSEAVVSAVSEAGAS |
| | w/KDEL | GITEAQARAIVNSALKLYSQDKTGMVDFALESGGGSILSTRCSETYETKTALMSLF |
| | | GIPLWYFSQSPRVVIQPDIYPGNCWAFKGSQGYLVVRLSMMIHPAAFTLEHIPKT |
| | | LSPTGNISSAPKDFAVYGLENEYQEEGQLLGQFTYDQDGESLQMFQALKRPDDT |
| | | AFQIVELRIFSNWGHPEYTCLYRFRVHGEPVKKDEL |
| 73 | SUN1(601-812) | TSEAVVSAVSEAGASGITEAQARAIVNSALKLYSQDKTGMVDFALESGGGSILST |
| | w/KDEL | RCSETYETKTALMSLFGIPLWYFSQSPRVVIQPDIYPGNCWAFKGSQGYLVVRLS |
| | | MMIHPAAFTLEHIPKTLSPTGNISSAPKDFAVYGLENEYQEEGQLLGQFTYDQDG |
| | | ESLQMFQALKRPDDTAFQIVELRIFSNWGHPEYTCLYRFRVHGEPVKKDEL |
| 74 | SUN1(616-812) | GITEAQARAIVNSALKLYSQDKTGMVDFALESGGGSILSTRCSETYETKTALMSLF |
| | w/KDEL | GIPLWYFSQSPRVVIQPDIYPGNCWAFKGSQGYLVVRLSMMIHPAAFTLEHIPKT |
| | | LSPTGNISSAPKDFAVYGLENEYQEEGQLLGQFTYDQDGESLQMFQALKRPDDT |
| | | AFQIVELRIFSNWGHPEYTCLYRFRVHGEPVKKDEL |
| 75 | SUN2(522-717) | GVTEEQVHHIVKQALQRYSEDRIGLADYALESGGASVISTRCSETYETKTALLSLF |
| | w/KDEL | GIPLWYHSQSPRVILQPDVHPGNCWAFQGPQGFAVVRLSARIRPTAVTLEHVPK |
| | | ALSPNSTISSAPKDFAIFGFDEDLQQEGTLLGKFTYDQDGEPIQTFHFQAPTMAT |
| | | YQVVELRILTNWGHPEYTCIYRFRVHGEPAHKDEL |
| 76 | Signal Peptide | MKWVTFISLLFLFSSAYSRGVFRRD |
| 77 | ER retention motif | KDEL |
| 78 | HA tag | YPYDVPDYA |
| 79 | ALFA Tag | SRLEEELRRRLTE |
| 80 | SUN1(360-812) | MKWVTFISLLFLFSSAYSRGVFRRDGSYPYDVPDYAPSRLEEELRRRLTEPTGD |
| | w/SP, HA tag, ALFA | DPQDVFKPTTSRLKQPLQGDSEAFPWHWMSGVEQQVASLSGQCHHHGENLRE |
| | tag, KDEL | LTTLLQKLQARVDQMEGGAAGPSASVRDAVGQPPRETDFMAFHQEHEVRMSHL |
| | | EDILGKLREKSEAIQKELEQTKQKTISAVGEQLLPTVEHLQLELDQLKSELSSWRH |
| | | VKTGCETVDAVQERVDVQVREMVKLLFSEDQQGGSLEQLLQRFSSQFVSKGDL |
| | | QTMLRDLQLQILRNVTHHVSVTKQLPTSEAVVSAVSEAGASGITEAQARAIVNSA |
| | | LKLYSQDKTGMVDFALESGGGSILSTRCSETYETKTALMSLFGIPLWYFSQSPRV |
| | | VIQPDIYPGNCWAFKGSQGYLVVRLSMMIHPAAFTLEHIPKTLSPTGNISSAPKDF |
| | | AVYGLENEYQEEGQLLGQFTYDQDGESLQMFQALKRPDDTAFQIVELRIFSNWG |
| | | HPEYTCLYRFRVHGEPVKKDEL |
| 81 | SUN1(404-812) | MKWVTFISLLFLFSSAYSRGVFRRDGSYPYDVPDYAPSRLEEELRRRLTEPTGH |
| | w/SP, HA tag, | HHGENLRELTTLLQKLQARVDQMEGGAAGPSASVRDAVGQPPRETDFMAFHQE |
| | ALFA tag, KDEL | HEVRMSHLEDILGKLREKSEAIQKELEQTKQKTISAVGEQLLPTVEHLQLELDQLK |
| | | SELSSWRHVKTGCETVDAVQERVDVQVREMVKLLFSEDQQGGSLEQLLQRFSS |
| | | QFVSKGDLQTMLRDLQLQILRNVTHHVSVTKQLPTSEAVVSAVSEAGASGITEAQ |
| | | ARAIVNSALKLYSQDKTGMVDFALESGGGSILSTRCSETYETKTALMSLFGIPLW |
| | | YFSQSPRVVIQPDIYPGNCWAFKGSQGYLVVRLSMMIHPAAFTLEHIPKTLSPTG |
| | | NISSAPKDFAVYGLENEYQEEGQLLGQFTYDQDGESLQMFQALKRPDDTAFQIV |
| | | ELRIFSNWGHPEYTCLYRFRVHGEPVKKDEL |
| 82 | SUN1(455-812) | MKWVTFISLLFLFSSAYSRGVFRRDGSYPYDVPDYAPSRLEEELRRRLTEPTGH |
| | w/SP, HA tag, ALFA | QEHEVRMSHLEDILGKLREKSEAIQKELEQTKQKTISAVGEQLLPTVEHLQLELDQ |
| | tag, KDEL | LKSELSSWRHVKTGCETVDAVQERVDVQVREMVKLLFSEDQQGGSLEQLLQRF |
| | | SSQFVSKGDLQTMLRDLQLQILRNVTHHVSVTKQLPTSEAVVSAVSEAGASGITE |
| | | AQARAIVNSALKLYSQDKTGMVDFALESGGGSILSTRCSETYETKTALMSLFGIPL |
| | | WYFSQSPRVVIQPDIYPGNCWAFKGSQGYLVVRLSMMIHPAAFTLEHIPKTLSPT |
| | | GNISSAPKDFAVYGLENEYQEEGQLLGQFTYDQDGESLQMFQALKRPDDTAFQI |
| | | VELRIFSNWGHPEYTCLYRFRVHGEPVKKDEL |
| 83 | SUN1(495-812) | MKWVTFISLLFLFSSAYSRGVFRRDGSYPYDVPDYAPSRLEEELRRRLTEPTGG |
| | w/SP, HA tag, | EQLLPTVEHLQLELDQLKSELSSWRHVKTGCETVDAVQERVDVQVREMVKLLFS |
| | ALFA tag, KDEL | EDQQGGSLEQLLQRFSSQFVSKGDLQTMLRDLQLQILRNVTHHVSVTKQLPTSE |
| | | AVVSAVSEAGASGITEAQARAIVNSALKLYSQDKTGMVDFALESGGGSILSTRCS |
| | | ETYETKTALMSLFGIPLWYFSQSPRVVIQPDIYPGNCWAFKGSQGYLVVRLSMMI |
| | | HPAAFTLEHIPKTLSPTGNISSAPKDFAVYGLENEYQEEGQLLGQFTYDQDGESL |
| | | QMFQALKRPDDTAFQIVELRIFSNWGHPEYTCLYRFRVHGEPVKKDEL |
| 84 | SUN1(562-812) | MKWVTFISLLFLFSSAYSRGVFRRDGSYPYDVPDYAPSRLEEELRRRLTEPTGQ |
| | w/SP, HA tag, | RFSSQFVSKGDLQTMLRDLQLQILRNVTHHVSVTKQLPTSEAVVSAVSEAGASGI |
| | ALFA tag, KDEL | TEAQARAIVNSALKLYSQDKTGMVDFALESGGGSILSTRCSETYETKTALMSLFGI |
| | | PLWYFSQSPRVVIQPDIYPGNCWAFKGSQGYLVVRLSMMIHPAAFTLEHIPKTLS |
| | | PTGNISSAPKDFAVYGLENEYQEEGQLLGQFTYDQDGESLQMFQALKRPDDTAF |
| | | QIVELRIFSNWGHPEYTCLYRFRVHGEPVKKDEL |
| 85 | SUN1(601-812) | MKWVTFISLLFLFSSAYSRGVFRRDGSYPYDVPDYAPSRLEEELRRRLTEPTGTS |
| | w/SP, HA tag, | EAVVSAVSEAGASGITEAQARAIVNSALKLYSQDKTGMVDFALESGGGSILSTRC |
| | ALFA tag, KDEL | SETYETKTALMSLFGIPLWYFSQSPRVVIQPDIYPGNCWAFKGSQGYLVVRLSM |
| | | MIHPAAFTLEHIPKTLSPTGNISSAPKDFAVYGLENEYQEEGQLLGQFTYDQDGE |
| | | SLQMFQALKRPDDTAFQIVELRIFSNWGHPEYTCLYRFRVHGEPVKKDEL |
| 86 | SUN1(616-812) | MKWVTFISLLFLFSSAYSRGVFRRDGSYPYDVPDYAPSRLEEELRRRLTEPTGGI |
| | w/SP, HA tag, | TEAQARAIVNSALKLYSQDKTGMVDFALESGGGSILSTRCSETYETKTALMSLFGI |
| | ALFA tag, KDEL | PLWYFSQSPRVVIQPDIYPGNCWAFKGSQGYLVVRLSMMIHPAAFTLEHIPKTLS |
| | | PTGNISSAPKDFAVYGLENEYQEEGQLLGQFTYDQDGESLQMFQALKRPDDTAF |
| | | QIVELRIFSNWGHPEYTCLYRFRVHGEPVKKDEL |
| 87 | SUN1(636-812) | MKWVTFISLLFLFSSAYSRGVFRRDGSYPYDVPDYAPSRLEEELRRRLTEPTGD |
| | w/SP, HA tag, | KTGMVDFALESGGGSILSTRCSETYETKTALMSLFGIPLWYFSQSPRVVIQPDIYP |
| | ALFA tag, KDEL | GNCWAFKGSQGYLVVRLSMMIHPAAFTLEHIPKTLSPTGNISSAPKDFAVYGLEN |
| | | EYQEEGQLLGQFTYDQDGESLQMFQALKRPDDTAFQIVELRIFSNWGHPEYTCL |
| | | YRFRVHGEPVKKDEL |
| 88 | Human SUN1 CC1, | GEQLLPTVEHLQLELDQLKSELSSWRHVKTGCETVDAVQERVDVQVREMVKLLF |
| | CC2 α1, CC2 α2, | SEDQQGGSLEQLLQRFSSQFVSKGDLQTMLRDLQLQILRNVTHHVSVTKQLPTS |
| | CC2 α3 | EAVVSAVSEAGASGITEAQARAIVNSALKL |
| 89 | Human SUN1 CC2 | KGDLQTMLRDLQLQILRNVTHHVSVTKQLPTSEAVVSAVSEAGASGITEAQARAI |
| | α1, CC2 α2, CC2 | VNSALKL |
| | α3 | |
| 90 | Human SUN1 CC2 | TSEAVVSAVSEAGASGITEAQARAIVNSALKL |
| | α2, CC2 α3 | |
| 91 | Human SUN2 CC1, | QESFQESSVKELRRLEDQLAGLQQELAALALKQSSVAEEVGLLPQQIQAVRDDV |
| | CC2 α1, CC2 α2, | ESQFPAWISQFLARGGGGRVGLLQREEMQAQLRELESKILTHVAEMQGKSARE |
| | CC2 α3 | AAASLSLTLQKEGVIGVTEEQVHHIVKQALQR |
| 92 | Human SUN2 CC2 | REEMQAQLRELESKILTHVAEMQGKSAREAAASLSLTLQKEGVIGVTEEQVHHIV |
| | α1, CC2 α2, CC2 | KQALQR |
| | α3 | |
| 93 | Human SUN2 CC2 | AAASLSLTLQKEGVIGVTEEQVHHIVKQALQR |
| | α2, CC2 α3 | |
| 94 | SUN1(483-812) | LEQTKQKTISAVGEQLLPTVEHLQLELDQLKSELSSWRHVKTGCETVDAVQERV |
| | | DVQVREMVKLLFSEDQQGGSLEQLLQRFSSQFVSKGDLQTMLRDLQLQILRNVT |
| | | HHVSVTKQLPTSEAVVSAVSEAGASGITEAQARAIVNSALKLYSQDKTGMVDFAL |
| | | ESGGGSILSTRCSETYETKTALMSLFGIPLWYFSQSPRVVIQPDIYPGNCWAFKG |
| | | SQGYLVVRLSMMIHPAAFTLEHIPKTLSPTGNISSAPKDFAVYGLENEYQEEGQLL |
| | | GQFTYDQDGESLQMFQALKRPDDTAFQIVELRIFSNWGHPEYTCLYRFRVHGEP |
| | | VK |
| 95 | SUN1(483-812) | LEQTKQKTISAVGEQLLPTVEHLQLELDQLKSELSSWRHVKTGCETVDAVQERV |
| | w/KDEL | DVQVREMVKLLFSEDQQGGSLEQLLQRFSSQFVSKGDLQTMLRDLQLQILRNVT |
| | | HHVSVTKQLPTSEAVVSAVSEAGASGITEAQARAIVNSALKLYSQDKTGMVDFAL |
| | | ESGGGSILSTRCSETYETKTALMSLFGIPLWYFSQSPRVVIQPDIYPGNCWAFKG |
| | | SQGYLVVRLSMMIHPAAFTLEHIPKTLSPTGNISSAPKDFAVYGLENEYQEEGQLL |
| | | GQFTYDQDGESLQMFQALKRPDDTAFQIVELRIFSNWGHPEYTCLYRFRVHGEP |
| | | VKKDEL |
| 96 | SUN1(483-812) | MKWVTFISLLFLFSSAYSRGVFRRDGSYPYDVPDYAPSRLEEELRRRLTEPTGLE |
| | w/SP, HA tag, | QTKQKTISAVGEQLLPTVEHLQLELDQLKSELSSWRHVKTGCETVDAVQERVDV |
| | ALFA tag, KDEL | QVREMVKLLFSEDQQGGSLEQLLQRFSSQFVSKGDLQTMLRDLQLQILRNVTHH |
| | | VSVTKQLPTSEAVVSAVSEAGASGITEAQARAIVNSALKLYSQDKTGMVDFALES |
| | | GGGSILSTRCSETYETKTALMSLFGIPLWYFSQSPRVVIQPDIYPGNCWAFKGSQ |
| | | GYLVVRLSMMIHPAAFTLEHIPKTLSPTGNISSAPKDFAVYGLENEYQEEGQLLG |
| | | QFTYDQDGESLQMFQALKRPDDTAFQIVELRIFSNWGHPEYTCLYRFRVHGEPV |
| | | KKDEL |
| 97 | Human SUN1 | LEQTKQKTISAVGEQLLPTVEHLQLELDQLKSELSSWRHVKTGCETVDAVQERV |
| | partial SMART coil- | DVQVREMVKLLFSEDQQGGSLEQLLQRFSSQFVSKGDLQTMLRDLQLQILRNVT |
| | coiled 2, CC1, CC2 | HHVSVTKQLPTSEAVVSAVSEAGASGITEAQARAIVNSALKL |
| | α1, CC2 α2, CC2 | |
| | α3 | |
| 98 | Human SUN2 | IQQLKSEWQSMTQESFQESSVKELRRLEDQLAGLQQELAALALKQSSVAEEVGL |
| | partial SMART coil- | LPQQIQAVRDDVESQFPAWISQFLARGGGGRVGLLQREEMQAQLRELESKILTH |
| | coiled 2, CC1, CC2 | VAEMQGKSAREAAASLSLTLQKEGVIGVTEEQVHHIVKQALQR |
| | α1, CC2 α2, CC2 | |
| | α3 | |
| 99 | SUN1(360-812) | DDPQDVFKPTTSRLKQPLQGDSEAFPWHWMSGVEQQVASLSGQCHHHGENLR |
| | w/KDEL | ELTTLLQKLQARVDQMEGGAAGPSASVRDAVGQPPRETDFMAFHQEHEVRMS |
| | | HLEDILGKLREKSEAIQKELEQTKQKTISAVGEQLLPTVEHLQLELDQLKSELSSW |
| | | RHVKTGCETVDAVQERVDVQVREMVKLLFSEDQQGGSLEQLLQRFSSQFVSKG |
| | | DLQTMLRDLQLQILRNVTHHVSVTKQLPTSEAVVSAVSEAGASGITEAQARAIVN |
| | | SALKLYSQDKTGMVDFALESGGGSILSTRCSETYETKTALMSLFGIPLWYFSQSP |
| | | RVVIQPDIYPGNCWAFKGSQGYLVVRLSMMIHPAAFTLEHIPKTLSPTGNISSAPK |
| | | DFAVYGLENEYQEEGQLLGQFTYDQDGESLQMFQALKRPDDTAFQIVELRIFSN |
| | | WGHPEYTCLYRFRVHGEPVKKDEL |
| 100 | Minimal human | MKWVTFISLLFLFSSAYS |
| | serum albumin | |
| | signal peptide | |
| 101 | SUN1(360-812) | MKWVTFISLLFLFSSAYSDDPQDVFKPTTSRLKQPLQGDSEAFPWHWMSGVEQ |
| | w/SP, KDEL | QVASLSGQCHHHGENLRELTTLLQKLQARVDQMEGGAAGPSASVRDAVGQPP |
| | | RETDFMAFHQEHEVRMSHLEDILGKLREKSEAIQKELEQTKQKTISAVGEQLLPT |
| | | VEHLQLELDQLKSELSSWRHVKTGCETVDAVQERVDVQVREMVKLLFSEDQQG |
| | | GSLEQLLQRFSSQFVSKGDLQTMLRDLQLQILRNVTHHVSVTKQLPTSEAVVSAV |
| | | SEAGASGITEAQARAIVNSALKLYSQDKTGMVDFALESGGGSILSTRCSETYETK |
| | | TALMSLFGIPLWYFSQSPRVVIQPDIYPGNCWAFKGSQGYLVVRLSMMIHPAAFT |
| | | LEHIPKTLSPTGNISSAPKDFAVYGLENEYQEEGQLLGQFTYDQDGESLQMFQAL |
| | | KRPDDTAFQIVELRIFSNWGHPEYTCLYRFRVHGEPVKKDEL |
| 102 | SUN1(404-812) | MKWVTFISLLFLFSSAYSHHHGENLRELTTLLQKLQARVDQMEGGAAGPSASVR |
| | w/SP, KDEL | DAVGQPPRETDFMAFHQEHEVRMSHLEDILGKLREKSEAIQKELEQTKQKTISAV |
| | | GEQLLPTVEHLQLELDQLKSELSSWRHVKTGCETVDAVQERVDVQVREMVKLLF |
| | | SEDQQGGSLEQLLQRFSSQFVSKGDLQTMLRDLQLQILRNVTHHVSVTKQLPTS |
| | | EAVVSAVSEAGASGITEAQARAIVNSALKLYSQDKTGMVDFALESGGGSILSTRC |
| | | SETYETKTALMSLFGIPLWYFSQSPRVVIQPDIYPGNCWAFKGSQGYLVVRLSM |
| | | MIHPAAFTLEHIPKTLSPTGNISSAPKDFAVYGLENEYQEEGQLLGQFTYDQDGE |
| | | SLQMFQALKRPDDTAFQIVELRIFSNWGHPEYTCLYRFRVHGEPVKKDEL |
| 103 | SUN1(455-812) | MKWVTFISLLFLFSSAYSHQEHEVRMSHLEDILGKLREKSEAIQKELEQTKQKTIS |
| | w/SP, KDEL | AVGEQLLPTVEHLQLELDQLKSELSSWRHVKTGCETVDAVQERVDVQVREMVK |
| | | LLFSEDQQGGSLEQLLQRFSSQFVSKGDLQTMLRDLQLQILRNVTHHVSVTKQL |
| | | PTSEAVVSAVSEAGASGITEAQARAIVNSALKLYSQDKTGMVDFALESGGGSILS |
| | | TRCSETYETKTALMSLFGIPLWYFSQSPRVVIQPDIYPGNCWAFKGSQGYLVVRL |
| | | SMMIHPAAFTLEHIPKTLSPTGNISSAPKDFAVYGLENEYQEEGQLLGQFTYDQD |
| | | GESLQMFQALKRPDDTAFQIVELRIFSNWGHPEYTCLYRFRVHGEPVKKDEL |
| 104 | SUN1(495-812) | MKWVTFISLLFLFSSAYSGEQLLPTVEHLQLELDQLKSELSSWRHVKTGCETVDA |
| | w/SP, KDEL | VQERVDVQVREMVKLLFSEDQQGGSLEQLLQRFSSQFVSKGDLQTMLRDLQLQ |
| | | ILRNVTHHVSVTKQLPTSEAVVSAVSEAGASGITEAQARAIVNSALKLYSQDKTG |
| | | MVDFALESGGGSILSTRCSETYETKTALMSLFGIPLWYFSQSPRVVIQPDIYPGN |
| | | CWAFKGSQGYLVVRLSMMIHPAAFTLEHIPKTLSPTGNISSAPKDFAVYGLENEY |
| | | QEEGQLLGQFTYDQDGESLQMFQALKRPDDTAFQIVELRIFSNWGHPEYTCLYR |
| | | FRVHGEPVKKDEL |
| 105 | SUN1(483-812) | MKWVTFISLLFLFSSAYSLEQTKQKTISAVGEQLLPTVEHLQLELDQLKSELSSWR |
| | w/SP, KDEL | HVKTGCETVDAVQERVDVQVREMVKLLFSEDQQGGSLEQLLQRFSSQFVSKGD |
| | | LQTMLRDLQLQILRNVTHHVSVTKQLPTSEAVVSAVSEAGASGITEAQARAIVNS |
| | | ALKLYSQDKTGMVDFALESGGGSILSTRCSETYETKTALMSLFGIPLWYFSQSPR |
| | | VVIQPDIYPGNCWAFKGSQGYLVVRLSMMIHPAAFTLEHIPKTLSPTGNISSAPKD |
| | | FAVYGLENEYQEEGQLLGQFTYDQDGESLQMFQALKRPDDTAFQIVELRIFSNW |
| | | GHPEYTCLYRFRVHGEPVKKDEL |
| 106 | SUN1(562-812) | MKWVTFISLLFLFSSAYSQRFSSQFVSKGDLQTMLRDLQLQILRNVTHHVSVTKQ |
| | w/SP, KDEL | LPTSEAVVSAVSEAGASGITEAQARAIVNSALKLYSQDKTGMVDFALESGGGSIL |
| | | STRCSETYETKTALMSLFGIPLWYFSQSPRVVIQPDIYPGNCWAFKGSQGYLVVR |
| | | LSMMIHPAAFTLEHIPKTLSPTGNISSAPKDFAVYGLENEYQEEGQLLGQFTYDQ |
| | | DGESLQMFQALKRPDDTAFQIVELRIFSNWGHPEYTCLYRFRVHGEPVKKDEL |
| 107 | SUN1(601-812) | MKWVTFISLLFLFSSAYSTSEAVVSAVSEAGASGITEAQARAIVNSALKLYSQDKT |
| | w/SP, KDEL | GMVDFALESGGGSILSTRCSETYETKTALMSLFGIPLWYFSQSPRVVIQPDIYPG |
| | | NCWAFKGSQGYLVVRLSMMIHPAAFTLEHIPKTLSPTGNISSAPKDFAVYGLENE |
| | | YQEEGQLLGQFTYDQDGESLQMFQALKRPDDTAFQIVELRIFSNWGHPEYTCLY |
| | | RFRVHGEPVKKDEL |
| 108 | SUN1(616-812) | MKWVTFISLLFLFSSAYSGITEAQARAIVNSALKLYSQDKTGMVDFALESGGGSIL |
| | w/SP, KDEL | STRCSETYETKTALMSLFGIPLWYFSQSPRVVIQPDIYPGNCWAFKGSQGYLVVR |
| | | LSMMIHPAAFTLEHIPKTLSPTGNISSAPKDFAVYGLENEYQEEGQLLGQFTYDQ |
| | | DGESLQMFQALKRPDDTAFQIVELRIFSNWGHPEYTCLYRFRVHGEPVKKDEL |
| 109 | SUN1(636-812) | MKWVTFISLLFLFSSAYSDKTGMVDFALESGGGSILSTRCSETYETKTALMSLFGI |
| | w/SP, KDEL | PLWYFSQSPRVVIQPDIYPGNCWAFKGSQGYLVVRLSMMIHPAAFTLEHIPKTLS |
| | | PTGNISSAPKDFAVYGLENEYQEEGQLLGQFTYDQDGESLQMFQALKRPDDTAF |
| | | QIVELRIFSNWGHPEYTCLYRFRVHGEPVKKDEL |

### Numbered Paragraphs

The following numbered paragraphs (paras) provide further statements of features and combinations of features which are contemplated in connection with the present invention:
1. A nucleic acid encoding a LINC complex inhibiting polypeptide, wherein the LINC complex inhibiting polypeptide comprises: (i) an inhibitory region comprising an amino acid sequence corresponding to the α3 helix of the CC2 region and the SUN domain of a SUN domain-containing protein, and (ii) an endoplasmic reticulum retention motif;
   and wherein the LINC complex inhibiting polypeptide does not comprise the amino acid sequence 10 of any one of SEQ ID NO:43, 45 or 58.
2. The nucleic acid according to para 1, wherein the inhibitory region of the LINC complex inhibiting polypeptide does not consist essentially of the amino acid sequence of SEQ ID NO:44 or 46.
3. The nucleic acid according to para 1 or para 2, wherein the inhibitory region of the LINC complex inhibiting polypeptide does not consist essentially of the amino acid sequence of any one of SEQ ID NOS: 49, 50, 51, 52, 53, 54, 55, 56, 57, 59, 60, 61 or 62.
4. The nucleic acid according to any one of paras 1 to 3, wherein the inhibitory region of the LINC complex inhibiting polypeptide consists essentially of an amino acid sequence having at least 80% amino acid sequence identity to the amino acid sequence of one of SEQ ID NOs: 63, 64, 65, 66, 67, 68, or 94.
5. The nucleic acid according to any one of paras 1 to 4, wherein the LINC complex inhibiting polypeptide comprises, or consists essentially of, an amino acid sequence having at least 80% amino acid sequence identity to the amino acid sequence of one of SEQ ID NOs: 69, 70, 71, 72, 73, 74 or 95.
6. The nucleic acid according to any one of paras 1 to 5, wherein the LINC complex inhibiting polypeptide comprises a signal peptide.
7. The nucleic acid according to any one of paras 1 to 6, wherein the nucleic acid is a vector suitable for delivering the nucleic acid encoding a LINC complex inhibiting polypeptide as a gene therapy.
8. The nucleic acid according to para 7, wherein the vector is an adeno-associated virus (AAV) vector.
9. A LINC complex inhibiting polypeptide, comprising: (i) an inhibitory region comprising an amino acid sequence corresponding to the α3 helix of the CC2 region and the SUN domain of a SUN domain-containing protein, and (ii) an endoplasmic reticulum retention motif;
   and wherein the LINC complex inhibiting polypeptide does not comprise the amino acid sequence of SEQ ID NO:43 or 45.
10. The LINC complex inhibiting polypeptide according to para 9, wherein the inhibitory region of the LINC complex inhibiting polypeptide does not consist essentially of the amino acid sequence of SEQ ID NO: 44 or 46.
11. The LINC complex inhibiting polypeptide according to para 9 or para 10, wherein the inhibitory region of the LINC complex inhibiting polypeptide does not consist essentially of the amino acid sequence of any one of SEQ ID NOs: 49, 50, 51, 52, 53, 54, 55, 56, 57, 58, 59, 60, 61 or 62.
12. The LINC complex inhibiting polypeptide according to any one of paras 9 to 11, wherein the inhibitory region of the LINC complex inhibiting polypeptide consists essentially of an amino acid sequence having at least 80% amino acid sequence identity to the amino acid sequence of one of SEQ ID NOs: 63, 64, 65, 66, 67, 68, or 94.
13. The LINC complex inhibiting polypeptide according to any one of paras 9 to 12, wherein the LINC complex inhibiting polypeptide comprises, or consists essentially of, an amino acid sequence having at least 80% amino acid sequence identity to the amino acid sequence of one of SEQ ID NOs: 69, 70, 71, 72, 73, 74, or 95.
14. The LINC complex inhibiting polypeptide according to any one of paras 9 to 13, wherein the LINC complex inhibiting polypeptide comprises a signal peptide.
15. A method for identifying a LINC complex inhibiting polypeptide, comprising:
   introducing nucleic acid encoding a candidate LINC complex inhibiting polypeptide, wherein the candidate LINC complex inhibiting polypeptide comprises an amino acid sequence corresponding to the SUN domain of a SUN domain-containing protein; and
   subsequently analysing the cells to determine the subcellular localisation of an interaction partner for a SUN domain-containing protein;
   wherein a candidate LINC complex inhibiting polypeptide is determined to be a LINC complex inhibiting polypeptide where a change in the subcellular localisation of an interaction partner for a SUN domain-containing protein is detected.
16. A method for identifying a LINC complex inhibiting polypeptide, comprising:
   introducing nucleic acid encoding a candidate LINC complex inhibiting polypeptide, wherein the candidate LINC complex inhibiting polypeptide comprises an amino acid sequence corresponding to the KASH domain of a KASH domain-containing protein; and
   subsequently analysing the cells to determine the subcellular localisation of an interaction partner for a KASH domain-containing protein;
   wherein a candidate LINC complex inhibiting polypeptide is determined to be a LINC complex inhibiting polypeptide where a change in the subcellular localisation of an interaction partner for a KASH domain-containing protein is detected.
17. The method according to para 15 or para 16, wherein a candidate LINC complex inhibiting polypeptide is determined to be a LINC complex inhibiting polypeptide where an increase in the proportion of the interaction partner for a SUN/KASH domain-containing protein localised to the endoplasmic reticulum is detected.
18. A LINC complex inhibiting polypeptide identified by the method according to any one of paras 15 to 17.
19. A nucleic acid encoding a LINC complex inhibiting polypeptide according to any one of paras 9 to 14 or para 18.
20. A vector comprising a nucleic acid according to para 19, wherein the vector is a vector suitable for delivering the nucleic acid encoding a LINC complex inhibiting polypeptide as a gene therapy.
21. The vector according to para 20, wherein the vector is an adeno-associated virus (AAV) vector.
22. A cell comprising a nucleic acid according to any one of paras 1 to 8 or para 19, a LINC complex inhibiting polypeptide according to any one of paras 9 to 14 or para 18, or a vector according to para 20 or para 21.
23. A pharmaceutical composition comprising a nucleic acid according to any one of paras 1 to 8 or para 19, a LINC complex inhibiting polypeptide according to any one of paras 9 to 14 or para 18, a vector according to para 20 or para 21, or a cell according to para 22.
24. A nucleic acid according to any one of paras 1 to 8 or para 19, a LINC complex inhibiting polypeptide according to any one of paras 9 to 14 or para 18, a vector according to para 20 or para 21, a cell according to para 22 or a pharmaceutical composition according to para 23, for use in a method of medical treatment or prophylaxis.
25. A nucleic acid according to any one of paras 1 to 8 or para 19, a LINC complex inhibiting polypeptide according to any one of paras 9 to 14 or para 18, a vector according to para 20 or para 21, a cell according to para 22 or a pharmaceutical composition according to para 23, for use in a method of treating or preventing a laminopathy.
26. Use of a nucleic acid according to any one of paras 1 to 8 or para 19, a LINC complex inhibiting polypeptide according to any one of paras 9 to 14 or para 18, a vector according to para 20 or para 21, a cell according to para 22 or a pharmaceutical composition according to para 23 in the manufacture of a medicament for treating or preventing a laminopathy.
27. A method of treating or preventing a laminopathy, comprising administering a therapeutically-or prophylactically-effective amount of a nucleic acid according to any one of paras 1 to 8 or para 19, a LINC complex inhibiting polypeptide according to any one of paras 9 to 14 or para 18, a vector according to para 20 or para 21, a cell according to para 22 or a pharmaceutical composition according to para 23 to a subject.
28. The nucleic acid, LINC complex inhibiting polypeptide, vector, cell or pharmaceutical composition for use according to para 25, the use according to para 26, or the method according to para 27, wherein the laminopathy is characterised by one or more of myopathy, cardiomyopathy, dilated cardiomyopathy, muscular dystrophy, cardiac muscular dystrophy, skeletal muscular dystrophy, progeria, neuropathy, lipoatrophy, skeletal dysplasia, lipodystrophy, leukodystrophy or dermopathy.
29. The nucleic acid, LINC complex inhibiting polypeptide, vector, cell or pharmaceutical composition for use, the use, or the method according to any one of paras 25 to 28, wherein the laminopathy is associated with mutation to LMNA.
30. The nucleic acid, LINC complex inhibiting polypeptide, vector, cell or pharmaceutical composition for use, the use, or the method according to any one of paras 25 to 29, wherein the laminopathy is selected from: Hutchinson-Gilford Progeria Syndrome; Dilated Cardiomyopathy; Muscular Dystrophy, Congenital, Lmna-Related; Emery-Dreifuss Muscular Dystrophy 2, Autosomal Dominant; Muscular Dystrophy; Mandibuloacral Dysplasia with Type a Lipodystrophy; Cardiomyopathy, Dilated, 1a; Charcot-Marie-Tooth Disease; Limb-Girdle Muscular Dystrophy; Cardiomyopathy, Dilated, with Hypergonadotropic Hypogonadism; Emery-Dreifuss Muscular Dystrophy 3, Autosomal Recessive; Lipodystrophy, Familial Partial, Type 2; Emery-Dreifuss Muscular Dystrophy; Charcot-Marie-Tooth Disease, Axonal, Type 2b1; Heart-Hand Syndrome, Slovenian Type; Aging; Familial Partial Lipodystrophy; Restrictive Dermopathy, Lethal; Arrhythmogenic Right Ventricular Cardiomyopathy; Tooth Disease; Heart Disease; Werner Syndrome; Hypertrophic Cardiomyopathy; Left Ventricular Noncompaction; Atrioventricular Block; Calcinosis; Acroosteolysis; Autosomal Dominant Limb-Girdle Muscular Dystrophy; Diabetes Mellitus, Noninsulin-Dependent; Osteoporosis; Atrial Fibrillation; Atrial Standstill 1; Acanthosis Nigricans; Cardiac Conduction Defect; Catecholaminergic Polymorphic Ventricular Tachycardia; Mandibular Hypoplasia, Deafness, Progeroid Features, and Lipodystrophy Syndrome; Sick Sinus Syndrome; Pelger-Huet Anomaly; Charcot-Marie-Tooth Disease, Axonal, Type 2e; Congenital Generalized Lipodystrophy; Restrictive Cardiomyopathy; Congenital Fiber-Type Disproportion; Lipodystrophy, Congenital Generalized, Type 1; Myofibrillar Myopathy; Lipodystrophy, Familial Partial, Type 1; Axonal Neuropathy; Atypical Werner Syndrome; Ovarian Cystadenoma; Fanconi Anemia, Complementation Group a; Body Mass Index Quantitative Trait Locus 11; Skin Disease; Rigid Spine Muscular Dystrophy 1; Neuromuscular Disease; Hallermann-Streiff Syndrome; Bethlem Myopathy 1; Acquired Generalized Lipodystrophy; Cardiomyopathy, Dilated, 1e; Lipodystrophy, Congenital Generalized, Type 4; Undifferentiated Pleomorphic Sarcoma; Lipodystrophy, Familial Partial, Type 3; Muscular Dystrophy, Congenital Merosin-Deficient, 1a; Proximal Spinal Muscular Atrophy; Muscular Dystrophy-Dystroglycanopathy, Type B, 5; Muscular Dystrophy, Congenital, 1b; Reynolds Syndrome; Wiedemann-Rautenstrauch Syndrome; Emery-Dreifuss Muscular Dystrophy 1, X-Linked; Lipodystrophy, Congenital Generalized, Type 2; Monogenic Diabetes; Cardiomyopathy, Dilated, 1d; Myopathy, Proximal, and Ophthalmoplegia; Muscle Tissue Disease; Lipodystrophy, Familial Partial, Type 4; Cardiomyopathy, Dilated, 1h; Second-Degree Atrioventricular Block; Median Neuropathy; Intrinsic Cardiomyopathy; Prolapse of Female Genital Organ; Complete Generalized Lipodystrophy; Rigid Spine Muscular Dystrophy; Emerinopathy; Ulnar Nerve Lesion; Limb-Girdle Muscular Dystrophy Type 1b; Lmna-Related Dilated Cardiomyopathy; Pelvic Muscle Wasting; Generalized Lipodystrophy-Associated Progeroid Syndrome; Muscular Disease; Cardiomyopathy, Dilated, 1b; Autosomal Genetic Disease; Familial Isolated Arrhythmogenic Ventricular Dysplasia, Right Dominant Form; Familial Isolated Arrhythmogenic Ventricular Dysplasia, Biventricular Form; Familial Isolated Arrhythmogenic Ventricular Dysplasia, Left Dominant Form; Lmna-Related Cardiocutaneous Progeria Syndrome; and Autosomal Semi-Dominant Severe Lipodystrophic Laminopathy.
31. A nucleic acid according to any one of paras 1 to 8 or para 19, a LINC complex inhibiting polypeptide according to any one of paras 9 to 14 or para 18, a vector according to para 20 or para 21, a cell according to para 22 or a pharmaceutical composition according to para 23, for use in a method of treating or preventing a disease characterised by hyperlipidemia.
32. Use of a nucleic acid according to any one of paras 1 to 8 or para 19, a LINC complex inhibiting polypeptide according to any one of paras 9 to 14 or para 18, a vector according to para 20 or para 21, a cell according to para 22 or a pharmaceutical composition according to para 23 in the manufacture of a medicament for treating or preventing a disease characterised by hyperlipidemia.
33. A method of treating or preventing a disease characterised by hyperlipidemia, comprising administering a therapeutically- or prophylactically-effective amount of a nucleic acid according to any one of paras 1 to 8 or para 19, a LINC complex inhibiting polypeptide according to any one of paras 9 to 14 or para 18, a vector according to para 20 or para 21, a cell according to para 22 or a pharmaceutical composition according to para 23 to a subject.
34. The nucleic acid, LINC complex inhibiting polypeptide, vector, cell or pharmaceutical composition for use according to para 31, the use according to para 32, or the method according to para 33, wherein the disease characterised by hyperlipidemia is selected from atherosclerosis, cardiovascular disease, stroke and a familial hyperlipidemia.

***

The invention includes the combination of the aspects and preferred features described except where such a combination is clearly impermissible or expressly avoided.

The section headings used herein are for organisational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise,’ and variations such as ‘comprises’ and ‘comprising,’ will be understood to imply the inclusion of a stated integer or step or group of integers or steps but not the exclusion of any other integer or step or group of integers or steps.

It must be noted that, as used in the specification and the appended claims, the singular forms ‘a,’ ‘an,’ and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about,’ it will be understood that the particular value forms another embodiment.

Where a nucleic acid sequence is disclosed herein, the reverse complement thereof is also expressly contemplated.

Methods described herein may be performed in vitro or in vivo. In some embodiments, methods described herein are performed in vitro. The term ‘in vitro’ is intended to encompass experiments with cells in culture whereas the term ‘in vivo’ is intended to encompass experiments with intact multi-cellular organisms.

### BRIEF DESCRIPTION OF THE FIGURES

Embodiments and experiments illustrating the principles of the invention will now be discussed with reference to the accompanying figures.

FIG. 1. Alignment of the amino acid sequences of mouse Sun1, mouse Sun2, human SUN1 and human SUN2. Amino acid sequences starting from 6 amino acids upstream of the beginning of the lumenal domain were aligned using ClustalOmega software. Residues and domains of interest are indicated.

FIG. 2. Schematic representation of human SUN1 protein, and of the various constructs characterised Example 1 (i.e. constructs (A) to (H)). The domains of the lumenal domain of human SUN1 that are comprised in the constructs are indicated.

FIG. 3. Schematics and micrographs showing the results of analysis of the ability of different putative dominant-negative SUN1 constructs to achieve LINC complex disruption, as determined by an increase in Nesprin-2 localised to the endoplasmic reticulum. Arrows in micrographs indicate cells expressing high levels of the relevant construct. Whether the construct was determined to result in Nesprin-2 localisation is indicated.

FIG. 4. Schematic representation of human SUN1 protein, and of the various constructs characterised Example 5 (i.e. constructs A, D, D2, E and G). The domains of the lumenal domain of human SUN1 that are comprised in the constructs are indicated. The length of the SUN1-dervided sequence+KDEL is indicated.

FIGS. 5A to 5D. Graphs showing the results of analysis of the therapeutic efficacy of different putative dominant-negative SUN1 constructs in a mouse model of dilated cardiomyopathy. Briefly, Lmna^{FF:mcm} mice harbouring cardiac-specific Cre were injected with tamoxifen (TMX) to induce deletion of Lmna. 17 days after treatment with TMX, mice were administered with a single dose (1*10¹⁴ vg/kg) of AAV9 encoding DNhSUN1_201aa (‘201aa’; construct G of FIG. 4), DNhSUN1_255aa (‘255aa’; construct E of FIG. 4), DNhSUN1_334aa (‘334aa’; construct D2 of FIG. 4) or DNhSUN1_457aa (‘457aa’; construct A of FIG. 4) under the control of the cTNT promoter. As control conditions, wildtype mice (WT) or Lmna^{FF:mcm} mice administered TMX from day 17 (DCM) were administered with a single dose (1*10¹⁴ vg/kg) of AAV9 encoding GFP under the control of the cTNT promoter. Survival was monitored, cardiac function was evaluated by echocardiography, and Left Ventricular Inner Diameter (LVID) and Left Ventricular Posterior Wall (LVPW) thickness in diastole (d) and systole(s) were evaluated by ultrasound. (5A) Shows percentage survival over time for the mice in the different treatment groups. For each group, N=6. *** Long-rank (Mantel-Cox) P=0.0005, pairwise comparison against DCM control. (5B) Shows Fractional Shortening (FS) and Ejection Fraction (EF) for mice in the different treatment groups, over time. ** Mann-Whitney P<0.0087, pairwise comparison against DCM control. Data are presented as mean±SD. (5C and 5D) respectively show Left Ventricular Inner Diameter (LVID; 5C) and Left Ventricular Posterior Wall (LVPW) thickness (5D) in diastole (d) and systole(s), over time. Data are presented as mean±SD.

FIGS. 6A to 6D. Graphs showing the results of analysis of the therapeutic efficacy of different putative dominant-negative SUN1 constructs in a mouse model of dilated cardiomyopathy. Briefly, Lmna^{FF:mcm} mice harbouring cardiac-specific Cre were injected with tamoxifen (TMX) to induce deletion of Lmna. 17 days after treatment with TMX, mice were administered with a single dose (1*10¹⁴ vg/kg) of AAV9 encoding DNhSUN1_322aa (‘322aa’; construct D of FIG. 4) or DNhSUN1_457aa (‘457aa’; construct A of FIG. 4) under the control of the cTNT promoter. As control conditions, wildtype mice (WT) or Lmna^{FF:mcm} mice administered TMX from day 17 (DCM) were administered with a single dose (1*10¹⁴ vg/kg) of AAV9 encoding GFP under the control of the cTNT promoter. Survival was monitored, cardiac function was evaluated by echocardiography, and Left ventricular inner diameter (LVID) and Left ventricular posterior wall (LVPW) thickness in diastole (d) and systole(s) were evaluated by ultrasound. (6A) Shows percentage survival over time for the mice in the different treatment groups. 322aa group N=6, 457aa group N=6, DCM group N=5, WT group N=7. *** Long-rank (Mantel-Cox) P<0.0007, pairwise comparison against DCM control. Data are presented as mean±SD. (6B) Shows Fractional Shortening (FS) and Ejection Fraction (EF) for mice in the different treatment groups, over time. ** Mann-Whitney P<0.0095, pairwise comparison against DCM control. Data are presented as mean±SD. (6C and 6D) respectively show Left Ventricular Inner Diameter (LVID; 6C) and Left Ventricular Posterior Wall (LVPW) thickness (6D) in diastole (d) and systole(s), over time. Data are presented as mean±SD.

### EXAMPLE 1: IN VITRO ANALYSIS OF LINC COMPLEX INHIBITION BY DIFFERENT DOMINANT-NEGATIVE SUN1 CONSTRUCTS

### 1.1 Materials and Methods

### 1.1.1 Bioinformatics

The amino acid sequences of SUN domain-containing proteins and their orthologs were obtained from NCBI and Uniprot. Sequence alignments were performed using ClustalOmega (https://www.ebi.ac.uk/Tools/msa/clustalo). Domain analysis was performed using SMART (http://smart.embl-heidelberg.de/).

### 1.1.2 Molecular Biology

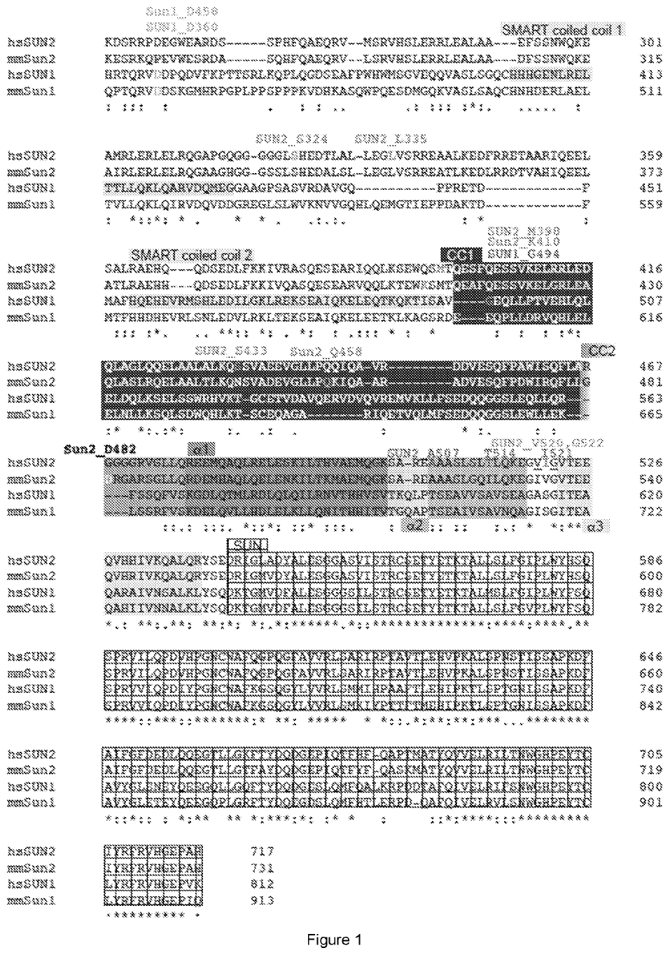

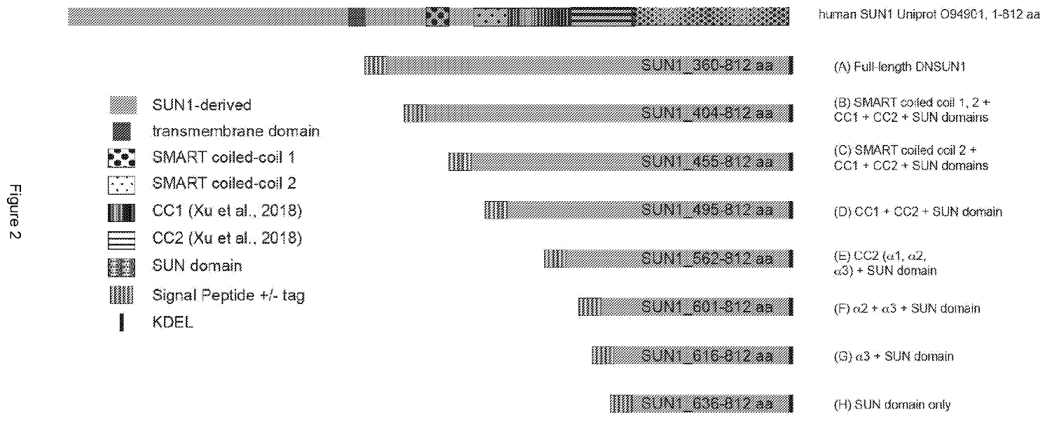

DNA sequences encoding the full-length DNSUN1 coding sequence, and truncated variants thereof, were synthesised, and cloned into pTwist EF1 Alpha plasmid. The nucleic acids encoding the DNSUN1 constructs were composed of a human serum albumin signal sequence at the N-terminus, followed by a HA tag and an ALFA tag, and then one of a variety of different truncated lumenal SUN1 domain sequences, followed by a KDEL sequence at the C-terminus. The amino acid sequences of the dominant-negative human SUN1 (DNhSUN1) constructs characterised in the Examples of the present disclosure are shown in SEQ ID NOS: 80 to 87 and 96, and their sequence features are represented schematically in FIGS. 1, 2 and 4.

| | | | LINC complex | |
|---|---|---|---|---|
| | Construct | | inhibitor polypeptide | Complete amino |
| | Ref in | Amino acid sequence | sequence (SUN- | acid sequence (incl. |
| | FIGS. 2 | of SUN-derived | derived inhibitory | SP, HA tag and |
| Protein Name | and 4 | inhibitory region | region + KDEL) | ALFA Tag) |
| DNhSUN1_457aa | A | SEQ ID NO: 43 | SEQ ID NO: 99 | SEQ ID NO: 80 |
| DNhSUN1_413aa | B | SEQ ID NO: 63 | SEQ ID NO: 69 | SEQ ID NO: 81 |
| DNhSUN1_362aa | C | SEQ ID NO: 64 | SEQ ID NO: 70 | SEQ ID NO: 82 |
| DNhSUN1_322aa | D | SEQ ID NO: 65 | SEQ ID NO: 71 | SEQ ID NO: 83 |
| DNhSUN1_334aa | D2 | SEQ ID NO: 94 | SEQ ID NO: 95 | SEQ ID NO: 96 |
| DNhSUN1_255aa | E | SEQ ID NO: 66 | SEQ ID NO: 72 | SEQ ID NO: 84 |
| DNhSUN1_216aa | F | SEQ ID NO: 67 | SEQ ID NO: 73 | SEQ ID NO: 85 |
| DNhSUN1_201aa | G | SEQ ID NO: 44 | SEQ ID NO: 74 | SEQ ID NO: 86 |
| DNhSUN1_181aa | H | SEQ ID NO: 68 | — | SEQ ID NO: 87 |

### 1.1.3 Cell Culture and Transfections

Hela cells were cultivated at 37° C. and 5% CO₂ in Dulbecco's modified Eagle's medium (DMEM) containing 4.5 g/L D-glucose, 4 mM L-glutamine, 1 mM sodium pyruvate, 10% fetal calf serum, and 100 U/ml penicillin; 100 mg/ml streptomycin. For immunofluorescence studies, cells were seeded on coverslips or 8-well slides (ibidi, Cat #80826). For each well in an 8-well slide, 200 ng of plasmid DNA encoding putative dominant-negative SUN1 constructs was transfected by addition of transfection complexes pre-formed for 15 min, containing 0.3 ml Lipofectamine 3000 (Invitrogen) and 0.2 ml P3000 reagent (Invitrogen) per 100 ng of plasmid DNA in Opti-MEM medium (Invitrogen).

### 1.1.4 Immunofluorescence Microscopy

The day after transfection, Hela cells were fixed in cold methanol at −20° C. for 10 min, and permeabilised and blocked in blocking buffer comprising 3% bovine serum albumin and 0.5% Trixon X-100 in phosphate buffered saline (PBS) for 30 min.

Cells were stained using an anti-Nesprin-2 mouse monoclonal antibody, anti-ALFA antibody (FluoTag®-X2 anti-ALFA conjugated to Abberior Star580, NanoTag Biotechnologies N1502-Ab580-L) to detect the relevant DNSUN1 construct, and for the nuclear envelope using a mouse monoclonal anti-lamin B2 antibody. Cells were incubated in primary antibodies in blocking buffer for 1-2 h at room temperature. Following primary antibody incubation, cells were washed with 0.5% Triton X-100 in PBS three times, before incubation with fluorophore-conjugated mouse antibody isotype-specific secondary antibodies (Thermo Fisher Scientific A-21121, Goat anti-Mouse IgG1 conjugated to Alexa Fluor 488 and A-21242, Goat anti-Mouse lgG2b conjugated to Alexa Fluor 647) and Hoechst to stain for DNA for 30 min-1 h at room temperature. Cells were washed again before mounting in Prolong Diamond or 1% DABCO (1,4-Diazabicyclo[2.2.2]octane) in 10% PBS/90% glycerol. Widefield epifluorescence imaging was performed on an Olympus IX-83 with a motorised stage and equipped with a 20x 0.45 NA LUCPlan FL N or a 40x 0.6 NA Ph2 LUCPlan FL N objectives. Digital images were captured using CellSens software and processed using Fiji software.

### 1.2 Results

The inventors evaluated the ability of different truncated variants of SUN1 comprising a C-terminal KDEL sequence, in order to determine which regions of the SUN1 are required in order for the protein to function as a dominant-negative SUN1 protein for achieving LINC complex inhibition. Specifically, the inventors investigated the contribution of 2 coiled-coil regions identified by SMART domain analysis, CC1 and CC2 domains described e.g. in Xu et al., 2018, as well as the involvement of the three alpha helices of the CC2 domain.

An alignment of the lumenal regions of human SUN1, human SUN1, mouse Sun1 and mouse Sun2 is shown in FIG. 1.

The different truncated variants investigated in the present Example are represented schematically in FIG. 2.

The ability of the different truncated SUN1 constructs to function as dominant-negative LINC complex inhibitors was evaluated by transiently expressing them in cells, and subsequently evaluating the subcellular localization of the KASH domain-containing protein Nesprin-2. At low levels of expression,

DNSUN1 is localised at the nuclear envelope in the perinuclear space. When expressed at high levels, DNSUN1 is localised throughout the endoplasmic reticulum lumen, which is the same membrane compartment as the perinuclear space. See Crisp et al., 2006.

In cells expressing high levels of a dominant-negative SUN domain-containing LINC complex inhibiting protein, the normal nuclear envelope localization of KASH domain-containing proteins is disrupted, and the KASH domain-containing proteins localise to the endoplasmic reticulum as the large excess of dominant-negative protein out-competes endogenous SUN domain-containing proteins for interaction with the KASH domain-containing proteins. This can be easily visualised using immunofluorescence microscopy using antibodies specific for the KASH domain-containing protein.

In the present studies, plasmids encoding the different truncated SUN1 constructs were expressed in Hela cells by transient transfection, and the cells were subsequently analyzed by immunofluorescence staining for DNSUN1 and Nesprin-2 (a KASH domain-containing protein that is highly-expressed in HeLa cells).

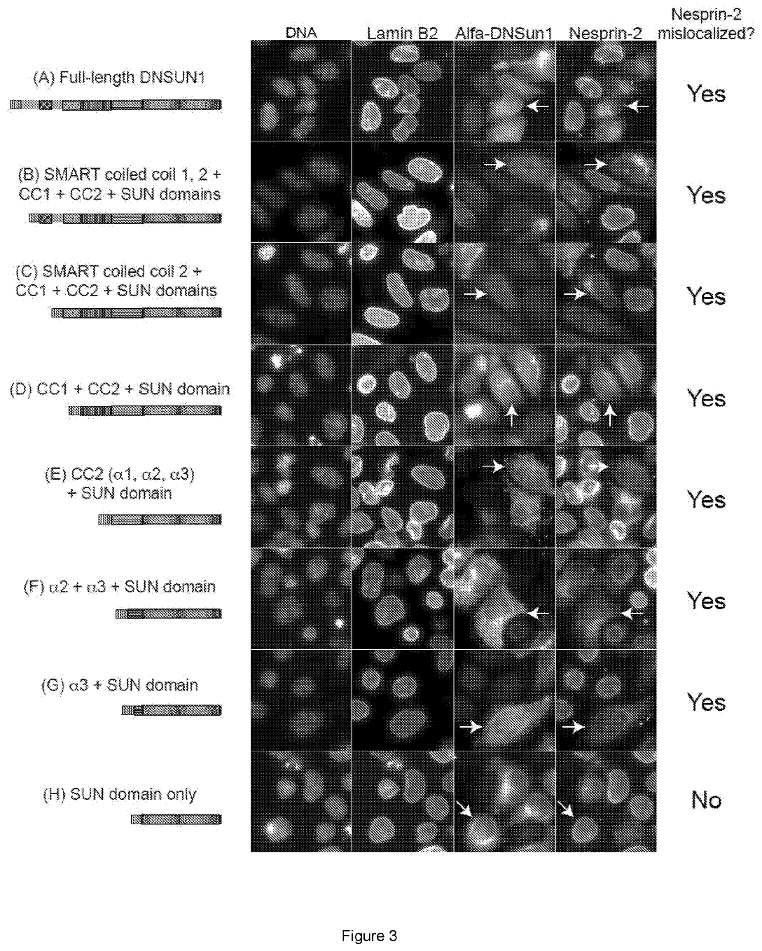

The results are shown in FIG. 3. As expected, full-length DNSUN1 (i.e. the protein having the amino acid sequence of SEQ ID NO:80; DNhSUN1_457aa)-which has previously been demonstrated to disrupt the LINC complex in Hela cells, and to suppress the pathology of LMNA-associated mutations in mouse models of laminopathies—was found to displace Nesprin-2 from the nuclear envelope. Also as expected, a DNSUN1 construct containing only the SUN domain (i.e. encoding the protein having the amino acid sequence of SEQ ID NO:87; DNhSUN1_181aa), lacking any preceding coiled-coil domains of alpha-helices thereof to promote multimerization, failed to displace Nesprin-2.

Unexpectedly, all other DNhSUN1constructs tested (DNhSUN1_413aa, DNhSUN1_362aa, DNhSUN1_322aa, DNhSUN1_255aa, DNhSUN1_216aa and DNhSUN1_201aa) were able to displace Nesprin-2 from the nuclear envelope and into the ER, even though it would have been expected from previous studies that certain constructs would be unable to interact with KASH domain-containing proteins.

Nie et al. 2016 and Jahed et al. 2018b disclose that a truncated variant of Sun2 comprising the complete CC2 domain and the SUN domain is inhibited by the CC2 domain from forming trimeric complexes required for interaction with the KASH domain of Nesprin-2. Similarly, Sosa et al. 2012 reports that a truncated variant of Sun2 comprising the α3 helix of CC2 domain and the SUN domain is deficient in KASH binding.

However, in the present studies, paralogous constructs based on SUN1 were found to disrupt normal localization of Nesprin-2, and thus to behave as LINC complex inhibitors.

### 1.3 Discussion

In the present Example, the inventors demonstrated that a series of SUN1 lumenal domain sequences of various lengths (shown in SEQ ID NOs: 43, 44, 63-67 and 94) are able to effect LINC complex disruption. Surprisingly, previous structural and biochemical studies on the lumenal domain of SUN proteins was not predictive of the effect of truncating the lumenal domain of SUN1 in the present DNSUN1 constructs.

The characterised dominant-negative SUN1 proteins may achieve LINC complex disruption through the formation of multimers (e.g. trimers) that behave as competitive inhibitors of endogenous SUN domain-containing proteins for binding to KASH domain-containing proteins. The fact that only low levels of the dominant-negative SUN1 proteins are localised to the nuclear envelope suggests that the dominant-negative SUN1 proteins may also achieve LINC complex disruption by forming hetero-oligomers with endogenous SUN domain-containing proteins, thereby weakening the linkage between the cytoskeleton and the nuclear lamina mediated by intact LINC complexes.

It is possible that dominant-negative SUN1 constructs form hetero-oligomers with endogenous SUN proteins even if they are unable to homotrimerise. This might explain why truncated lumenal SUN proteins that are reported to be monomeric and unable to bind KASH in vitro might still be competent to disrupt LINC complexes, as demonstrated in the present Examples. Alternatively, the in vitro context in which binding and gel filtration experiments were conducted may simply be different biochemically from the cellular environment and hence not predictive of how truncated lumenal SUN proteins function in cells.

### Example 2: Further In Vitro Analysis of LINC Complex Inhibition by Different Dominant-Negative SUN Constructs

In further experiments, the inventors characterise all possible truncation mutants of the human SUN1 and human SUN2 lumenal domain for their ability to function as LINC complex inhibitors.

Briefly, constructs are prepared as described in Example 1.1.2 (i.e. comprising an N-terminal signal peptide, a HA/ALFA tag and a SUN domain-containing protein-derived region and a C-terminal KDEL motif), except that every possible truncation mutant of the SUN1 lumenal domain from SUN1 (361-812) to SUN1 (636-812) is prepared (i.e. SUN1 (361-812), SUN1 (363-812), SUN1 (363-812), SUN1 (364-812) et seq.).

Similarly, every possible truncation mutant of the SUN2 lumenal domain from SUN2 (258-717) to SUN2 (522-717) is prepared (i.e. SUN2 (259-717), SUN2 (260-717), SUN2 (261-717), SUN2 (262-717) et seq.).

The different constructs are evaluated for their ability to disrupt normal subcellular localization of Nesprin-2 in Hela cells as described in Example 1.1.

All of the constructs evaluated comprising at least the α3 helix of the CC2 region and the SUN domain (i.e. constructs comprising at least positions 616-812 of human SUN1, and constructs comprising at least positions 522-717 of human SUN2) are determined to displace Nesprin-2 from the nuclear envelope and into the endoplasmic reticulum, and thus to behave as LINC complex inhibitors.

### Example 3: In Vivo Confirmation of the Utility of LINC Complex Inhibiting Polypeptides Characterised in Examples 1 and 2 for the Treatment of Laminopathy Associated with Mutation to Lmna

Mice having mouse models of Lmna mutation-associated cardiomyopathy, muscular dystrophy and progeria are administered with AAV or lentiviral vectors encoding nucleic acid encoding LINC complex inhibiting polypeptides described in Example 1 and 2, or nanoparticles comprising such nucleic acid.

The mouse models of Lmna mutation-associated muscular dystrophy include the conditional Lmna knockout mouse. The generation of Lmna^{Fix/Fix} mice and MLC-Cre mice is described in Wang et al., Differentiation. (2015) 89 (1-2): 11-21 and Mourkioti et al., Genesis. (2008) 46 (8): 424-30, respectively. Lmna^{Fix/Fix} mice are crossed to MLC-Cre mice to obtain Lmna^{Fix/+}; MLC-Cre mice, which are then crossed with Lmna^{Fix/+} mice to generate Lmna^{Fix/FIX}; MLC-Cre, providing for conditional knockout of Lmna in muscle cells.

Mouse models of Lmna mutation-associated cardiomyopathy and muscular dystrophy include the Lmna-H222P mouse described e.g. in Arimura et al., Hum Mol Genet. (2005) 14 (1): 155-69, the Lmna-N195K mouse described e.g. in Mounkes et al., Hum Mol Genet. (2005) 14 (15): 2167-80, and the conditional Lmna deletion-associated mouse model of dilated cardiomyopathy, described e.g. in Chai et al., Nat Commun (2021) 12 (1): 4722 (see Example 5.1 below).

Mouse models of Lmna mutation-associated progeria include the Lmna-G609G mouse described in Osori et al., Sci Transl Med. (2011) 3 (106): 106ra107.

Mice having Lmna mutation-associated cardiomyopathy administered nucleic acid encoding a LINC complex inhibiting polypeptide described in Examples 1 or 2 have improved cardiac function (e.g. increased myocardial contractility, increased ejection fraction and/or increased fractional shortening) as compared to untreated mice, or mice treated with vehicle only (e.g. empty vector).

Mice having Lmna mutation-associated muscular dystrophy or Lmna mutation-associated progeria administered nucleic acid encoding a LINC complex inhibiting polypeptide described in Examples 1 or 2 have an extended lifespan as compared to untreated mice, or mice treated with vehicle only (e.g. empty vector).

### Example 4: In Vivo Confirmation of the Utility of LINC Complex Inhibiting Polypeptides Characterised in Examples 1 and 2 for the Treatment of Hyperlipidemia

Mice having mouse models of atherosclerosis and familial hypercholesterolemia are administered with AAV or lentiviral vectors encoding nucleic acid encoding LINC complex inhibiting polypeptides described in Example 1 and 2, or nanoparticles comprising such nucleic acid.

A mouse model of atherosclerosis and familial hypercholesterolemia is established by feeding Ldlr knockout mice (C57BL/6JInv, Jackson Laboratory) a Western-type diet (WTD; D12079B, Research Diets, NJ), for 15 weeks.

Ldlr/-mice having atherosclerosis and familial hypercholesterolemia administered nucleic acid encoding a LINC complex inhibiting polypeptide described in Examples 1 or 2 have smaller, less advanced and less complex atherosclerotic lesions as compared to untreated mice, or mice treated with vehicle only (e.g. empty vector).

### Example 5: In Vivo Evaluation of the Therapeutic Efficacy of Various Different LINC Complex Inhibiting Polypeptides

The inventors next investigated the ability of different DNhSUN1 LINC complex inhibiting polypeptides to rescue the deleterious effects in a LNMA mutation-associated dilated cardiomyopathy in vivo, in a mouse model.

### 5.1 Materials and Methods

### Preparation of AAV Encoding LINC Complex Inhibiting Polypeptides

Coding sequences for DNhSUN1 constructs were PCR amplified from synthesized genes, restriction digested, and ligated into an AAV transfer plasmid containing the cTnT cardiac promoter, a chimeric intron, a WPRE (Woodchuck Hepatitis Virus Posttranscriptional Regulatory Element) and a bovine growth hormone polyA tail. An AAV transfer plasmid instead comprising the coding sequence for GFP was also prepared, for the preparation of AAV vectoring GFP, for use as an experimental control. The LINC complex inhibiting polypeptides used in the experiments of the present Example differ from those of Example 1 in that they employ the minimal human serum albumin signal peptide (SEQ ID NO: 100), and lack HA and ALFA tags:

| | | | LINC complex | |
|---|---|---|---|---|
| | Construct | | inhibitor polypeptide | |
| | Ref in | Amino acid sequence | sequence (SUN- | Complete amino |
| | FIGS. 2 | of SUN-derived | derived inhibitory | acid sequence (incl. |
| Protein Name | and 4 | inhibitory region | region + KDEL) | SP) |
| DNhSUN1_457aa | A | SEQ ID NO: 43 | SEQ ID NO: 99 | SEQ ID NO: 101 |
| DNhSUN1_322aa | D | SEQ ID NO: 65 | SEQ ID NO: 71 | SEQ ID NO: 104 |
| DNhSUN1_334aa | D2 | SEQ ID NO: 94 | SEQ ID NO: 95 | SEQ ID NO: 105 |
| DNhSUN1_255aa | E | SEQ ID NO: 66 | SEQ ID NO: 72 | SEQ ID NO: 106 |
| DNhSUN1_201aa | G | SEQ ID NO: 44 | SEQ ID NO: 74 | SEQ ID NO: 108 |

Adeno-associated viruses were produced by transient triple transfection of HEK293T cells with an adenoviral helper plasmid, a rep/cap plasmid encoding AAV2 Rep and AAV9 cap, and transgene-specific plasmids. 4 days after transfection, the cell pellet and supernatant were harvested. The supernatant was clarified by filtration and applied by gravity flow to POROS™ CaptureSelect™ AAV9 Affinity Resin (Thermo Fisher Scientific, Waltham, MA, USA). Cells were resuspended in lysis buffer (phosphate-buffered saline, 200 mM NaCl, 0.001% Pluronic F-68), lysed by 4-5 rounds of freeze/thaw, sonicated, and treated with benzonase to shear and digest DNA. Cell debris was pelleted by centrifugation, and cell lysates were collected and filtered through 0.45 μm syringe filters. Filtered lysates were then applied to AAV9 affinity resin by gravity flow. Following washing with wash buffer (phosphate-buffered saline, 500 mM NaCl), AAV9 virions were eluted using 100 mM glycine, pH 2.5, and collected in microfuge tubes containing 1/10th volume of 1 M Tris, pH8. Following 2 rounds of buffer exchange into PBS containing 0.01% Pluronic F-68 and concentration via Amicon® Ultra 100 KDA concentrators (Merck KGaA, Darmstadt, Germany), solution containing AAV virions was filtered through 0.22 μm 4 mm Millex syringe filter units (Merck KGaA, Darmstadt, Germany) and stored in 4° C. or −80° C. Viral titre was determined using alkaline gel electrophoresis with a plasmid DNA standard (Kohlbrenner and Weber, Methods Mol. Biol. (2017) 1521:91-107), gel imaging on a ChemiDoc imaging system (Bio-Rad Laboratories, Hercules, CA, USA) and image quantification with Image Lab software (Bio-Rad Laboratories).

### Lmna Deletion Dilated Cardiomyopathy (DCM) Mouse Model

Mouse strains were maintained on a 12 h light/dark cycle in ventilated animal barrier facilities with the temperature set to 21±1° C., humidity at 55-70% and with food and water provide ad libitum, in accordance with all ethical regulations.

To generate the Lmna deletion-associated mouse model of dilated cardiomyopathy, mice of the conditional “knockout” or “floxed” Lmna^{Fix/Fix} mouse strain described in Wang et al., Differentiation. (2015) 89 (1-2): 11-21 were crossed with mice harbouring a tamoxifen-inducible Cre recombinase under control of a cardiomyocyte-specific alpha-myosin heavy chain promoter (aMHC-MerCreMer, abbreviated to mcm) purchased from the Jackson Laboratory (JAX stock number 005657; see also Chai et al., Nat Commun (2021) 12 (1): 4722). To induce dilated cardiomyopathy in these Lmna^{FF:mcm} mice, 40 mg/kg tamoxifen was injected with intraperitoneally at postnatal day 14 (P14), resulting in deletion of Lmna specifically in cardiomyocytes. Tamoxifen (Merck) solution (16 g/L) was prepared in corn oil (Sigma).

### In Vivo Delivery of Adeno-Associated Virus

17 days after tamoxifen treatment, mice were injected retro-orbitally with different AAV9 at a dose of 1×10¹⁴ vg/kg. Animals were selected randomly. TMX and AAV9 injections were performed under anaesthesia using 1.5% isoflurane mixed with oxygen. AAV9 dilutions were prepared freshly before administration with PBS containing 0.001% Pluronic F-68.

### Echocardiography

Cardiac function was evaluated by ultrasound recordings using Prospect-T1 (Sharp-S). Mice were shaved 1 day before ultrasound examination. Animals were anaesthetized with 1.5% isoflurane mixed with oxygen. Readings of β-mode and M-mode were taken at heart rates between 450 bpm and 350 bpm. Fractional Shortening (FS) and Ejection Fraction (EF) were calculated from the parasternal long axis using the equipment's software analysis tool. Cardiac measurements of the Left Ventricular Inner Diameter (LVID) and Left Ventricular Posterior Wall (LVPW) were taken from the parasternal short-axis for the diastolic (d) and systolic(s) state.

### 5.2 Results and Discussion

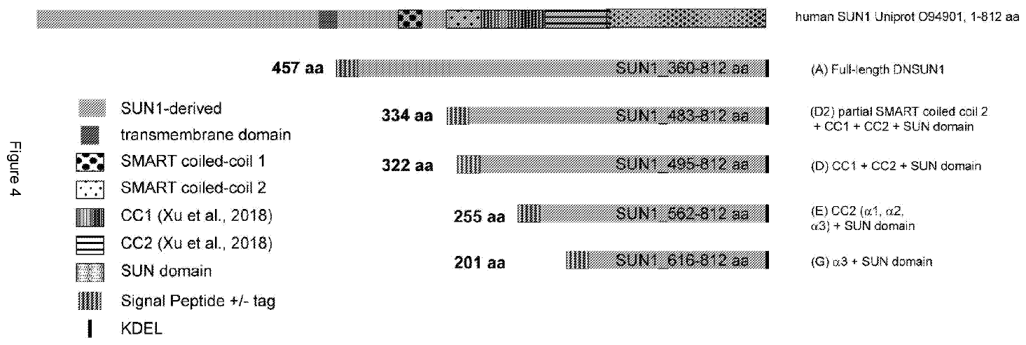

In a first experiment, P14 Lmna^{FF:mcm} mice harbouring cardiac-specific Cre recombinase were injected with tamoxifen (TMX) to induce deletion of the Lmna gene. 17 days after treatment with TMX, mice were administered with a single dose (1×10¹⁴ vg/kg) of AAV9 encoding DNhSUN1_201aa (construct G of FIG. 4), DNhSUN1_255aa (construct E of FIG. 4), DNhSUN1_334aa (construct D2 of FIG. 4) or DNhSUN1_457aa (construct A of FIG. 4) under the control of the cTNT promoter. As control conditions, wildtype mice (WT) or Lmna^{FF:mcm} mice administered TMX from day 17 (DCM) were administered with a single dose (1×10¹⁴ vg/kg) of AAV9 encoding GFP under the control of the cTNT promoter. Survival was monitored, cardiac function was evaluated by echocardiography, and Left Ventricular Inner Diameter (LVID) and Left ventricular posterior wall (LVPW) thickness in diastole (d) and systole(s) were evaluated by ultrasound.

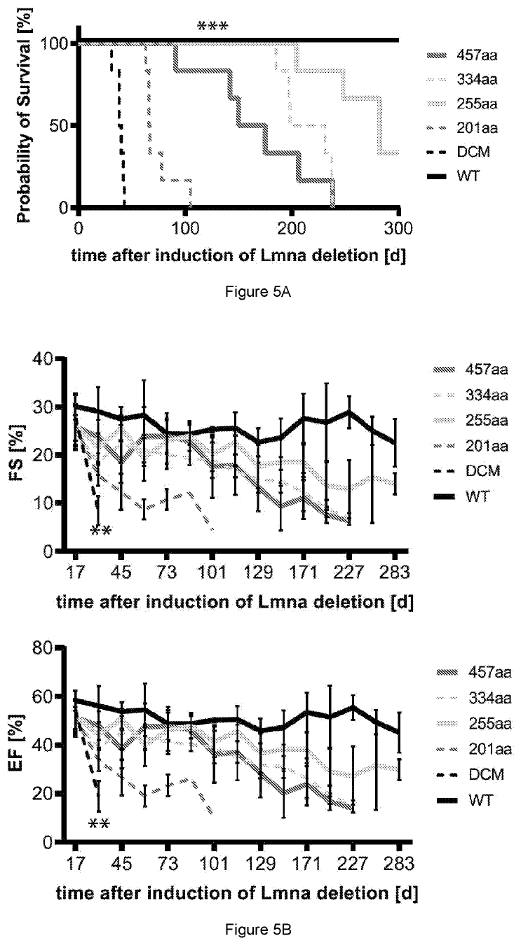

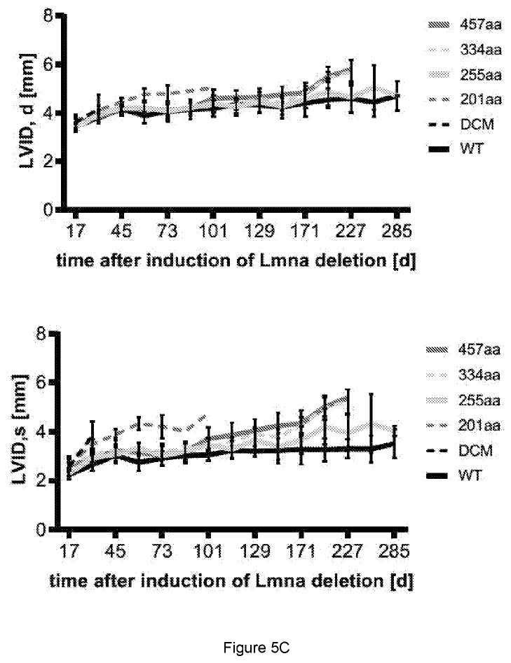

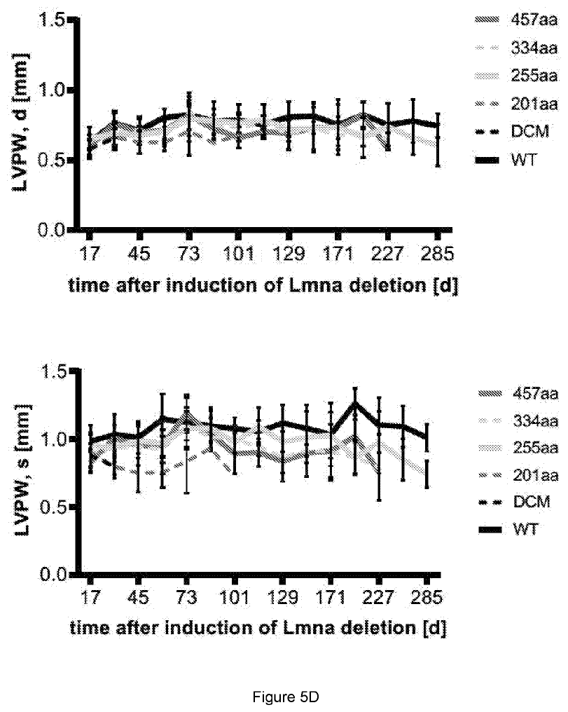

The results are shown in FIGS. 5A to 5D.

FIG. 5A shows that treatment with all DNhSUN1 variants resulted in extension of median lifespan compared to the DCM control animals (39 days). The shortest construct DNhSUN1_201aa (construct G) resulted in the shortest lifespan extension, at 66.5 days. Mice treated with the DNhSUN1_334aa (construct D2) survived a median of 214.5 days, surpassing the full-length, DNhSUN1_457aa (construct A), which survived 162.5 days. Animals treated with DNhSUN1_255aa (construct E) displayed the longest lifespan extension; only 2 animals died, and after 275 days 4 mice were still alive.

FIG. 5B shows that treatment with AAV encoding DNhSUN1_201aa, DNhSUN1_255aa, DNhSUN1_334aa or DNhSUN1_457aa improved cardiac function in Lmna DCM mice. Echocardiography analysis revealed significant improvement of Fractional Shortening (FS) and Ejection Fraction (EF) in Lmna^{FF:mcm} treated with all AAV9-cTnT-DNhSUN1 truncation variants compared to AAV9-cTNT-GFP treated mice (DCM) at 17 days post administration of AAV9 (i.e. day 31 post administration of TMX).

FIGS. 5C and 5D show that LVID increases and LVPW decreases in Lmna DCM mice; this is more obvious in systole. Dilation of the left ventricle and thinning of the heart wall occurs earlier in mice treated with DNhSUN1_201aa (construct G) than in other DNhSUN1 variants (DNhSUN1_255aa/construct E, DNhSUN1_334aa/construct D2, DNhSUN1_457aa/construct A), which correlates with the decline of cardiac function and earlier onset of DCM in the mice treated with AAV delivering DNhSUN1_201aa.

Thus, AAV encoding DNhSUN1_201aa, DNhSUN1_255aa, DNhSUN1_334aa or DNhSUN1_457aa were found to treat Lmna deletion-associated DCM. DNhSUN1_201aa was found to extend lifespan to a lesser extent than DNhSUN1_457aa, while DNhSUN1_334aa and DNhSUN1_255aa extended lifespan to a greater extent than DNhSUN1_457aa.

In a second experiment, the efficacy of the DNhSUN1_322aa was compared to the DNhSUN1_457aa. Briefly, P14 Lmna^{FF:mcm} mice harbouring cardiac-specific Cre recombinase were injected with tamoxifen

(TMX) to induce deletion of the Lmna gene. 17 days after treatment with TMX, mice were administered with a single dose (1×10¹⁴ vg/kg) of AAV9 encoding DNhSUN1_322aa (construct D of FIG. 4) or DNhSUN1_457aa (construct A of FIG. 4) under the control of the cTNT promoter. As control conditions, wildtype mice (WT) or Lmna^{FF:mcm} mice administered TMX from day 17 (DCM) were administered with a single dose (1×10¹⁴ vg/kg) of AAV9 encoding GFP under the control of the cTNT promoter. Survival was monitored, cardiac function was evaluated by echocardiography, and Left Ventricular Inner Diameter (LVID) and Left ventricular posterior wall (LVPW) thickness in diastole (d) and systole(s) were evaluated by ultrasound.

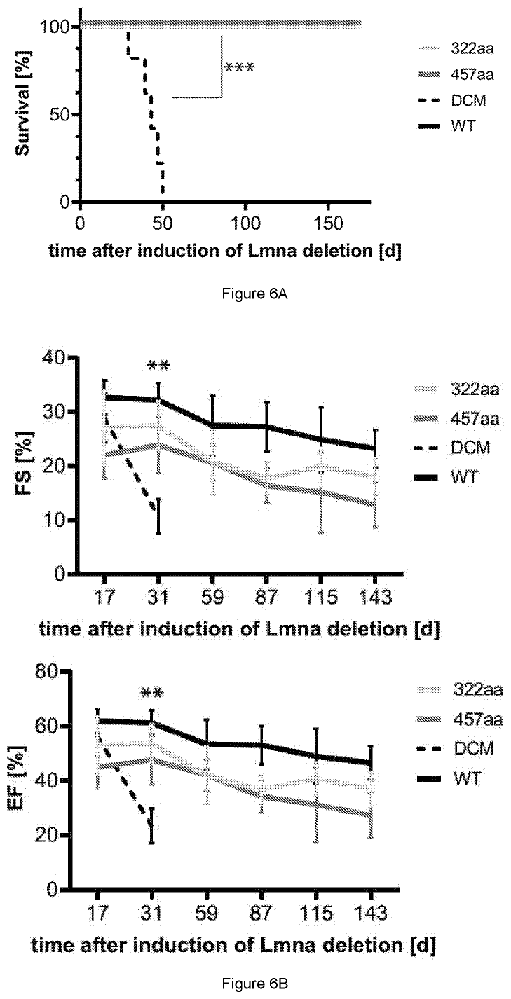

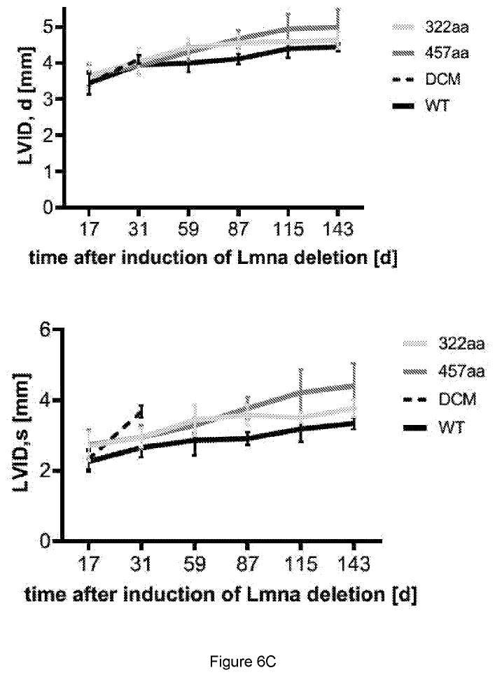

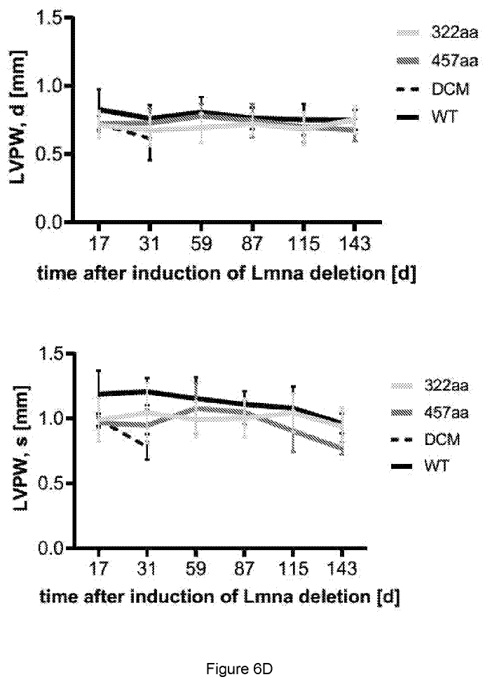

The results are shown in FIGS. 6A to 6D.

FIG. 6A shows that treatment with AAV encoding DNhSUN1_322aa or DNhSUN1_457aa extended median lifespan compared to the DCM control animals, which had an average survival of 39 days. All mice in groups administered with AAV encoding DNhSUN1_322aa or DNhSUN1_457aa were still alive at day 170.

FIG. 6B shows that treatment with AAV encoding DNhSUN1_322aa or DNhSUN1_457aa improved cardiac function in Lmna DCM mice. Echocardiography analysis revealed significant improvement of Fractional Shortening (FS) and Ejection Fraction (EF) in Lmna^{FF:mcm} treated with AAV9 delivering the 322aa or DNhSUN1_457aa variants compared to AAV9-cTNT-GFP treated mice (DCM) at 17 days post administration of AAV9 (i.e. day 31 post administration of TMX).

FIGS. 6C and 6D show that LVID increases and LVPW decreases in Lmna DCM mice; this is more obvious in systole. For the AAV9-cTnT-DNhSUN1 treated animals, which survive longer than DCM control animals, LVID increases while LVPW stabilizes and does not decrease.

Thus, AAV encoding DNhSUN1_322aa or DNhSUN1_457aa were found to treat Lmna deletion-associated DCM.

### REFERENCES

Burke, B., and C.L. Stewart. 2013. The nuclear lamins: flexibility in function. Nat Rev Mol Cell Biol. 14:13-24. doi: 10.1038/nrm3488.
Crisp, M., Q. Liu, K. Roux, J. B. Rattner, C. Shanahan, B. Burke, P.D. Stahl, and D. Hodzic. 2006. Coupling of the nucleus and cytoplasm: role of the LINC complex. J. Cell Biol. 172:41-53. doi: 10.1083/jcb.200509124.
Gurusaran, M., and O. R. Davies. 2021. A molecular mechanism for LINC complex branching by structurally diverse SUN-KASH 6:6 assemblies. eLife. 10: e60175. doi: 10.7554/eLife.60175.
Jahed, Z., D. Fadavi, U.T. Vu, E. Asgari, G. W. G. Luxton, and M. R. K. Mofrad. 2018a. Molecular Insights into the Mechanisms of SUN1 Oligomerization in the Nuclear Envelope. Biophysical Journal. 114:1190-1203. doi: 10.1016/j.bpj.2018.01.015.
Jahed, Z., U. T. Vu, D. Fadavi, H. Ke, A. Rathish, S. C. J. Kim, W. Feng, M. R. K. Mofrad, and V. M. Weaver. 2018b. A molecular model for LINC complex regulation: activation of SUN2 for KASH binding. MBoC. 29:2012-2023. doi: 10.1091/mbc.E18-04-0266.
Lee, Y. L., and B. Burke. 2018. LINC complexes and nuclear positioning. Seminars in Cell & Developmental Biology. 82:67-76. doi: 10.1016/j.semcdb.2017.11.008.
McCarty, D. M. 2008. Self-complementary AAV Vectors; Advances and Applications. Molecular Therapy. 16:1648-1656. doi: 10.1038/mt.2008.171.
Nie, S., H. Ke, F. Gao, J. Ren, M. Wang, L. Huo, W. Gong, and W. Feng. 2016. Coiled-Coil Domains of SUN Proteins as Intrinsic Dynamic Regulators. Structure. 24:80-91. doi: 10.1016/j.str.2015.10.024.
Sosa, B. A., A. Rothballer, U. Kutay, and T.U. Schwartz. 2012. LINC complexes form by binding of three KASH peptides to domain interfaces of trimeric SUN proteins. Cell. 149:1035-1047. doi: 10.1016/j.cell.2012.03.046.
Wang, W., Z. Shi, S. Jiao, C. Chen, H. Wang, G. Liu, Q. Wang, Y. Zhao, M.I. Greene, and Z. Zhou. 2012. Structural insights into SUN-KASH complexes across the nuclear envelope. Cell Res. 22:1440-1452. doi: 10.1038/cr.2012.126.
Xu, Y., W. Li, H. Ke, and W. Feng. 2018. Structural conservation of the autoinhibitory domain in SUN proteins. Biochem. Biophys. Res. Commun. 496:1337-1343. doi: 10.1016/j.bbrc.2018.02.015.
Zhou, Z., X. Du, Z. Cai, X. Song, H. Zhang, T. Mizuno, E. Suzuki, M.R. Yee, A. Berezov, R. Murali, S.-L. Wu, B. L. Karger, M. I. Greene, and Q. Wang. 2012. Structure of Sad1-UNC84 Homology (SUN) Domain Defines Features of Molecular Bridge in Nuclear Envelope. J. Biol. Chem. 287:5317-5326. doi: 10.1074/jbc.M111.304543.

1-30. (canceled)

31. A LINC complex inhibiting polypeptide consisting of:
(i) (a) an amino acid sequence having at least 80% identity to the amino acid sequence of SEQ ID NO: 65 and (b) an endoplasmic reticulum retention motif; or
(ii) (a) a signal peptide, (b) an amino acid sequence having at least 80% identity to SEQ ID NO: 65, and (c) an endoplasmic reticulum retention motif.

32. The LINC complex inhibiting polypeptide according to claim 31, wherein the endoplasmic reticulum retention motif is a KDEL motif, or a variant thereof selected from CDEL, KCEL, or HVEL.

33. The LINC complex inhibiting polypeptide according to claim 31, wherein the signal peptide consists of an amino acid sequence having at least 80% sequence identity to the amino acid sequence of SEQ ID NO: 100.

34. The LINC complex inhibiting polypeptide according to claim 31, wherein the LINC complex inhibiting polypeptide consists of an amino acid sequence having at least 80% amino acid sequence identity to the amino acid sequence of SEQ ID NO:71 or SEQ ID NO:104.

35. The LINC complex inhibiting polypeptide according to claim 31, wherein the LINC complex inhibiting polypeptide consists of the amino acid sequence of SEQ ID NO:71 or SEQ ID NO: 104.

36. A polynucleotide encoding a LINC complex inhibiting polypeptide, wherein the LINC complex inhibiting polypeptide consists of:
(i) (a) an amino acid sequence having at least 80% identity to the amino acid sequence of SEQ ID NO: 65 and (b) an endoplasmic reticulum retention motif; or
(ii) (a) a signal peptide, (b) an amino acid sequence having at least 80% identity to SEQ ID NO: 65, and (c) an endoplasmic reticulum retention motif.

37. The polynucleotide according to claim 36, further comprising a promoter providing for expression of the polynucleotide in cardiac and/or skeletal muscle cells or tissue.

38. The polynucleotide according to claim 37, wherein the promoter is:
(i) a cardiac or cardiomyocyte-specific promoter, optionally a cTNT, a-MHC or MLC2v promoter;
or
(ii) a skeletal muscle/striated muscle cell-specific promoter, optionally a MCK, MHCK7 or desmin promoter.

39. A vector comprising a polynucleotide, wherein the polynucleotide encodes a LINC complex inhibiting polypeptide, wherein the LINC complex inhibiting polypeptide consists of:
(i) (a) an amino acid sequence having at least 80% identity to the amino acid sequence of SEQ ID NO: 65 and (b) an endoplasmic reticulum retention motif; or
(ii) (a) a signal peptide, (b) an amino acid sequence having at least 80% identity to SEQ ID NO: 65, and (c) an endoplasmic reticulum retention motif; and
wherein the vector is suitable for delivering the polynucleotide encoding a LINC complex inhibiting polypeptide as a gene therapy.

40. The vector according to claim 39, wherein the vector is an adeno-associated virus (AAV) vector.

41. The vector according to claim 39, wherein the vector is an AAV vector selected from AAV2, AAV1, AAV218, AAV5, AAV6, AAV8, AAV9, AAV9.45, AAV10 or AAVrh74 serotype.

42. A method of treating or preventing a laminopathy or a disease characterised by hyperlipidemia, comprising administering a therapeutically-or prophylactically effective amount of a polynucleotide encoding a LINC complex inhibiting polypeptide, wherein the LINC complex inhibiting polypeptide consists of:
(i) (a) an amino acid sequence having at least 80% identity to the amino acid sequence of SEQ ID NO: 65 and (b) an endoplasmic reticulum retention motif; or
(ii) (a) a signal peptide, (b) an amino acid sequence having at least 80% identity to SEQ ID NO: 65, and (c) an endoplasmic reticulum retention motif.

43. The method according to claim 42, wherein the laminopathy is characterised by one or more of myopathy, cardiomyopathy, dilated cardiomyopathy, muscular dystrophy, cardiac muscular dystrophy, skeletal muscular dystrophy, progeria, neuropathy, lipoatrophy, skeletal dysplasia, lipodystrophy, leukodystrophy or dermopathy.

44. The method according to claim 42, wherein the laminopathy is associated with mutation to LMNA.

45. The method according to claim 42, wherein the laminopathy is selected from: Dilated Cardiomyopathy; Hutchinson-Gilford Progeria Syndrome; Muscular Dystrophy, Congenital, Lmna-Related; Emery-Dreifuss Muscular Dystrophy 2, Autosomal Dominant; Muscular Dystrophy; Mandibuloacral Dysplasia with Type a Lipodystrophy; Cardiomyopathy, Dilated, la; Charcot-Marie-Tooth Disease; Limb-Girdle Muscular Dystrophy; Cardiomyopathy, Dilated, with Hypergonadotropic Hypogonadism; Emery-Dreifuss Muscular Dystrophy 3, Autosomal Recessive; Lipodystrophy, Familial Partial, Type 2; Emery-Dreifuss Muscular Dystrophy; Charcot-Marie-Tooth Disease, Axonal, Type 2b1; Heart-Hand Syndrome, Slovenian Type; Aging; Familial Partial Lipodystrophy; Restrictive Dermopathy, Lethal; Arrhythmogenic Right Ventricular Cardiomyopathy; Tooth Disease; Heart Disease; Werner Syndrome; Hypertrophic Cardiomyopathy; Left Ventricular Noncompaction; Atrioventricular Block; Calcinosis; Acroosteolysis; Autosomal Dominant Limb-Girdle Muscular Dystrophy; Diabetes Mellitus, Noninsulin-Dependent; Osteoporosis; Atrial Fibrillation; Atrial Standstill 1; Acanthosis Nigricans; Cardiac Conduction Defect; Catecholaminergic Polymorphic Ventricular Tachycardia; Mandibular Hypoplasia, Deafness, Progeroid Features, and Lipodystrophy Syndrome; Sick Sinus Syndrome; Pelger-Huet Anomaly; Charcot-Marie-Tooth Disease, Axonal, Type 2e; Congenital Generalized Lipodystrophy; Restrictive Cardiomyopathy; Congenital Fiber-Type Disproportion; Lipodystrophy, Congenital Generalized, Type 1; Myofibrillar Myopathy; Lipodystrophy, Familial Partial, Type 1; Axonal Neuropathy; Atypical Werner Syndrome; Ovarian Cystadenoma; Fanconi Anemia, Complementation Group a; Body Mass Index Quantitative Trait Locus 11; Skin Disease; Rigid Spine Muscular Dystrophy 1; Neuromuscular Disease; Hallermann-Streiff Syndrome; Bethlem Myopathy 1; Acquired Generalized Lipodystrophy; Cardiomyopathy, Dilated, le; Lipodystrophy, Congenital Generalized, Type 4; Undifferentiated Pleomorphic Sarcoma; Lipodystrophy, Familial Partial, Type 3; Muscular Dystrophy, Congenital Merosin-Deficient, la; Proximal Spinal Muscular Atrophy; Muscular Dystrophy-Dystroglycanopathy, Type B, 5; Muscular Dystrophy, Congenital, 1b; Reynolds Syndrome; Wiedemann-Rautenstrauch Syndrome; Emery-Dreifuss Muscular Dystrophy 1, X-Linked; Lipodystrophy, Congenital Generalized, Type 2; Monogenic Diabetes; Cardiomyopathy, Dilated, 1d; Myopathy, Proximal, and Ophthalmoplegia; Muscle Tissue Disease; Lipodystrophy, Familial Partial, Type 4; Cardiomyopathy, Dilated, 1h; Second-Degree Atrioventricular Block; Median Neuropathy; Intrinsic Cardiomyopathy; Prolapse of Female Genital Organ; Complete Generalized Lipodystrophy; Rigid Spine Muscular Dystrophy; Emerinopathy; Ulnar Nerve Lesion; Limb-Girdle Muscular Dystrophy Type 1b; Lmna-Related Dilated Cardiomyopathy; Pelvic Muscle Wasting; Generalized Lipodystrophy-Associated Progeroid Syndrome; Muscular Disease; Cardiomyopathy, Dilated, 1b; Autosomal Genetic Disease; Familial Isolated Arrhythmogenic Ventricular Dysplasia, Right Dominant Form; Familial Isolated Arrhythmogenic Ventricular Dysplasia, Biventricular Form; Familial Isolated Arrhythmogenic Ventricular Dysplasia, Left Dominant Form; Lmna-Related Cardiocutaneous Progeria Syndrome; and Autosomal Semi-Dominant Severe Lipodystrophic Laminopathy.

46. The method according to claim 42, wherein the disease characterised by hyperlipidemia is selected from atherosclerosis, cardiovascular disease, stroke and a familial hyperlipidemia.
